SEQUENCE LISTING (457 sequences)

SEQ ID NO: 1 - protein, 310 aa, Homo sapiens
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHQASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 2 - protein, 310 aa, synthetic construct
  REGION 1..310: note = Description of Artificial Sequence: Syntheticpolypeptide
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHLASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 3 - protein, 310 aa, synthetic construct [sequence omitted]

SEQ ID NO: 4 - DNA, 933 bp, Homo sapiens
  source 1..933: mol_type = genomic DNA
atgaggcgcgcggcgctctggctctggctgtgcgcgctggcgctgagcctgcagccggccctgccgcaaattgtggctactaatttgccccctgaagatcaagatggctctggggatgactctgacaacttctccggctcaggtgcaggtgctttgcaagatatcaccttgtcacagcagaccccctccacttggaaggacacgcagctcctgacggctattcccacgtctccagaacccaccggcctggaggctacagctgcctccacctccaccctgccggctggagaggggcccaaggagggagaggctgtagtcctgccagaagtggagcctggcctcaccgcccgggagcaggaggccaccccccgacccagggagaccacacagctcccgaccactcatcaggcctcaacgaccacagccaccacggcccaggagcccgccacctcccacccccacagggacatgcagcctggccaccatgagacctcaacccctgcaggacccagccaagctgaccttcacactccccacacagaggatggaggtccttctgccaccgagagggctgctgaggatggagcctccagtcagctcccagcagcagagggctctggggagcaggacttcacctttgaaacctcgggggagaatacggctgtagtggccgtggagcctgaccgccggaaccagtccccagtggatcagggggccacgggggcctcacagggcctcctggacaggaaagaggtgctgggaggggtcattgccggaggcctcgtggggctcatctttgctgtgtgcctggtgggtttcatgctgtaccgcatgaagaagaaggacgaaggcagctactccttggaggagccgaaacaagccaacggcggggcctaccagaagcccaccaaacaggaggaattctatgcctga

SEQ ID NO: 5 - DNA, 933 bp, Homo sapiens; identical to SEQ ID NO: 4
  source 1..933: mol_type = genomic DNA

SEQ ID NO: 6 - protein, 311 aa, Mus musculus
MRRAALWLWLCALALRLQPALPQIVAVNVPPEDQDGSGDDSDNFSGSGTGALPDTLSRQTPSTWKDVWLLTATPTAPEPTSSNTETAFTSVLPAGEKPEEGEPVLHVEAEPGFTARDKEKEVTTRPRETVQLPITQRASTVRVTTAQAAVTSHPHGGMQPGLHETSAPTAPGQPDHQPPRVEGGGTSVIKEVVEDGTANQLPAGEGSGEQDFTFETSGENTAVAAVEPGLRNQPPVDEGATGASQSLLDRKEVLGGVIAGGLVGLIFAVCLVAFMLYRMKKKDEGSYSLEEPKQANGGAYQKPTKQEEFYA

SEQ ID NO: 7 - DNA, 936 bp, Mus musculus
  source 1..936: mol_type = genomic DNA
atgagacgcgcggcgctctggctctggctctgcgcgctggcgctgcgcctgcagcctgccctcccgcaaattgtggctgtaaatgttcctcctgaagatcaggatggctctggggatgactctgacaacttctctggctctggcacaggtgctttgccagatactttgtcacggcagacaccttccacttggaaggacgtgtggctgttgacagccacgcccacagctccagagcccaccagcagcaacaccgagactgcttttacctctgtcctgccagccggagagaagcccgaggagggagagcctgtgctccatgtagaagcagagcctggcttcactgctcgggacaaggaaaaggaggtcaccaccaggcccagggagaccgtgcagctccccatcacccaacgggcctcaacagtcagagtcaccacagcccaggcagctgtcacatctcatccgcacgggggcatgcaacctggcctccatgagacctcggctcccacagcacctggtcaacctgaccatcagcctccacgtgtggagggtggcggcacttctgtcatcaaagaggttgtcgaggatggaactgccaatcagcttcccgcaggagagggctctggagaacaagacttcacctttgaaacatctggggagaacacagctgtggctgccgtagagcccggcctgcggaatcagcccccggtggacgaaggagccacaggtgcttctcagagccttttggacaggaaggaagtgctgggaggtgtcattgccggaggcctagtgggcctcatctttgctgtgtgcctggtggctttcatgctgtaccggatgaagaagaaggacgaaggcagctactccttggaggagcccaaacaagccaatggcggtgcctaccagaaacccaccaagcaggaggagttctacgcctga

SEQ ID NO: 8 - protein, 39 aa, Homo sapiens
QIVATNLPPEDQDGSGDDSDNFSGSGAGALQDITLSQQT

SEQ ID NO: 9 - protein, 45 aa, Homo sapiens
ALQDITLSQQTPSTWKDTQLLTAIPTSPEPTGLEATAASTSTLPA

SEQ ID NO: 10 - protein, 34 aa, Homo sapiens
ASTSTLPAGEGPKEGEAVVLPEVEPGLTAREQEA

SEQ ID NO: 11 - protein, 15 aa, Homo sapiens
ASTSTLPAGEGPKEG

SEQ ID NO: 12 - protein, 40 aa, Homo sapiens
EPGLTAREQEATPRPRETTQLPTTHQASTTTATTAQEPAT

SEQ ID NO: 13 - protein, 35 aa, Homo sapiens
QEPATSHPHRDMQPGHHETSTPAGPSQADLHTPHT

SEQ ID NO: 14 - protein, 75 aa, Homo sapiens
HTPHTEDGGPSATERAAEDGASSQLPAAEGSGEQDFTFETSGENTAVVAVEPDRRNQSPVDQGATGASQGLLDRK

SEQ ID NO: 15 - protein, 39 aa, Homo sapiens
HTPHTEDGGPSATERAAEDGASSQLPAAEGSGEQDFTFE

SEQ ID NO: 16 - protein, 41 aa, Homo sapiens
DFTFETSGENTAVVAVEPDRRNQSPVDQGATGASQGLLDRK

SEQ ID NO: 17 - protein, 26 aa, Homo sapiens
TAVVAVEPDRRNQSPVDQGATGASQG

SEQ ID NO: 18 - protein, 4 aa, Homo sapiens
DFTF

SEQ ID NO: 19 - protein, 4 aa, Homo sapiens
FTFE

SEQ ID NO: 20 - protein, 4 aa, Homo sapiens
TFET

SEQ ID NO: 21 - protein, 4 aa, Homo sapiens
FETS

SEQ ID NO: 22 - protein, 4 aa, Homo sapiens
ETSG

SEQ ID NO: 23 - protein, 4 aa, Homo sapiens
TSGE

SEQ ID NO: 24 - protein, 4 aa, Homo sapiens
SGEN

SEQ ID NO: 25 - protein, 4 aa, Homo sapiens
GENT

SEQ ID NO: 26 - protein, 4 aa, Homo sapiens
ENTA

SEQ ID NO: 27 - protein, 4 aa, Homo sapiens
NTAV

SEQ ID NO: 28 - protein, 4 aa, Homo sapiens
TAVV

SEQ ID NO: 29 - protein, 4 aa, Homo sapiens
AVVA

SEQ ID NO: 30 - protein, 4 aa, Homo sapiens
VVAV

SEQ ID NO: 31 - protein, 4 aa, Homo sapiens
VAVE

SEQ ID NO: 32 - protein, 4 aa, Homo sapiens
AVEP

SEQ ID NO: 33 - protein, 4 aa, Homo sapiens
VEPD

SEQ ID NO: 34 - protein, 4 aa, Homo sapiens
EPDR

SEQ ID NO: 35 - protein, 4 aa, Homo sapiens
PDRR

SEQ ID NO: 36 - protein, 4 aa, Homo sapiens
DRRN

SEQ ID NO: 37 - protein, 4 aa, Homo sapiens
RRNQ

SEQ ID NO: 38 - protein, 4 aa, Homo sapiens
RNQS

SEQ ID NO: 39 - protein, 4 aa, Homo sapiens
NQSP

SEQ ID NO: 40 - protein, 4 aa, Homo sapiens
QSPV

SEQ ID NO: 41 - protein, 4 aa, Homo sapiens
SPVD

SEQ ID NO: 42 - protein, 4 aa, Homo sapiens
PVDQ

SEQ ID NO: 43 - protein, 4 aa, Homo sapiens
VDQG

SEQ ID NO: 44 - protein, 4 aa, Homo sapiens
DQGA

SEQ ID NO: 45 - protein, 4 aa, Homo sapiens
QGAT

SEQ ID NO: 46 - protein, 4 aa, Homo sapiens
GATG

SEQ ID NO: 47 - protein, 4 aa, Homo sapiens
ATGA

SEQ ID NO: 48 - protein, 4 aa, Homo sapiens
TGAS

SEQ ID NO: 49 - protein, 4 aa, Homo sapiens
GASQ

SEQ ID NO: 50 - protein, 4 aa, Homo sapiens
ASQG

SEQ ID NO: 51 - protein, 4 aa, Homo sapiens
SQGL

SEQ ID NO: 52 - protein, 4 aa, Homo sapiens
QGLL

SEQ ID NO: 53 - protein, 4 aa, Homo sapiens
GLLD

SEQ ID NO: 54 - protein, 4 aa, Homo sapiens
LLDR

SEQ ID NO: 55 - protein, 4 aa, Homo sapiens
LDRK

SEQ ID NO: 56 - protein, 5 aa, Homo sapiens
DFTFE

SEQ ID NO: 57 - protein, 5 aa, Homo sapiens
FTFET

SEQ ID NO: 58 - protein, 5 aa, Homo sapiens
TFETS

SEQ ID NO: 59 - protein, 5 aa, Homo sapiens
FETSG

SEQ ID NO: 60 - protein, 5 aa, Homo sapiens
ETSGE

SEQ ID NO: 61 - protein, 5 aa, Homo sapiens
TSGEN

SEQ ID NO: 62 - protein, 5 aa, Homo sapiens
SGENT

SEQ ID NO: 63 - protein, 5 aa, Homo sapiens
GENTA

SEQ ID NO: 64 - protein, 5 aa, Homo sapiens
ENTAV

SEQ ID NO: 65 - protein, 5 aa, Homo sapiens
NTAVV

SEQ ID NO: 66 - protein, 5 aa, Homo sapiens
TAVVA

SEQ ID NO: 67 - protein, 5 aa, Homo sapiens
AVVAV

SEQ ID NO: 68 - protein, 5 aa, Homo sapiens
VVAVE

SEQ ID NO: 69 - protein, 5 aa, Homo sapiens
VAVEP

SEQ ID NO: 70 - protein, 5 aa, Homo sapiens
AVEPD

SEQ ID NO: 71 - protein, 5 aa, Homo sapiens
VEPDR

SEQ ID NO: 72 - protein, 5 aa, Homo sapiens
EPDRR

SEQ ID NO: 73 - protein, 5 aa, Homo sapiens
PDRRN

SEQ ID NO: 74 - protein, 5 aa, Homo sapiens
DRRNQ

SEQ ID NO: 75 - protein, 5 aa, Homo sapiens
RRNQS

SEQ ID NO: 76 - protein, 5 aa, Homo sapiens
RNQSP

SEQ ID NO: 77 - protein, 5 aa, Homo sapiens
NQSPV

SEQ ID NO: 78 - protein, 5 aa, Homo sapiens
QSPVD

SEQ ID NO: 79 - protein, 5 aa, Homo sapiens
SPVDQ

SEQ ID NO: 80 - protein, 5 aa, Homo sapiens
PVDQG

SEQ ID NO: 81 - protein, 5 aa, Homo sapiens
VDQGA

SEQ ID NO: 82 - protein, 5 aa, Homo sapiens
DQGAT

SEQ ID NO: 83 - protein, 5 aa, Homo sapiens
QGATG

SEQ ID NO: 84 - protein, 5 aa, Homo sapiens
GATGA

SEQ ID NO: 85 - protein, 5 aa, Homo sapiens
ATGAS

SEQ ID NO: 86 - protein, 5 aa, Homo sapiens
TGASQ

SEQ ID NO: 87 - protein, 5 aa, Homo sapiens
GASQG

SEQ ID NO: 88 - protein, 5 aa, Homo sapiens
ASQGL

SEQ ID NO: 89 - protein, 5 aa, Homo sapiens
SQGLL

SEQ ID NO: 90 - protein, 5 aa, Homo sapiens
QGLLD

SEQ ID NO: 91 - protein, 5 aa, Homo sapiens
GLLDR

SEQ ID NO: 92 - protein, 5 aa, Homo sapiens
LLDRK

SEQ ID NO: 93 - protein, 6 aa, Homo sapiens
DFTFET

SEQ ID NO: 94 - protein, 6 aa, Homo sapiens
FTFETS

SEQ ID NO: 95 - protein, 6 aa, Homo sapiens
TFETSG

SEQ ID NO: 96 - protein, 6 aa, Homo sapiens
FETSGE

SEQ ID NO: 97 - protein, 6 aa, Homo sapiens
ETSGEN

SEQ ID NO: 98 - protein, 6 aa, Homo sapiens
TSGENT

SEQ ID NO: 99 - protein, 6 aa, Homo sapiens
SGENTA

SEQ ID NO: 100 - protein, 6 aa, Homo sapiens
GENTAV

SEQ ID NO: 101 - protein, 6 aa, Homo sapiens
ENTAVV

SEQ ID NO: 102 - protein, 6 aa, Homo sapiens
NTAVVA

SEQ ID NO: 103 - protein, 6 aa, Homo sapiens
TAVVAV

SEQ ID NO: 104 - protein, 6 aa, Homo sapiens
AVVAVE

SEQ ID NO: 105 - protein, 6 aa, Homo sapiens
VVAVEP

SEQ ID NO: 106 - protein, 6 aa, Homo sapiens
VAVEPD

SEQ ID NO: 107 - protein, 6 aa, Homo sapiens
AVEPDR

SEQ ID NO: 108 - protein, 6 aa, Homo sapiens
VEPDRR

SEQ ID NO: 109 - protein, 6 aa, Homo sapiens
EPDRRN

SEQ ID NO: 110 - protein, 6 aa, Homo sapiens
PDRRNQ

SEQ ID NO: 111 - protein, 6 aa, Homo sapiens
DRRNQS

SEQ ID NO: 112 - protein, 6 aa, Homo sapiens
RRNQSP

SEQ ID NO: 113 - protein, 6 aa, Homo sapiens
RNQSPV

SEQ ID NO: 114 - protein, 6 aa, Homo sapiens
NQSPVD

SEQ ID NO: 115 - protein, 6 aa, Homo sapiens
QSPVDQ

SEQ ID NO: 116 - protein, 6 aa, Homo sapiens
SPVDQG

SEQ ID NO: 117 - protein, 6 aa, Homo sapiens
PVDQGA

SEQ ID NO: 118 - protein, 6 aa, Homo sapiens
VDQGAT

SEQ ID NO: 119 - protein, 6 aa, Homo sapiens
DQGATG

SEQ ID NO: 120 - protein, 6 aa, Homo sapiens
QGATGA

SEQ ID NO: 121 - protein, 6 aa, Homo sapiens
GATGAS

SEQ ID NO: 122 - protein, 6 aa, Homo sapiens
ATGASQ

SEQ ID NO: 123 - protein, 6 aa, Homo sapiens
TGASQG

SEQ ID NO: 124 - protein, 6 aa, Homo sapiens
GASQGL

SEQ ID NO: 125 - protein, 6 aa, Homo sapiens
ASQGLL

SEQ ID NO: 126 - protein, 6 aa, Homo sapiens
SQGLLD

SEQ ID NO: 127 - protein, 6 aa, Homo sapiens
QGLLDR

SEQ ID NO: 128 - protein, 6 aa, Homo sapiens
GLLDRK

SEQ ID NO: 129 - protein, 4 aa, Homo sapiens
HTPH

SEQ ID NO: 130 - protein, 4 aa, Homo sapiens
TPHT

SEQ ID NO: 131 - protein, 4 aa, Homo sapiens
PHTE

SEQ ID NO: 132 - protein, 4 aa, Homo sapiens
HTED

SEQ ID NO: 133 - protein, 4 aa, Homo sapiens
TEDG

SEQ ID NO: 134 - protein, 4 aa, Homo sapiens
EDGG

SEQ ID NO: 135 - protein, 4 aa, Homo sapiens
DGGP

SEQ ID NO: 136 - protein, 4 aa, Homo sapiens
GGPS

SEQ ID NO: 137 - protein, 4 aa, Homo sapiens
GPSA

SEQ ID NO: 138 - protein, 4 aa, Homo sapiens
PSAT

SEQ ID NO: 139 - protein, 4 aa, Homo sapiens
SATE

SEQ ID NO: 140 - protein, 4 aa, Homo sapiens
ATER

SEQ ID NO: 141 - protein, 4 aa, Homo sapiens
TERA

SEQ ID NO: 142 - protein, 4 aa, Homo sapiens
ERAA

SEQ ID NO: 143 - protein, 4 aa, Homo sapiens
RAAE

SEQ ID NO: 144 - protein, 4 aa, Homo sapiens
AAED

SEQ ID NO: 145 - protein, 4 aa, Homo sapiens
AEDG

SEQ ID NO: 146 - protein, 4 aa, Homo sapiens
EDGA

SEQ ID NO: 147 - protein, 4 aa, Homo sapiens
DGAS

SEQ ID NO: 148 - protein, 4 aa, Homo sapiens
GASS

SEQ ID NO: 149 - protein, 4 aa, Homo sapiens
ASSQ

SEQ ID NO: 150 - protein, 4 aa, Homo sapiens
SSQL

SEQ ID NO: 151 - protein, 4 aa, Homo sapiens
SQLP

SEQ ID NO: 152 - protein, 4 aa, Homo sapiens
QLPA

SEQ ID NO: 153 - protein, 4 aa, Homo sapiens
LPAA

SEQ ID NO: 154 - protein, 4 aa, Homo sapiens
PAAE

SEQ ID NO: 155 - protein, 4 aa, Homo sapiens
AAEG

SEQ ID NO: 156 - protein, 4 aa, Homo sapiens
AEGS

SEQ ID NO: 157 - protein, 4 aa, Homo sapiens
EGSG

SEQ ID NO: 158 - protein, 4 aa, Homo sapiens
GSGE

SEQ ID NO: 159 - protein, 4 aa, Homo sapiens
SGEQ

SEQ ID NO: 160 - protein, 4 aa, Homo sapiens
GEQD

SEQ ID NO: 161 - protein, 4 aa, Homo sapiens
EQDF

SEQ ID NO: 162 - protein, 4 aa, Homo sapiens
QDFT

SEQ ID NO: 163 - protein, 5 aa, Homo sapiens
HTPHT

SEQ ID NO: 164 - protein, 5 aa, Homo sapiens
TPHTE

SEQ ID NO: 165 - protein, 5 aa, Homo sapiens
PHTED

SEQ ID NO: 166 - protein, 5 aa, Homo sapiens
HTEDG

SEQ ID NO: 167 - protein, 5 aa, Homo sapiens
TEDGG

SEQ ID NO: 168 - protein, 5 aa, Homo sapiens
EDGGP

SEQ ID NO: 169 - protein, 5 aa, Homo sapiens
DGGPS

SEQ ID NO: 170 - protein, 5 aa, Homo sapiens
GGPSA

SEQ ID NO: 171 - protein, 5 aa, Homo sapiens
GPSAT

SEQ ID NO: 172 - protein, 5 aa, Homo sapiens
PSATE

SEQ ID NO: 173 - protein, 5 aa, Homo sapiens
SATER

SEQ ID NO: 174 - protein, 5 aa, Homo sapiens
ATERA

SEQ ID NO: 175 - protein, 5 aa, Homo sapiens
TERAA

SEQ ID NO: 176 - protein, 5 aa, Homo sapiens
ERAAE

SEQ ID NO: 177 - protein, 5 aa, Homo sapiens
RAAED

SEQ ID NO: 178 - protein, 5 aa, Homo sapiens
AAEDG

SEQ ID NO: 179 - protein, 5 aa, Homo sapiens
AEDGA

SEQ ID NO: 180 - protein, 5 aa, Homo sapiens
EDGAS

SEQ ID NO: 181 - protein, 5 aa, Homo sapiens
DGASS

SEQ ID NO: 182 - protein, 5 aa, Homo sapiens
GASSQ

SEQ ID NO: 183 - protein, 5 aa, Homo sapiens
ASSQL

SEQ ID NO: 184 - protein, 5 aa, Homo sapiens
SSQLP

SEQ ID NO: 185 - protein, 5 aa, Homo sapiens
SQLPA

SEQ ID NO: 186 - protein, 5 aa, Homo sapiens
QLPAA

SEQ ID NO: 187 - protein, 5 aa, Homo sapiens
LPAAE

SEQ ID NO: 188 - protein, 5 aa, Homo sapiens
PAAEG

SEQ ID NO: 189 - protein, 5 aa, Homo sapiens
AAEGS

SEQ ID NO: 190 - protein, 5 aa, Homo sapiens
AEGSG

SEQ ID NO: 191 - protein, 5 aa, Homo sapiens
EGSGE

SEQ ID NO: 192 - protein, 5 aa, Homo sapiens
GSGEQ

SEQ ID NO: 193 - protein, 5 aa, Homo sapiens
SGEQD

SEQ ID NO: 194 - protein, 5 aa, Homo sapiens
GEQDF

SEQ ID NO: 195 - protein, 5 aa, Homo sapiens
EQDFT

SEQ ID NO: 196 - protein, 5 aa, Homo sapiens
QDFTF

SEQ ID NO: 197 - protein, 6 aa, Homo sapiens
HTPHTE

SEQ ID NO: 198 - protein, 6 aa, Homo sapiens
TPHTED

SEQ ID NO: 199 - protein, 6 aa, Homo sapiens
PHTEDG

SEQ ID NO: 200 - protein, 6 aa, Homo sapiens
HTEDGG

SEQ ID NO: 201 - protein, 6 aa, Homo sapiens
TEDGGP

SEQ ID NO: 202 - protein, 6 aa, Homo sapiens
EDGGPS

SEQ ID NO: 203 - protein, 6 aa, Homo sapiens
DGGPSA

SEQ ID NO: 204 - protein, 6 aa, Homo sapiens
GGPSAT

SEQ ID NO: 205 - protein, 6 aa, Homo sapiens
GPSATE

SEQ ID NO: 206 - protein, 6 aa, Homo sapiens
PSATER

SEQ ID NO: 207 - protein, 6 aa, Homo sapiens
SATERA

SEQ ID NO: 208 - protein, 6 aa, Homo sapiens
ATERAA

SEQ ID NO: 209 - protein, 6 aa, Homo sapiens
TERAAE

SEQ ID NO: 210 - protein, 6 aa, Homo sapiens
ERAAED

SEQ ID NO: 211 - protein, 6 aa, Homo sapiens
RAAEDG

SEQ ID NO: 212 - protein, 6 aa, Homo sapiens
AAEDGA

SEQ ID NO: 213 - protein, 6 aa, Homo sapiens
AEDGAS

SEQ ID NO: 214 - protein, 6 aa, Homo sapiens
EDGASS

SEQ ID NO: 215 - protein, 6 aa, Homo sapiens
DGASSQ

SEQ ID NO: 216 - protein, 6 aa, Homo sapiens
GASSQL

SEQ ID NO: 217 - protein, 6 aa, Homo sapiens
ASSQLP

SEQ ID NO: 218 - protein, 6 aa, Homo sapiens
SSQLPA

SEQ ID NO: 219 - protein, 6 aa, Homo sapiens
SQLPAA

SEQ ID NO: 220 - protein, 6 aa, Homo sapiens
QLPAAE

SEQ ID NO: 221 - protein, 6 aa, Homo sapiens
LPAAEG

SEQ ID NO: 222 - protein, 6 aa, Homo sapiens
PAAEGS

SEQ ID NO: 223 - protein, 6 aa, Homo sapiens
AAEGSG

SEQ ID NO: 224 - protein, 6 aa, Homo sapiens
AEGSGE

SEQ ID NO: 225 - protein, 6 aa, Homo sapiens
EGSGEQ

SEQ ID NO: 226 - protein, 6 aa, Homo sapiens
GSGEQD

SEQ ID NO: 227 - protein, 6 aa, Homo sapiens
SGEQDF

SEQ ID NO: 228 - protein, 6 aa, Homo sapiens
GEQDFT

SEQ ID NO: 229 - protein, 6 aa, Homo sapiens
EQDFTF

SEQ ID NO: 230 - protein, 6 aa, Homo sapiens
QDFTFE

SEQ ID NO: 231 - protein, 4 aa, Homo sapiens
ASTS

SEQ ID NO: 232 - protein, 4 aa, Homo sapiens
STST

SEQ ID NO: 233 - protein, 4 aa, Homo sapiens
TSTL

SEQ ID NO: 234 - protein, 4 aa, Homo sapiens
STLP

SEQ ID NO: 235 - protein, 4 aa, Homo sapiens
TLPA

SEQ ID NO: 236 - protein, 4 aa, Homo sapiens
LPAG

SEQ ID NO: 237 - protein, 4 aa, Homo sapiens
PAGE

SEQ ID NO: 238 - protein, 4 aa, Homo sapiens
AGEG

SEQ ID NO: 239 - protein, 4 aa, Homo sapiens
GEGP

SEQ ID NO: 240 - protein, 4 aa, Homo sapiens
EGPK

SEQ ID NO: 241 - protein, 4 aa, Homo sapiens
GPKE

SEQ ID NO: 242 - protein, 4 aa, Homo sapiens
PKEG

SEQ ID NO: 243 - protein, 4 aa, Homo sapiens
KEGE

SEQ ID NO: 244 - protein, 4 aa, Homo sapiens
EGEA

SEQ ID NO: 245 - protein, 4 aa, Homo sapiens
GEAV

SEQ ID NO: 246 - protein, 4 aa, Homo sapiens
EAVV

SEQ ID NO: 247 - protein, 4 aa, Homo sapiens
AVVL

SEQ ID NO: 248 - protein, 4 aa, Homo sapiens
VVLP

SEQ ID NO: 249 - protein, 4 aa, Homo sapiens
VLPE

SEQ ID NO: 250 - protein, 4 aa, Homo sapiens
LPEV

SEQ ID NO: 251 - protein, 4 aa, Homo sapiens
PEVE

SEQ ID NO: 252 - protein, 4 aa, Homo sapiens
EVEP

SEQ ID NO: 253 - protein, 4 aa, Homo sapiens
VEPG

SEQ ID NO: 254 - protein, 4 aa, Homo sapiens
EPGL

SEQ ID NO: 255 - protein, 4 aa, Homo sapiens
PGLT

SEQ ID NO: 256 - protein, 4 aa, Homo sapiens
GLTA

SEQ ID NO: 257 - protein, 4 aa, Homo sapiens
LTAR

SEQ ID NO: 258 - protein, 4 aa, Homo sapiens
TARE

SEQ ID NO: 259 - protein, 4 aa, Homo sapiens
AREQ

SEQ ID NO: 260 - protein, 4 aa, Homo sapiens
REQE

SEQ ID NO: 261 - protein, 4 aa, Homo sapiens
EQEA

SEQ ID NO: 262 - protein, 114 aa, synthetic construct
  REGION 1..114: note = Description of Artificial Sequence: Syntheticpolypeptide
EVQLQQSGPELVKPGASVKISCETSGFSFTAHHMHWVKQSPEKSLEWIGEIDPNTGSTTYNQKFRAKATLTVDKSSNTTYMQLKSLTFEDSAVYYCYSNWFPYWGQGTLVTVSA

SEQ ID NO: 263 - protein, 112 aa, synthetic construct
  REGION 1..112: note = Description of Artificial Sequence: Syntheticpolypeptide
DVVMTQTPLTLSATIGQPASIYCKSSQSLLDGDGKTYLNWLLQRPGQSPKRLIYLVSKLDSGVPDRFTGSGSGTDFTLKISRVEAEDLGVYYCWQGTHFPRTFGGGTKLEIK

SEQ ID NO: 264 - protein, 115 aa, synthetic construct
  REGION 1..115: note = Description of Artificial Sequence: Syntheticpolypeptide
QVQLQQPGAELVKPGASVKLSCKASGFSFITYWMNWIKQRPGRGLEWIGRIHPSDSATQYNQKFKTKATLTVDKSSSTAYIQLSSLTSEDSAVYYCARSTEGAHWGQGTLVTVSA

SEQ ID NO: 265 - protein, 112 aa, synthetic construct
  REGION 1..112: note = Description of Artificial Sequence: Syntheticpolypeptide
DVVMTQTPLTLSVTIGQPASISCKSSQSLLHSDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 266 - protein, 117 aa, synthetic construct
  REGION 1..117: note = Description of Artificial Sequence: Syntheticpolypeptide
QVQLQQPGAELVKPGASVKLSCKASGYTFTSFWMHWVKQRPGQGLEWIGEIYPSSGVTNYNERFKNKATLTVDKSSRTAYMQLSSLTSEDSAVYFCTPNYYYDGLYWGQGTLVTVSA

SEQ ID NO: 267 - protein, 112 aa, synthetic construct
  REGION 1..112: note = Description of Artificial Sequence: Syntheticpolypeptide
DVVMTQTPLTLSVTIGQPASISCKSSHSLLYTNGETYLNWLLQRPGQSPKRLIYLVSNLDSGVPDRFSGSGSGTDFTLKISRVEAEDLGIYYCLQSTHFPRTFGGGTKLEIK

SEQ ID NO: 268 - protein, 115 aa, synthetic construct
  REGION 1..115: note = Description of Artificial Sequence: Syntheticpolypeptide
QVQLQQPGAELVKPGASVKLSCKASGFSFTRYWMNWVKQRPGRGLEWIGRIHPSDSASQYNQKFKSKATLTVDKSSSTAYIQLSSLTSEDSAVYYCGRSTEGAYWGQGTLVTVSA

SEQ ID NO: 269 - protein, 115 aa, synthetic construct
  REGION 1..115: note = Description of Artificial Sequence: Syntheticpolypeptide
QVQLQQPGAELVKPGASVKLSCKASGFSFITYWMNWIKQRPGRGLEWIGRIHPSDSATQYDQKFKTKATLTVDKSSSTAYIQLSSLTSEDSAVYYCARSTEGAHWGPGTLVTVSA

SEQ ID NO: 270 - protein, 119 aa, synthetic construct
  REGION 1..119: note = Description of Artificial Sequence: Syntheticpolypeptide
EIQLQQSGTELVKPGASVKISCKTSGYSFTDYNMNWVKQSHGKSLEWIGNINPYYGSTGYTQNFEGKATLTVDKSSSTAYMQLNSLTSEDSALYYCAREGHDYYAMDYWGQGTSVTVSA

SEQ ID NO: 271 - protein, 113 aa, synthetic construct
  REGION 1..113: note = Description of Artificial Sequence: Syntheticpolypeptide
QVQLQLPGAELVKPGASVKVSCKASGYTFTSYWMHWVKQRPGQGLEWIGRIHPSDSDTNYNQNFKGKATLIVDKSSSTAYMQLSSLTSEDSAVYYCATGFSFWGQGTLVTVSA

SEQ ID NO: 272 - protein, 113 aa, synthetic construct
  REGION 1..113: note = Description of Artificial Sequence: Syntheticpolypeptide
QVQVQVPGAELVKPGASVKVSCKASGYTFTSYWMHWMKKRPGQGLEWIGRIHPSDSDTNYNQNFKGKATLTVDKSSSTAYMQLSSLTSEDSAVYFCATGFSFWGQGTLVTVSA

SEQ ID NO: 273 - protein, 112 aa, synthetic construct
  REGION 1..112: note = Description of Artificial Sequence: Syntheticpolypeptide
DVVMTQTPLTLSVTIGQPASISCKSSQSLLYSDGKTYLNWLLQRPGESPKLLIYLVSKLDSGVPDRFTGSGSGTDFTLKISRVEAEDLGVYYCLQTTSFPYTFGGGTKLDIK

SEQ ID NO: 274 - protein, 115 aa, synthetic construct
  REGION 1..115: note = Description of Artificial Sequence: Syntheticpolypeptide
QVQLQQPGAELVKPGASVKVSCKASGYNFINYWMHWVKQRPGQGLEWIGRIHPSDSYTNYNQKFKGKATLTVDKSSSTAYMQLSSLTSEDSAVYYCASPISTLYWGQGTTLTVSS

SEQ ID NO: 275 - protein, 112 aa, synthetic construct
  REGION 1..112: note = Description of Artificial Sequence: Syntheticpolypeptide
DVVMTQTPLTLSVTIGQPASISCKSSQSLLDSDGKTYLNWLLQRPGESPKLLIYLVSKLDSGVPDRFTGSGSGTDFTLKISRVEAEDLGVYYCLQATHFPQTFGGGTKLEIK

SEQ ID NO: 276 - protein, 113 aa, synthetic construct
  REGION 1..113: note = Description of Artificial Sequence: Syntheticpolypeptide
QVQLQLPGAELVRPGTSVKVSCKASDYTFTTYWMHWVKQRPGQGLDWIGRIHPSDSDTNYNQNFKGKATLTVDKSSSTAYMHLSSLTSEDSAVYYCATGFSFWGQGTLVTVSA

SEQ ID NO: 277 - protein, 113 aa, synthetic construct
  REGION 1..113: note = Description of Artificial Sequence: Syntheticpolypeptide
QVQVQLPGAELVKPGASVKVSCKASGYTFTSYWMHWVKKRPGQGLEWIGRIHPSDSDTNYNQNFKGKATLTVDKSSSTAYMLLSSLTSEDSAVYYCATGFSFWGQGTLITVSA

SEQ ID NO: 278 - protein, 112 aa, synthetic construct
  REGION 1..112: note = Description of Artificial Sequence: Syntheticpolypeptide
DVVMTQTPLTLSVTIGQPASISCKSSQSLLYSDGKTYLNWLLQRPGESPELLIYLVSKMDSGVPDRFHGHGSGTAFTMKISRMGGGGLGNYYCLPRTSFPYTFGGGTKLEIK

SEQ ID NO: 279 - protein, 113 aa, synthetic construct
  REGION 1..113: note = Description of Artificial Sequence: Syntheticpolypeptide
QVQLQLPGAELVKPGASVKVSCKASGYTFTSYWMHWVKQRPGQGLEWIGRIHPSDSDTNYNQNFKGKATLTVDKSSNTAYMQLSSLTSEDSAVYYCATGFSFWGQGTLVTVSA

SEQ ID NO: 280 - protein, 112 aa, synthetic construct
  REGION 1..112: note = Description of Artificial Sequence: Syntheticpolypeptide
DVVMTPTSLHLLVTIGQPGFLFCKSSQNLLYNEGKTYLKWLLPEPGAFSKVLIYLVFKMGFGVPDRFHGHGSGTDFPMKISRMGGGGLGGYLCLPSTPFPYTFGGGTKLEIK

SEQ ID NO: 281 - protein, 122 aa, synthetic construct
  REGION 1..122: note = Description of Artificial Sequence: Syntheticpolypeptide
QIHLVQSGPELKKPGETVRISCKASGYTFTTYGMSWVKQAPGKALKWMGWINTYSGVPTYADDFKGRFAFSLETSASTAYLQINNLKNEDTATYFCTREGSTMVTRYYFDYWGQGTTLTVSS

SEQ ID NO: 282 - protein, 112 aa, synthetic construct
  REGION 1..112: note = Description of Artificial Sequence: Syntheticpolypeptide
DIVMTQAAPSVPVTPGESVSISCRSSKSLLHSNGNTYLYWFLQRPGQSPQLLIYRMSNLASGVPDRFSGSGSGTAFTLRISRVEAEDVGVYHCMQHLESPYTFGGGTTLEIK

SEQ ID NO: 283 - protein, 113 aa, synthetic construct
  REGION 1..113: note = Description of Artificial Sequence: Syntheticpolypeptide
QVQLQLPGAELVKPGASVKVSCKASAYTFTSYWMHWVKQRPGQGLEWIGRIHPSDSDTNYNQNFKGKATLTVDKSSNTAYMQLSSLTSEDSAVYYCATGFSFWGQGTLVTVSA

SEQ ID NO: 284 - protein, 107 aa, synthetic construct
  REGION 1..107: note = Description of Artificial Sequence: Syntheticpolypeptide
DIVMTQSHKFMSTSVGDRVSITCKASQDVSTTVAWYQQKPGQSPKLLIYSASYRYTGVPDRFTGSGSGTDFTFTISSVQAEDLAVYYCQQHYSTRPTFGGGTKLEIK

SEQ ID NO: 285 - protein, 122 aa, synthetic construct
  REGION 1..122: note = Description of Artificial Sequence: Syntheticpolypeptide
QIQLVQSGPELKKPGETVKISCKASGYTFTTYGMSWVKQAPGKGLKWMGWINTYSGVPTYADDFKGRFAFSLETSASTAYLQINNLKNEDTATYFCAREGSTMVTRYYFDYWGQGTTLTVSS

SEQ ID NO: 286 - protein, 112 aa, synthetic construct
  REGION 1..112: note = Description of Artificial Sequence: Syntheticpolypeptide
DIVMTQAAPSVPVTPGESVSISCRSSKSLLHSNGNTYLYWFLQRPGQSPQVLIYRMSNLASGVPDRFSGSGSGTAFTLRISRVEAEDVGVYYCMQHLESPYTFGGGTKLEIK

SEQ ID NO: 287 - protein, 122 aa, synthetic construct
  REGION 1..122: note = Description of Artificial Sequence: Syntheticpolypeptide
QIQLVQSGPELKKPGETVKISCKASGYTFTTYGMSWVKQAPGKGLKWMGWINTYSGVPTYADDFKGRFAFSLETSASTAYLQINNLKNEDTATFFCAREGSTMVTRYYFDYWGQGTTLTVSS

SEQ ID NO: 288 - protein, 112 aa, synthetic construct
  REGION 1..112: note = Description of Artificial Sequence: Syntheticpolypeptide
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPSTFGGGTKLEIK

SEQ ID NO: 289 - protein, 113 aa, synthetic construct
  REGION 1..113: note = Description of Artificial Sequence: Syntheticpolypeptide
QVQVQLPGAELVKPGASVKVSCKASGYTFTSYWMHWVKKRPGQGLEWIGRIHPSDSDTNYNQNFKGKATLTVDKSSSTAYMQLTSLTSEDFAVYYCSTGFSFWGQGTLVTVSA

SEQ ID NO: 290 - protein, 112 aa, synthetic construct
  REGION 1..112: note = Description of Artificial Sequence: Syntheticpolypeptide
DVVMTQTPLTLSVTIGQPASISCKSSQSLLYSDGKTYLNWLLQRPGESPKLLIYLVSKLDSGVPDRFTGSGSGTDFTLKISRVEAEDLGVYYCLQTTYFPYTFGGGTKLEIK

SEQ ID NO: 291 - protein, 112 aa, synthetic construct
  REGION 1..112: note = Description of Artificial Sequence: Syntheticpolypeptide
QVQLHQPGTSLVKPGASVKLSCKASGYNFSSYYMHWVKQRPGQGLEWIGTIHPSDSTTNCNQKFKGKATLTVDKSSRTAYMQLNSLTFEDSAVYYCANFVYWGQGTSVTVSS

SEQ ID NO: 292 - protein, 112 aa, synthetic construct
  REGION 1..112: note = Description of Artificial Sequence: Syntheticpolypeptide
DIVITQDELSNPVTSGDSVSISCRSSKSLLYKDGKTYLNWFLQRPGQSPQLLIYVVSTRASGVSDRFSGSGSGTDFTLEISRVKAEDVGVYYCQQLVEYPYTFGGGTKLEIK

SEQ ID NO: 293 - protein, 112 aa, synthetic construct
  REGION 1..112: note = Description of Artificial Sequence: Syntheticpolypeptide
QVQLHQPGTSLVKPGASVKLSCKASGYNFSSYYMHWVKQRPGQGLEWIGTIHPSDSTTNYNQKFKGKATLTVDKSSRTAYMQLNSLTFEDSAVYYCANFVYWGQGTSVTVSS

SEQ ID NO: 294 - protein, 112 aa, synthetic construct
  REGION 1..112: note = Description of Artificial Sequence: Syntheticpolypeptide
QVQLHQPGTSLVKPGASVKLSCKASGYSFSSYYMHWVKQRPGQGLEWIGTIHPSDSTTNCNQKFKGKATLTVDKSSRTAYMQLNSLTFEDSAVYYCANFVYWGQGTSVTVSS

SEQ ID NO: 295 - protein, 112 aa, synthetic construct
  REGION 1..112: note = Description of Artificial Sequence: Syntheticpolypeptide
QVQLHQPGTSLVKPGASVKLSCKASGYTFSSYYMHWVKQRPGQGLEWIGTIHPSDSTTNCNQKFKGKATLTVDKSSRTAYMQLNSLTFEDSAVYYCANFVYWGQGTSVTVSS

SEQ ID NO: 296 - protein, 112 aa, synthetic construct
  REGION 1..112: note = Description of Artificial Sequence: Syntheticpolypeptide
QVQLHQPGTSLVKPGASVKLSCKASGYSFSSYYMHWVKQRPGQGLEWIGTIHPSDSTTNYNQKFKGKATLTVDKSSRTAYMQLNSLTFEDSAVYYCANFVYWGQGTSVTVSS

SEQ ID NO: 297 - protein, 112 aa, synthetic construct
  REGION 1..112: note = Description of Artificial Sequence: Syntheticpolypeptide
QVQLHQPGTSLVKPGASVKLSCKASGYTFSSYYMHWVKQRPGQGLEWIGTIHPSDSTTNYNQKFKGKATLTVDKSSRTAYMQLNSLTFEDSAVYYCANFVYWGQGTSVTVSS

SEQ ID NO: 298 - protein, 121 aa, synthetic construct
  REGION 1..121: note = Description of Artificial Sequence: Syntheticpolypeptide
EVQLVESGGGLVQPKGSLKLSCAASGFTFNTYAMHWVRQAPGKGLEWVARIRSKSSNYATYYADSVKDRFTISRDDSQSMLYLQMNNLKTEDTAMYYCVRELRLRYAMDYWGQGTSVTVSS

SEQ ID NO: 299 - protein, 112 aa, synthetic construct
  REGION 1..112: note = Description of Artificial Sequence: Syntheticpolypeptide
DILMTQTPLTLSVTIGQPASISCKSSQSLLYTNGKTYLNWLLQRPGQSPKRLIYLVSKLDSGVPDRFSGSGSGTDFTLKISRVEAEDLGVYYCLQSTHFPLTFGAGTKLELK

SEQ ID NO: 300 - protein, 7 aa, synthetic construct
  REGION 1..7: note = Description of Artificial Sequence: Syntheticpeptide
GFSFTAH

SEQ ID NO: 301 - protein, 6 aa, synthetic construct
  REGION 1..6: note = Description of Artificial Sequence: Syntheticpeptide
DPNTGS

SEQ ID NO: 302 - protein, 5 aa, synthetic construct
  REGION 1..5: note = Description of Artificial Sequence: Syntheticpeptide
NWFPY

SEQ ID NO: 303 - protein, 16 aa, synthetic construct
  REGION 1..16: note = Description of Artificial Sequence: Syntheticpeptide
KSSQSLLDGDGKTYLN

SEQ ID NO: 304 - protein, 7 aa, synthetic construct
  REGION 1..7: note = Description of Artificial Sequence: Syntheticpeptide
LVSKLDS

SEQ ID NO: 305 - protein, 9 aa, synthetic construct
  REGION 1..9: note = Description of Artificial Sequence: Syntheticpeptide
WQGTHFPRT

SEQ ID NO: 306 - protein, 7 aa, synthetic construct
  REGION 1..7: note = Description of Artificial Sequence: Syntheticpeptide
GFSFITY

SEQ ID NO: 307 - protein, 6 aa, synthetic construct
  REGION 1..6: note = Description of Artificial Sequence: Syntheticpeptide
HPSDSA

SEQ ID NO: 308 - protein, 6 aa, synthetic construct
  REGION 1..6: note = Description of Artificial Sequence: Syntheticpeptide
STEGAH

SEQ ID NO: 309 - protein, 16 aa, synthetic construct
  REGION 1..16: note = Description of Artificial Sequence: Syntheticpeptide
KSSQSLLHSDGKTYLN

SEQ ID NO: 310 - protein, 9 aa, synthetic construct
  REGION 1..9: note = Description of Artificial Sequence: Syntheticpeptide
WQGTHFPQT

SEQ ID NO: 311 - protein, 7 aa, synthetic construct
  REGION 1..7: note = Description of Artificial Sequence: Syntheticpeptide
GYTFTSF

SEQ ID NO: 312 - protein, 6 aa, synthetic construct
  REGION 1..6: note = Description of Artificial Sequence: Syntheticpeptide
YPSSGV

SEQ ID NO: 313 - protein, 8 aa, synthetic construct
  REGION 1..8: note = Description of Artificial Sequence: Syntheticpeptide
NYYYDGLY

SEQ ID NO: 314 - protein, 16 aa, synthetic construct
  REGION 1..16: note = Description of Artificial Sequence: Syntheticpeptide
KSSHSLLYTNGETYLN

SEQ ID NO: 315 - protein, 7 aa, synthetic construct
  REGION 1..7: note = Description of Artificial Sequence: Syntheticpeptide
LVSNLDS

SEQ ID NO: 316 - protein, 9 aa, synthetic construct
  REGION 1..9: note = Description of Artificial Sequence: Syntheticpeptide
LQSTHFPRT

SEQ ID NO: 317 - protein, 7 aa, synthetic construct
  REGION 1..7: note = Description of Artificial Sequence: Syntheticpeptide
GFSFTRY

SEQ ID NO: 318 - protein, 6 aa, synthetic construct
  REGION 1..6: note = Description of Artificial Sequence: Syntheticpeptide
STEGAY

SEQ ID NO: 319 - protein, 7 aa, synthetic construct
  REGION 1..7: note = Description of Artificial Sequence: Syntheticpeptide
GYSFTDY

SEQ ID NO: 320 - protein, 6 aa, synthetic construct
  REGION 1..6: note = Description of Artificial Sequence: Syntheticpeptide
NPYYGS

SEQ ID NO: 321 - protein, 10 aa, synthetic construct
  REGION 1..10: note = Description of Artificial Sequence: Syntheticpeptide
EGHDYYAMDY

SEQ ID NO: 322 - protein, 7 aa, synthetic construct
  REGION 1..7: note = Description of Artificial Sequence: Syntheticpeptide
GYTFTSY

SEQ ID NO: 323 - protein, 6 aa, synthetic construct
  REGION 1..6: note = Description of Artificial Sequence: Syntheticpeptide
HPSDSD

SEQ ID NO: 324 - protein, 4 aa, synthetic construct
  REGION 1..4: note = Description of Artificial Sequence: Syntheticpeptide
GFSF

SEQ ID NO: 325 - protein, 16 aa, synthetic construct
  REGION 1..16: note = Description of Artificial Sequence: Syntheticpeptide
KSSQSLLYSDGKTYLN

SEQ ID NO: 326 - protein, 9 aa, synthetic construct
  REGION 1..9: note = Description of Artificial Sequence: Syntheticpeptide
LQTTSFPYT

SEQ ID NO: 327 - protein, 7 aa, synthetic construct
  REGION 1..7: note = Description of Artificial Sequence: Syntheticpeptide
GYNFINY

SEQ ID NO: 328 - protein, 6 aa, synthetic construct
  REGION 1..6: note = Description of Artificial Sequence: Syntheticpeptide
HPSDSY

SEQ ID NO: 329 - protein, 6 aa, synthetic construct
  REGION 1..6: note = Description of Artificial Sequence: Syntheticpeptide
PISTLY

SEQ ID NO: 330 - protein, 16 aa, synthetic construct
  REGION 1..16: note = Description of Artificial Sequence: Syntheticpeptide
KSSQSLLDSDGKTYLN

SEQ ID NO: 331 - protein, 9 aa, synthetic construct
  REGION 1..9: note = Description of Artificial Sequence: Syntheticpeptide
LQATHFPQT

SEQ ID NO: 332 - protein, 7 aa, synthetic construct
  REGION 1..7: note = Description of Artificial Sequence: Syntheticpeptide
DYTFTTY

SEQ ID NO: 333 - protein, 7 aa, synthetic construct
  REGION 1..7: note = Description of Artificial Sequence: Syntheticpeptide
LVSKMDS

SEQ ID NO: 334 - protein, 9 aa, synthetic construct
  REGION 1..9: note = Description of Artificial Sequence: Syntheticpeptide
LPRTSFPYT

SEQ ID NO: 335 - protein, 16 aa, synthetic construct
  REGION 1..16: note = Description of Artificial Sequence: Syntheticpeptide
KSSQNLLYNEGKTYLK

SEQ ID NO: 336 - protein, 7 aa, synthetic construct
  REGION 1..7: note = Description of Artificial Sequence: Syntheticpeptide
LVFKMGF

SEQ ID NO: 337 - protein, 9 aa, synthetic construct
  REGION 1..9: note = Description of Artificial Sequence: Syntheticpeptide
LPSTPFPYT

SEQ ID NO: 338 - protein, 7 aa, synthetic construct
  REGION 1..7: note = Description of Artificial Sequence: Syntheticpeptide
GYTFTTY

SEQ ID NO: 339 - protein, 6 aa, synthetic construct
  REGION 1..6: note = Description of Artificial Sequence: Syntheticpeptide
NTYSGV

SEQ ID NO: 340 - protein, 13 aa, synthetic construct
  REGION 1..13: note = Description of Artificial Sequence: Syntheticpeptide
EGSTMVTRYYFDY

SEQ ID NO: 341 - protein, 16 aa, synthetic construct
  REGION 1..16: note = Description of Artificial Sequence: Syntheticpeptide
RSSKSLLHSNGNTYLY

SEQ ID NO: 342 - protein, 7 aa, synthetic construct
  REGION 1..7: note = Description of Artificial Sequence: Syntheticpeptide
RMSNLAS

SEQ ID NO: 343 - protein, 9 aa, synthetic construct
  REGION 1..9: note = Description of Artificial Sequence: Syntheticpeptide
MQHLESPYT

SEQ ID NO: 344 - protein, 7 aa, synthetic construct
  REGION 1..7: note = Description of Artificial Sequence: Syntheticpeptide
AYTFTSY

SEQ ID NO: 345 - protein, 11 aa, synthetic construct
  REGION 1..11: note = Description of Artificial Sequence: Syntheticpeptide
KASQDVSTTVA

SEQ ID NO: 346 - protein, 7 aa, synthetic construct
  REGION 1..7: note = Description of Artificial Sequence: Syntheticpeptide
SASYRYT

SEQ ID NO: 347 - protein, 9 aa, synthetic construct
  REGION 1..9: note = Description of Artificial Sequence: Syntheticpeptide
QQHYSTRPT

SEQ ID NO: 348 - protein, 9 aa, synthetic construct
  REGION 1..9: note = Description of Artificial Sequence: Syntheticpeptide
MQHLEYPST

SEQ ID NO: 349 - protein, 9 aa, synthetic construct
  REGION 1..9: note = Description of Artificial Sequence: Syntheticpeptide
LQTTYFPYT

SEQ ID NO: 350 - protein, 7 aa, synthetic construct
  REGION 1..7: note = Description of Artificial Sequence: Syntheticpeptide
GYNFSSY

SEQ ID NO: 351 - protein, 6 aa, synthetic construct
  REGION 1..6: note = Description of Artificial Sequence: Syntheticpeptide
HPSDST

SEQ ID NO: 352 - protein, 16 aa, synthetic construct
  REGION 1..16: note = Description of Artificial Sequence: Syntheticpeptide
RSSKSLLYKDGKTYLN

SEQ ID NO: 353 - protein, 7 aa, synthetic construct
  REGION 1..7: note = Description of Artificial Sequence: Syntheticpeptide
VVSTRAS

SEQ ID NO: 354 - protein, 9 aa, synthetic construct
  REGION 1..9: note = Description of Artificial Sequence: Syntheticpeptide
QQLVEYPYT

SEQ ID NO: 355 - protein, 7 aa, synthetic construct
  REGION 1..7: note = Description of Artificial Sequence: Syntheticpeptide
GYSFSSY

SEQ ID NO: 356 - protein, 7 aa, synthetic construct
  REGION 1..7: note = Description of Artificial Sequence: Syntheticpeptide
GYTFSSY

SEQ ID NO: 357 - protein, 7 aa, synthetic construct
  REGION 1..7: note = Description of Artificial Sequence: Syntheticpeptide
GFTFNTY

SEQ ID NO: 358 - protein, 8 aa, synthetic construct
  REGION 1..8: note = Description of Artificial Sequence: Syntheticpeptide
RSKSSNYA

SEQ ID NO: 359 - protein, 10 aa, synthetic construct
  REGION 1..10: note = Description of Artificial Sequence: Syntheticpeptide
ELRLRYAMDY

SEQ ID NO: 360 - protein, 16 aa, synthetic construct
  REGION 1..16: note = Description of Artificial Sequence: Syntheticpeptide
KSSQSLLYTNGKTYLN

SEQ ID NO: 361 - protein, 9 aa, synthetic construct
  REGION 1..9: note = Description of Artificial Sequence: Syntheticpeptide
LQSTHFPLT

SEQ ID NO: 362 - protein, 5 aa, synthetic construct
  REGION 1..5: note = Description of Artificial Sequence: Syntheticpeptide
AHHMH

SEQ ID NO: 363 - protein, 17 aa, synthetic construct
  REGION 1..17: note = Description of Artificial Sequence: Syntheticpeptide
EIDPNTGSTTYNQKFRA

SEQ ID NO: 364 - protein, 5 aa, synthetic construct
  REGION 1..5: note = Description of Artificial Sequence: Syntheticpeptide
TYWMN

SEQ ID NO: 365 - protein, 17 aa, synthetic construct
  REGION 1..17: note = Description of Artificial Sequence: Syntheticpeptide
RIHPSDSATQYNQKFKT

SEQ ID NO: 366 - protein, 5 aa, synthetic construct
  REGION 1..5: note = Description of Artificial Sequence: Syntheticpeptide
SFWMH

SEQ ID NO: 367 - protein, 17 aa, synthetic construct
  REGION 1..17: note = Description of Artificial Sequence: Syntheticpeptide
EIYPSSGVTNYNERFKN

SEQ ID NO: 368 - protein, 5 aa, synthetic construct
  REGION 1..5: note = Description of Artificial Sequence: Syntheticpeptide
RYWMN

SEQ ID NO: 369 - protein, 17 aa, synthetic construct
  REGION 1..17: note = Description of Artificial Sequence: Syntheticpeptide
RIHPSDSASQYNQKFKS

SEQ ID NO: 370 - protein, 17 aa, synthetic construct
  REGION 1..17: note = Description of Artificial Sequence: Syntheticpeptide
RIHPSDSATQYDQKFKT

SEQ ID NO: 371 - protein, 5 aa, synthetic construct
  REGION 1..5: note = Description of Artificial Sequence: Syntheticpeptide
DYNMN

SEQ ID NO: 372 - protein, 17 aa, synthetic construct
  REGION 1..17: note = Description of Artificial Sequence: Syntheticpeptide
NINPYYGSTGYTQNFEG

SEQ ID NO: 373 - protein, 5 aa, synthetic construct
  REGION 1..5: note = Description of Artificial Sequence: Syntheticpeptide
SYWMH

SEQ ID NO: 374 - protein, 17 aa, synthetic construct
  REGION 1..17: note = Description of Artificial Sequence: Syntheticpeptide
RIHPSDSDTNYNQNFKG

SEQ ID NO: 375 - protein, 5 aa, synthetic construct
  REGION 1..5: note = Description of Artificial Sequence: Syntheticpeptide
NYWMH

SEQ ID NO: 376 - protein, 17 aa, synthetic construct
  REGION 1..17: note = Description of Artificial Sequence: Syntheticpeptide
RIHPSDSYTNYNQKFKG

SEQ ID NO: 377 - protein, 5 aa, synthetic construct
  REGION 1..5: note = Description of Artificial Sequence: Syntheticpeptide
TYWMH

SEQ ID NO: 378 - protein, 5 aa, synthetic construct
  REGION 1..5: note = Description of Artificial Sequence: Syntheticpeptide
TYGMS

SEQ ID NO: 379 - protein, 17 aa, synthetic construct
  REGION 1..17: note = Description of Artificial Sequence: Syntheticpeptide
WINTYSGVPTYADDFKG

SEQ ID NO: 380 - protein, 5 aa, synthetic construct
  REGION 1..5: note = Description of Artificial Sequence: Syntheticpeptide
SYYMH

SEQ ID NO: 381 - protein, 17 aa, synthetic construct
  REGION 1..17: note = Description of Artificial Sequence: Syntheticpeptide
TIHPSDSTTNCNQKFKG

SEQ ID NO: 382 - protein, 17 aa, synthetic construct
  REGION 1..17: note = Description of Artificial Sequence: Syntheticpeptide
TIHPSDSTTNYNQKFKG

SEQ ID NO: 383 - protein, 5 aa, synthetic construct
  REGION 1..5: note = Description of Artificial Sequence: Syntheticpeptide
TYAMH

SEQ ID NO: 384 - protein, 19 aa, synthetic construct
  REGION 1..19: note = Description of Artificial Sequence: Syntheticpeptide
RIRSKSSNYATYYADSVKD

SEQ ID NO: 385 - DNA, 342 bp, synthetic construct
  misc_feature 1..342: note = Description of Artificial Sequence: Syntheticpolynucleotide
  source 1..342: mol_type = other DNA
gaagtacagttgcagcaatctgggcctgagctggtgaagcccggtgcttccgtgaaaatttcctgcgaaacttcaggattctcatttactgcacatcatatgcactgggtaaaacaatctccagagaaatcactcgaatggataggcgagattgatccaaataccgggtccaccacatacaatcagaaatttcgcgctaaggccaccctgactgtcgataaaagttctaacactacatacatgcagcttaaatcccttacattcgaagacagtgcagtgtactactgttactctaactggtttccatattggggacagggaacactggtaaccgtttccgct

SEQ ID NO: 386 - DNA, 336 bp, synthetic construct
  misc_feature 1..336: note = Description of Artificial Sequence: Syntheticpolynucleotide
  source 1..336: mol_type = other DNA
gacgtagttatgactcagacaccacttacactctctgctactatcggacaaccagcctcaatctattgcaagtcctcacaatctttgcttgatggcgacgggaagacctatctcaattggcttctccaacgacctgggcaaagccccaagagactcatatatctcgtttccaagctggacagtggggtgccagatagatttactgggtcaggtagtggtactgactttactttgaaaatatcaagagtagaggctgaggacctcggagtctattactgctggcaaggaacccatttcccccgcaccttcggaggagggacaaaattggaaataaaa

SEQ ID NO: 387 - DNA, 345 bp, synthetic construct
  misc_feature 1..345: note = Description of Artificial Sequence: Syntheticpolynucleotide
  source 1..345: mol_type = other DNA
caagtgcaacttcagcaacccggcgccgagcttgtgaagcctggtgcctccgttaaactttcttgcaaggcatccggtttctcattcattacctactggatgaactggatcaaacaaagacctggacgtggtctggagtggattgggcggattcacccctcagactccgcaacccaatacaatcagaaattcaaaacaaaggccaccttgaccgttgataaaagcagttctaccgcttatattcaactgtcctctctgacctcagaagactccgcagtgtattactgcgctcgctctactgagggtgcccattggggtcagggaacattggtgactgttagtgct

SEQ ID NO: 388 - DNA, 336 bp, synthetic construct
  misc_feature 1..336: note = Description of Artificial Sequence: Syntheticpolynucleotide
  source 1..336: mol_type = other DNA
gatgttgttatgacccaaactcccctgacactttctgtaacaataggtcagcctgcctctatctcatgcaagtcctcacagagtctgctgcactctgatgggaagacttatttgaactggttgctccagcgccccggacagtctcctaaacgcctgatttatttggtgagcaagttggacagtggcgtaccagaccgattcaccggatctggctccgggacagactttactttgaaaataagtcgtgtcgaggctgaggatcttggcgtgtactactgctggcaggggacacacttcccccagacctttggaggtggaactaagctcgaaatcaaa

SEQ ID NO: 389 - DNA, 350 bp, synthetic construct
  misc_feature 1..350: note = Description of Artificial Sequence: Syntheticpolynucleotide
  source 1..350: mol_type = other DNA
aagtacagcttcagcagccaggagcagaacttgttaagcccggtgcttctgtgaagctgtcctgtaaagctagtggttacactttcactagcttttggatgcactgggtgaaacagaggccaggacaaggcttggagtggattggagagatataccctagcagcggtgtgaccaactacaatgaaagatttaagaataaagccaccctgacagttgataaatcctcacggacagcatacatgcaactctcatctctgacatccgaggacagcgccgtctatttttgtaccccaaactattactacgacggcttgtactgggggcaggggactttggtcacagtgtccgct

SEQ ID NO: 390 - DNA, 336 bp, synthetic construct
  misc_feature 1..336: note = Description of Artificial Sequence: Syntheticpolynucleotide
  source 1..336: mol_type = other DNA
gatgtggtaatgactcaaacaccacttacactcagtgtaactatcggccaacctgccagcatctcctgcaaatccagtcatagcttgttgtataccaatggcgagacctatctcaactggcttctccagaggccaggacagtctcccaaaagacttatatatttggtgtctaacttggactctggtgtgcccgatagattttcagggtctgggtctggcaccgattttacattgaaaatatccagggtggaagccgaagaccttggaatatactactgtctccaatcaacccattttcctcgcacattcggcggcggcactaaactcgaaataaag

SEQ ID NO: 391 - DNA, 345 bp, synthetic construct
  misc_feature 1..345: note = Description of Artificial Sequence: Syntheticpolynucleotide
  source 1..345: mol_type = other DNA
caggtacagctccagcaaccaggggcagagttggtaaagcccggagccagtgtcaagctctcatgcaaggcttccggcttcagtttcaccagatactggatgaattgggttaaacagcgcccaggacgagggcttgaatggataggtaggattcatccctcagactcagcaagtcagtacaatcagaagtttaagtccaaagcaacactgacagtagacaaaagcagcagcacagcttacattcagttgagtagcttgacatcagaggatagcgcagtttattattgtggccgtagtacagaaggggcttattgggggcaaggaacacttgtcacagtgagtgca

SEQ ID NO: 392 - DNA, 345 bp, synthetic construct
  misc_feature 1..345: note = Description of Artificial Sequence: Syntheticpolynucleotide
  source 1..345: mol_type = other DNA
caagttcaattgcagcagcctggtgctgagctggtgaagccaggtgcaagtgttaaactttcatgcaaggcaagcggattctccttcatcacttattggatgaattggatcaaacaacgtcctgggcggggcctggagtggattggtcgcatacacccatctgactccgctacccaatatgaccagaaattcaaaaccaaagcaaccctcactgtggataaaagcagcagcaccgcatacatacaactcagctccctcacttccgaggactctgccgtttactattgcgcacgaagcactgaaggggctcattggggtccaggaacattggtaacagtcagcgca

SEQ ID NO: 393 - DNA, 357 bp, synthetic construct
  misc_feature 1..357: note = Description of Artificial Sequence: Syntheticpolynucleotide
  source 1..357: mol_type = other DNA
gaaatacagcttcagcagtcaggcactgaactggtgaaacccggtgcttcagtgaagatttcctgtaagaccagtggttacagtttcactgattacaacatgaactgggtgaaacaatcccacggaaaaagtctcgaatggataggtaatataaacccttattacggaagcaccggctacactcagaattttgaaggtaaggctactttgaccgtggataaatcttctagtacagcatatatgcagcttaactcacttacttctgaggacagcgccttgtactactgcgctcgtgaagggcatgactactacgctatggactactggggtcaaggcacatctgtcacagtcagctca

SEQ ID NO: 394 - DNA, 339 bp, synthetic construct
  misc_feature 1..339: note = Description of Artificial Sequence: Syntheticpolynucleotide
  source 1..339: mol_type = other DNA
caggtccaacttcagctgcccggagctgaactggtaaaacccggtgcttccgttaaggtgtcttgcaaagcatcaggctacacatttactagctactggatgcactgggtaaagcaacgtccaggtcagggccttgaatggatcggtcgtatacatccttcagactcagataccaattacaatcaaaactttaagggtaaagctactttgattgtcgataagtcttcttcaactgcatacatgcagttgtcttctcttacatccgaggacagtgcagtgtattactgcgctacaggtttctctttttggggacagggaaccctcgtaaccgtgagtgcc

SEQ ID NO: 395 - DNA, 339 bp, synthetic construct
  misc_feature 1..339: note = Description of Artificial Sequence: Syntheticpolynucleotide
  source 1..339: mol_type = other DNA
caggtacaagtgcaggtgccaggagctgagttggtcaagccaggcgctagtgtgaaagtctcatgtaaggccagcggctatactttcactagttactggatgcactggatgaagaagagacccggacaggggctcgaatggatagggcgaatccacccatctgacagcgatacaaattacaaccagaactttaaaggaaaggcaacacttacagttgataagtctagcagcacagcatacatgcagcttagttcactcacatcagaagattccgctgtctatttttgtgctactggtttcagcttttggggtcagggaactctcgtaactgtgtccgca

SEQ ID NO: 396 - DNA, 336 bp, synthetic construct
  misc_feature 1..336: note = Description of Artificial Sequence: Syntheticpolynucleotide
  source 1..336: mol_type = other DNA
gatgtcgttatgacccagactccattgactctgtctgtcaccataggacaacccgcatctatctcctgcaaatcatcacagagcttgctgtattctgacggaaagacatatttgaactggctgctccaacggcctggggagtcccctaaactccttatctatctcgtttctaaacttgacagtggcgtccctgatcgttttaccggctccgggtctggcactgattttacactcaagatcagccgggtggaagcagaggatttgggtgtctactattgtcttcagaccacttccttcccatataccttcggcggcggaactaaattggaaatcaaa

SEQ ID NO: 397 - DNA, 345 bp, synthetic construct
  misc_feature 1..345: note = Description of Artificial Sequence: Syntheticpolynucleotide
  source 1..345: mol_type = other DNA
caagtccagttgcagcagcccggtgctgagcttgtcaaacccggcgcctcagttaaagtctcatgcaaggcttctggctataactttataaattactggatgcactgggtcaaacagcgaccaggacagggcctcgaatggattggtagaatacacccatcagatagttacactaattacaatcagaagtttaaaggtaaggcaacactgactgtggacaaaagcagctcaactgcctacatgcagctcagttctctcacctccgaggatagtgctgtgtactattgtgccagtcccatatccactctttattgggggcagggcaccaccttgaccgtatcctca

SEQ ID NO: 398 - DNA, 336 bp, synthetic construct
  misc_feature 1..336: note = Description of Artificial Sequence: Syntheticpolynucleotide
  source 1..336: mol_type = other DNA
gatgtcgtgatgactcaaactccattgactctgagcgtcactattgggcaacctgctagtatatcatgcaagtcctctcagtctctgttggactccgacgggaagacttatctcaactggttgctgcaacgtcctggtgagagccccaagctccttatatacctggtatcaaaactggattctggggttccagaccgtttcactgggagcgggagcggcacagactttaccctcaagatttcacgggtagaagctgaagacctgggagtgtattactgccttcaagccacacattttcctcaaacatttgggggtggtactaagctggaaattaag

SEQ ID NO: 399 - DNA, 339 bp, synthetic construct
  misc_feature 1..339: note = Description of Artificial Sequence: Syntheticpolynucleotide
  source 1..339: mol_type = other DNA
caagttcagttgcagcttcctggagctgagttggttcggccaggtacatcagttaaagtaagctgcaaagcaagcgactacaccttcaccacatattggatgcactgggtcaaacagcggcctggacaggggctggactggatcgggaggatacatcctagcgattctgatactaactacaatcagaatttcaaaggtaaagccacactcactgtggacaaatcctcttcaaccgcttacatgcacttgtcatccttgacatccgaggactcagcagtttattactgcgctaccggtttcagcttttggggacagggtactttggtgacagtgagcgcc

SEQ ID NO: 400 - DNA, 339 bp, synthetic construct
  misc_feature 1..339: note = Description of Artificial Sequence: Syntheticpolynucleotide
  source 1..339: mol_type = other DNA
caggttcaagtgcaactccctggtgccgaacttgtgaagcccggagccagtgtgaaggttagctgtaaggcctctgggtacacatttacttcctactggatgcactgggtaaaaaagcggccaggacagggactcgaatggataggacgtattcacccttccgactctgacacaaactacaaccaaaacttcaaaggtaaagccactctcaccgtagacaaatcatcatcaaccgcatacatgctcctctcatccctgacatcagaagacagtgctgtttattattgcgctacagggtttagtttttggggccaaggaaccttgattaccgtgtccgca

SEQ ID NO: 401 - DNA, 336 bp, synthetic construct
  misc_feature 1..336: note = Description of Artificial Sequence: Syntheticpolynucleotide
  source 1..336: mol_type = other DNA
gacgtggtgatgactcagacacctctgaccctgtctgtaaccattggccagccagccagtattagttgtaaatcatctcaaagtctcctctactcagacggcaagacctatttgaactggttgctccagcggccaggcgaatcacccgagctgctcatttacttggtctccaagatggattccggtgtgccagatagatttcatggtcacggaagtgggacagccttcacaatgaagatttcccggatgggcggcggtggattgggaaactattactgtctccctcgtacctccttcccttacactttcggtggtgggacaaaactcgagataaaa

SEQ ID NO: 402 - DNA, 339 bp, synthetic construct
  misc_feature 1..339: note = Description of Artificial Sequence: Syntheticpolynucleotide
  source 1..339: mol_type = other DNA
caagtgcagttgcagctccccggtgccgaactcgtaaaacccggcgcaagcgtgaaagtttcctgtaaggcatccggctatacattcacatcatattggatgcattgggtcaaacagcgtcctgggcagggtcttgaatggattgggcggatacatccatctgacagtgataccaactacaatcaaaattttaaagggaaggccaccctcacagttgacaagtctagtaatacagcctacatgcagctttctagcctgactagcgaggattctgctgtttactactgtgcaaccggattcagtttttggggacaaggaactttggtgacagtatccgcc

SEQ ID NO: 403 - DNA, 336 bp, synthetic construct
  misc_feature 1..336: note = Description of Artificial Sequence: Syntheticpolynucleotide
  source 1..336: mol_type = other DNA
gacgtggtgatgaccccaacatcacttcatttgcttgttactatagggcaacccggctttttgttctgtaaaagttcacagaatctcctctacaatgaaggaaaaacatacttgaagtggcttttgcctgagccaggtgctttctccaaggtacttatataccttgtcttcaagatgggatttggggttcctgatcgcttccacggccacggatctggcaccgacttccctatgaaaataagccgaatgggagggggcggccttgggggctacctttgccttccctctaccccctttccttataccttcggcgggggtactaaacttgaaataaaa

SEQ ID NO: 404 - DNA, 366 bp, synthetic construct
  misc_feature 1..366: note = Description of Artificial Sequence: Syntheticpolynucleotide
  source 1..366: mol_type = other DNA
cagatccacttggtacagtctggacctgagctgaagaagcctggagagacagtcaggatctcctgcaaggcttctgggtataccttcacaacctatggaatgagctgggtgaagcaggctccaggaaaggctttaaagtggatgggctggataaacacctactctggagtgccaacatatgctgatgacttcaagggacggtttgccttctctttggaaacctctgccagcactgcctatttgcagatcaacaacctcaaaaatgaggacacggctacatatttctgtacaagagagggatctactatggttacgaggtactactttgactactggggccaaggcaccactctcacagtctcctca

SEQ ID NO: 405 - DNA, 336 bp, synthetic construct
  misc_feature 1..336: note = Description of Artificial Sequence: Syntheticpolynucleotide
  source 1..336: mol_type = other DNA
gacattgttatgacccaagccgccccaagcgtaccagttactcctggcgagagtgtctccattagttgtcggtcttcaaaaagtttgctccactccaatgggaatacttacctttattggttccttcagcgtcctggtcaatctccacagctgctgatttatcgaatgagtaacctggcctcaggagtccctgatcgcttcagtggttcagggtccggtactgcctttacacttaggatctccagggtagaagccgaggatgtaggcgtctaccattgtatgcaacatctcgaatcaccctatactttcggtggaggtacaaaactcgaaataaaa

SEQ ID NO: 406 - DNA, 339 bp, synthetic construct
  misc_feature 1..339: note = Description of Artificial Sequence: Syntheticpolynucleotide
  source 1..339: mol_type = other DNA
caagtacaactgcaactcccaggcgccgagttggttaaacctggcgcttcagtgaaggtatcctgcaaagcatctgcctacactttcacatcttactggatgcactgggtaaaacagcgaccagggcagggacttgaatggattggacgcattcatccttccgatagcgacactaactataaccaaaattttaaggggaaggccaccttgactgtggataaatctagcaacacagcctacatgcaactcagttcactgacttctgaggattctgccgtttattattgtgccacaggcttctccttctgggggcaaggaaccttggtgaccgtgtcagct

SEQ ID NO: 407 - DNA, 321 bp, synthetic construct
  misc_feature 1..321: note = Description of Artificial Sequence: Syntheticpolynucleotide
  source 1..321: mol_type = other DNA
gacatagtaatgactcaaagccacaaattcatgtccaccagtgttggtgaccgcgtatcaatcacttgcaaggccagtcaggacgtatccacaacagttgcatggtatcagcaaaagccaggacaatcacccaaacttctgatttacagtgccagttatcgatacactggggttcccgacagattcacaggatcaggcagcggaactgattttaccttcaccattagctcagtgcaagccgaagatctggccgtgtattattgtcaacagcactatagtaccaggcccaccttcggcgggggaactaaattggaaataaag

SEQ ID NO: 408 - DNA, 366 bp, synthetic construct
  misc_feature 1..366: note = Description of Artificial Sequence: Syntheticpolynucleotide
  source 1..366: mol_type = other DNA
cagatccagttggtacagtctggacctgagctgaagaagcctggagagacagtcaagatctcctgcaaggcttctgggtataccttcacaacctatggaatgagctgggtgaaacaggctccaggaaagggtttaaagtggatgggctggataaacacctactctggagtgccaacatatgctgatgacttcaagggacggtttgccttctctttggaaacctctgccagcactgcctatttgcagatcaacaacctcaaaaatgaggacacggctacatatttctgtgcaagagagggatctactatggttacgaggtactactttgactactggggccaaggcaccactctcacagtctcctca

SEQ ID NO: 409 - DNA, 336 bp, synthetic construct
  misc_feature 1..336: note = Description of Artificial Sequence: Syntheticpolynucleotide
  source 1..336: mol_type = other DNA
gatattgtgatgacccaagctgccccctccgtccccgtcacacccggtgagtccgtgtctataagctgtcgtagttccaagagcttgcttcactcaaatggcaatacatacctttattggttcctgcaacgccccggccagagcccacaggtgttgatttatcgtatgtcaaacctggcctccggcgttcccgacaggttttccggcagtggaagcgggaccgcatttacactgcgaatatctcgtgttgaggcagaagacgttggagtctattactgtatgcaacacctcgaaagcccatacactttcggcggtgggactaagctggaaattaaa

SEQ ID NO: 410 - DNA, 366 bp, synthetic construct
  misc_feature 1..366: note = Description of Artificial Sequence: Syntheticpolynucleotide
  source 1..366: mol_type = other DNA
cagatccagttggttcagtctggacctgagctgaagaagcctggagagacagtcaagatctcctgcaaggcttctgggtataccttcacaacctatggaatgagctgggtgaaacaggctccaggaaagggtttaaagtggatgggctggataaacacctactctggagtgccaacatatgctgatgacttcaagggacggtttgccttctctttggaaacctctgccagcactgcctatttgcagatcaacaacctcaaaaatgaggacacggctacgtttttctgtgcaagagagggatctactatggttacgaggtactactttgactactggggccaaggcaccactctcacagtctcctca

SEQ ID NO: 411 - DNA, 336 bp, synthetic construct
  misc_feature 1..336: note = Description of Artificial Sequence: Syntheticpolynucleotide
  source 1..336: mol_type = other DNA
gatattgtcatgacccaggcagcccccagtgtccccgtgactcctggagaaagtgttagtattagctgtcgatcaagtaaatcacttcttcatagtaacggaaatacttacttgtattggttcctccaaaggccaggccagtctccacagttgctcatctatcgcatgagtaatcttgcttcaggtgtgcctgatcgcttcagtggcagtggatcaggtactgctttcacactccgtataagtagggtggaagccgaggatgtcggtgtctactattgtatgcagcacctggagtatccctcaacatttggtggggggacaaaactggagattaag

SEQ ID NO: 412 - DNA, 339 bp, synthetic construct
  misc_feature 1..339: note = Description of Artificial Sequence: Syntheticpolynucleotide
  source 1..339: mol_type = other DNA
caagtccaggtgcaactgcctggcgccgaacttgtgaaacccggagcctccgttaaggtctcctgcaaggctagtggctatacctttacatcttattggatgcactgggtgaaaaaacgcccagggcagggcctcgaatggatcggccgcatccacccatctgatagcgacactaactataaccagaactttaaaggcaaggctactctgaccgttgataaaagcagttccactgcctacatgcaactgacatcccttaccagtgaggatttcgccgtgtactactgctccacagggttctccttctggggccaggggacccttgttaccgtgtccgca

SEQ ID NO: 413 - DNA, 336 bp, synthetic construct
  misc_feature 1..336: note = Description of Artificial Sequence: Syntheticpolynucleotide
  source 1..336: mol_type = other DNA
gatgtcgttatgacccagactccattgactctgtctgtcaccataggacaacccgcatctatctcctgcaaatcatcacagagcttgctgtattctgacggaaagacatatttgaactggctgctccaacggcctggggagtcccctaaactccttatctatctcgtttctaaacttgacagtggcgtccctgatcgttttaccggctccgggtctggcactgattttacactcaagatcagccgggtggaagcagaggatttgggtgtctactattgtcttcagaccacttacttcccatataccttcggcggcggaactaaattggaaatcaaa

SEQ ID NO: 414 - DNA, 336 bp, synthetic construct
  misc_feature 1..336: note = Description of Artificial Sequence: Syntheticpolynucleotide
  source 1..336: mol_type = other DNA
caagttcagttgcaccaacctggtacaagcctcgttaagcccggtgcgagtgtcaaacttagctgcaaagcatctggttacaatttttccagttattacatgcactgggttaaacagcggcccggccaaggactggagtggatcggaaccatccacccctcagactcaactacgaactgcaatcagaagttcaaggggaaggccacgcttaccgtggacaagtcaagtaggactgcttacatgcaactcaatagcttgacattcgaggattccgcggtctattattgtgcgaatttcgtctattggggacaaggtaccagcgtgacggtctccagc

SEQ ID NO: 415 - DNA, 336 bp, synthetic construct
  misc_feature 1..336: note = Description of Artificial Sequence: Syntheticpolynucleotide
  source 1..336: mol_type = other DNA
gacattgttattacgcaagacgagctgtcaaaccctgttacgagtggtgattctgtatccatatcctgtcgctcctcaaaaagtctgttgtacaaggatggaaaaacttatctgaactggtttctgcaacggccaggccaatctcctcaattgcttatatacgtcgtttcaacgagagcctcaggagtgtctgacagattttccggctccggctctgggaccgattttactctcgaaatcagccgggttaaggccgaagacgttggtgtgtattattgccaacagctcgtagagtacccatatacattcggcgggggcacaaaactcgaaataaag

SEQ ID NO: 416 - DNA, 336 bp, synthetic construct
  misc_feature 1..336: note = Description of Artificial Sequence: Syntheticpolynucleotide
  source 1..336: mol_type = other DNA
caagttcagttgcaccaacctggtacaagcctcgttaagcccggtgcgagtgtcaaacttagctgcaaagcatctggttacaatttttccagttattacatgcactgggttaaacagcggcccggccaaggactggagtggatcggaaccatccacccctcagactcaactacgaactacaatcagaagttcaaggggaaggccacgcttaccgtggacaagtcaagtaggactgcttacatgcaactcaatagcttgacattcgaggattccgcggtctattattgtgcgaatttcgtctattggggacaaggtaccagcgtgacggtctccagc

SEQ ID NO: 417 - DNA, 336 bp, synthetic construct
  misc_feature 1..336: note = Description of Artificial Sequence: Syntheticpolynucleotide
  source 1..336: mol_type = other DNA
caagttcagttgcaccaacctggtacaagcctcgttaagcccggtgcgagtgtcaaacttagctgcaaagcatctggttacagcttttccagttattacatgcactgggttaaacagcggcccggccaaggactggagtggatcggaaccatccacccctcagactcaactacgaactgcaatcagaagttcaaggggaaggccacgcttaccgtggacaagtcaagtaggactgcttacatgcaactcaatagcttgacattcgaggattccgcggtctattattgtgcgaatttcgtctattggggacaaggtaccagcgtgacggtctccagc

SEQ ID NO: 418 - DNA, 336 bp, synthetic construct
  misc_feature 1..336: note = Description of Artificial Sequence: Syntheticpolynucleotide
  source 1..336: mol_type = other DNA
caagttcagttgcaccaacctggtacaagcctcgttaagcccggtgcgagtgtcaaacttagctgcaaagcatctggttacaccttttccagttattacatgcactgggttaaacagcggcccggccaaggactggagtggatcggaaccatccacccctcagactcaactacgaactgcaatcagaagttcaaggggaaggccacgcttaccgtggacaagtcaagtaggactgcttacatgcaactcaatagcttgacattcgaggattccgcggtctattattgtgcgaatttcgtctattggggacaaggtaccagcgtgacggtctccagc

SEQ ID NO: 419 - DNA, 336 bp, synthetic construct
  misc_feature 1..336: note = Description of Artificial Sequence: Syntheticpolynucleotide
  source 1..336: mol_type = other DNA
caagttcagttgcaccaacctggtacaagcctcgttaagcccggtgcgagtgtcaaacttagctgcaaagcatctggttacagcttttccagttattacatgcactgggttaaacagcggcccggccaaggactggagtggatcggaaccatccacccctcagactcaactacgaactacaatcagaagttcaaggggaaggccacgcttaccgtggacaagtcaagtaggactgcttacatgcaactcaatagcttgacattcgaggattccgcggtctattattgtgcgaatttcgtctattggggacaaggtaccagcgtgacggtctccagc

SEQ ID NO: 420 - DNA, 336 bp, synthetic construct
  misc_feature 1..336: note = Description of Artificial Sequence: Syntheticpolynucleotide
  source 1..336: mol_type = other DNA
caagttcagttgcaccaacctggtacaagcctcgttaagcccggtgcgagtgtcaaacttagctgcaaagcatctggttacaccttttccagttattacatgcactgggttaaacagcggcccggccaaggactggagtggatcggaaccatccacccctcagactcaactacgaactacaatcagaagttcaaggggaaggccacgcttaccgtggacaagtcaagtaggactgcttacatgcaactcaatagcttgacattcgaggattccgcggtctattattgtgcgaatttcgtctattggggacaaggtaccagcgtgacggtctccagc

SEQ ID NO: 421 - DNA, 363 bp, synthetic construct
  misc_feature 1..363: note = Description of Artificial Sequence: Syntheticpolynucleotide
  source 1..363: mol_type = other DNA
gaagttcaattggttgagtcagggggcggtcttgttcaacctaaaggctccctcaagttgtcctgtgcagcctctggatttacgtttaacacttatgctatgcactgggttcggcaagcaccggggaaagggctcgagtgggtggcccgcattagatcaaaatcatccaactatgccacctactatgccgattccgtgaaggacagattcacaatatcacgcgatgatagccaaagtatgctctatttgcaaatgaataatcttaaaaccgaagacacagctatgtattattgtgtcagagagttgagacttaggtatgctatggattactggggccaaggtacttcagtgaccgtttcatcc

SEQ ID NO: 422 - DNA, 336 bp, synthetic construct
  misc_feature 1..336: note = Description of Artificial Sequence: Syntheticpolynucleotide
  source 1..336: mol_type = other DNA
gatatactgatgacccaaactccactgactctgtctgtcaccatcggtcagcccgcatcaatcagttgtaaatctagtcagtccctgctgtatactaacggaaagacttatctgaattggcttttgcaacggcccggtcaatcacccaaaaggcttatatacctggtaagcaagttggacagtggagttccggatcgcttcagtggctctggtagtgggacagattttacgctcaaaattagtagggtggaggccgaggatcttggcgtctattattgcctccaatctacgcactttccactcacgtttggggccggaaccaaactcgaacttaaa

SEQ ID NO: 423 - protein, 122 aa, synthetic construct
  REGION 1..122: note = Description of Artificial Sequence: Syntheticpolypeptide
QVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSS

SEQ ID NO: 424 - protein, 107 aa, synthetic construct
  REGION 1..107: note = Description of Artificial Sequence: Syntheticpolypeptide
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIK

SEQ ID NO: 425 - protein, 7 aa, synthetic construct
  REGION 1..7: note = Description of Artificial Sequence: Syntheticpeptide
GYTFSNY

SEQ ID NO: 426 - protein, 6 aa, synthetic construct
  REGION 1..6: note = Description of Artificial Sequence: Syntheticpeptide
LPGTGR

SEQ ID NO: 427 - protein, 13 aa, synthetic construct
  REGION 1..13: note = Description of Artificial Sequence: Syntheticpeptide
RDYYGNFYYAMDY

SEQ ID NO: 428 - protein, 11 aa, synthetic construct
  REGION 1..11: note = Description of Artificial Sequence: Syntheticpeptide
SASQGINNYLN

SEQ ID NO: 429 - protein, 7 aa, synthetic construct
  REGION 1..7: note = Description of Artificial Sequence: Syntheticpeptide
YTSTLQS

SEQ ID NO: 430 - protein, 9 aa, synthetic construct
  REGION 1..9: note = Description of Artificial Sequence: Syntheticpeptide
QQYSKLPRT

SEQ ID NO: 431 - protein, 5 aa, synthetic construct
  REGION 1..5: note = Description of Artificial Sequence: Syntheticpeptide
NYWIE

SEQ ID NO: 432 - protein, 17 aa, synthetic construct
  REGION 1..17: note = Description of Artificial Sequence: Syntheticpeptide
EILPGTGRTIYNEKFKG

SEQ ID NO: 433 - DNA, 366 bp, synthetic construct
  misc_feature 1..366: note = Description of Artificial Sequence: Syntheticpolynucleotide
  source 1..366: mol_type = other DNA
caggttcagttgcagcagtctggttccgaattgatgatgccaggagcttccgtgaagataagctgtaaggccacaggttacactttcagtaactattggatagaatgggtaaagcaaagacctggtcacggtttggaatggatcggggagatactgcctggtaccggcagaactatctacaacgagaaatttaagggtaaagccacttttacagcagacatatccagtaatacagttcaaatgcagctgtcatcactcaccagtgaagatagcgccgtgtattactgcgccaggcgcgattattacggcaacttttattatgctatggattactggggccaaggtacttctgtaactgtaagctcc

SEQ ID NO: 434 - DNA, 321 bp, synthetic construct
  misc_feature 1..321: note = Description of Artificial Sequence: Syntheticpolynucleotide
  source 1..321: mol_type = other DNA
gatatacagatgacgcagtctacttcttccctctctgcgtcccttggcgaccgggtcacaataagctgttctgcttcccagggtataaataactacctgaattggtatcagcaaaaaccggatgggacggtcgaactcctgatatattacacatctacacttcagtctggtgtcccctctcgcttttcaggttccggttccggcactgattatagccttacaattagcaacctcgaaccggaggacatcggaacatattattgccagcaatatagtaaactgcccaggacgtttggcggtggcaccaagttggaaatcaaa

SEQ ID NO: 435 - protein, 16 aa, synthetic construct
  REGION 1..16: note = Description of Artificial Sequence: Syntheticpeptide
  MOD_RES 4: note = Gln or His
  MOD_RES 8: note = Asp, His or Tyr
  MOD_RES 9: note = Gly, Ser or Thr
  MOD_RES 10: note = Asp or Asn
  MOD_RES 12: note = Lys or Glu
KSSXSLLXXXGXTYLN

SEQ ID NO: 436 - protein, 7 aa, synthetic construct
  REGION 1..7: note = Description of Artificial Sequence: Syntheticpeptide
  MOD_RES 4: note = Lys or Asn
LVSXLDS

SEQ ID NO: 437 - protein, 9 aa, synthetic construct
  REGION 1..9: note = Description of Artificial Sequence: Syntheticpeptide
  MOD_RES 1: note = Trp or Leu
  MOD_RES 3: note = Gly or Ser
  MOD_RES 8: note = Arg or Gln
XQXTHFPXT

SEQ ID NO: 438 - protein, 7 aa, synthetic construct
  REGION 1..7: note = Description of Artificial Sequence: Syntheticpeptide
  MOD_RES 3: note = Asn, Ser or Thr
GYXFSSY

SEQ ID NO: 439 - protein, 17 aa, synthetic construct
  REGION 1..17: note = Description of Artificial Sequence: Syntheticpeptide
  MOD_RES 11: note = Cys or Tyr
TIHPSDSTTNXNQKFKG

SEQ ID NO: 440 - protein, 37 aa, Homo sapiens
ENTAVVAVEPDRRNQSPVDQGATGASQGLLDRKEVLG

SEQ ID NO: 441 - protein, 25 aa, Homo sapiens
TAVVAVEPDRRNQSPVDQGATGASQ

SEQ ID NO: 442 - protein, 24 aa, Homo sapiens
ENTAVVAVEPDRRNQSPVDQGATG

SEQ ID NO: 443 - protein, 15 aa, Homo sapiens
ENTAVVAVEPDRRNQ

SEQ ID NO: 444 - protein, 25 aa, Homo sapiens
RNQSPVDQGATGASQGLLDRKEVLG

SEQ ID NO: 445 - protein, 15 aa, Homo sapiens
GEAVVLPEVEPGLTA

SEQ ID NO: 446 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 447 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 448 - protein, 9 aa, Homo sapiens
GLVGLIFAV

SEQ ID NO: 449 - protein, 20 aa, Homo sapiens
GEAVVLPEVEPGLTAREQEA

SEQ ID NO: 450 - protein, 310 aa, Homo sapiens; identical to SEQ ID NO: 2

SEQ ID NO: 451 - protein, 330 aa, synthetic construct
  REGION 1..330: note = Description of Artificial Sequence: Syntheticpolypeptide
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVAAAVEPGLTAREQEATPRPRETTQLPTTHLASTTTATTAQEPATSHPHRDMQPGHHETSTPAGPSQEGEAVVLPEVEPGLTAREQEADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 452 - protein, 330 aa, synthetic construct
  REGION 1..330: note = Description of Artificial Sequence: Syntheticpolypeptide
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVAAAVEPGLTAREQEATPRPRETTQLPTTHLASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDEGEAVVLPEVEPGLTAREQEQGATGASQGLLDRKEVLGGVIAGGLVGLIFAVCLVGFMLYRMKKKDEGSYSLEEPKQANGGAYQKPTKQEEFYA

SEQ ID NO: 453 - protein, 329 aa, synthetic construct
  REGION 1..329: note = Description of Artificial Sequence: Syntheticpolypeptide
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVAAAVEPGLTAREQEATPRPRETTQLPTTHLASTTTATTAQEPATSHPHRDMQPGHHETSTPAGPSQADLHTPHTEDGGPSATERAAEDGASSQLPAAEGEAVVLPEVEPGLTAREQEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 454 - protein, 310 aa, synthetic construct
  REGION 1..310: note = Description of Artificial Sequence: Syntheticpolypeptide
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVAAAVEPGLTAREQEATPRPRETTQLPTTHLASTTTATTAQEPATSHPHRDMQPGHHETSTPAGPSQADLHTPHTEDGGPSATERAAEDGASSQLPAAEGSGEQDFTFETSGENTAVVAVLPEVENQSPVDQGATGASQGLLDRKEVLGGVIAGGLVGLIFAVCLVGFMLYRMKKKDEGSYSLEEPKQANGGAYQKPTKQEEFYA

SEQ ID NO: 455 - protein, 310 aa, synthetic construct
  REGION 1..310: note = Description of Artificial Sequence: Syntheticpolypeptide
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVAAAVEPGLTAREQEATPRPRETTQLPTTHLASTTTATTAQEPATSHPHRDMQPGHHETSTPAGPSQADLHTPHTEDGGPSATERAAEDGASSQLPAAEGSGEQDFTFETSGENTAVVAVEPDRRNQLPEVEGATGASQGLLDRKEVLGGVIAGGLVGLIFAVCLVGFMLYRMKKKDEGSYSLEEPKQANGGAYQKPTKQEEFYA

SEQ ID NO: 456 - protein, 54 aa, synthetic construct
  REGION 1..54: note = Description of Artificial Sequence: Syntheticpolypeptide
SPEPTGLEATAASTSTLPAGEGPKEGEAVVLPEVEPGLTAREQEATPRPRETTQ

SEQ ID NO: 457 - protein, 50 aa, synthetic construct
  REGION 1..50: note = Description of Artificial Sequence: Syntheticpolypeptide
GSGEQDFTFETSGENTAVVAVEPDRRNQSPVDQGATGASQGLLDRKEVLG

(19) United States
(12) Patent Application Publication
(10) Pub. No.: US 2024/0301065 A1
(43) Pub. Date: Sep. 12, 2024

(54) ANTIBODY MOLECULES TO CD138 AND USES THEREOF

(71) Applicant: Visterra, Inc., Waltham, MA (US)

(72) Inventors: Bharat Chaganty, Concord, MA (US); Boopathy Ramakrishnan, Braintree, MA (US); Hedy Adari-Hall, Sudbury, MA (US); Karthik Viswanathan, Acton, MA (US); James R. Myette, Waltham, MA (US); Zachary Shriver, Winchester, MA (US)

(21) Appl. No.: 18/581,992

(22) Filed: Feb. 20, 2024

Related U.S. Application Data
(63) Continuation of application No. 16/149,854, filed on Oct. 2, 2018, now Pat. No. 11,945,868.
(60) Provisional application No. 62/725,880, filed on Aug. 31, 2018.
(60) Provisional application No. 62/566,936, filed on Oct. 2, 2017.

(51) Int. Cl. C07K 16/28; A61K 39/00; A61K 39/395; A61P 35/00; C12N 15/62

(52) U.S. Cl. CPC C07K 16/2818; A61K 39/39558; A61P 35/00; C07K 16/2896; C12N 15/62; A61K 2039/572; C07K 2317/32; C07K 2317/34; C07K 2317/76; C07K 2317/92

(57) ABSTRACT

Antibody molecules that specifically bind to CD138 are disclosed. The antibody molecules can be used to treat, prevent, and/or diagnose disorders, such as multiple myeloma.

### CROSS REFERENCE TO RELATED APPLICATIONS

This application is a Continuation of U.S. patent application Ser. No. 16/149,854, filed Oct. 2, 2018, which claims the benefit of U.S. Provisional Application No. 62/566,936, filed Oct. 2, 2017, and U.S. Provisional Application No. 62/725,880, filed Aug. 31, 2018. The contents of the aforesaid applications are hereby incorporated by reference in their entirety.

### SEQUENCE LISTING

The present application is being filed along with a Sequence Listing in electronic XML file format. The Sequence Listing is provided as a file entitled P2029-701720_SL.xml, created on Feb. 19, 2024, which is 500,157 bytes in size. The information in the electronic format of the sequence listing is incorporated herein by reference in its entirety.

### BACKGROUND

Multiple myeloma (MM) is a cancer formed by malignant plasma cells. These tumors generally develop in bone, but occasionally are found in other tissues. Disease with a single plasma cell tumor is known as an isolated (or solitary) plasmacytoma. When more than one plasmacytoma is present, it is known as multiple myeloma. In the United States, the estimated new cases are about 30,000 in 2017 and more than 10,000 deaths are expected to occur. Despite treatment advances in multiple myeloma therapy, multiple myeloma remains an incurable disease in most patients.

There is a need for developing new approaches for treating, preventing and diagnosing multiple myeloma and other disorders that share similar disease mechanisms.

### SUMMARY

This disclosure provides, at least in part, antibody molecules that bind to CD138, e.g., human CD138, and that comprise one or more functional and structural properties disclosed herein. In an embodiment, the antibody molecule is capable of causing an effector function (e.g., an antibody-dependent cellular cytotoxicity (ADCC) activity) on a cell expressing CD138. In an embodiment, the antibody molecule preferentially binds to a membrane-bound CD138 versus a soluble CD138. In an embodiment, the antibody molecule binds to an epitope in an extracellular region of CD138 that is proximal to the transmembrane domain. In an embodiment, the antibody molecule does not bind to the integrin binding domain (IBD) of CD138. In an embodiment, the antibody molecule does not bind exclusively to the IBD of CD138. While not wishing to be bound by theory, it is believed that in an embodiment, improved or optimal cytotoxicity can be achieved, by targeting certain extracellular region(s) on membrane-bound CD138 that is proximal to the cell membrane.

In an embodiment, the antibody molecule is selected from Table 1, or competes for binding to CD138 with an anti-CD138 monoclonal antibody selected from Table 1. In an embodiment, the antibody molecule binds to the same or overlapping epitope as the epitope recognized by an anti-CD138 monoclonal antibody selected from Table 1. In an embodiment, the antibody molecule comprises one or more heavy chain variable regions and/or one or more light chain variable regions described in Table 1. In an embodiment, the antibody molecule comprises one or more heavy chain CDRs and/or one or more light chain CDRs described in Table 1.

In an embodiment, antibody molecule-drug conjugates (ADCs), nucleic acid molecules encoding the antibody molecules, expression vectors, host cells, compositions (e.g., pharmaceutical compositions), kits, containers, and methods for making the antibody molecules, are also provided. The antibody molecules disclosed herein can be used (alone or in combination with other agents or therapeutic modalities) to treat, prevent and/or diagnose disorders associated with CD138, e.g., cancer or precancerous conditions (e.g., multiple myeloma or smoldering myeloma).

Accordingly, in certain aspects, this disclosure provides an antibody molecule, e.g., an antibody molecule described herein, having one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or all) of the following properties a)-dd):
a) Binds to CD138 (e.g., human CD138) with high affinity, e.g., with a dissociation constant (K_{D}) of less than about 100 nM, typically about 10 nM, and more typically, about 10-0.001 nM, about 10-0.01 nM, about 5-0.01 nM, about 3-0.05 nM, about 1-0.1 nM, or stronger, e.g., less than about 80, 70, 60, 50, 40, 30, 20, 10, 8, 6, 4, 3, 2, 1, 0.5, 0.2, 0.1, 0.05, 0.01, 0.005, or 0.001 nM,
b) Binds to a membrane-bound CD138 with high affinity, e.g., with a dissociation constant (K_{D}) of less than about 100 nM, typically about 10 nM, and more typically, about 10-0.001 nM, about 10-0.01 nM, about 5-0.01 nM, about 3-0.05 nM, about 1-0.1 nM, or stronger, e.g., less than about 80, 70, 60, 50, 40, 30, 20, 10, 8, 6, 4, 3, 2, 1, 0.5, 0.2, 0.1, 0.05, 0.01, 0.005, or 0.001 nM,
c) Binds to a soluble CD138 i) with high affinity, e.g., with a dissociation constant (K_{D}) of less than about 100 nM, typically about 10 nM, and more typically, about 10-0.001 nM, about 10-0.01 nM, about 5-0.01 nM, about 3-0.05 nM, about 1-0.1 nM, or stronger, e.g., less than about 80, 70, 60, 50, 40, 30, 20, 10, 8, 6, 4, 3, 2, 1, 0.5, 0.2, 0.1, 0.05, 0.01, 0.005, or 0.001 nM; or ii) with low affinity, e.g., with a dissociation constant (K_{D}) of greater than about 100 nM, e.g., greater than about 200, 300, 400, or 500 nM,
d) Binds to a membrane-bound CD138, or an intact ectodomain of CD138, i) preferably over a soluble CD138, e.g., the binding affinity to a membrane-bound CD138, or an intact ectodomain of CD138, is at least 2, 3, 4, 5, 6, 7, 8, 9, or 10-fold higher than the binding affinity to a soluble CD138; or ii) with a binding affinity similar to the binding affinity to a soluble CD138, e.g., the binding affinity to a membrane-bound CD138, or an intact ectodomain of CD138, is less than about 10%, 20%, 30%, 40%, 50%, 60%, 70%, 80%, 90%, or 100% higher than the binding affinity to a soluble CD138,
e) Binds to one or more (e.g., 2, 3, 4, 5, 6, 7, 8, 9, 10, 11, 12, 13, 14, 15, 20, 25, 30, 35, 40, 45, 50, 55, 60, 65, 70, 75, or more) amino acid residues of CD138 in an extracellular region proximal to the transmembrane domain of CD138, e.g., within 75, 70, 65, 60, 55, 50, 45, 40, 35, 30, 25, 20, 15, 10, or 5 amino acids from the N-terminus of the transmembrane domain, f) i) Binds to an extracellular region of CD138 distant from the transmembrane domain, e.g., the C-terminus of the region is at least 100, 110, 120, 130, 140, 150, 160, 170, 180, 190, or 200 amino acids from the N-terminus of the transmembrane domain; or ii) does not bind, or binds with low affinity, to an extracellular region of CD138 distant from the transmembrane domain, e.g., the C-terminus of the region is at least 100, 110, 120, 130, 140, 150, 160, 170, 180, 190, or 200 amino acids from the N-terminus of the transmembrane domain,
g) Binds to the integrin binding domain (IBD) of CD138 or a region N-terminal to the IDB; or ii) does not bind, or binds with low affinity, to the IBD of CD138 or a region N-terminal to the IDB,
h) Binds to an epitope on CD138 comprising four or more (e.g., 5, 6, 7, 8, 9, 10, 11, 12, 13, 14, 15, 20, 25, 30, 35, 40, 45, 50, 55, 60, 65, 70, 75, or more) consecutive amino acid residues in an extracellular region proximal to the transmembrane domain, e.g., a region comprising amino acids 176-250 (e.g., 176-214 or 210-250) of any of SEQ ID NOS: 1-3 or 450, optionally, wherein the epitope further comprises four or more (e.g., 5, 6, 7, 8, 9, 10, 11, 12, 13, 14, 15, 20, 25, 30, 35, or more) consecutive amino acid residues in an extracellular region distant from the transmembrane domain, e.g., a region comprising amino acids 23-50, 51-95, 88-121, 88-102, or 111-150 of any of SEQ ID NOS: 1-3 or 450,
i) Binds to two or more different regions in CD138, e.g., a multivalent (e.g., bivalent, trivalent, or tetravalent) antibody molecule comprising two sets of identical, or substantially identical, VH-VL pairs that each bind to the same two or more regions, or comprising different sets of VH-VL pairs that each independently bind to different regions,
j) Does not bind to an epitope on CD138 comprising four or more (e.g., 5, 6, 7, 8, 9, 10, 11, 12, 13, 14, 15, 20, 25, 30, 35, or more) consecutive amino acid residues in an extracellular region distant from the transmembrane domain, e.g., a region comprising amino acids 23-50, 51-95, 88-121, 88-101, or 111-150 of any of SEQ ID NOS: 1-3 or 450,
k) Binds to a cancer or precancerous cell (e.g., a myeloma cell) expressing CD138 with high affinity,
l) Binds to an Fc receptor (FcR) (e.g., one or more of FcγRI, FcγRIIa, FcγRIIb, FcγRIIc, FcγRIIIa, or FcγRIIIb) on the surface of an immune cell (e.g., a natural killer (NK) cell, a macrophage, a monocyte, or an eosinophil),
m) Causes an effector function (e.g., an ADCC activity) on a target cell expressing CD138, n) Binds to C1q and causes complement-dependent cytotoxicity (CDC) on a target cell expressing CD138,
o) Mediates homotypic adhesion of one or more CD138-expressing cells,
p) Inhibits the action of a protease on a membrane-bound CD138, e.g., to reduce shedding of CD138;
q) Reduces (e.g., inhibits) one or more biological activities of a cell expressing CD138, in vitro, ex vivo, or in vivo,
r) Reduces (e.g., inhibits) one or more functions of CD138 (e.g., binding of CD138 to a ligand), in vitro, ex vivo, or in vivo,
s) Reduces (e.g., inhibits) proliferation of a cancer or precancerous cell expressing CD138, t) Binds to the same, similar, or overlapping epitope on CD138 as the epitope recognized by an anti-CD138 monoclonal antibody described herein,
u) Shows the same or similar binding affinity or specificity, or both, as an anti-CD138 monoclonal antibody described herein,
v) Shows the same or similar binding affinity or specificity, or both, as an antibody molecule comprising a heavy chain variable region and/or light chain variable region described herein, e.g., a heavy chain variable region and/or light chain variable region of any of the anti-CD138 monoclonal antibodies described herein,
w) Shows the same or similar binding affinity or specificity, or both, as an antibody molecule comprising one or more (e.g., two or three) heavy chain CDRs and/or one or more (e.g., two or three) light chain CDRs described herein, e.g., one or more (e.g., two or three) heavy chain CDRs and/or one or more (two or three) light chain CDRs of any of the anti-CD138 monoclonal antibodies described herein,
x) Shows the same or similar binding affinity or specificity, or both, as an antibody molecule comprising an amino acid sequence described herein,
y) Shows the same or similar binding affinity or specificity, or both, as an antibody molecule comprising an amino acid sequence encoded by a nucleotide sequence described herein,
z) Inhibits, e.g., competitively inhibits, the binding of a second antibody molecule to CD138, wherein the second antibody molecule is an antibody molecule described herein, e.g., any of the anti-CD138 monoclonal antibodies described herein,
aa) Competes for binding with a second antibody molecule to CD138, wherein the second antibody molecule is an anti-CD138 monoclonal antibody described herein,
bb) Has one or more biological properties of an anti-CD138 monoclonal antibody described herein,
cc) Has one or more structural properties of an anti-CD138 monoclonal antibody described herein, or
dd) Has one or more pharmacokinetic properties of an anti-CD138 monoclonal antibody described herein.

In an aspect, this disclosure features an anti-CD138 antibody molecule, which: (i) binds, or substantially binds, to CD138 in an extracellular region proximal to the transmembrane domain of CD138; and (ii) causes an antibody-dependent cellular cytotoxicity (ADCC) activity on a cell expressing CD138.

In an embodiment, the C-terminus of the extracellular region proximal to the transmembrane domain is within 70, 65, 60, 55, 50, 45, 40, 35, 30, 25, 20, 15, 10, or 5 amino acids from the N-terminus of the transmembrane domain. In an embodiment, the N-terminus of the extracellular region proximal to the transmembrane domain is within 75, 70, 65, 60, 55, 50, 45, 40, 35, 30, 25, 20, 15, 10, or 5 amino acids from the N-terminus of the transmembrane domain.

In an embodiment, the antibody molecule binds to an epitope on CD138 comprising five or more (e.g., 6, 7, 8, 9, 10, 11, 12, 13, 14, 15, 20, 25, 30, 35, 40, 45, 50, 55, 60, 65, 70, 75, or more) consecutive amino acid residues in the extracellular region.

In an embodiment, the extracellular region proximal to the transmembrane domain comprises, or consists of, amino acids 210-250 or 220-245 of any of SEQ ID NOS: 1-3 or 450.

In an embodiment, the antibody molecule binds to an Fc receptor (FcR) (e.g., one or more of FcγRI, FcγRIIa, FcγRIIb, FcγRIIc, FcγRIIIa, or FcγRIIIb) on the surface of an immune cell (e.g., a natural killer (NK) cell, a macrophage, a monocyte, or an eosinophil).

In an embodiment, the cell expressing CD138 is a cancer cell or precancerous cell. In an embodiment, the cancer or precancerous cell is a myeloma cell.

In an embodiment, the antibody molecule further binds, or binds with higher affinity, to an extracellular region of CD138 distant from the transmembrane domain. In an embodiment, the antibody molecule binds to an epitope on CD138 comprising five or more (e.g., 6, 7, 8, 9, 10, 11, 12, 13, 14, 15, 20, 25, 30, 35, or more) consecutive amino acid residues in an extracellular region distant from the transmembrane domain. In an embodiment, the C-terminus of the extracellular region distant from the transmembrane domain is at least 100, 110, 120, 130, 140, 150, 160, 170, 180, 190, or 200 amino acids away from the N-terminus of the transmembrane domain. In an embodiment, the extracellular region distant from the transmembrane domain comprises amino acids 23-50, 51-95, 88-121, or 111-150 of any of SEQ ID NOS: 1-3 or 450. In some embodiments, the extracellular region distant from the transmembrane domain comprises amino acids 88-121 or 101-121 of any of SEQ ID NOS: 1-3 or 450.

In an embodiment, the antibody molecule further binds, or binds with higher affinity, to the integrin binding domain (IBD) of CD138. In an embodiment, the antibody molecule further binds, or binds with high affinity, to a region N-terminal to the IBD of CD138.

In an embodiment, the antibody molecule does not bind, or binds with low affinity, to an extracellular region of CD138 distant from the transmembrane domain. In an embodiment, the antibody molecule does not bind to an epitope on CD138 comprising five or more (e.g., 6, 7, 8, 9, 10, 11, 12, 13, 14, 15, 20, 25, 30, 35, or more) consecutive amino acid residues in an extracellular region distant from the transmembrane domain. In an embodiment, the C-terminus of the extracellular region distant from the transmembrane domain is at least 100, 110, 120, 130, 140, 150, 160, 170, 180, 190, or 200 amino acids away from the N-terminus of the transmembrane domain. In an embodiment, the extracellular region distant from the transmembrane domain comprises amino acids 23-50, 51-95, 88-121, or 111-150 of any of SEQ ID NOS: 1-3 or 450.

In an embodiment, the antibody molecule does not bind, or binds with low affinity, to the integrin binding domain (IBD) of CD138. In an embodiment, the antibody molecule does not bind, or binds with low affinity, to a region N-terminal to the IBD of CD138.

In an embodiment, the antibody molecule binds to CD138 with a disassociation constant (K_{D}) of less than about 100, 90, 80, 70, 60, 50, 40, 30, 20, 10, 8, 6, 4, 3, 2, 1, 0.5, 0.2, 0.1, 0.05, 0.01, 0.005, or 0.001 nM, or about 10-0.001, 10-0.01, 5-0.01, 3-0.05, or 1-0.1 nM.

In an embodiment, the binding affinity of the antibody molecule to a membrane-bound CD138 is at least 2, 3, 4, 5, 6, 7, 8, 9, 10, 20, 50, 100, 200, or 500-fold higher than the binding affinity to a soluble CD138. In an embodiment, the antibody molecule binds to a membrane-bound CD138 with a K_{D} less than about 100, 90, 80, 70, 60, 50, 40, 30, 20, 10, 8, 6, 4, 3, 2, 1, 0.5, 0.2, 0.1, 0.05, 0.01, 0.005, or 0.001 nM, or about 10-0.001, 10-0.01, 5-0.01, 3-0.05, or 1-0.1 nM.

In an embodiment, the binding affinity of the antibody molecule to a membrane-bound CD138 is similar its binding affinity to a soluble CD138, e.g., the binding affinity to a membrane-bound CD138 is within about ±10%, ±20%, ±30%, ±40%, ±50%, ±60%, ±70%, ±80%, ±90%, ±100% of, e.g., less than about 10%, 20%, 30%, 40%, 50%, 60%, 70%, 80%, 90%, or 100% higher than, the binding affinity to a soluble CD138. In an embodiment, the antibody molecule binds to a soluble CD138 with a K_{D} of less than about 100 nM, typically about 10 nM, and more typically, about 10-0.001 nM, about 10-0.01 nM, about 5-0.01 nM, about 3-0.05 nM, about 1-0.1 nM, or stronger, e.g., less than about 80, 70, 60, 50, 40, 30, 20, 10, 8, 6, 4, 3, 2, 1, 0.5, 0.2, 0.1, 0.05, 0.01, 0.005, or 0.001 nM. In an embodiment, the antibody molecule binds to a soluble CD138 with a K_{D} of greater than about 100, 200, 300, 400, or 500 nM.

In an embodiment, the antibody molecule binds to a membrane-bound CD138 preferably over a soluble CD138, e.g., the binding affinity to a membrane-bound CD138 is at least 2, 3, 4, 5, 6, 7, 8, 9, or 10-fold higher than the binding affinity to a soluble CD138. In an embodiment, the antibody molecule binds to a soluble CD138 preferably over a membrane-bound CD138, e.g., the binding affinity to a soluble CD138 is at least 2, 3, 4, 5, 6, 7, 8, 9, or 10-fold higher than the binding affinity to a membrane-bound CD138. In an embodiment, the antibody molecule binds to both soluble CD138 and to membrane-bound CD138.

In an embodiment, the antibody molecule binds to C1q and causes a complement-dependent cytotoxicity (CDC) activity on a cell expressing CD138. In an embodiment, the antibody molecule reduces (e.g., inhibits, blocks, or neutralizes) one or more biological activities of a cell expressing CD138 in vitro, ex vivo, or in vivo. In an embodiment, the antibody molecule mediates homotypic adhesion of one or more CD138-expressing cells. In an embodiment, the antibody molecule inhibits the action of a protease on a membrane-bound CD138, e.g., to reduce shedding of CD138. In an embodiment, the antibody molecule reduces (e.g., inhibits) proliferation of a cancer or precancerous cell expressing CD138.

In an embodiment, the antibody molecule comprises one or more (e.g., two or three) heavy chain CDRs and/or one or more (e.g., two or three) light chain CDRs of an anti-CD138 monoclonal antibody described herein. In an embodiment, the antibody molecule comprises a heavy chain variable region (VH) and/or light chain variable region (VL) of an anti-CD138 monoclonal antibody described herein. In an embodiment, the antibody molecule comprises an Fc region.

In an aspect, the disclosure features an anti-CD138 antibody molecule, which binds, or substantially binds, to an epitope on CD138 comprising four or more (e.g., 5, 6, 7, 8, 9, 10, 11, 12, 13, 14, 15, 20, 25, 30, 35, 40, 45, 50, 55, 60, 65, 70, 75, or more) consecutive amino acid residues in an extracellular region proximal to the transmembrane domain of CD138.

In an embodiment, the C-terminus of the extracellular region proximal to the transmembrane domain is within 70, 65, 60, 55, 50, 45, 40, 35, 30, 25, 20, 15, 10, or 5 amino acids from the N-terminus of the transmembrane domain. In an embodiment, the N-terminus of the extracellular region proximal to the transmembrane domain is within 75, 70, 65, 60, 55, 50, 45, 40, 35, 30, 25, 20, 15, 10, or 5 amino acids from the N-terminus of the transmembrane domain. In an embodiment, the extracellular region proximal to the transmembrane domain comprises, or consists of, amino acids 176-250 of any of SEQ ID NOS: 1-3 or 450.

In an embodiment, the antibody molecule binds to an Fc receptor (FcR) (e.g., one or more of FcγRI, FcγRIIa, FcγRIIb, FcγRIIc, FcγRIIIa, or FcγRIIIb) on the surface of an immune cell (e.g., a natural killer (NK) cell, a macrophage, a monocyte, or an eosinophil). In an embodiment, the antibody molecule is capable of causing (e.g., promoting or inducing) an ADCC activity on a cell expressing CD138. In an embodiment, the antibody molecule is capable of causing (e.g., promoting or inducing) antibody dependent cellular phagocytosis (ADCP) activity on a cell expressing CD138. In an embodiment, the cell expressing CD138 is a cancer cell or precancerous cell. In an embodiment, the cancer or precancerous cell is a myeloma cell.

In an embodiment, the antibody molecule further binds, or binds with higher affinity, to an extracellular region of CD138 distant from the transmembrane domain. In an embodiment, the antibody molecule binds to an epitope on CD138 comprising five or more (e.g., 6, 7, 8, 9, 10, 11, 12, 13, 14, 15, 20, 25, 30, 35, or more) consecutive amino acid residues in an extracellular region distant from the transmembrane domain. In an embodiment, the C-terminus of the extracellular region distant from the transmembrane domain is at least 100, 110, 120, 130, 140, 150, 160, 170, 180, 190, or 200 amino acids away from the N-terminus of the transmembrane domain. In an embodiment, the extracellular region distant from the transmembrane domain comprises amino acids 23-50, 51-95, 88-121, or 111-150 of any of SEQ ID NOS: 1-3 or 450.

In an embodiment, the antibody molecule further binds, or binds with higher affinity, to the integrin binding domain (IBD) of CD138. In an embodiment, the antibody molecule further binds, or binds with high affinity, to a region N-terminal to the IBD of CD138.

In an embodiment, the antibody molecule does not bind, or binds with low affinity, to an extracellular region of CD138 distant from the transmembrane domain.

In an embodiment, the epitope does not comprise five or more (e.g., 6, 7, 8, 9, 10, 11, 12, 13, 14, 15, 20, 25, 30, 35, or more) consecutive amino acid residues in an extracellular region distant from the transmembrane domain. In an embodiment, the C-terminus of the extracellular region distant from the transmembrane domain is at least 100, 110, 120, 130, 140, 150, 160, 170, 180, 190, or 200 amino acids away from the N-terminus of the transmembrane domain. In an embodiment, the extracellular region distant from the transmembrane domain comprises amino acids 23-50, 51-95, 88-121, or 111-150 of any of SEQ ID NOS: 1-3 or 450.

In an embodiment, the antibody molecule does not bind, or binds with low affinity, to the integrin binding domain (IBD) of CD138. In an embodiment, the antibody molecule does not bind, or binds with low affinity, to a region N-terminal to the IBD of CD138.

In an embodiment, the antibody molecule binds to CD138 with a disassociation constant (K_{D}) of less than about 100, 90, 80, 70, 60, 50, 40, 30, 20, 10, 8, 6, 4, 3, 2, 1, 0.5, 0.2, 0.1, 0.05, 0.01, 0.005, or 0.001 nM, or about 10-0.001, 10-0.01, 5-0.01, 3-0.05, or 1-0.1 nM.

In an embodiment, the binding affinity of the antibody molecule to a membrane-bound CD138 is at least 2, 3, 4, 5, 6, 7, 8, 9, 10, 20, 50, 100, 200, or 500-fold higher than the binding affinity to a soluble CD138. In an embodiment, the antibody molecule binds to a membrane-bound CD138 with a K_{D} less than about 100, 90, 80, 70, 60, 50, 40, 30, 20, 10, 8, 6, 4, 3, 2, 1, 0.5, 0.2, 0.1, 0.05, 0.01, 0.005, or 0.001 nM, or about 10-0.001, 10-0.01, 5-0.01, 3-0.05, or 1-0.1 nM. In an embodiment, the antibody molecule binds to a soluble CD138 with a K_{D} of greater than about 100, 200, 300, 400, or 500 nM.

In an embodiment, the binding affinity of the antibody molecule to a membrane-bound CD138 is similar its binding affinity to a soluble CD138, e.g., the binding affinity to a membrane-bound CD138 is within about ±10%, ±20%, ±30%, ±40%, ±50%, ±60%, ±70%, ±80%, ±90%, ±100% of, e.g., less than about 10%, 20%, 30%, 40%, 50%, 60%, 70%, 80%, 90%, or 100% higher than, the binding affinity to a soluble CD138. In an embodiment, the antibody molecule binds to a soluble CD138 with a K_{D} of less than about 100 nM, typically about 10 nM, and more typically, about 10-0.001 nM, about 10-0.01 nM, about 5-0.01 nM, about 3-0.05 nM, about 1-0.1 nM, or stronger, e.g., less than about 80, 70, 60, 50, 40, 30, 20, 10, 8, 6, 4, 3, 2, 1, 0.5, 0.2, 0.1, 0.05, 0.01, 0.005, or 0.001 nM. In an embodiment, the antibody molecule binds to a soluble CD138 with a K_{D} of greater than about 100, 200, 300, 400, or 500 nM.

In an embodiment, the antibody molecule binds to a membrane-bound CD138 preferably over a soluble CD138, e.g., the binding affinity to a membrane-bound CD138 is at least 2, 3, 4, 5, 6, 7, 8, 9, or 10-fold higher than the binding affinity to a soluble CD138. In an embodiment, the antibody molecule binds to a soluble CD138 preferably over a membrane-bound CD138, e.g., the binding affinity to a soluble CD138 is at least 2, 3, 4, 5, 6, 7, 8, 9, or 10-fold higher than the binding affinity to a membrane-bound CD138. In an embodiment, the antibody molecule binds to both soluble CD138 and to membrane-bound CD138.

In an embodiment, the antibody molecule binds to C1q and causes a complement-dependent cytotoxicity (CDC) activity on a cell expressing CD138. In an embodiment, the antibody molecule reduces (e.g., inhibits, blocks, or neutralizes) one or more biological activities of a cell expressing CD138 in vitro, ex vivo, or in vivo. In an embodiment, the antibody molecule mediates homotypic adhesion of one or more CD138-expressing cells. In an embodiment, the antibody molecule inhibits the action of a protease on a membrane-bound CD138, e.g., to reduce shedding of CD138. In an embodiment, the antibody molecule reduces (e.g., inhibits) proliferation of a cancer or precancerous cell expressing CD138.

In an embodiment, the antibody molecule comprises one or more (e.g., two or three) heavy chain CDRs and/or one or more (e.g., two or three) light chain CDRs of an anti-CD138 monoclonal antibody described herein. In an embodiment, the antibody molecule comprises a heavy chain variable region (VH) and/or light chain variable region (VL) of an anti-CD138 monoclonal antibody described herein. In an embodiment, the antibody molecule comprises an Fc region.

In an aspect, the disclosure features an anti-CD138 antibody molecule comprising one or both of:
(a) a heavy chain variable region (VH), wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of an anti-CD138 monoclonal antibody described herein (e.g., any of antibodies CD001, CD002, CD003, CD004, CD005, CD006, 602, 603, 604, 607, 613, 614, 617, 624, 632, 616, 619, 623, 1610, 2510, 2610, 2710, 2810, 2910, or 1409); (ii) an HCDR2 comprising an amino acid sequence that differs by no more than 1, 2, or 3 amino acid residues from, or has at least 85, 90, 95, 99 or 100% homology with, the amino acid sequence of the HCDR2 of the anti-CD138 antibody; or (iii) an HCDR3 comprising an amino acid sequence that differs by no more than 1, 2, or 3 amino acid residues from, or has at least 85, 90, 95, 99 or 100% homology with, the amino acid sequence of the HCDR3 of the anti-CD138 antibody; or
(b) a light chain variable region (VL), wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of the anti-CD138 antibody; (ii) an LCDR2 comprising an amino acid sequence that differs by no more than 1, 2, or 3 amino acid residues from, or has at least 85, 90, 95, 99 or 100% homology with, the amino acid sequence of the LCDR2 of the anti-CD138 antibody; or (iii) an LCDR3 comprising an amino acid sequence that differs by no more than 1, 2, or 3 amino acid residues from, or has at least 85, 90, 95, 99 or 100% homology with, the amino acid sequence of the LCDR3 of the anti-CD138 antibody.

In an embodiment, the VH comprises: (i) an HCDR1 comprising an amino acid sequence that differs by no more than 1, 2, or 3 amino acid residues from, or has at least 85, 90, 95, 99 or 100% homology with, the amino acid sequence of the HCDR1 of the anti-CD138 antibody; (ii) an HCDR2 comprising an amino acid sequence that differs by no more than 1, 2, or 3 amino acid residues from, or has at least 85, 90, 95, 99 or 100% homology with, the amino acid sequence of the HCDR2 of the anti-CD138 antibody; and (iii) an HCDR3 comprising an amino acid sequence that differs by no more than 1, 2, or 3 amino acid residues from, or has at least 85, 90, 95, 99 or 100% homology with, the amino acid sequence of the HCDR3 of the anti-CD138 antibody.

In an embodiment, the VH comprises: (i) an HCDR1 comprising the amino acid sequence of the HCDR1 of the anti-CD138 antibody; (ii) an HCDR2 comprising the amino acid sequence of the HCDR2 of the anti-CD138 antibody; and (iii) an HCDR3 comprising the amino acid sequence of the HCDR3 of the anti-CD138 antibody.

In an embodiment, the VL comprises: (i) an LCDR1 comprising an amino acid sequence that differs by no more than 1, 2, or 3 amino acid residues from, or has at least 85, 90, 95, 99 or 100% homology with, the amino acid sequence of the LCDR1 of the anti-CD138 antibody; (ii) an LCDR2 comprising an amino acid sequence that differs by no more than 1, 2, or 3 amino acid residues from, or has at least 85, 90, 95, 99 or 100% homology with, the amino acid sequence of the LCDR2 of the anti-CD138 antibody; and (iii) an LCDR3 comprising an amino acid sequence that differs by no more than 1, 2, or 3 amino acid residues from, or has at least 85, 90, 95, 99 or 100% homology with, the amino acid sequence of the LCDR3 of the anti-CD138 antibody.

In an embodiment, the VL comprises: (i) an LCDR1 comprising the amino acid sequence of the LCDR1 of the anti-CD138 antibody; (ii) an LCDR2 comprising the amino acid sequence of the LCDR2 of the anti-CD138 antibody; and (iii) an LCDR3 comprising the amino acid sequence of the LCDR3 of the anti-CD138 antibody.

In an embodiment, the antibody molecule comprises:
(a) a VH comprising: (i) an HCDR1 comprising an amino acid sequence that differs by no more than 1, 2, or 3 amino acid residues from, or has at least 85, 90, 95, 99 or 100% homology with, the amino acid sequence of the HCDR1 of the anti-CD138 antibody; (ii) an HCDR2 comprising an amino acid sequence that differs by no more than 1, 2, or 3 amino acid residues from, or has at least 85, 90, 95, 99 or 100% homology with, the amino acid sequence of the HCDR2 of the anti-CD138 antibody; and (iii) an HCDR3 comprising an amino acid sequence that differs by no more than 1, 2, or 3 amino acid residues from, or has at least 85, 90, 95, 99 or 100% homology with, the amino acid sequence of the HCDR3 of the anti-CD138 antibody, and
(b) a VL comprising: (i) an LCDR1 comprising an amino acid sequence that differs by no more than 1, 2, or 3 amino acid residues from, or has at least 85, 90, 95, 99 or 100% homology with, the amino acid sequence of the LCDR1 of the anti-CD138 antibody; (ii) an LCDR2 comprising an amino acid sequence that differs by no more than 1, 2, or 3 amino acid residues from, or has at least 85, 90, 95, 99 or 100% homology with, the amino acid sequence of the LCDR2 of the anti-CD138 antibody; and (iii) an LCDR3 comprising an amino acid sequence that differs by no more than 1, 2, or 3 amino acid residues from, or has at least 85, 90, 95, 99 or 100% homology with, the amino acid sequence of the LCDR3 of the anti-CD138 antibody.

In an embodiment, the antibody molecule comprises: (a) a VH comprising: (i) an HCDR1 comprising the amino acid sequence of the HCDR1 of the anti-CD138 antibody; (ii) an HCDR2 comprising the amino acid sequence of the HCDR2 of the anti-CD138 antibody; and (iii) an HCDR3 comprising the amino acid sequence of the HCDR3 of the anti-CD138 antibody, and (b) a VL comprising: (i) an LCDR1 comprising the amino acid sequence of the LCDR1 of the anti-CD138 antibody; (ii) an LCDR2 comprising the amino acid sequence of the LCDR2 of the anti-CD138 antibody; and (iii) an LCDR3 comprising the amino acid sequence of the LCDR3 of the anti-CD138 antibody.

In an embodiment, the VH comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VH of the anti-CD138 antibody. In an embodiment, the antibody molecule the VH comprises the amino acid sequence of the VH of the anti-CD138 antibody.

In an embodiment, the VL comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VL of the anti-CD138 antibody. In an embodiment, the VL comprises the amino acid sequence of the VL of the anti-CD138 antibody.

In an embodiment, (a) the VH comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VH of the anti-CD138 antibody; and (b) the VL comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VH of the anti-CD138 antibody.

In an embodiment, the VH comprises the amino acid sequence of the VH of the anti-CD138 antibody and the VL comprises the amino acid sequence of the VL of the anti-CD138 antibody.

In an embodiment, the antibody molecule comprises an Fc region.

In an aspect, the disclosure features an antibody molecule, which competes for binding to CD138 with an anti-CD138 monoclonal antibody described herein (e.g., any of antibodies CD001, CD002, CD003, CD004, CD005, CD006, 602, 603, 604, 607, 613, 614, 617, 624, 632, 616, 619, 623, 1610, 2510, 2610, 2710, 2810, 2910, or 1409).

In an aspect, the disclosure features an antibody molecule, which binds, or substantially binds, to an epitope that completely or partially overlaps with the epitope of an anti-CD138 monoclonal antibody described herein (e.g., any of antibodies CD001, CD002, CD003, CD004, CD005, CD006, 602, 603, 604, 607, 613, 614, 617, 624, 632, 616, 619, 623, 1610, 2510, 2610, 2710, 2810, 2910, or 1409).

In an aspect, the disclosure features an antibody-molecule drug conjugate (ADC) comprising an antibody molecule described herein, optionally comprising a cytotoxic agent, further optionally comprising a linker.

In an aspect, the disclosure features a composition comprising an antibody molecule described herein, or an ADC described herein, optionally, wherein the composition is a pharmaceutical composition.

In an embodiment, the composition further comprises a pharmaceutically acceptable carrier.

In an aspect, the disclosure features a nucleic acid molecule encoding a heavy chain variable region (VH), a light chain variable region (VL), or both, of an antibody molecule described herein.

In an aspect, the disclosure features a vector comprising a nucleic acid molecule described herein.

In an aspect, the disclosure features a cell comprising a nucleic acid molecule described herein or a vector described herein, optionally, wherein the cell is an isolated cell.

In an aspect, the disclosure features a kit comprising an antibody molecule described herein, an ADC described herein, or a composition described herein, and instructions to use of the antibody molecule or composition.

In an aspect, the disclosure features a container comprising an antibody molecule described herein, an ADC described herein, or a composition described herein.

In an aspect, the disclosure features a method of producing an anti-CD138 antibody molecule, the method comprising culturing a cell described herein under conditions that allow production of an antibody molecule, thereby producing the antibody molecule.

In an embodiment, the method further comprises isolating or purifying the antibody molecule.

In an aspect, the disclosure features an antibody molecule of described herein, an ADC described herein, or a composition described herein, for use in a method of treating a cancer in a subject.

In an embodiment, the cancer is a hematological cancer. In an embodiment, the cancer is a multiple myeloma. In an embodiment, the cancer is a solid tumor, e.g., a solid tumor described herein.

In an embodiment, the antibody molecule, ADC, or composition is administered to the subject intravenously.

In an embodiment, the antibody molecule, ADC, or composition is administered to the subject at a dose between 0.1 mg/kg and 50 mg/kg, between 0.2 mg/kg and 25 mg/kg, between 0.5 mg/kg and 10 mg/kg, between 0.5 mg/kg and 5 mg/kg, between 0.5 mg/kg and 3 mg/kg, between 0.5 mg/kg and 2.5 mg/kg, between 0.5 mg/kg and 2 mg/kg, between 0.5 mg/kg and 1.5 mg/kg, between 0.5 mg/kg and 1 mg/kg, between 1 mg/kg and 1.5 mg/kg, between 1 mg/kg and 2 mg/kg, between 1 mg/kg and 2.5 mg/kg, between 1 mg/kg and 3 mg/kg, between 1 mg/kg and 2.5 mg/kg, or between 1 mg/kg and 5 mg/kg.

In an embodiment, the antibody molecule, ADC, or composition is administered to the subject at a fixed dose between 10 mg and 1000 mg, between 10 mg and 500 mg, between 10 mg and 250 mg, between 10 mg and 150 mg, between 10 mg and 100 mg, between 10 mg and 50 mg, between 250 mg and 500 mg, between 150 mg and 500 mg, between 100 mg and 500 mg, between 50 mg and 500 mg, between 25 mg and 250 mg, between 50 mg and 150 mg, between 50 mg and 100 mg, between 100 mg and 150 mg, between 100 mg and 200 mg, or between 150 mg and 250 mg.

In an embodiment, the antibody molecule, ADC, or composition is administered once a week, twice a week, once every two weeks, once every three weeks, or once every four weeks.

In an embodiment, the use further comprises determining the level of CD138 in a sample from the subject. In an embodiment, the use further comprises administering to the subject a second therapy for cancer.

In an aspect, the disclosure features an antibody molecule described herein, an ADC described herein, or a composition described herein, for use in a method of treating a precancerous condition or preventing a cancer.

In an embodiment, the precancerous condition is smoldering myeloma or monoclonal gammopathy of undetermined significance (MGUS). In an embodiment, the cancer is multiple myeloma.

In an aspect, the disclosure features a method of causing an ADCC activity, the method comprising contacting a cell or subject an antibody molecule described herein, an ADC described herein, or a composition described herein, thereby causing the ADCC activity.

In an aspect, the disclosure features a method of treating a cancer, the method comprising administering to a subject in need thereof an effective amount of an antibody molecule described herein, an ADC described herein, or a composition described herein, thereby treating the cancer.

In an aspect, the disclosure features a method of treating a precancerous condition or preventing a cancer, the method comprising administering to a subject in need thereof an effective amount of an antibody molecule described herein, an ADC described herein, or a composition described herein, thereby treating the precancerous condition or preventing the cancer.

In an aspect, the disclosure features, a method of detecting an anti-CD138 molecule, the method comprising contacting a cell or a subject with an antibody molecule described herein, thereby detecting the CD138 molecule.

In an embodiment, the antibody molecule is coupled with a detectable label. In an embodiment, the CD138 molecule is detected in vitro, ex vivo, or in vivo.

The disclosure contemplates all combinations of any one or more of the foregoing aspects and/or embodiments, as well as combinations with any one or more of the embodiments set forth in the detailed description and examples.

Other features, objects, and advantages of the compositions and methods herein will be apparent from the description and drawings, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depict an exemplary amino acid sequence of human CD138 (UniProt ID: P18827). The signal peptide includes residues 1-22 (shown in italics); the extracellular domain includes residues 23-254; the transmembrane domain includes residues 255-275; and the cytoplasmic domain includes residues 276-310. The integrin binding domain (IBD) includes residues 88-122. Known O-linked heparin sulfate chains are located at residues 37, 45 and 47 (underlined); and known O-linked chondroitin sulfate chains are located at residues 206 and 216 (underlined). A possible N-linked glycan is located at residue 43. The inferred B-B4 antibody hot spot epitope residues are Leu 107, Pro108, Glu109 and Val110 (shown in bold). Exemplary peptide region that can be targeted by an anti-CD138 antibody molecules described herein includes residues Gly217 to Glu251 (shown in bold and italics). FIG. 1 discloses SEQ ID NO: 450.

FIG. 2 depicts the peptides used to identify anti-CD138 antibodies that bind to a desired epitope.

FIG. 3 depicts the characterization of anti-CD138 antibody B-B4 for complement-dependent cytotoxicity (CDC) in human myeloma RPMI 8226 cells.

FIG. 4 depicts the characterization of anti-CD138 antibody B-B4 for antibody dependent cellular cytotoxicity (ADCC) in human myeloma RPMI 8226 cells.

FIG. 5 depicts the ability of rabbit polyclonal anti-CD138 antibody to induce ADCC in human multiple myeloma U266 cells.

FIG. 6A depicts the constructs which include the transposition of B-B4 epitope at different positions.

FIG. 6B depicts the amino acid sequences of mutated CD138 in Clones 1-3. FIG. 6B discloses SEQ ID NOS 451-453, respectively, in order of appearance.

FIG. 6C depicts the amino acid sequences of mutated CD138 in Clones 4 and 5. FIG. 6C discloses SEQ ID NOS 454-455, respectively, in order of appearance.

FIG. 7A is a line graph showing the ability of B-B4 to induce ADCC activity when the epitope is moved proximal to the cell membrane.

FIG. 7B is a bar graph showing the ability of B-B4 to induce ADCC activity when the epitope is moved proximal to the cell membrane. BB4-Mid: hCD138 with BB-4 epitope at midway through the ectodomain. BB4-MP: hCD138 with BB-4 epitope at membrane proximal region. CD138: human CD138 wild-type. R-PAb: rabbit polyclonal anti-CD138 antibody.

FIG. 7C depicts the further enhancement of ADCC potential by Fc engineering.

FIG. 8 is a graph showing binding of various anti-CD138 antibodies to a soluble form of the CD138 extracellular domain (amino acids 23-250, SEQ ID NO: 1) as measured by ELISA.

FIGS. 9A-9C are a series of graphs showing binding of various anti-CD138 antibodies to peptide fragments of CD138 as measured by ELISA: (A) Peptide 2a. (B) Peptide 5, (C) Peptide 6.

FIG. 9D shows the structure of the CD138 polypeptide. The locations of Peptides 2a, 5, and 6 are indicated.

FIG. 10 is a graph showing binding of anti-CD138 antibodies 1610, 624, and B-B4 (also referred to as BB4 herein) to Peptides 2a and 6 of CD138 as measured by ELISA under higher stringency conditions of antibody-antigen binding.

FIGS. 11A-11C are a series of diagrams showing binding of antibody 1610 to cell surface CD138 expressed on U266 multiple myeloma cells (A) or to soluble CD138 extracellular domain (B). (C) EC50 values for antibody 1610 binding to soluble or membrane-bound (cell surface) CD138.

FIG. 12 is a series of graphs showing binding kinetics for anti-CD138 antibodies 1610, 624, and B-B4 to recombinant CD138 extracellular domain as measured by bio-layer interferometry.

FIG. 13 is a series of graphs showing binding kinetics for anti-CD138 antibody 1610 to CD138 peptide fragments (top panel: Peptides 2A (SEQ ID NO: 10), 2C (SEQ ID NO: 449); bottom panel: Peptides 6B (SEQ ID NO: 440), 6E (SEQ ID NO: 444)) as measured by bio-layer interferometry using biotinylated peptides.

FIGS. 14A-14B are a series of diagrams showing comparative binding kinetics for anti-CD138 antibody 1610 (A) and B-B4 (B) to CD138 peptide fragments (Peptides 2A and 6B) as measured by bio-layer interferometry. The ability of mAb 1610 (but not B-B4) to bind to both peptides 2A and 6B with differential kinetics is noted.

FIGS. 15A-15C are a series of graphs showing competition for binding to cell surface CD138 between biotinylated test antibodies (anti-CD138 antibodies 1610, 624, and B-B4) and varying concentrations of corresponding, unlabeled antibodies. Differentiated profiles by epitope binning are indicated.

FIG. 16 is a graph showing induction of ADCC activity by afucosylated anti-CD138 antibodies 1610, 624, and B-B4 in U266 cells.

FIG. 17 is a table showing the mutations made in anti-CD138 antibody variants 2510, 2610, 2710, 2810, and 2910 relative to the parental antibody 1610. The protein titers produced for each of these antibodies from transiently-transfected HEK293 cells are also shown.

FIGS. 18A-18D are a series of diagrams showing binding of antibody 1610 and its variants, 2510, 2610, and 2810 to each of recombinant CD138 extracellular domain (A), Peptide 2a (B), and Peptide 6 (C) as measured by ELISA. EC50 values for each antibody variant are shown in FIG. 17D. Improvement of binding of mAb 1610 variants (relative to parental antibody 1610) to membrane proximal region (as represented by peptide 6) are noted.

FIG. 19A is a graph showing that afucosylated versions of antibody 1610 and its variants bound to cell surface CD138 expressed by U266 cells.

FIG. 19B is a series of graphs showing representative flow cytometry results for the cell binding assays summarized in FIG. 18A.

FIG. 20 is a graph showing that afucosylated versions of antibody 1610 and its variants induce ADCC activity in CD138+ U266 cells. Rabbit polyclonal (PAb) anti-CD138 antibody used as an assay control.

FIG. 21 is a graph showing the binding of CD138 peptide fragments (peptide 2A and peptide 6B) by antibody 2810 compared to BB4. Binding was measured by ELISA in a modified format in which antibody is captured directly on the ELISA plate and binding of CD138 peptides is measured at varying concentrations.

FIGS. 22A-22C are a series of diagrams showing that the antibody variant 2810 (A) binds to different portions of CD138 compared to antibody B-B4 (B). Peptide sequences are described in C. FIG. 22C discloses SEQ ID NOS 456, 10, 449, 445, 457, 440, 444, 443, 443 and 449, respectively, in order of appearance.

### DETAILED DESCRIPTION

Disclosed herein are antibody molecules that bind to CD138, e.g., human CD138. Advantageously, at least several of the antibody molecules describe herein have improved ability to inhibit cells expressing CD138, e.g., by eliciting an effector function. Without wishing to be bound by theory, it is believed that in an embodiment, anti-CD138 antibodies that bind to a desired epitope described herein have increased effector functions and preferential binding to the membrane-associated form of CD138. Targeting CD138 effectively can result in broad activity and favorable therapeutic index across myelomas and other cancers. Antibody-drug conjugates (ADCs), nucleic acid molecules encoding the antibody molecules, expression vectors, host cells, compositions (e.g., pharmaceutical compositions), kits, and methods for making the antibody molecules, are also provided. The antibody molecules and pharmaceutical compositions disclosed herein can be used (alone or in combination with other agents or therapeutic modalities) to treat, prevent and/or diagnose disorders and conditions, e.g., disorders and conditions associated with CD138, e.g., cancer or precancerous conditions.

### Definitions

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values. When “about” or “approximately” is present before a series of numbers or a range, it is understood that “about” or “approximately” can modify each of the numbers in the series or range. Similarly, when “at least,” “more than,” “no more than,” “less than,” “no less than,” or “within” is present before a series of numbers or a range, it is understood that “at least,” “more than,” “no more than,” “less than,” “no less than,” or “within” can modify each of the numbers in the series or range. As used herein, ranges include both the upper and lower limit.

The compositions and methods disclosed herein encompass polypeptides and nucleic acids having the sequences specified, or sequences substantially identical or similar thereto, e.g., sequences at least 85%, 90%, 95% identical or higher to the sequence specified.

In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a typical embodiment, the length of a reference sequence aligned for comparison purposes is at least 30%, e.g., at least 40%, 50%, 60%, e.g., at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch ((1970) J. Mol. Biol. 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In certain embodiments, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. One suitable set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid as described herein. BLAST protein searches can be performed with the XBLAST program, score=50, wordlength=3 to obtain amino acid sequences homologous to protein molecules described herein. To obtain gapped alignments for comparison purposes, Gapped BLAST can be utilized as described in Altschul et al., (1997) Nucleic Acids Res. 25:3389-3402. When utilizing BLAST and gapped BLAST programs, the default parameters of the respective programs (e.g., XBLAST and NBLAST) can be used. See www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC. 0.1% SDS at 60° C.; 3) high stringency hybridization conditions in 6×SSC at about 45° C., followed by one or more washes in 0.2×SSC. 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions 4) are suitable conditions and the ones that should be used unless otherwise specified.

It is understood that the molecules described herein may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide.” “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence.” “nucleotide sequence.” or “polynucleotide sequence.” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “treat,” a disorder, e.g., a myeloma, means that a subject (e.g., a human) who has a disorder, e.g., a myeloma, and/or experiences a symptom of a disorder, e.g., a myeloma, will, in an embodiment, suffer less a severe symptom and/or recover faster when an antibody molecule is administered than if the antibody molecule were never administered. In an embodiment, when a myeloma is treated, a bone marrow biopsy will show fewer clonal plasma cells, after effective treatment for myeloma. For example, a diagnostic assay will detect fewer clonal plasma cells in a biological sample of a subject after administration of an antibody molecule described herein for the effective treatment of a myeloma. Other assays, urine tests, or blood tests, can also be used to monitor treatment in a patient, or to detect the presence, e.g., decreased presence (or absence), of a symptom of a myeloma, after treatment of a myeloma in the subject. In an embodiment, when a myeloma is treated, the level of β2 microglobulin (β2M) in serum or urine will be decreased, after effective treatment for myeloma. Treatment can, e.g., partially or completely, alleviate, ameliorate, relieve, inhibit, or reduce the severity of, and/or reduce incidence, and optionally, delay onset of, one or more manifestations of the effects or symptoms, features, and/or causes of a disorder, e.g., a myeloma. In an embodiment, treatment is of a subject who does not exhibit certain signs of a disorder, e.g., a myeloma, and/or of a subject who exhibits only early signs of a disorder, e.g., nephropathy. In an embodiment, treatment is of a subject who exhibits one or more established signs of a disorder, e.g., a myeloma. In an embodiment, treatment is of a subject diagnosed as suffering from a disorder, e.g., a myeloma.

As used herein, the term “prevent,” a disorder, e.g., a myeloma, means that a subject (e.g., a human) is less likely to have the disorder, e.g., a myeloma, if the subject receives the antibody molecule.

Various aspects of the compositions and methods herein are described in further detail below. Additional definitions are set out throughout the specification.

### CD138

CD138 is a protein which in human is encoded by the SDC1 gene. CD138 is also known as Syndecan 1. Syndecan Proteoglycan 1. CD138 Antigen, SYND1, SDC, Syndecan-1, or Syndecan.

CD138 is a transmembrane (type I) heparan sulfate proteoglycan and is a member of the syndecan proteoglycan family. The syndecans mediate cell binding, cell signaling, and cytoskeletal organization, and syndecan receptors are required for internalization of the HIV-1 tat protein. CD138 functions as an integral membrane protein and participates in cell proliferation, cell migration and cell-matrix interactions via its receptor for extracellular matrix proteins. Altered CD138 expression has been detected in several different tumor types.

The core of CD138 includes three major domains: 1) short cytoplasmic domain; 2) plasma membrane-spanning hydrophobic domain; and 3) long extracellular domain. The functions of CD138 domains are described, e.g., in Stepp et al. Adv Wound Care (New Rochelle). 2015; 4(4):235-249). The cytoplasmic domains can transmit signals and also bind to anchoring molecules including PDZ family members. The heparan sulfate chains of CD138 also serve important biological functions. In mammals, CD138 is a major heparan sulfate proteoglycan (HSPG) on epithelial cells with high levels of expression (Fuki et al. J Clin Invest. 1997; 100(6):1611-1622). Without wishing to be bound by theory, it is believed that the HSPGs of CD138 allow the proteoglycan to bind to the heparin-binding sites present on a number of ECM proteins, growth factors, cytokines, and other proteins (Stepp et al. Adv Wound Care (New Rochelle). 2015; 4(4):235-249).

For example, the signal peptide comprises residues 1-22; the extracellular domain comprises residues 23-254; the transmembrane domain comprises residues 255-275; the cytoplasmic domain comprises residues 276-310; or the integrin binding domain (IBD) comprises residues 88-122, of a human CD138 protein, e.g., any of SEQ ID NOS: 1-3 or 450.

In an embodiment, an anti-CD138 antibody molecule described herein can modulate (e.g., inhibit) the binding of CD138 to one or more proteins that interact (e.g., bind directly or indirectly) with the extracellular domain of CD138. In an embodiment, an anti-CD138 antibody molecule described herein can modulate (e.g., inhibit) a function associated with a protein that interacts (e.g., bind directly or indirectly) with the extracellular domain of CD138. In an embodiment, a CD138-interacting protein binds to the extracellular domain of CD138 directly. In an embodiment, a CD138-interacting protein binds to the extracellular domain of CD138 through a glycosaminoglycan (GAG) chain.

Exemplary of CD138-interacting proteins and their functions are described, e.g., in Stepp et al. Adv Wound Care (New Rochelle). 2015; 4(4):235-249, the content of which is incorporated by reference in its entirety.

For example, proteins that are capable of interacting with the extracellular domain of CD138 directly or indirectly include, but are not limited to, a matrix protein (e.g., a laminin, a fibronectin, thrombospondin, collagen, fibrin, HB-GAM, tenascin, vitronectin, fibrillin, or tropoelastin), a protease (e.g., MMP7, MMP9, ADAMTS4, MT1-PPT, neutrophil elastase, cathepsin G, or carboxypeptidase), a receptor (e.g., an integrin, αᵥβ₃, αᵥβ₅, α₆β₄, α₂β₁, α₃β₁, or α_{M}β₂), a cytokine or growth factor (e.g., a morphogen (e.g., activin, BMP-2, BMP-4, chordin, Sonic Hedgehog, a Frizzled related protein, a Sprouty peptide, any of Wnt1 to Wnt13, an antiangiogenic factor (e.g., angistatin or endostatin), a growth factor (e.g., amphiregulin, batacellulin, HB-EGF, neuregulin, any of FGF1 to FGF23, PDGF, GDNF, an VEGF, HGF, TGFB1, TGFβ2, TPA, or PAI-1), or a cytokine (e.g., GM-CSF, IL-2, IL-3, IL-4, IL-5, IL-7, IL-12, interferon, TNF-α, a CC chemokine, or a CXC chemokine), a protein associated with energy balance (e.g., ApoB, ApoE, or lipoprotein lipase), a complement or coagulation protein (e.g., antithrombin II, tissue factor (TF), pathway inhibitor, Factor IX, Factor X, Factor XI, or Factor XII), or a viral or parasite coat protein (e.g., HIV-1-tat, HIV-1 gp41, HIV-1 gp120, HSV gB, HSV gC HSV gD, a coat protein of HHV-6 or HHV-8, or G-protein of RSV).

CD138 expressed on the cell surface can be cleaved by specific proteases and the shed CD138 is responsible for mediating paracrine and autocrine functions. Shed CD138 is soluble and secreted ectodomain (ECD) in blood and matrix. Shed CD138 is an indicator of poor prognosis in multiple myeloma patients and enhanced tumor progression in myeloma mouse models. Typically, shed CD138 is not considered to be primarily responsible for the disease manifestation. Translocation of CD138 to the cell nucleus can correlate to the differentiation and proliferation of certain tumor cells. In an embodiment, the anti-CD138 antibody molecules described herein preferentially target membrane-associated CD138 over soluble CD138.

CD138 is generally not present on B lymphocytes and it is expressed after the onset of plasma cell differentiation. CD138 is highly expressed on malignant plasma cells (myeloma) and has a causal role in disease progression. CD138 is implicated in various biological functions. For example, it can bind to extracellular proteins, growth factors, and chemokines; engage and activate the αVβ3 and αVβ5 integrin when clustered; regulate the biogenesis of exosomes; and regulate bone marrow microenvironment that supports myeloma growth and metastasis. Multiple signals can be attenuated by targeting CD138.

CD138 is upregulated in multiple myeloma (Tassone et al. Blood. 104(12): 3688-3696). It is overexpressed on malignant plasma cells. Multiple myeloma cells typically express between 50-200 fold higher levels of CD138. Soluble CD138 (sCD138) levels are generally from less than 60 ng/ml in normal serum to 200-1500 ng/mL in sera of multiple myeloma patients. CD138 is overexpressed in about 80% multiple myeloma patients.

CD138 can be used as a primary diagnostic marker for multiple myeloma. Increased levels of shed CD138 in serum correlated to increased tumor burden and poorer outcomes. CD138+ myeloma cells show higher proliferation and CD138+ myeloma patients have lower overall survival rates. CD138+ myeloma cells aberrantly express angiogenic factors, e.g., HGF, IL-15, ANG, APRIL, CTGF, or TGFA (Hose et al. Blood. 2009; 114(1): 128-143). Expression levels of CD138 and its released extracellular domain correlate with tumor malignancy, phenotype, and metastatic potential for both solid and hematological tumors. CD138 expression varies among cancer types, but the differential expression signatures between normal and cancer cells in epithelial and stromal compartments are directly associated with aggressiveness of tumors and patient's clinical outcome and survival.

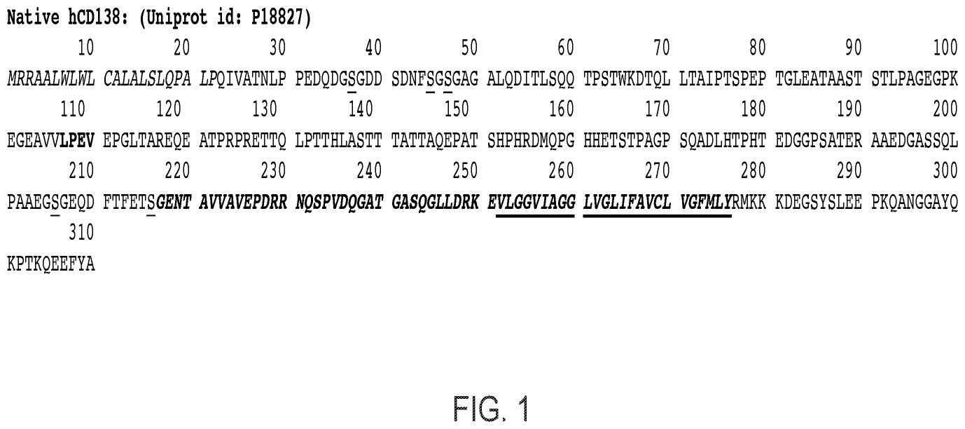

Exemplary amino acid and nucleotide sequences of human CD138 are described, e.g., in Mali et al. J Biol Chem. 1990; 265(12): 6884-6889; Lories et al. J Biol Chem. 1992; 267(2): 1116-1122; and in FIG. 1.

The amino acid sequence of an exemplary human CD138 precursor (SEQ ID NO: 1) is provided as follows.

MRRAALWLWLCALALSLQPALPQIVATNLPPEDQDGSGDDSDNFSGSGAG
ALQDITLSQQTPSTWKDTQLLTAIPTSPEPTGLEATAASTSTLPAGEGPK
EGEAVVLPEVEPGLTAREQEATPRPRETTQLPTTHQASTTTATTAQEPAT
SHPHRDMQPGHHETSTPAGPSQADLHTPHTEDGGPSATERAAEDGASSQL
PAAEGSGEQDFTFETSGENTAVVAVEPDRRNQSPVDQGATGASQGLLDRK
EVLGGVIAGGLVGLIFAVCLVGFMLYRMKKKDEGSYSLEEPKQANGGAYQ
KPTKQEEFYA

The amino acid sequence of an exemplary human CD138 precursor variant (Q136L) (SEQ ID NO: 2) is provided as follows.

MRRAALWLWLCALALSLQPALPQIVATNLPPEDQDGSGDDSDNFSGSGAG
ALQDITLSQQTPSTWKDTQLLTAIPTSPEPTGLEATAASTSTLPAGEGPK
EGEAVVLPEVEPGLTAREQEATPRPRETTQLPTTHLASTTTATTAQEPAT
SHPHRDMQPGHHETSTPAGPSQADLHTPHTEDGGPSATERAAEDGASSQL
PAAEGSGEQDFTFETSGENTAVVAVEPDRRNQSPVDQGATGASQGLLDRK
EVLGGVIAGGLVGLIFAVCLVGFMLYRMKKKDEGSYSLEEPKQANGGAYQ
KPTKQEEFYA

The amino acid sequence of an exemplary human CD138 precursor variant (T76M) (SEQ ID NO: 3) is provided as follows.

MRRAALWLWLCALALSLQPALPQIVATNLPPEDQDGSGDDSDNFSGSGAG
ALQDITLSQQTPSTWKDTQLLTAIPMSPEPTGLEATAASTSTLPAGEGPK
EGEAVVLPEVEPGLTAREQEATPRPRETTQLPTTHQASTTTATTAQEPAT
SHPHRDMQPGHHETSTPAGPSQADLHTPHTEDGGPSATERAAEDGASSQL
PAAEGSGEQDFTFETSGENTAVVAVEPDRRNQSPVDQGATGASQGLLDRK
EVLGGVIAGGLVGLIFAVCLVGFMLYRMKKKDEGSYSLEEPKQANGGAYQ
KPTKQEEFYA

The signal peptide includes amino acids 1-22 of any of SEQ ID NOs: 1-3. The mature peptide includes amino acids 23-310 of any of SEQ ID NOs: 1-3. The extracellular domain includes amino acids 23-254 of any of SEQ ID NOs: 1-3. The transmembrane domain includes amino acids 255-275 of any of SEQ ID NOs: 1-3. The cytoplasmic domain includes amino acids 276-310 of any of SEQ ID NOs: 1-3.

An exemplary coding nucleotide sequence of human CD138 (SEQ ID NO: 4) is provided as follows. This nucleotide sequence encodes the amino acid sequence of SEQ ID NO: 1.

ATGAGGCGCGCGGCGCTCTGGCTCTGGCTGTGCGCGCTGGCGCTGAGCCT
GCAGCCGGCCCTGCCGCAAATTGTGGCTACTAATTTGCCCCCTGAAGATC
AAGATGGCTCTGGGGATGACTCTGACAACTTCTCCGGCTCAGGTGCAGGT
GCTTTGCAAGATATCACCTTGTCACAGCAGACCCCCTCCACTTGGAAGGA
CACGCAGCTCCTGACGGCTATTCCCACGTCTCCAGAACCCACCGGCCTGG
AGGCTACAGCTGCCTCCACCTCCACCCTGCCGGCTGGAGAGGGGCCCAAG
GAGGGAGAGGCTGTAGTCCTGCCAGAAGTGGAGCCTGGCCTCACCGCCCG
GGAGCAGGAGGCCACCCCCCGACCCAGGGAGACCACACAGCTCCCGACCA
CTCATCAGGCCTCAACGACCACAGCCACCACGGCCCAGGAGCCCGCCACC
TCCCACCCCCACAGGGACATGCAGCCTGGCCACCATGAGACCTCAACCCC
TGCAGGACCCAGCCAAGCTGACCTTCACACTCCCCACACAGAGGATGGAG
GTCCTTCTGCCACCGAGAGGGCTGCTGAGGATGGAGCCTCCAGTCAGCTC
CCAGCAGCAGAGGGCTCTGGGGAGCAGGACTTCACCTTTGAAACCTCGGG
GGAGAATACGGCTGTAGTGGCCGTGGAGCCTGACCGCCGGAACCAGTCCC
CAGTGGATCAGGGGGCCACGGGGGCCTCACAGGGCCTCCTGGACAGGAAA
GAGGTGCTGGGAGGGGTCATTGCCGGAGGCCTCGTGGGGCTCATCTTTGC
TGTGTGCCTGGTGGGTTTCATGCTGTACCGCATGAAGAAGAAGGACGAAG
GCAGCTACTCCTTGGAGGAGCCGAAACAAGCCAACGGCGGGGCCTACCAG
AAGCCCACCAAACAGGAGGAATTCTATGCCTGA

Another exemplary coding nucleotide sequence of human CD138 (SEQ ID NO: 5) is provided as follows. This nucleotide sequence also encodes the amino acid sequence of SEQ ID NO: 1.

ATGAGGCGCGCGGCGCTCTGGCTCTGGCTGTGCGCGCTGGCGCTGAGCCT
GCAGCCGGCCCTGCCGCAAATTGTGGCTACTAATTTGCCCCCTGAAGATC
AAGATGGCTCTGGGGATGACTCTGACAACTTCTCCGGCTCAGGTGCAGGT
GCTTTGCAAGATATCACCTTGTCACAGCAGACCCCCTCCACTTGGAAGGA
CACGCAGCTCCTGACGGCTATTCCCACGTCTCCAGAACCCACCGGCCTGG
AGGCTACAGCTGCCTCCACCTCCACCCTGCCGGCTGGAGAGGGGCCCAAG
GAGGGAGAGGCTGTAGTCCTGCCAGAAGTGGAGCCTGGCCTCACCGCCCG
GGAGCAGGAGGCCACCCCCCGACCCAGGGAGACCACACAGCTCCCGACCA
CTCATCAGGCCTCAACGACCACAGCCACCACGGCCCAGGAGCCCGCCACC
TCCCACCCCCACAGGGACATGCAGCCTGGCCACCATGAGACCTCAACCCC
TGCAGGACCCAGCCAAGCTGACCTTCACACTCCCCACACAGAGGATGGAG
GTCCTTCTGCCACCGAGAGGGCTGCTGAGGATGGAGCCTCCAGTCAGCTC
CCAGCAGCAGAGGGCTCTGGGGAGCAGGACTTCACCTTTGAAACCTCGGG
GGAGAATACGGCTGTAGTGGCCGTGGAGCCTGACCGCCGGAACCAGTCCC
CAGTGGATCAGGGGGCCACGGGGGCCTCACAGGGCCTCCTGGACAGGAAA
GAGGTGCTGGGAGGGGTCATTGCCGGAGGCCTCGTGGGGCTCATCTTTGC
TGTGTGCCTGGTGGGTTTCATGCTGTACCGCATGAAGAAGAAGGACGAAG
GCAGCTACTCCTTGGAGGAGCCGAAACAAGCCAACGGCGGGGCCTACCAG
AAGCCCACCAAACAGGAGGAATTCTATGCCTGA

As used herein, when an anti-CD138 antibody molecule binds, or substantially binds, to human CD138, it binds, or substantially binds, to one or more isoforms of human CD138. In an embodiment, the antibody molecule binds or substantially binds to human CD138 having an amino acid sequence described herein, or encoded by a nucleotide sequence described herein. In an embodiment, the antibody molecule binds or substantially binds to human CD138 comprising amino acids 23-254 of any of SEQ ID NOs: 1-3.

Exemplary amino acid and nucleotide sequences of mouse CD138 are described, e.g., in Saunders et al. J Cell Biol. 1989; 108(4): 1547-1556; and Vihinen et al. J Biol Chem. 1993; 268(23): 17261-17269.

The amino acid sequence of an exemplary mouse CD138 precursor (SEQ ID NO: 6) is provided as follows.

MRRAALWLWLCALALRLQPALPQIVAVNVPPEDQDGSGDDSDNFSGSGTG
ALPDTLSRQTPSTWKDVWLLTATPTAPEPTSSNTETAFTSVLPAGEKPEE
GEPVLHVEAEPGFTARDKEKEVTTRPRETVQLPITQRASTVRVTTAQAAV
TSHPHGGMQPGLHETSAPTAPGQPDHQPPRVEGGGTSVIKEVVEDGTANQ
LPAGEGSGEQDFTFETSGENTAVAAVEPGLRNQPPVDEGATGASQSLLDR
KEVLGGVIAGGLVGLIFAVCLVAFMLYRMKKKDEGSYSLEEPKQANGGAY
QKPTKQEEFYA

The signal peptide includes amino acids 1-22 of SEQ ID NO: 6. The mature peptide includes amino acids 23-311 of SEQ ID NO: 6. The extracellular domain includes amino acids 23-255 of SEQ ID NO: 6. The transmembrane domain includes amino acids 256-276 of SEQ ID NO: 4. The cytoplasmic domain includes amino acids 277-311 of SEQ ID NO: 6.

An exemplary coding nucleotide sequence of mouse CD138 (SEQ ID NO: 7) is provided as follows.

ATGAGACGCGCGGCGCTCTGGCTCTGGCTCTGCGCGCTGGCGCTGCGCCT
GCAGCCTGCCCTCCCGCAAATTGTGGCTGTAAATGTTCCTCCTGAAGATC
AGGATGGCTCTGGGGATGACTCTGACAACTTCTCTGGCTCTGGCACAGGT
GCTTTGCCAGATACTTTGTCACGGCAGACACCTTCCACTTGGAAGGACGT
GTGGCTGTTGACAGCCACGCCCACAGCTCCAGAGCCCACCAGCAGCAACA
CCGAGACTGCTTTTACCTCTGTCCTGCCAGCCGGAGAGAAGCCCGAGGAG
GGAGAGCCTGTGCTCCATGTAGAAGCAGAGCCTGGCTTCACTGCTCGGGA
CAAGGAAAAGGAGGTCACCACCAGGCCCAGGGAGACCGTGCAGCTCCCCA
TCACCCAACGGGCCTCAACAGTCAGAGTCACCACAGCCCAGGCAGCTGTC
ACATCTCATCCGCACGGGGGCATGCAACCTGGCCTCCATGAGACCTCGGC
TCCCACAGCACCTGGTCAACCTGACCATCAGCCTCCACGTGTGGAGGGTG
GCGGCACTTCTGTCATCAAAGAGGTTGTCGAGGATGGAACTGCCAATCAG
CTTCCCGCAGGAGAGGGCTCTGGAGAACAAGACTTCACCTTTGAAACATC
TGGGGAGAACACAGCTGTGGCTGCCGTAGAGCCCGGCCTGCGGAATCAGC
CCCCGGTGGACGAAGGAGCCACAGGTGCTTCTCAGAGCCTTTTGGACAGG
AAGGAAGTGCTGGGAGGTGTCATTGCCGGAGGCCTAGTGGGCCTCATCTT
TGCTGTGTGCCTGGTGGCTTTCATGCTGTACCGGATGAAGAAGAAGGACG
AAGGCAGCTACTCCTTGGAGGAGCCCAAACAAGCCAATGGCGGTGCCTAC
CAGAAACCCACCAAGCAGGAGGAGTTCTACGCCTGA

As used herein, when an anti-CD138 antibody molecule binds, or substantially binds, to mouse CD138, it binds, or substantially binds, to one or more isoforms of mouse CD138. In an embodiment, the antibody molecule binds or substantially binds to human CD138 having an amino acid sequence described herein, or encoded by a nucleotide sequence described herein. In an embodiment, the antibody molecule binds or substantially binds to mouse CD138 comprising amino acids 23-255 of SEQ ID NO: 6.

### Epitope

The antibody molecule described herein can bind to an epitope on CD138 (e.g., human CD138). For example, an epitope bound by an antibody molecule described herein can include one or more epitope contact points described herein.

Without wishing to be bound by theory, it is believed that in an embodiment, an antibody bound to the IBD (e.g., residues 88-122 of any of SEQ ID NOS: 1-3 or 450) or any region distant from the membrane of CD138 may not be effective in signaling transduction for NK cell activation and/or may not efficiently deliver molecules such as perforins and/or granzymes for cytotoxicity.

In some embodiments, the anti-CD138 antibody molecules described herein have one, two, or all of the following properties: optimal distance of epitope from the cell membrane (e.g., not on the N-terminal of IDB); appropriate orientation of the Fc region for CD16 engagement; or proper CD138 engagement that allows for CD16 clustering on NK cells (e.g., to overcome the effect of high amount of glycosylation on CD138 molecules that may restrict the access of NK cells).

Without wishing to be bound by theory, it is believed that in an embodiment altering the position of the antibody epitope can change certain effector mechanisms engaged. For example, antibody-dependent cellular cytotoxicity (ADCC) and complement-dependent cytotoxicity (CDC) may favor a membrane-proximal epitope versus a membrane-distal epitope (Cleary et al. J Immunol. 2017; 198(10): 3999-4011). In an embodiment, antibodies designed to delete target cells through specific effector mechanisms can be selected by altering the position of the antibody epitope (e.g., the distance of epitope from membrane).

In an embodiment, the mode of engagement can affect the ability of the antibody to mediate effector functions. For example, the angle of antibody binding to the extracellular loop with regard to the membrane surface may be different (e.g., parallel or perpendicular to the membrane surface) between antibodies that bind to the same peptide epitopes.

In an embodiment, the anti-CD138 antibody molecules described herein bind to an epitope that has one, two, or all of the following properties: proximal to the cell membrane; not restricted or occluded by the glycosaminoglycan (GAG) chains; or preferentially present on membrane-associated CD138. In an embodiment, the anti-CD138 antibody molecules described herein can bind to a desired epitope region and engage with the optimal pose relative to the membrane. In an embodiment, the epitope is a linear epitope. In an embodiment, the antibody molecule binds to an extracellular region of CD138 distant from the transmembrane region. In an embodiment, the epitope is a non-contiguous or conformational epitope.

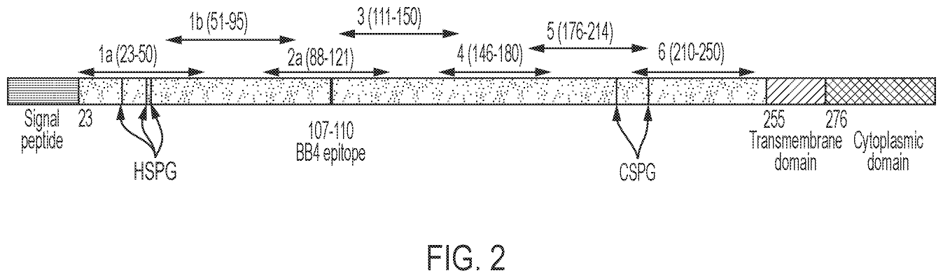

FIG. 2 shows peptides for identification of desired epitopes for anti-CD138 antibodies. Without wishing to be bound by theory, it is believed that in an embodiment, the anti-CD138 antibody molecules described herein target a peptide region between residues Gly217 to Glu251 of human CD138, e.g., as shown in FIG. 1. This region is expected to have a linear random coil conformation. In an embodiment, the anti-CD138 antibody molecule binds to at least one linear tetrapeptide in the aforesaid region. In an embodiment, the anti-CD138 antibody molecule binds to a combination of linear tetrapeptides (e.g., two, three, four, or more adjacent tetrapeptides) in the aforesaid region.

The amino acid sequences of the aforesaid peptides are shown in Table 3.

TABLE 3
Peptides for Identification of CD138 Epitopes

| | | | SEQ | |
|---|---|---|---|---|
| Peptide | Region | Amino Acid Sequence | ID NO | Length |
| Pep1a | 23-50 | QIVATNLPPEDQDGSGDDSDNFSGSGAGALQDTTLSQQT | 8 | 39 |
| Pep1b | 51-95 | ALQDTTLSQQTPSTWKDTQLLTAIPTSPEPTGLEATAASTSTLPA | 9 | 45 |
| Pep2a | 88-121 | ASTSTLPAGEGPKEGEAVVLPEVEPGLTAREQEA | 10 | 34 |
| Pep2b | 88-102 | ASTSTLPAGEGPKEG | 11 | 15 |
| Pep3 | 111-150 | EPGLTAREQEATPRPRETTQLPTTHQASTTTATTAQEPAT | 12 | 40 |
| Pep4 | 146-180 | QEPATSHPHRDMQPGHHETSTPAGPSQADLHTPHT | 13 | 35 |
| Pep5-6 | 176-250 | HTPHTEDGGPSATERAAEDGASSQLPAAEGSGEQDFTFETSGENT | 14 | 75 |
| | | AVVAVEPDRRNQSPVDQGATGASQGLLDRK | | |
| Pep5 | 176-214 | HTPHTEDGGPSATERAAEDGASSQLPAAEGSGEQDFTFE | 15 | 39 |
| Pep6 | 210-250 | DFTFETSGENTAVVAVEPDRRNQSPVDQGATGASQGLLDRK | 16 | 41 |
| Pep6a | 220-245 | TAVVAVEPDRRNQSPVDQGATGASQG | 17 | 26 |

In Table 3, the overlapping amino acids among the peptides are shown in bold; the BB4 epitope residues are shown in italic; the glycosaminoglycan (heparan sulfate, chondroitin sulfate) chain carrying serine residues are underlined. The terms “Peptide” and “Pep” are used interchangeably herein. For peptide designations, the lower case and upper-case letters are intended to have the same meaning. For example, the terms “Peptide 1A,” “Peptide 1a,” “Pep1A,” and “Pep1a” can be used to refer to the same peptide.

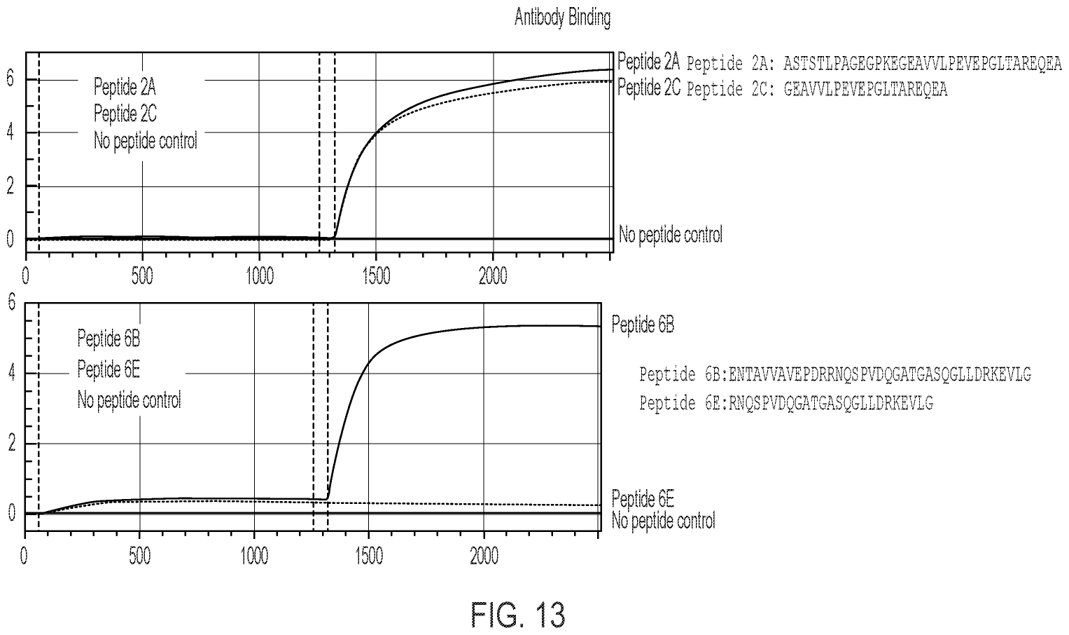

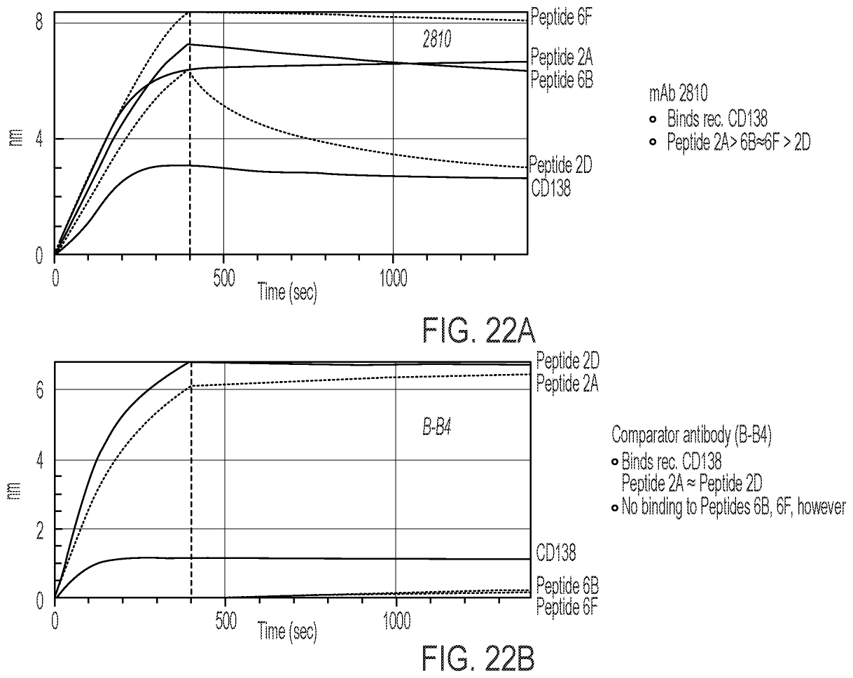

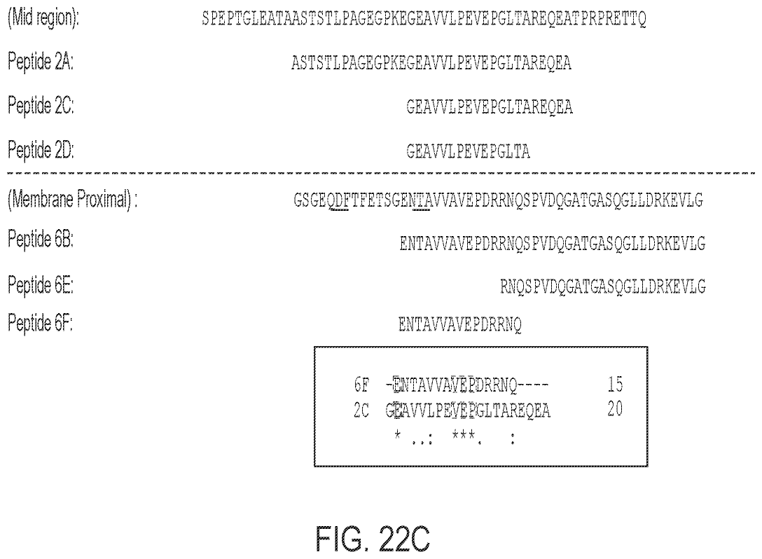

Other exemplary peptides used for identification of desired epitopes for anti-CD138 antibodies are described herein, e.g., in FIGS. 13 and 22C.

In an embodiment, the antibody molecule contacts (e.g., binds, or substantially binds, to) a region in CD138 corresponding to one or more peptides as described in Table 3, FIG. 13 or 22C. In an embodiment, the peptide is Pep6. In an embodiment, the peptide is Pep6a. In an embodiment, the peptide is Pep5. In an embodiment, the peptide is Pep4. In an embodiment, the antibody molecule contacts Pep6 or Pep6a and does not contact Pep4. In an embodiment, the antibody molecule does not contact any of Pep1a, Pep1b, Pep2a, Pep2b, Pep3, Pep4, or Pep5. In an embodiment, the antibody molecule does not contact Pep2a. In an embodiment, the antibody molecule contacts Pep2a but does not bind to the same epitope as BB4.

In an embodiment, the antibody molecule contacts Pep2a and Pep6. In an embodiment, the antibody molecule contacts Pep2a and Pep2c. In an embodiment, the antibody molecule contacts Pep6b. In an embodiment, the antibody molecule contacts Pep2a, Pep2c, and Pep6b. In an embodiment, the antibody molecule does not contact Pep6e. In an embodiment, the antibody molecule contacts Pep6b and does not contact Pep6e. In an embodiment, the antibody molecule contacts Pep2a and Pep2c and does not contact Pep6e. In an embodiment, the antibody molecule contacts Pep2a, Pep2c, and Pep6b and does not contact Pep6e.

In an embodiment, the antibody molecule contacts Pep2a and Pep2d. In an embodiment, the antibody molecule contacts Pep6b and Pep6f. In an embodiment, the antibody molecule contacts Pep2a, Pep2d, Pep6b, and Pep6f.

In an embodiment, the antibody molecule binds, or substantially binds, to CD138 in an extracellular region proximal to the transmembrane domain of CD138. In an embodiment, the C-terminus of the extracellular region proximal to the transmembrane domain is within 70, 65, 60, 55, 50, 45, 40, 35, 30, 25, 20, 15, 10, or 5 amino acids from the N-terminus of the transmembrane domain. In an embodiment, the N-terminus of the extracellular region proximal to the transmembrane domain is within 75, 70, 65, 60, 55, 50, 45, 40, 35, 30, 25, 20, 15, 10, or 5 amino acids from the N-terminus of the transmembrane domain.

In an embodiment, the antibody molecule binds to an epitope on CD138 comprising four or more (e.g., 5, 6, 7, 8, 9, 10, 11, 12, 13, 14, 15, 20, 25, 30, 35, 40, 45, 50, 55, 60, 65, 70, 75, or more) consecutive amino acid residues in the extracellular region proximal to the transmembrane domain.

In an embodiment, the antibody molecule binds to an epitope on CD138 comprising five or more consecutive amino acid residues in the extracellular region proximal to the transmembrane domain. In an embodiment, the antibody molecule binds to an epitope on CD138 comprising six or more consecutive amino acid residues in the extracellular region proximal to the transmembrane domain. In an embodiment, the antibody molecule binds to an epitope on CD138 comprising seven or more consecutive amino acid residues in the extracellular region proximal to the transmembrane domain. In an embodiment, the antibody molecule binds to an epitope on CD138 comprising eight or more consecutive amino acid residues in the extracellular region proximal to the transmembrane domain. In an embodiment, the antibody molecule binds to an epitope on CD138 comprising nine or more consecutive amino acid residues in the extracellular region proximal to the transmembrane domain. In an embodiment, the antibody molecule binds to an epitope on CD138 comprising ten or more consecutive amino acid residues in the extracellular region proximal to the transmembrane domain. In an embodiment, the antibody molecule binds to an epitope on CD138 comprising eleven or more consecutive amino acid residues in the extracellular region proximal to the transmembrane domain. In an embodiment, the antibody molecule binds to an epitope on CD138 comprising twelve or more consecutive amino acid residues in the extracellular region proximal to the transmembrane domain.

In an embodiment, the extracellular region proximal to the transmembrane domain corresponds to (e.g., comprises or consists of) Pep6. In an embodiment, the extracellular region proximal to the transmembrane domain corresponds to (e.g., comprises or consists of) Pep6a, 6b, 6e, and/or 6f. In an embodiment, the extracellular region proximal to the transmembrane domain corresponds to (e.g., comprises or consists of) Pep5.

In an embodiment, the antibody molecule contacts four or more (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41) consecutive amino acid residues in Pep6. In an embodiment, the antibody molecule contacts four or more (e.g., 5, 6, 7, 8, 9, 10, 11, 12, 13, 14, 15, 16, 17, 18, 19, 20, 21, 22, 23, 24, 25, or 26) consecutive amino acid residues in Pep6a.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38) of the following peptides (e.g., from Pep6a): DFTF (SEQ ID NO: 18); FTFE (SEQ ID NO: 19); TFET (SEQ ID NO: 20); FETS (SEQ ID NO: 21); ETSG (SEQ ID NO: 22); TSGE (SEQ ID NO: 23); SGEN (SEQ ID NO: 24); GENT (SEQ ID NO: 25); ENTA (SEQ ID NO: 26); NTAV (SEQ ID NO: 27); TAVV (SEQ ID NO: 28); AVVA (SEQ ID NO: 29); VVAV (SEQ ID NO: 30); VAVE (SEQ ID NO: 31); AVEP (SEQ ID NO: 32); VEPD (SEQ ID NO: 33); EPDR (SEQ ID NO: 34); PDRR (SEQ ID NO: 35); DRRN (SEQ ID NO: 36); RRNQ (SEQ ID NO: 37); RNQS (SEQ ID NO: 38); NQSP (SEQ ID NO: 39); QSPV (SEQ ID NO: 40); SPVD (SEQ ID NO: 41); PVDQ (SEQ ID NO: 42); VDQG (SEQ ID NO: 43); DQGA (SEQ ID NO: 44); QGAT (SEQ ID NO: 45); GATG (SEQ ID NO: 46); ATGA (SEQ ID NO: 47); TGAS (SEQ ID NO: 48); GASQ (SEQ ID NO: 49); ASQG (SEQ ID NO: 50); SQGL (SEQ ID NO: 51); QGLL (SEQ ID NO: 52); GLLD (SEQ ID NO: 53); LLDR (SEQ ID NO: 54); or LDRK (SEQ ID NO: 55).

In an embodiment, the antibody molecule contacts five or more (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41) consecutive amino acid residues in Pep6a.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37) of the following peptides (e.g., from Pep6a): DFTFE (SEQ ID NO: 56); FTFET (SEQ ID NO: 57); TFETS (SEQ ID NO: 58); FETSG (SEQ ID NO: 59); ETSGE (SEQ ID NO: 60); TSGEN (SEQ ID NO: 61); SGENT (SEQ ID NO: 62); GENTA (SEQ ID NO: 63); ENTAV (SEQ ID NO: 64); NTAVV (SEQ ID NO: 65); TAVVA (SEQ ID NO: 66); AVVAV (SEQ ID NO: 67); VVAVE (SEQ ID NO: 68); VAVEP (SEQ ID NO: 69); AVEPD (SEQ ID NO: 70); VEPDR (SEQ ID NO: 71); EPDRR (SEQ ID NO: 72); PDRRN (SEQ ID NO: 73); DRRNQ (SEQ ID NO: 74); RRNQS (SEQ ID NO: 75); RNQSP (SEQ ID NO: 76); NQSPV (SEQ ID NO: 77); QSPVD (SEQ ID NO: 78); SPVDQ (SEQ ID NO: 79); PVDQG (SEQ ID NO: 80); VDQGA (SEQ ID NO: 81); DQGAT (SEQ ID NO: 82); QGATG (SEQ ID NO: 83); GATGA (SEQ ID NO: 84); ATGAS (SEQ ID NO: 85); TGASQ (SEQ ID NO: 86); GASQG (SEQ ID NO: 87); ASQGL (SEQ ID NO: 88); SQGLL (SEQ ID NO: 89); QGLLD (SEQ ID NO: 90); GLLDR (SEQ ID NO: 91); or LLDRK (SEQ ID NO: 92).

In an embodiment, the antibody molecule contacts six or more (e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41) consecutive amino acid residues in Pep6a.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36) of the following peptides (e.g., from Pep6a): DFTFET (SEQ ID NO: 93); FTFETS (SEQ ID NO: 94); TFETSG (SEQ ID NO: 95); FETSGE (SEQ ID NO: 96); ETSGEN (SEQ ID NO: 97); TSGENT (SEQ ID NO: 98); SGENTA (SEQ ID NO: 99); GENTAV (SEQ ID NO: 100); ENTAVV (SEQ ID NO: 101); NTAVVA (SEQ ID NO: 102); TAVVAV (SEQ ID NO: 103); AVVAVE (SEQ ID NO: 104); VVAVEP (SEQ ID NO: 105); VAVEPD (SEQ ID NO: 106); AVEPDR (SEQ ID NO: 107); VEPDRR (SEQ ID NO: 108); EPDRRN (SEQ ID NO: 109); PDRRNQ (SEQ ID NO: 110); DRRNQS (SEQ ID NO: 111); RRNQSP (SEQ ID NO: 112); RNQSPV (SEQ ID NO: 113); NQSPVD (SEQ ID NO: 114); QSPVDQ (SEQ ID NO: 115); SPVDQG (SEQ ID NO: 116); PVDQGA (SEQ ID NO: 117); VDQGAT (SEQ ID NO: 118); DQGATG (SEQ ID NO: 119); QGATGA (SEQ ID NO: 120); GATGAS (SEQ ID NO: 121); ATGASQ (SEQ ID NO: 122); TGASQG (SEQ ID NO: 123); GASQGL (SEQ ID NO: 124); ASQGLL (SEQ ID NO: 125); SQGLLD (SEQ ID NO: 126); QGLLDR (SEQ ID NO: 127); or GLLDRK (SEQ ID NO: 128).

In an embodiment, the antibody molecule contacts four or more (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36) consecutive amino acid residues in Pep5.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36) of the following peptides (e.g., from Pep5): HTPH (SEQ ID NO: 129), TPHT (SEQ ID NO: 130), PHTE (SEQ ID NO: 131), HTED (SEQ ID NO: 132), TEDG (SEQ ID NO: 133), EDGG (SEQ ID NO: 134), DGGP (SEQ ID NO: 135), GGPS (SEQ ID NO: 136), GPSA (SEQ ID NO: 137), PSAT (SEQ ID NO: 138), SATE (SEQ ID NO: 139), ATER (SEQ ID NO: 140), TERA (SEQ ID NO: 141), ERAA (SEQ ID NO: 142), RAAE (SEQ ID NO: 143), AAED (SEQ ID NO: 144), AEDG (SEQ ID NO: 145), EDGA (SEQ ID NO: 146), DGAS (SEQ ID NO: 147), GASS (SEQ ID NO: 148), ASSQ (SEQ ID NO: 149), SSQL (SEQ ID NO: 150), SQLP (SEQ ID NO: 151), QLPA (SEQ ID NO: 152), LPAA (SEQ ID NO: 153), PAAE (SEQ ID NO: 154), AAEG (SEQ ID NO: 155), AEGS (SEQ ID NO: 156), EGSG (SEQ ID NO: 157), GSGE (SEQ ID NO: 158), SGEQ (SEQ ID NO: 159), GEQD (SEQ ID NO: 160), EQDF (SEQ ID NO: 161), QDFT (SEQ ID NO: 162), DFTF (SEQ ID NO: 18), or FTFE (SEQ ID NO: 19).

In an embodiment, the antibody molecule contacts five or more (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35) consecutive amino acid residues in Pep5.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35) of the following peptides (e.g., from Pep5): HTPHT (SEQ ID NO: 163), TPHTE (SEQ ID NO: 164), PHTED (SEQ ID NO: 165), HTEDG (SEQ ID NO: 166), TEDGG (SEQ ID NO: 167), EDGGP (SEQ ID NO: 168), DGGPS (SEQ ID NO: 169), GGPSA (SEQ ID NO: 170), GPSAT (SEQ ID NO: 171), PSATE (SEQ ID NO: 172), SATER (SEQ ID NO: 173), ATERA (SEQ ID NO: 174), TERAA (SEQ ID NO: 175), ERAAE (SEQ ID NO: 176), RAAED (SEQ ID NO: 177), AAEDG (SEQ ID NO: 178), AEDGA (SEQ ID NO: 179), EDGAS (SEQ ID NO: 180), DGASS (SEQ ID NO: 181), GASSQ (SEQ ID NO: 182), ASSQL (SEQ ID NO: 183), SSQLP (SEQ ID NO: 184), SQLPA (SEQ ID NO: 185), QLPAA (SEQ ID NO: 186), LPAAE (SEQ ID NO: 187), PAAEG (SEQ ID NO: 188), AAEGS (SEQ ID NO: 189), AEGSG (SEQ ID NO: 190), EGSGE (SEQ ID NO: 191), GSGEQ (SEQ ID NO: 192), SGEQD (SEQ ID NO: 193), GEQDF (SEQ ID NO: 194), EQDFT (SEQ ID NO: 195), QDFTF (SEQ ID NO: 196), or DFTFE (SEQ ID NO: 56).

In an embodiment, the antibody molecule contacts six or more (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34) consecutive amino acid residues in Pep5.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34) of the following peptides (e.g., from Pep5): HTPHTE (SEQ ID NO: 197), TPHTED (SEQ ID NO: 198), PHTEDG (SEQ ID NO: 199), HTEDGG (SEQ ID NO: 200), TEDGGP (SEQ ID NO: 201), EDGGPS (SEQ ID NO: 202), DGGPSA (SEQ ID NO: 203), GGPSAT (SEQ ID NO: 204), GPSATE (SEQ ID NO: 205), PSATER (SEQ ID NO: 206), SATERA (SEQ ID NO: 207), ATERAA (SEQ ID NO: 208), TERAAE (SEQ ID NO: 209), ERAAED (SEQ ID NO: 210), RAAEDG (SEQ ID NO: 211), AAEDGA (SEQ ID NO: 212), AEDGAS (SEQ ID NO: 213), EDGASS (SEQ ID NO: 214), DGASSQ (SEQ ID NO: 215), GASSQL (SEQ ID NO: 216), ASSQLP (SEQ ID NO: 217), SSQLPA (SEQ ID NO: 218), SQLPAA (SEQ ID NO: 219), QLPAAE (SEQ ID NO: 220), LPAAEG (SEQ ID NO: 221), PAAEGS (SEQ ID NO: 222), AAEGSG (SEQ ID NO: 223), AEGSGE (SEQ ID NO: 224), EGSGEQ (SEQ ID NO: 225), GSGEQD (SEQ ID NO: 226), SGEQDF (SEQ ID NO: 227), GEQDFT (SEQ ID NO: 228), EQDFTF (SEQ ID NO: 229), or QDFTFE (SEQ ID NO: 230).

In an embodiment, the antibody molecule does not bind, or binds with low affinity, to an extracellular region of CD138 distant from the transmembrane domain. In an embodiment, the antibody molecule does not bind to an epitope on CD138 comprising four or more (e.g., 5, 6, 7, 8, 9, 10, 11, 12, 13, 14, 15, 20, 25, 30, 35, or more) consecutive amino acid residues in an extracellular region distant from the transmembrane domain. In an embodiment, the C-terminus of the extracellular region distant from the transmembrane domain is at least 100, 110, 120, 130, 140, 150, 160, 170, 180, 190, or 200 amino acids away from the N-terminus of the transmembrane domain. In an embodiment, the extracellular region distant from the transmembrane domain corresponds to Pep1a, Pep1b, Pep2a, Pep2b, Pep2c, Pep2d, Pep3, Pep4, or a combination thereof. In an embodiment, the antibody molecule does not bind, or binds with low affinity, to the integrin binding domain (IBD) of CD138. In an embodiment, the antibody molecule does not bind, or binds with low affinity, to a region N-terminal to the IBD of CD138.

In an embodiment, the antibody molecule binds, or substantially binds, to an extracellular region of CD138 distant from the transmembrane domain. In an embodiment, the C-terminus of the extracellular region distant from the transmembrane domain is at least 100, 110, 120, 130, 140, 150, 160, 170, 180, 190, or 200 amino acids away from the N-terminus of the transmembrane domain. In an embodiment, the extracellular region distant from the transmembrane domain corresponds to Pep1a, Pep1b, Pep2a, Pep2b, Pep2c, Pep2d, Pep3, Pep4, or a combination thereof. In an embodiment, the antibody molecule binds, or substantially binds, to the integrin binding domain (IBD) of CD138. In an embodiment, the antibody molecule binds, or substantially binds, to a region N-terminal to the IBD of CD138. In an embodiment, the antibody molecule does not bind, or binds with low affinity, to the epitope of BB4.

In an embodiment, the antibody molecule binds to an epitope on CD138 comprising four or more (e.g., 5, 6, 7, 8, 9, 10, 11, 12, 13, 14, 15, 20, 25, 30, 35, 40, 45, 50, 55, 60, 65, 70, 75, or more) consecutive amino acid residues in the extracellular region distant from the transmembrane domain.

In an embodiment, the antibody molecule binds to an epitope on CD138 comprising five or more consecutive amino acid residues in the extracellular region distant to the transmembrane domain. In an embodiment, the antibody molecule binds to an epitope on CD138 comprising six or more consecutive amino acid residues in the extracellular region distant to the transmembrane domain. In an embodiment, the antibody molecule binds to an epitope on CD138 comprising seven or more consecutive amino acid residues in the extracellular region distant to the transmembrane domain. In an embodiment, the antibody molecule binds to an epitope on CD138 comprising eight or more consecutive amino acid residues in the extracellular region distant to the transmembrane domain. In an embodiment, the antibody molecule binds to an epitope on CD138 comprising nine or more consecutive amino acid residues in the extracellular region distant to the transmembrane domain. In an embodiment, the antibody molecule binds to an epitope on CD138 comprising ten or more consecutive amino acid residues in the extracellular region distant to the transmembrane domain. In an embodiment, the antibody molecule binds to an epitope on CD138 comprising eleven or more consecutive amino acid residues in the extracellular region distant to the transmembrane domain. In an embodiment, the antibody molecule binds to an epitope on CD138 comprising twelve or more consecutive amino acid residues in the extracellular region distant to the transmembrane domain.

In an embodiment, the extracellular region distant to the transmembrane domain corresponds to (e.g., comprises or consists of) Pep2a.

In an embodiment, the antibody molecule contacts four or more (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34) consecutive amino acid residues in Pep2a.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31) of the following peptides (e.g., from Pep2a): ASTS (SEQ ID NO: 231), STST (SEQ ID NO: 232), TSTL (SEQ ID NO: 233), STLP (SEQ ID NO: 234), TLPA (SEQ ID NO: 235), LPAG (SEQ ID NO: 236), PAGE (SEQ ID NO: 237), AGEG (SEQ ID NO: 238), GEGP (SEQ ID NO: 239), EGPK (SEQ ID NO: 240), GPKE (SEQ ID NO: 241), PKEG (SEQ ID NO: 242), KEGE (SEQ ID NO: 243), EGEA (SEQ ID NO: 244), GEAV (SEQ ID NO: 245), EAVV (SEQ ID NO: 246), AVVL (SEQ ID NO: 247), VVLP (SEQ ID NO: 248), VLPE (SEQ ID NO: 249), LPEV (SEQ ID NO: 250), PEVE (SEQ ID NO: 251), EVEP (SEQ ID NO: 252), VEPG (SEQ ID NO: 253), EPGL (SEQ ID NO: 254), PGLT (SEQ ID NO: 255), GLTA (SEQ ID NO: 256), LTAR (SEQ ID NO: 257), TARE (SEQ ID NO: 258), AREQ (SEQ ID NO: 259), REQE (SEQ ID NO: 260), or EQEA (SEQ ID NO: 261). In an embodiment, the antibody molecule does not contact LPEV (SEQ ID NO: 250).

In an embodiment, the antibody molecule contacts five or more (e.g., 6, 7, 8, 9, 10, 11, 12, 13, 14, 15, 16, 17, 18, 19, 20, 21, 22, 23, 24, 25, 26, 27, 28, 29, or 30) consecutive amino acid residues in Pep2a.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33) of the following peptides (e.g., from Pep2a): ASTS (SEQ ID NO: 231), STST (SEQ ID NO: 232), TSTL (SEQ ID NO: 233), STLP (SEQ ID NO: 234), TLPA (SEQ ID NO: 235), LPAG (SEQ ID NO: 236), PAGE (SEQ ID NO: 237), AGEG (SEQ ID NO: 238), GEGP (SEQ ID NO: 239), EGPK (SEQ ID NO: 240), GPKE (SEQ ID NO: 241), PKEG (SEQ ID NO: 242), KEGE (SEQ ID NO: 243), EGEA (SEQ ID NO: 244), GEAV (SEQ ID NO: 245), EAVV (SEQ ID NO: 246), AVVL (SEQ ID NO: 247), VVLP (SEQ ID NO: 248), VLPE (SEQ ID NO: 249), LPEV (SEQ ID NO: 250), PEVE (SEQ ID NO: 251), EVEP (SEQ ID NO: 252), VEPG (SEQ ID NO: 253), EPGL (SEQ ID NO: 254), PGLT (SEQ ID NO: 255), GLTA (SEQ ID NO: 256), LTAR (SEQ ID NO: 257), TARE (SEQ ID NO: 258), AREQ (SEQ ID NO: 259), REQE (SEQ ID NO: 260), or EQEA (SEQ ID NO: 261). In an embodiment, the antibody molecule does not contact a peptide comprising LPEV (SEQ ID NO: 250).

In an embodiment, the antibody molecule contacts six or more (e.g., 7, 8, 9, 10, 11, 12, 13, 14, 15, 16, 17, 18, 19, 20, 21, 22, 23, 24, 25, 26, 27, 28, or 29) consecutive amino acid residues in Pep2a.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32) of the following peptides (e.g., from Pep2a): ASTS (SEQ ID NO: 231), STST (SEQ ID NO: 232), TSTL (SEQ ID NO: 233), STLP (SEQ ID NO: 234), TLPA (SEQ ID NO: 235), LPAG (SEQ ID NO: 236), PAGE (SEQ ID NO: 237), AGEG (SEQ ID NO: 238), GEGP (SEQ ID NO: 239), EGPK (SEQ ID NO: 240), GPKE (SEQ ID NO: 241), PKEG (SEQ ID NO: 242), KEGE (SEQ ID NO: 243), EGEA (SEQ ID NO: 244), GEAV (SEQ ID NO: 245), EAVV (SEQ ID NO: 246), AVVL (SEQ ID NO: 247), VVLP (SEQ ID NO: 248), VLPE (SEQ ID NO: 249), LPEV (SEQ ID NO: 250), PEVE (SEQ ID NO: 251), EVEP (SEQ ID NO: 252), VEPG (SEQ ID NO: 253), EPGL (SEQ ID NO: 254), PGLT (SEQ ID NO: 255), GLTA (SEQ ID NO: 256), LTAR (SEQ ID NO: 257), TARE (SEQ ID NO: 258), AREQ (SEQ ID NO: 259), REQE (SEQ ID NO: 260), EQEA (SEQ ID NO: 261). In an embodiment, the antibody molecule does not contact a peptide comprising LPEV (SEQ ID NO: 250).

In an embodiment, the antibody molecule binds, or substantially binds, to an extracellular region of CD138 proximal to the transmembrane domain (e.g., an extracellular region described herein) and an extracellular region of CD138 distant from the transmembrane domain (e.g., an extracellular region described herein). In an embodiment, the antibody molecule binds to the extracellular region of CD138 proximal to the transmembrane domain with a binding affinity that is higher (e.g., at least 2, 3, 4, 5, 6, 7, 8, 9, 10, 20, 30, 40, 50, 100, 200, 300, 400, or 500-fold higher) than the binding affinity to the extracellular region of CD138 distant from the transmembrane domain. In an embodiment, the antibody molecule binds to the extracellular region of CD138 distant from the transmembrane domain with a binding affinity that is higher (e.g., at least 2, 3, 4, 5, 6, 7, 8, 9, 10, 20, 30, 40, 50, 100, 200, 300, 400, or 500-fold higher) than the binding affinity to the extracellular region of CD138 proximal to the transmembrane domain.

### Antibody Molecules

Disclosed herein are antibody molecules that bind to CD138, e.g., a CD138 molecule described herein.

As used herein, the term “antibody molecule” refers to a protein, e.g., an immunoglobulin chain or a fragment thereof, comprising at least one immunoglobulin variable domain sequence. The term “antibody molecule” includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibody molecules can be monoclonal or polyclonal. The antibody molecule can also be a human, humanized, CDR-grafted, or in vitro generated antibody. The antibody molecule can have a heavy chain constant region chosen from, e.g., IgG1. IgG2, IgG3, or IgG4. The antibody molecule can also have a light chain chosen from, e.g., kappa or lambda. The term “immunoglobulin” (Ig) is used interchangeably with the term “antibody” herein.

Examples of antigen-binding fragments include: (i) a Fab fragment, a monovalent fragment consisting of the VL, VH, CL and CHI domains; (ii) a F(ab′)2 fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments may be obtained using any suitable method, including several conventional techniques known to those with skill in the art, and the fragments can be screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

The antibody molecule can be a single chain antibody. A single-chain antibody (scFv) may be engineered (see e.g., Colcher et al. (1999) Ann N Y Acad Sci 880: 263-280; and Reiter & Pastan (1996) Clin Cancer Res 2: 245-252). The single chain antibody can be dimerized or multimerized to generate multivalent antibodies having specificities for different epitopes of the same target protein.

The antibody molecules disclosed herein can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to some aspects, a single domain antibody is a naturally occurring single domain antibody known as heavy chain antibody devoid of light chains. Such single domain antibodies are disclosed in WO 94/04678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are also contemplated.

The VH and VL regions can be subdivided into regions of hypervariability, termed “complementarity determining regions” (CDR), interspersed with regions that are more conserved, termed “framework regions” (FR or FW). The terms “complementarity determining region,” and “CDR,” as used herein refer to the sequences of amino acids within antibody variable regions which confer antigen specificity and binding affinity. As used herein, the terms “framework,” “FW” and “FR” are used interchangeably.

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). In an embodiment, the following definitions are used: AbM definition of CDR1 of the heavy chain variable domain and Kabat definitions for the other CDRs. In an embodiment, Kabat definitions are used for all CDRs. In addition, embodiments described with respect to Kabat or AbM CDRs may also be implemented using Chothia hypervariable loops. Each VH and VL typically includes three CDRs and four FRs, arranged from amino-terminus to carboxy-terminus in the following order: FR1, CDR1, FR2, CDR2, FR3, CDR3, and FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding region” refers to the part of an antibody molecule that comprises determinants that form an interface that binds to an antigen, e.g., CD138, or an epitope thereof. With respect to proteins (or protein mimetics), the antigen-binding region typically includes one or more loops (of at least, e.g., four amino acids or amino acid mimics) that form an interface that binds to the antigen, e.g., CD138. Typically, the antigen-binding region of an antibody molecule includes at least one or two CDRs and/or hypervariable loops, or more typically at least three, four, five or six CDRs and/or hypervariable loops.

The terms “compete” or “cross-compete” are used interchangeably herein to refer to the ability of an antibody molecule to interfere with binding of an anti-CD138 antibody molecule, e.g., an anti-CD138 antibody molecule provided herein, to a target, e.g., CD138. The interference with binding can be direct or indirect (e.g., through an allosteric modulation of the antibody molecule or the target). The extent to which an antibody molecule is able to interfere with the binding of another antibody molecule to the target, and therefore whether it can be said to compete, can be determined using a competition binding assay, for example, a FACS assay, an ELISA or BIACORE assay. In an embodiment, a competition binding assay is a quantitative competition assay. In an embodiment, a first anti-CD138 antibody molecule is said to compete for binding to the target with a second anti-CD138 antibody molecule when the binding of the first antibody molecule to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see e.g., Saleh et al., Cancer Immunol. Immunother., 32:180-190 (1990)) and also because of potential allergic reactions (see e.g., LoBuglio et al., Hybridoma, 5:5117-5123 (1986)).

The antibody molecule can be a polyclonal or a monoclonal antibody. In some embodiments, the antibody can be recombinantly produced, e.g., produced by any suitable phage display or combinatorial methods.

Various phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In an embodiment, the antibody molecule is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. In an embodiment, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by any suitable recombinant DNA technique. Several are known in the art (see Robinson et al., International Patent Application Publication No. WO1987/002671; Akira, et al., European Patent Application Publication No. 184,187; Taniguchi, M., European Patent Application Publication No. 171,496; Morrison et al., European Patent Application Publication No. 173,494; Neuberger et al., International Patent Application Publication No. WO 86/01533; Cabilly et al. U.S. Pat. No. 4,816,567; Cabilly et al., European Patent Application Publication No. 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to lipopolysaccharide. In an embodiment, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In some embodiments, the donor immunoglobulin is a non-human (e.g., rodent). The acceptor framework is typically a naturally-occurring (e.g., a human) framework or a consensus framework, or a sequence about 85% or higher, e.g., 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by any suitable method, and several such methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhocyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare humanized antibodies (UK Patent Application GB 2188638A, filed on Mar. 26, 1987; Winter U.S. Pat. No. 5,225,539), the contents of which is expressly incorporated by reference.

Also provided are humanized antibodies in which specific amino acids have been substituted, deleted or added. Criteria for selecting amino acids from the donor are described in, e.g., U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In an embodiment, the antibody molecule has a heavy chain constant region chosen from, e.g., the heavy chain constant regions of IgG1, IgG2 (e.g., IgG2a), IgG3, IgG4, IgM, IgA1, IgA2, IgD, and IgE; particularly, chosen from, e.g., the (e.g., human) heavy chain constant regions of IgG1. IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody molecule (e.g., to increase or decrease one or more of: Fc receptor binding, antibody glycosylation, the number of cysteine residues, effector cell function, and/or complement function). In an embodiment, the antibody molecule has effector function and can fix complement. In another embodiment, the antibody molecule does not recruit effector cells or fix complement. In certain embodiments, the antibody molecule has reduced or no ability to bind an Fc receptor. For example, it may be an isotype or subtype, fragment or other mutant, which does not support binding to an Fc receptor, e.g., it has a mutagenized or deleted Fc receptor binding region.

In an embodiment, a constant region of the antibody molecule is altered. Methods for altering an antibody constant region are known in the art. Antibody molecules s with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Amino acid mutations which stabilize antibody structure, such as S228P (EU nomenclature, S241P in Kabat nomenclature) in human IgG4 are also contemplated. Similar type of alterations could be described which if applied to the murine, or other species immunoglobulin would reduce or eliminate these functions.

In an embodiment, the Fc region is altered to extend half-life. For example, the Fc region can contain one or more of: FcMut183 (T256D-Q311V-A378V), FcMut197 (H285N-T307Q-N315D), FcMut213 (H285D-T307Q-A378V), FcMut215 (T307Q-Q311V-A378V), or FcMut228 (T256D-N286D-T307R-Q311V-A378V).

In an embodiment, the Fc region is altered to enhance ADCC. For example, the Fc region can contain one or more of: A330L-1332E-S239D, F243L-R292P-Y300L-V305I-P396L, or S298A-E333A-K334A. In an embodiment, afucosylation can be achieved by expression in a cell line such as CHO in which fucosyltransferase (FucT8) is knocked out.

In an embodiment, the Fc region is altered to enhance CDC. For example, the Fc region contains S267E-H268F-S324T.

In an embodiment, the Fc region is altered to enhance antibody-dependent cellular phagocytosis (ADCP). For example, the Fc region contains S239D-1332E-A330L.

In an embodiment, the only amino acids in the antibody molecule are canonical amino acids. In an embodiment, the antibody molecule comprises naturally-occurring amino acids; analogs, derivatives and congeners thereof; amino acid analogs having variant side chains; and/or all stereoisomers of any of any of the foregoing. The antibody molecule may comprise the D- or L-optical isomers of amino acids and peptidomimetics.

A polypeptide of an antibody molecule described herein may be linear or branched, it may comprise modified amino acids, and it may be interrupted by non-amino acids. The antibody molecule may also be modified; for example, by disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The antibody molecule described herein can be used alone in unconjugated form, or can be bound to a substance, e.g., a toxin or moiety (e.g., a therapeutic drug; a compound emitting radiation; molecules of plant, fungal, or bacterial origin; or a biological protein (e.g., a protein toxin) or particle (e.g., a recombinant viral particle, e.g., via a viral coat protein). For example, the anti-CD138 antibody can be coupled to a radioactive isotope such as an α-, β-, or γ-emitter, or a β- and γ-emitter.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a toxin, a pharmaceutical agent, and/or a protein or peptide that can mediate association of the antibody or antibody portion with another molecule (such as a streptavidin core region or a polyhistidine tag).

Some types of derivatized antibody molecule are produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an anti-CD138 antibody molecule may be derivatized (or labeled) to include fluorescent compounds, various enzymes, prosthetic groups, luminescent materials, bioluminescent materials, fluorescent emitting metal atoms, e.g., europium (Eu), and other anthanides, and radioactive materials (described below). Exemplary fluorescent detectable agents include fluorescein, fluorescein isothiocyanate, rhodamine, 5dimethylamine-1-napthalenesulfonyl chloride, phycoerythrin and the like. An antibody may also be derivatized with detectable enzymes, such as alkaline phosphatase, horseradish peroxidase, β-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody molecule may also be derivatized with a prosthetic group (e.g., streptavidin/biotin and avidin/biotin). For example, an antibody may be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibody molecules can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An antibody molecule may be conjugated to another molecular entity, typically a label or a therapeutic (e.g., antimicrobial (e.g., antibacterial or bactericidal), immunomodulatory, immunostimularoty, cytotoxic, or cytostatic) agent or moiety. Radioactive isotopes can be used in diagnostic or therapeutic applications. Radioactive isotopes that can be coupled to the antibody molecules include, but are not limited to α-, β-, or γ-emitters, or β- and γ-emitters. Such radioactive isotopes include, but are not limited to iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹mTc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), prascodymium, astatine (²¹¹At), rhenium (¹⁸⁶Rc), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³H), or one or more of the therapeutic isotopes listed above.

The present disclosure provides radiolabeled antibody molecules and methods of labeling the same. In an embodiment, a method of labeling an antibody molecule is disclosed. The method includes contacting an antibody molecule, with a chelating agent, to thereby produce a conjugated antibody. The conjugated antibody is radiolabeled with a radioisotope, e.g., ¹¹¹Indium, ⁹⁰Yttrium and ¹⁷⁷Lutetium, to thereby produce a labeled antibody molecule.

In some aspects, this disclosure provides a method of making an antibody molecule disclosed herein. The method includes: providing an antigen, e.g., CD138 or a fragment thereof; obtaining an antibody molecule that specifically binds to the antigen; evaluating efficacy of the antibody molecule in modulating activity of the antigen and/or organism expressing the antigen, e.g., CD138. The method can further include administering the antibody molecule, including a derivative thereof (e.g., a humanized antibody molecule) to a subject, e.g., a human.

This disclosure provides an isolated nucleic acid molecule encoding the above antibody molecule, vectors and host cells thereof. The nucleic acid molecule includes, but is not limited to, RNA, genomic DNA and cDNA.

Amino acid and nucleotide sequences of exemplary antibody molecules are described in Tables 1 and 2, respectively.

TABLE 1
The amino acid sequences of the heavy chain variable region (VH) and light chain
region (VL) of the exemplary anti-CD138 antibodies are provided as follows.
CDRs, defined according to the Kabat or Chothia system, are indicated.

| | | | SEQ | | | SEQ | | | SEQ |
|---|---|---|---|---|---|---|---|---|---|
| | | | ID | | | ID | | | ID |
| Antibody | Chain | Amino Acid Sequence | NO | | | NO | | | NO |
| CD001 | VH | EVQLQQSGPELVKPGASVKISCETSGFSFT | 262 | HCDR1 | GFSFTAH | 300 | HCDR1 | AHHMH | 362 |
| | | AHHMHWVKQSPEKSLEWIGEIDPNTGSTTY | | HCDR2 | DPNTGS | 301 | HCDR2 | EIDPNTGSTTYNQK | 363 |
| | | NQKFRAKATLTVDKSSNTTYMQLKSLTFED | | | | | | FRA | |
| | | SAVYYCYSNWFPYWGQGTLVTVSA | | HCDR3 | NWFPY | 302 | HCDR3 | NWFPY | 302 |
| | VL | DVVMTQTPLTLSATIGQPASIYCKSSQSLL | 263 | LCDR1 | KSSQSLLSGDGKTYLN | 303 | LCDR1 | KSSQSLLDGDGKTY | 303 |
| | | DGDGKTYLNWLLQRPGQSPKRLIYLVSKLD | | | | | | LN | |
| | | SGVPDRFTGSGSGTDFTLKISRVEAEDLGV | | LCDR2 | LVSKLDS | 304 | LCDR2 | LVDKSLD | 304 |
| | | YYCWQGTHFPRTFGGGTKLEIK | | LCDR3 | WQGTHFPRT | 305 | LCDR3 | WQGTHFPRT | 305 |
| CD002 | VH | QVQLQQPGAELVKPGASVKLSCKASGFSFI | 264 | HCDR1 | GFSFITY | 306 | HCDR1 | TYWMN | 364 |
| | | TYWMNWIKQRPGRGLEWIGRIHPSDSATQY | | HCDR2 | HPSDSA | 307 | HCDR2 | RIHPSDSATQYNQK | 365 |
| | | NQKFKTKATLTVDKSSSTAYIQLSSLTSED | | | | | | FKT | |
| | | SAVYYCARSTEGAHWGQGTLVTVSA | | HCDR3 | STEGAH | 308 | HCDR3 | STEGAH | 308 |
| | VL | DVVMTQTPLTLSVTIGQPASISCKSSQSLL | 265 | LCDR1 | KSSQSLLHSDGKTYLN | 309 | LCDR1 | KSSQSLLHSDGKTY | 309 |
| | | HSDGKTYLNWLLQRPGQSPKRLIYLVSKLD | | | | | | LN | |
| | | SGVPDRFTGSGSGTDFTLKISRVEAEDLGV | | LCDR2 | LVSKLDS | 304 | LCDR2 | LVSKLDS | 304 |
| | | YYCWQGTHFPQTFGGGTKLEIK | | LCDR3 | WQGTHFPQT | 3100 | LCDR3 | WQGTHFPQT | 310 |
| CD003 | VH | QVQLQQPGAELVKPGASVKLSCKASGYTFT | 266 | HCDR1 | GYTFTSY | 311 | HCDR1 | SFWMH | 366 |
| | | SFWMHWVKQRPGQGLEWIGEIYPSSGVTNY | | HCDR2 | YPSSGV | 312 | HCDR2 | EIYPSSGVTNYNER | 367 |
| | | NERFKNKATLTVDKSSRTAYMQLSSLTSED | | | | | | FKN | |
| | | SAVYFCTPNYYYDGLYWGQGTLVTVSA | | HCDR3 | NYYYDGLY | 313 | HCDR3 | NYYYDGLY | 313 |
| | VL | DVVMTQTPLTLSVTIGQPASISCKSSHSLL | 267 | LCDR1 | KSSHSLLYTNGETYLN | 314 | LCDR1 | KSSHSLLYTNGETY | 314 |
| | | YTNGETYLNWLLQRPGQSPKRLIYLVSNLD | | | | | | LN | |
| | | SGVPDRFSGSGSGTDFTLKISRVEAEDLGI | | LCDR2 | LVSNLDS | 315 | LCDR2 | LVSNLDS | 315 |
| | | YYCLQSTHFPRTFGGGTKLEIK | | LCDR3 | LQSTHFPRT | 316 | LCDR3 | LQSTHFPRT | 316 |
| CD004 | VH | QVQLQQPGAELVKPGASVKLSCKASGFSFT | 268 | HCDR1 | GFSFTRY | 317 | HCDR1 | RYWMN | 368 |
| | | RYWMNWVKQRPGRGLEWIGRIHPSDSASQY | | HCDR2 | HPSDSA | 307 | HCDR2 | RIHPSDSASQYNQK | 369 |
| | | NQKFKSKATLTVDKSSSTAYIQLSSLTSED | | | | | | FKS | |
| | | SAVYYCGRSTEGAYWGQGTLVTVSA | | HCDR3 | STEGAY | 318 | HCDR3 | STEGAY | 318 |
| | VL | DVVMTQTPLTLSVTIGQPASISCKSSQSLL | 265 | LCDR1 | KSSQSLLHSDGKTYLN | 309 | LCDR1 | KSSQSLLHSDGKTY | 309 |
| | | HSDGKTYLNWLLQRPGQSPKRLIYLVSKLD | | | | | | LN | |
| | | SGVPDRFTGSGSGTDFTLKISRVEAEDLGV | | LCDR2 | LVSKLDS | 304 | LCDR2 | LVSKLDS | 304 |
| | | YYCWQGTHFPQTFGGGTKLEIK | | LCDR3 | WQGTHFPQT | 310 | LCDR3 | WQGTHFPQT | 310- |
| CD005 | VH | QVQLQQPGAELVKPGASVKLSCKASGFSFI | 269 | HCDR1 | GFSFITY | 306 | HCDR1 | TYWMN | 364 |
| | | TYWMNWIKQRPGRGLEWIGRIHPSDSATQY | | HCDR2 | HPSDSA | 307 | HCDR2 | RIHPSDSATQYDQK | 370 |
| | | DQKFKTKATLTVDKSSSTAYIQLSSLTSED | | | | | | FKT | |
| | | SAVYYCARSTEGAHWGPGTLVTVSA | | HCDR3 | STEGAH | 308 | HCDR3 | STEGAH | 308 |
| | VL | DVVMTQTPLTLSVTIGQPASISCKSSHSLL | 267 | LCDR1 | KSSHSLLYTNGETYLN | 314 | LCDR1 | KSSHSLLYTNGETY | 314 |
| | | YTNGETYLNWLLQRPGQSPKRLIYLVSNLD | | | | | | LN | |
| | | SGVPDRFSGSGSGTDFTLKISRVEAEDLGI | | LCDR2 | LVSNLDS | 315 | LCDR2 | LVSNLDS | 315 |
| | | YYCLQSTHFPRTFGGGTKLEIK | | LCDR3 | LQSTHFPRT | 316 | LCDR3 | LQSTHFPRT | 316 |
| CD006 | VH | EIQLQQSGTELVKPGASVKISCKTSGYSFT | 270 | HCDR1 | GYSFTDY | 319 | HCDR1 | DYNMN | 371 |
| | | DYNMNWVKQSHGKSLEWIGNINPYYGSTGY | | HCDR2 | NPYYGS | 320 | HCDR2 | NINPYYGSTGYTQN | 372 |
| | | TQNFEGKATLTVDKSSSTAYMQLNSLTSED | | | | | | FEG | |
| | | SALYYCAREGHDYYAMDYWGQGTSVTVSA | | HCDR3 | EGHDYYAMDY | 321 | HCDR3 | EGHDYYAMDY | 321 |
| | VL | DVVMTQTPLTLSVTIGQPASISCKSSQSLL | 265 | LCDR1 | KSSQSLLHSDGKTYLN | 309 | LCDR1 | KSSQSLLHSDGKTY | 309 |
| | | HSDGKTYLNWLLQRPGQSPKRLIYLVSKLD | | | | | | LN | |
| | | SGVPDRFTGSGSGTDFTLKISRVEAEDLGV | | LCDR2 | LVSKLDS | 304 | LCDR2 | LVSKLDS | 304 |
| | | YYCWQGTHFPQTFGGGTKLEIK | | LCDR3 | WQGTHFPQT | 310 | LCDR3 | WQGTHFPQT | 310 |
| 602 | VH | QVQLQLPGAELVKPGASVKVSCKASGYTFT | 271 | HCDR1 | GYFTFSY | 322 | HCDR1 | SYWMN | 373 |
| | | SYWMHWVKQRPGQGLEWIGRIHPSDSDTNY | | HCDR2 | HPSDSD | 323 | HCDR2 | RIHPSDSDTNYNQN | 374 |
| | | NQNFKGKATLIVDKSSSTAYMQLSSLTSED | | | | | | FKG | |
| | | SAVYYCATGFSFWGQGTLVTVSA | | HCDR3 | GFSF | 324 | HCDR3 | GFSF | 324 |
| 603 | VH | QVQVQVPGAELVKPGASVKVSCKASGYTFT | 272 | HCDR1 | GYFTFSY | 322 | HCDR1 | SYWMH | 373 |
| | | SYWMHWMKKRPGQGLEWIGRIHPSDS | | HCDR2 | NPSDSD | 323 | HCDR2 | RIHSDSDTNYNQNF | 374 |
| | | DTNYNQNFKGKATLTVDKSSSTAYMQLSSL | | | | | | KG | |
| | | TSEDSAVYFCATGFSFWGQGTLVTVSA | | HCDR3 | GFSF | 324 | HCDR3 | GFSF | 324 |
| | VL | DVVMTQTPLTLSVTIGQPASISCKSSQSLL | 273 | LCDR1 | KSSQSLLYSDGKTYLN | 325 | LCDR1 | KSSQSLLYSDGKTY | 325 |
| | | YSDGKTYLNWLLQRPGESPKLLIYLVSKLD | | | | | | LN | |
| | | SGVPDRFTGSGSGTDFTLKISRVEAEDLGV | | LCDR2 | LVSKLDS | 304 | LCDR2 | LVSKLDS | 304 |
| | | YYCLQTTSFPYTFGGGTKLDIK | | LCDR3 | LQTTSFPYT | 326 | LCDR3 | LQTTSFPYT | 326 |
| 604 | VH | QVQLQQPGAELVKPGASVKVSCKASGYNFI | 274 | HCDR1 | FYNFINY | 327 | HCDR1 | NYWMH | 375 |
| | | NYWMHWVKQRPGQGLEWIGRIHPSDSYTNY | | HCDR2 | HPSDSY | 328 | HCDR2 | RIHPSDSYTNYNQK | 376 |
| | | NQKFKGKATLTVDKSSSTAYMQLSSLTSED | | | | | | FKG | |
| | | SAVYYCASPISTLYWGQGTTLTVSS | | HCDR3 | PISTLY | 329 | HCDR3 | PISTLY | 329 |
| | VL | DVVMTQTPLTLSVTIGQPASISCKSSQSLL | 275 | LCDR1 | KSSQSLLDSDGKTYLN | 330 | LCDR1 | KSSQSLLDSDGKTY | 330 |
| | | DSDGKTYLNWLLQRPGESPKLLIYLVSKLD | | | | | | LN | |
| | | SGVPDRFTGSGSGTDFTLKISRVEAEDLGV | | LCDR2 | LVSKLDS | 304 | LCDR2 | LVSKLDS | 304 |
| | | YYCLQATHFPQTFGGGTKLEIK | | LCDR3 | LQATHFPQT | 331 | LCDR3 | LQATHFPQT | 331 |
| 607 | VH | QVQLQLPGAELVRPGTSVKVSCKASDYTFT | 276 | HCDR1 | DYTFTTY | 332 | HCDR1 | TYWMN | 377 |
| | | TYWMHWVKQRPGQGLDWIGRIHPSDSDTNY | | HCDR2 | HPSDSD | 323 | HCDR2 | RIHPSDSDTNYNQN | 374 |
| | | NQNFKGKATLTVDKSSSTAYMHLSSLTSED | | | | | | FKG | |
| | | SAVYYCATGFSFWGQGTLVTVSA | | HCDR3 | GFSF | 324 | HCDR3 | GFSF | 324 |
| 613 | VH | QVQVQLPGAELVKPGASVKVSCKASGYTFT | 277 | HCDR1 | GYTFTSY | 322 | HCDR1 | SYWMN | 373 |
| | | SYWMHWVKKRPGQGLEWIGRIHPSDSDTNY | | HCDR2 | HPSDSD | 323 | HCDR2 | RIHPSDSDTNYNQN | 374 |
| | | NQNFKGKATLTVDKSSSTAYMLLSSLTSED | | | | | | FKG | |
| | | SAVYYCATGFSFWGQGTLI | | HCDR3 | GFSF | 324 | HCDR3 | GFSF | 324 |
| | VL | DVVMTQTPLTLSVTIGQPASISCKSSQSLL | 278 | LCDR1 | KSSQSLLYSDGKTYLN | 325 | LCDR1 | KSSQSLLYSDGKTY | 325 |
| | | YSDGKTYLNWLLQRPGESPELLIYLVSKMD | | | | | | LN | |
| | | SGVPDRFHGHGSGTAFTMKISRMGGGGLGN | | LCDR2 | LVSKMDS | 333 | LCDR2 | LVSKMDS | 333 |
| | | YYCLPRTSFPYTFGGGTKLEIK | | LCDR3 | LPRTSFPYT | 334 | LCDR3 | LPRTSFPYT | 334 |
| 614 | VH | QVQLQLPGAELVKPGASVKVSCKASGYTFT | 279 | HCDR1 | GYTFTSY | 322 | HCDR1 | SYWMH | 373 |
| | | SYWMHWVKQRPGQGLEWIGRIHPSDSDTNY | | HCDR2 | HPSDSD | 323 | HCDR2 | RIHPSDSDTNYNQN | 374 |
| | | NQNFKGKATLTVDKSSNTAYMQLSSLTSED | | | | | | FKG | |
| | | SAVYYCATGFSFWGQGTLVTVSA | | HCDR3 | GFSF | 324 | HCDR3 | GFSF | 324 |
| | VL | DVVMTPTSLHLLVTIGQPGFLFCKSSQNLL | 280 | LCDR1 | KSSQNLLYNEGKTYLK | 335 | LCDR1 | KSSQNLLYNEGKTY | 335 |
| | | YNEGKTYLKWLLPEPGAFSKVLIYLVFKMG | | | | | | LK | |
| | | FGVPDRFHGHGSGTDFPMKISRMGGGGLGG | | LCDR2 | LVFKMGF | 336 | LCDR2 | LVFKMGF | 336 |
| | | YLCLPSTPFPYTFGGGTKLEIK | | LCDR3 | LPSTPFPYT | 337 | LCDR3 | LPSTPFPYT | 337 |
| 616 | VH | QIHLVQSGPELKKPGETVRISCKASGYTFT | 281 | HCDR1 | GYTFTTY | 338 | HCDR1 | TYGMS | 378 |
| | | TYGMSWVKQAPGKALKWMGWINTYSGVPTY | | HCDR2 | NTYSGV | 339 | HCDR2 | WINTYSGVPTYADD | 379 |
| | | ADDFKGRFAFSLETSASTAYLQINNLKNED | | | | | | FKG | |
| | | TATYFCTREGSTMVTRYYFDYWGQGTTLTV | | HCDR3 | EGSTMVTRYYFDY | 340 | HCDR3 | EGSTMVTRYYFDY | 340 |
| | | SS | | | | | | | |
| | VL | DIVMTQAAPSVPVTPGESVSISCRSSKSLL | 282 | LCDR1 | RSSKSLLHSNGNTYLY | 341 | LCDR1 | RSSKSLLHSNGNTY | 341 |
| | | HSNGNTYLYWFLQRPGQSPQLLIYRMSNLA | | | | | | LY | |
| | | SGVPDRFSGSGSGTAFTLRISRVEAEDVGV | | LCDR2 | RMSNLAS | 342 | LCDR2 | RMSNLAS | 342 |
| | | YHCMQHLESPYTFGGGTTLEIK | | LCDR3 | MQHLESPYT | 343 | LCDR3 | MQHLESPYT | 343 |
| 617 | VH | QVQLQLPGAELVKPGASVKVSCKASAYTFT | 283 | HCDR1 | AYTFTSY | 344 | HCDR1 | SYWMH | 373 |
| | | SYWMHWVKQRPGQGLEWIGRIHPSDSDTNY | | HCDR2 | HPSDSD | 323 | HCDR2 | RIHPSDSDTNYNQN | 374 |
| | | NQNFKGKATLTVDKSSNTAYMQLSSLTSED | | | | | | FKG | |
| | | SAVYYCATGFSFWGQGTLVTVSA | | HCDR3 | GFSF | 324 | HCDR3 | GFSF | 324 |
| | VL | DIVMTQSHKFMSTSVGDRVSITCKASQDVS | 284 | LCDR1 | KASQDVSTTVA | 345 | LCDR1 | KASQDVSTTVA | 345 |
| | | TTVAWYQQKPGQSPKLLIYSASYRYTGVPD | | LCDR2 | SASYRYT | 346 | LCDR2 | SASYRYT | 346 |
| | | RFTGSGSGTDFTFTISSVQAEDLAVYYCQQ | | LCDR3 | QQHYSTRPT | 347 | LCDR3 | QQHYSTRPT | 347 |
| | | HYSTRPTFGGGTKLEIK | | | | | | | |
| 619 | VH | QIQLVQSGPELKKPGETVKISCKASGYTFT | 285 | HCDR1 | GYTFTTY | 338 | HCDR1 | TYGMS | 378 |
| | | TYGMSWVKQAPGKGLKWMGWINTYSGVPTY | | HCDR2 | NTYSGV | 339 | HCDR2 | WINTYSGVPTYADD | 379 |
| | | ADDFKGRFAFSLETSASTAYLQINNLKNED | | | | | | FKG | |
| | | TATYFCAREGSTMVTRYYFDYWGQGTTLTV | | HCDR3 | EGSTMVTRYYFDY | 340 | HCDR3 | EGSTMVTRYYFDY | 340 |
| | | SS | | | | | | | |
| | VL | DIVMTQAAPSVPVTPGESVSISCRSSKSLL | 286 | LCDR1 | RSSKSLLHSNGNTYLY | 341 | LCDR1 | RSSKSLLHSNGNTY | 341 |
| | | HSNGNTYLYWFLQRPGQSPQVLIYRMSNLA | | | | | | LY | |
| | | SGVPDRFSGSGSGTAFTLRISRVEAEDVGV | | LCDR2 | RMSNLAS | 342 | LCDR2 | RMSNALS | 342 |
| | | YYCMQHLESPYTFGGGTKLEIK | | LCDR3 | MQHSESPYT | 343 | LCDR3 | MQHLESPYT | 343 |
| 623 | VH | QIQLVQSGPELKKPGETVKISCKASGYTFT | 287 | HCDR1 | GYTFTTY | 338 | HCDR1 | TYGMS | 378 |
| | | TYGMSWVKQAPGKGLKWMGWINTYSGVPTY | | HCDR2 | NTYSGV | 339 | HCDR2 | WINTYSGVPTYADD | 379 |
| | | ADDFKGRFAFSLETSASTAYLQINNLKNED | | | | | | FKG | |
| | | TATFFCAREGSTMVTRYYFDYWGQGTTLTV | | HCDR3 | EGSTMVTRYYFDY | 340 | HCDR3 | EGSTMVTRYYFDY | 340 |
| | | SS | | | | | | | |
| | VL | DIVMTQAAPSVPVTPGESVSISCRSSKSLL | 288 | LCDR1 | RSSKSLLHSNGNTYLY | 341 | LCDR1 | RSSKSLLHSNGNTY | 341 |
| | | HSNGNTYLYWFLQRPGQSPQLLIYRMSNLA | | | | | | LY | |
| | | SGVPDRFSGSGSGTAFTLRISRVEAEDVGV | | LCDR2 | RMSNLAS | 342 | LCDR2 | RMSNLAS | 342 |
| | | YYCMQHLEYPSTFGGGTKLEIK | | LCDR3 | MQHLEPYST | 348 | LCDR3 | MQHLEYPST | 348 |
| 624 | VH | QVQVQLPGAELVKPGASVKVSCKASGYTFT | 289 | HCDR1 | GYTFTSY | 322 | HCDR1 | SYWMH | 373 |
| | | SYWMHWVKKRPGQGLEWIGRIHPSDSDTNY | | HCDR2 | HPSDSD | 323 | HCDR2 | RIHPSDSDTNYNQN | 374 |
| | | NQNFKGKATLTVDKSSSTAYMQLTSLTSED | | | | | | FKG | |
| | | FAVYYCSTGFSFWGQGTLVTVSA | | HCDR3 | GFSF | 324 | HCDR3 | GFSF | 324 |
| | VL | DVVMTQTPLTLSVTIGQPASISCKSSQSLL | 290 | LCDR1 | KSSQSLLYSDGKTYLN | 325 | LCDR1 | KSSQSLLYSDGKTY | 325 |
| | | YSDGKTYLNWLLQRPGESPKLLIYLVSKLD | | | | | | LN | |
| | | SGVPDRFTGSGSGTDFTLKISRVEAEDLGV | | LCDR2 | KVSKLDS | 304 | LCDR2 | LVSKLDS | 304 |
| | | YYCLQTTYFPYTFGGGTKLEIK | | LCDR3 | LQTTYFPYT | 349 | LCDR3 | LQTTYFPYT | 349 |
| 1610 | VH | QVQLHQPGTSLVKPGASVKLSCKASGYNFS | 291 | HCDR1 | GYNFSSY | 350 | HCDR1 | SYYMH | 380 |
| | | SYYMHWVKQRPGQGLEWIGTIHPSDSTTNC | | HCDR2 | HPSDST | 351 | HCDR2 | TIHPSDSTTNCNQK | 381 |
| | | NQKFKGKATLTVDKSSRTAYMQLNSLTFED | | | | | | FKG | |
| | | SAVYYCANFVYWGQGTSVTVSS | | HCDR3 | FVY | | HCDR3 | FVY | |
| | VL | DIVITQDELSNPVTSGDSVSISCRSSKSLL | 292 | LCDR1 | RSSKSLLYKDGKTYLN | 352 | LCDR1 | RSSKSLLYKDGKTY | 352 |
| | | YKDGKTYLNWFLQRPGQSPQLLIYVVSTRA | | | | | | LN | |
| | | SGVSDRFSGSGSGTDFTLEISRVKAEDVGV | | LCDR2 | VVSTRAS | 353 | LCDR2 | VVSTRAS | 353 |
| | | YYCQQLVEYPYTFGGGTKLEIK | | LCDR3 | QQLVEYPYT | 354 | LCDR3 | QQLVEYPYT | 354 |
| 2510 | VH | QVQLHQPGTSLVKPGASVKLSCKASGYNFS | 293 | HCDR1 | GYNFSSY | 350 | HCDR1 | SYYMH | 380 |
| | | SYYMHWVKQRPGQGLEWIGTIHPSDSTTNY | | HCDR2 | HPSDST | 351 | HCDR2 | TIHPSDSTTNYNQK | 382 |
| | | NQKFKGKATLTVDKSSRTAYMQLNSLTFED | | | | | | FKG | |
| | | SAVYYCANFVYWGQGTSVTVSS | | HCDR3 | FVY | | HCDR3 | FVY | |
| | VL | DIVITQDELSNPVTSGDSVSISCRSSKSLL | 292 | LCDR1 | RSSKSLLYKDGKTYLN | 352 | LCDR1 | RSSKSLLYKDGKTY | 352 |
| | | YKDGKTYLNWFLQRPGQSPQLLIYVVSTRA | | | | | | YN | |
| | | SGVSDRFSGSGSGTDFTLEISRVKAEDVGV | | LCDR2 | VVSTRAS | 353 | LCDR2 | VVSTRAS | 353 |
| | | YYCQQLVEYPYTFGGGTKLEIK | | LCDR3 | QQLVEYPYT | 354 | LCDR3 | QQLVEYPYT | 354 |
| 2610 | VH | QVQLHQPGTSLVKPGASVKLSCKASGYSFS | 294 | HCDR1 | GYSFSSY | 355 | HCDR1 | SYYMH | 380 |
| | | SYYMHWVKQRPGQGLEWIGTIHPSDSTTNC | | HCDR2 | HPSDST | 351 | HCDR2 | TIHPSDSTTNCNQK | 381 |
| | | NQKFKGKATLTVDKSSRTAYMQLNSLTFED | | | | | | FKG | |
| | | SAVYYCANFVYWGQGTSVTVSS | | HCDR3 | FVY | | HCDR3 | FVY | |
| | VL | DIVITQDELSNPVTSGDSVSISCRSSKSLL | 292 | LCDR1 | RSSKSLLYKDGKTYLN | 352 | LCDR1 | RSSKSLLYKDGKTY | 352 |
| | | YKDGKTYLNWFLQRPGQSPQLLIYVVSTRA | | | | | | LN | |
| | | SGVSDRFSGSGSGTDFTLEISRVKAEDVGV | | LCDR2 | VVSTRAS | 353 | LCDR2 | VVSTRAS | 353 |
| | | YYCQQLVEYPYTFGGGTKLEIK | | LCDR3 | QQLVEYPYT | 354 | LCDR3 | QQLVEYPYT | 354 |
| 2710 | VH | QVQLHQPGTSLVKPGASVKLSCKASGYTFS | 295 | HCDR1 | GYTFSSY | 356 | HCDR1 | SYYMH | 380 |
| | | SYYMHWVKQRPGQGLEWIGTIHPSDSTTNC | | HCDR2 | HPSDST | 351 | HCDR2 | TIHPSDSTTNCNQK | 381 |
| | | NQKFKGKATLTVDKSSRTAYMQLNSLTFED | | | | | | FKG | |
| | | SAVYYCANFVYWGQGTSVTVSS | | HCDR3 | FVY | | HCDR3 | FVY | |
| | VL | DIVITQDELSNPVTSGDSVSISCRSSKSLL | 292 | LCDR1 | RSSKSLLYKDGKTYLN | 352 | LCDR1 | RSSKSLLYKDGKTY | 352 |
| | | YKDGKTYLNWFLQRPGQSPQLLIYVVSTRA | | | | | | LN | |
| | | SGVSDRFSGSGSGTDFTLEISRVKAEDVGV | | LCDR2 | VVSTRAS | 353 | LCDR2 | VVSTRAS | 353 |
| | | YYCQQLVEYPYTFGGGTKLEIK | | LCDR3 | QQLVEYPYT | 354 | LCDR3 | QQLVEYPYT | 354 |
| 2810 | VH | QVQLHQPGTSLVKPGASVKLSCKASGYSFS | 296 | HCDR1 | GYSFSSY | 355 | HCDR1 | SYYMH | 380 |
| | | SYYMHWVKQRPGQGLEWIGTIHPSDSTTNY | | HCDR2 | HPSDST | 351 | HCDR2 | TIHPSDSTTNYNQK | 382 |
| | | NQKFKGKATLTVDKSSRTAYMQLNSLTFED | | | | | | FKG | |
| | | SAVYYCANFVYWGQGTSVTVSS | | HCDR3 | FVY | | HCDR3 | FVY | |
| | VL | DIVITQDELSNPVTSGDSVSISCRSSKSLL | 292 | LCDR1 | RSSKSLLYKDGKTYLN | 352 | LCDR1 | RSSKSLLYKDGKTY | 352 |
| | | YKDGKTYLNWFLQRPGQSPQLLIYVVSTRA | | | | | | LN | |
| | | SGVSDRFSGSGSGTDFTLEISRVKAEDVGV | | LCDR2 | VVSTRAS | 353 | LCDR2 | VVSTRAS | 353 |
| | | YYCQQLVEYPYTFGGGTKLEIK | | LCDR3 | QQLVEYPYT | 354 | LCDR3 | QQLVEYPYT | 354 |
| 2910 | VH | QVQLHQPGTSLVKPGASVKLSCKASGYTFS | 297 | HCDR1 | CYTFSSY | 356 | HCDR1 | SYYMH | 380 |
| | | SYYMHWVKQRPGQGLEWIGTIHPSDSTTNY | | HCDR2 | HPSDST | 351 | HCDR2 | TIHPSDSTTNYNQK | 382 |
| | | NQKFKGKATLTVDKSSRTAYMQLNSLTFED | | | | | | FKG | |
| | | SAVYYCANFVYWGQGTSVTVSS | | HCDR3 | FVY | | HCDR3 | FVY | |
| | VL | DIVITQDELSNPVTSGDSVSISCRSSKSLL | 292 | LCDR1 | RSSKSLLYKDGKTYLN | 352 | LCDR1 | RSSKSLLYKDGKTY | 352 |
| | | YKDGKTYLNWFLQRPGQSPQLLIYVVSTRA | | | | | | LN | |
| | | SGVSDRFSGSGSGTDFTLEISRVKAEDVGV | | LCDR2 | VVSTRAS | 353 | LCDR2 | VVSTRAS | 353 |
| | | YYCQQLVEYPYTFGGGTKLEIK | | LCDR3 | QQLVEYPYT | 354 | LCDR3 | QQLVEYPYT | 354 |
| 1409 | VH | EVQLVESGGGLVQPKGSLKLSCAASGFTFN | 298 | HCDR1 | GFTFNTY | 357 | HCDR1 | TYAMH | 383 |
| | | TYAMHWVRQAPGKGLEWVARIRSKSSNYAT | | HCDR2 | RSKSSNYA | 358 | HCDR2 | RIRSKSSNYATYYA | 384 |
| | | YYADSVKDRFTISRDDSQSMLYLQMNNLKT | | | | | | DSVKD | |
| | | EDTAMYYCVRELRLRYAMDYWGQGTSVTVS | | HCDR3 | ELRLRAMDY | 359 | HCDR3 | KSSQSLLYTNGKTY | 359 |
| | | S | | | | | | LN | |
| | VL | DILMTQTPLTLSVTIGQPASISCKSSQSLL | 299 | LCDR1 | KSSQSLLYTNGKTYLN | 360 | LCDR1 | KSSQSLLYTNGKTY | 360 |
| | | YTNGKTYLNWLLQRPGQSPKRLIYLVSKLD | | | | | | LN | |
| | | SGVPDRFSGSGSGTDFTLKISRVEAEDLGV | | LCDR2 | LVSKLDS | 304 | LCDR2 | LVSKLDS | 304 |
| | | YYCLQSTHFPLTFGAGTKLELK | | LCDR3 | LQSTHFPLT | 361 | LCDR3 | LQSTHFPLT | 361 |

TABLE 2
Nucleotide sequences of heavy chain variable regions
(VHs) and light chain variable regions (VLs) of exemplary anti-CD138 antibodies

| Antibody | Chain | Nucleotide Sequence | SEQ ID NO |
|---|---|---|---|
| CD001 | VH | GAAGTACAGTTGCAGCAATCTGGGCCTGAGCTGGTGAAGCCCGGTGCTTCCGTGAAAATTTCCTGCGAAAC | 385 |
| | | TTCAGGATTCTCATTTACTGCACATCATATGCACTGGGTAAAACAATCTCCAGAGAAATCACTCGAATGGA | |
| | | TAGGCGAGATTGATCCAAATACCGGGTCCACCACATACAATCAGAAATTTCGCGCTAAGGCCACCCTGACT | |
| | | GTCGATAAAAGTTCTAACACTACATACATGCAGCTTAAATCCCTTACATTCGAAGACAGTGCAGTGTACTA | |
| | | CTGTTACTCTAACTGGTTTCCATATTGGGGACAGGGAACACTGGTAACCGTTTCCGCT | |
| | VL | GACGTAGTTATGACTCAGACACCACTTACACTCTCTGCTACTATCGGACAACCAGCCTCAATCTATTGCAA | 386 |
| | | GTCCTCACAATCTTTGCTTGATGGCGACGGGAAGACCTATCTCAATTGGCTTCTCCAACGACCTGGGCAAA | |
| | | GCCCCAAGAGACTCATATATCTCGTTTCCAAGCTGGACAGTGGGGTGCCAGATAGATTTACTGGGTCAGGT | |
| | | AGTGGTACTGACTTTACTTTGAAAATATCAAGAGTAGAGGCTGAGGACCTCGGAGTCTATTACTGCTGGCA | |
| | | AGGAACCCATTTCCCCCGCACCTTCGGAGGAGGGACAAAATTGGAAATAAAA | |
| CD002 | VH | CAAGTGCAACTTCAGCAACCCGGCGCCGAGCTTGTGAAGCCTGGTGCCTCCGTTAAACTTTCTTGCAAGGC | 387 |
| | | ATCCGGTTTCTCATTCATTACCTACTGGATGAACTGGATCAAACAAAGACCTGGACGTGGTCTGGAGTGGA | |
| | | TTGGGCGGATTCACCCCTCAGACTCCGCAACCCAATACAATCAGAAATTCAAAACAAAGGCCACCTTGACC | |
| | | GTTGATAAAAGCAGTTCTACCGCTTATATTCAACTGTCCTCTCTGACCTCAGAAGACTCCGCAGTGTATTA | |
| | | CTGCGCTCGCTCTACTGAGGGTGCCCATTGGGGTCAGGGAACATTGGTGACTGTTAGTGCT | |
| | VL | GATGTTGTTATGACCCAAACTCCCCTGACACTTTCTGTAACAATAGGTCAGCCTGCCTCTATCTCATGCAA | 388 |
| | | GTCCTCACAGAGTCTGCTGCACTCTGATGGGAAGACTTATTTGAACTGGTTGCTCCAGCGCCCCGGACAGT | |
| | | CTCCTAAACGCCTGATTTATTTGGTGAGCAAGTTGGACAGTGGCGTACCAGACCGATTCACCGGATCTGGC | |
| | | TCCGGGACAGACTTTACTTTGAAAATAAGTCGTGTCGAGGCTGAGGATCTTGGCGTGTACTACTGCTGGCA | |
| | | GGGGACACACTTCCCCCAGACCTTTGGAGGTGGAACTAAGCTCGAAATCAAA | |
| CD003 | VH | AAGTACAGCTTCAGCAGCCAGGAGCAGAACTTGTTAAGCCCGGTGCTTCTGTGAAGCTGTCCTGTAAAGCT | 389 |
| | | AGTGGTTACACTTTCACTAGCTTTTGGATGCACTGGGTGAAACAGAGGCCAGGACAAGGCTTGGAGTGGAT | |
| | | TGGAGAGATATACCCTAGCAGCGGTGTGACCAACTACAATGAAAGATTTAAGAATAAAGCCACCCTGACAG | |
| | | TTGATAAATCCTCACGGACAGCATACATGCAACTCTCATCTCTGACATCCGAGGACAGCGCCGTCTATTTT | |
| | | TGTACCCCAAACTATTACTACGACGGCTTGTACTGGGGGCAGGGGACTTTGGTCACAGTGTCCGCT | |
| | VL | GATGTGGTAATGACTCAAACACCACTTACACTCAGTGTAACTATCGGCCAACCTGCCAGCATCTCCTGCAA | 390 |
| | | ATCCAGTCATAGCTTGTTGTATACCAATGGCGAGACCTATCTCAACTGGCTTCTCCAGAGGCCAGGACAGT | |
| | | CTCCCAAAAGACTTATATATTTGGTGTCTAACTTGGACTCTGGTGTGCCCGATAGATTTTCAGGGTCTGGG | |
| | | TCTGGCACCGATTTTACATTGAAAATATCCAGGGTGGAAGCCGAAGACCTTGGAATATACTACTGTCTCCA | |
| | | ATCAACCCATTTTCCTCGCACATTCGGCGGCGGCACTAAACTCGAAATAAAG | |
| CD004 | VH | CAGGTACAGCTCCAGCAACCAGGGGCAGAGTTGGTAAAGCCCGGAGCCAGTGTCAAGCTCTCATGCAAGGC | 391 |
| | | TTCCGGCTTCAGTTTCACCAGATACTGGATGAATTGGGTTAAACAGCGCCCAGGACGAGGGCTTGAATGGA | |
| | | TAGGTAGGATTCATCCCTCAGACTCAGCAAGTCAGTACAATCAGAAGTTTAAGTCCAAAGCAACACTGACA | |
| | | GTAGACAAAAGCAGCAGCACAGCTTACATTCAGTTGAGTAGCTTGACATCAGAGGATAGCGCAGTTTATTA | |
| | | TTGTGGCCGTAGTACAGAAGGGGCTTATTGGGGGCAAGGAACACTTGTCACAGTGAGTGCA | |
| | VL | GATGTTGTTATGACCCAAACTCCCCTGACACTTTCTGTAACAATAGGTCAGCCTGCCTCTATCTCATGCAA | 388 |
| | | GTCCTCACAGAGTCTGCTGCACTCTGATGGGAAGACTTATTTGAACTGGTTGCTCCAGCGCCCCGGACAGT | |
| | | CTCCTAAACGCCTGATTTATTTGGTGAGCAAGTTGGACAGTGGCGTACCAGACCGATTCACCGGATCTGGC | |
| | | TCCGGGACAGACTTTACTTTGAAAATAAGTCGTGTCGAGGCTGAGGATCTTGGCGTGTACTACTGCTGGCA | |
| | | GGGGACACACTTCCCCCAGACCTTTGGAGGTGGAACTAAGCTCGAAATCAAA | |
| CD005 | VH | CAAGTTCAATTGCAGCAGCCTGGTGCTGAGCTGGTGAAGCCAGGTGCAAGTGTTAAACTTTCATGCAAGGC | 392 |
| | | AAGCGGATTCTCCTTCATCACTTATTGGATGAATTGGATCAAACAACGTCCTGGGCGGGGCCTGGAGTGGA | |
| | | TTGGTCGCATACACCCATCTGACTCCGCTACCCAATATGACCAGAAATTCAAAACCAAAGCAACCCTCACT | |
| | | GTGGATAAAAGCAGCAGCACCGCATACATACAACTCAGCTCCCTCACTTCCGAGGACTCTGCCGTTTACTA | |
| | | TTGCGCACGAAGCACTGAAGGGGCTCATTGGGGTCCAGGAACATTGGTAACAGTCAGCGCA | |
| | VL | GATGTGGTAATGACTCAAACACCACTTACACTCAGTGTAACTATCGGCCAACCTGCCAGCATCTCCTGCAA | 390 |
| | | ATCCAGTCATAGCTTGTTGTATACCAATGGCGAGACCTATCTCAACTGGCTTCTCCAGAGGCCAGGACAGT | |
| | | CTCCCAAAAGACTTATATATTTGGTGTCTAACTTGGACTCTGGTGTGCCCGATAGATTTTCAGGGTCTGGG | |
| | | TCTGGCACCGATTTTACATTGAAAATATCCAGGGTGGAAGCCGAAGACCTTGGAATATACTACTGTCTCCA | |
| | | ATCAACCCATTTTCCTCGCACATTCGGCGGCGGCACTAAACTCGAAATAAAG | |
| CD006 | VH | GAAATACAGCTTCAGCAGTCAGGCACTGAACTGGTGAAACCCGGTGCTTCAGTGAAGATTTCCTGTAAGAC | 393 |
| | | CAGTGGTTACAGTTTCACTGATTACAACATGAACTGGGTGAAACAATCCCACGGAAAAAGTCTCGAATGGA | |
| | | TAGGTAATATAAACCCTTATTACGGAAGCACCGGCTACACTCAGAATTTTGAAGGTAAGGCTACTTTGACC | |
| | | GTGGATAAATCTTCTAGTACAGCATATATGCAGCTTAACTCACTTACTTCTGAGGACAGCGCCTTGTACTA | |
| | | CTGCGCTCGTGAAGGGCATGACTACTACGCTATGGACTACTGGGGTCAAGGCACATCTGTCACAGTCAGCT | |
| | | CA | |
| | VL | GATGTTGTTATGACCCAAACTCCCCTGACACTTTCTGTAACAATAGGTCAGCCTGCCTCTATCTCATGCAA | 388 |
| | | GTCCTCACAGAGTCTGCTGCACTCTGATGGGAAGACTTATTTGAACTGGTTGCTCCAGCGCCCCGGACAGT | |
| | | CTCCTAAACGCCTGATTTATTTGGTGAGCAAGTTGGACAGTGGCGTACCAGACCGATTCACCGGATCTGGC | |
| | | TCCGGGACAGACTTTACTTTGAAAATAAGTCGTGTCGAGGCTGAGGATCTTGGCGTGTACTACTGCTGGCA | |
| | | GGGGACACACTTCCCCCAGACCTTTGGAGGTGGAACTAAGCTCGAAATCAAA | |
| 602 | VH | CAGGTCCAACTTCAGCTGCCCGGAGCTGAACTGGTAAAACCCGGTGCTTCCGTTAAGGTGTCTTGCAAAGC | 394 |
| | | ATCAGGCTACACATTTACTAGCTACTGGATGCACTGGGTAAAGCAACGTCCAGGTCAGGGCCTTGAATGGA | |
| | | TCGGTCGTATACATCCTTCAGACTCAGATACCAATTACAATCAAAACTTTAAGGGTAAAGCTACTTTGATT | |
| | | GTCGATAAGTCTTCTTCAACTGCATACATGCAGTTGTCTTCTCTTACATCCGAGGACAGTGCAGTGTATTA | |
| | | CTGCGCTACAGGTTTCTCTTTTTGGGGACAGGGAACCCTCGTAACCGTGAGTGCC | |
| 603 | VH | CAGGTACAAGTGCAGGTGCCAGGAGCTGAGTTGGTCAAGCCAGGCGCTAGTGTGAAAGTCTCATGTAAGGC | 395 |
| | | CAGCGGCTATACTTTCACTAGTTACTGGATGCACTGGATGAAGAAGAGACCCGGACAGGGGCTCGAATGGA | |
| | | TAGGGCGAATCCACCCATCTGACAGCGATACAAATTACAACCAGAACTTTAAAGGAAAGGCAACACTTACA | |
| | | GTTGATAAGTCTAGCAGCACAGCATACATGCAGCTTAGTTCACTCACATCAGAAGATTCCGCTGTCTATTT | |
| | | TTGTGCTACTGGTTTCAGCTTTTGGGGTCAGGGAACTCTCGTAACTGTGTCCGCA | |
| | VL | GATGTCGTTATGACCCAGACTCCATTGACTCTGTCTGTCACCATAGGACAACCCGCATCTATCTCCTGCAA | 396 |
| | | ATCATCACAGAGCTTGCTGTATTCTGACGGAAAGACATATTTGAACTGGCTGCTCCAACGGCCTGGGGAGT | |
| | | CCCCTAAACTCCTTATCTATCTCGTTTCTAAACTTGACAGTGGCGTCCCTGATCGTTTTACCGGCTCCGGG | |
| | | TCTGGCACTGATTTTACACTCAAGATCAGCCGGGTGGAAGCAGAGGATTTGGGTGTCTACTATTGTCTTCA | |
| | | GACCACTTCCTTCCCATATACCTTCGGCGGCGGAACTAAATTGGAAATCAAA | |
| 604 | VH | CAAGTCCAGTTGCAGCAGCCCGGTGCTGAGCTTGTCAAACCCGGCGCCTCAGTTAAAGTCTCATGCAAGGC | 397 |
| | | TTCTGGCTATAACTTTATAAATTACTGGATGCACTGGGTCAAACAGCGACCAGGACAGGGCCTCGAATGGA | |
| | | TTGGTAGAATACACCCATCAGATAGTTACACTAATTACAATCAGAAGTTTAAAGGTAAGGCAACACTGACT | |
| | | GTGGACAAAAGCAGCTCAACTGCCTACATGCAGCTCAGTTCTCTCACCTCCGAGGATAGTGCTGTGTACTA | |
| | | TTGTGCCAGTCCCATATCCACTCTTTATTGGGGGCAGGGCACCACCTTGACCGTATCCTCA | |
| | VL | GATGTCGTGATGACTCAAACTCCATTGACTCTGAGCGTCACTATTGGGCAACCTGCTAGTATATCATGCAA | 398 |
| | | GTCCTCTCAGTCTCTGTTGGACTCCGACGGGAAGACTTATCTCAACTGGTTGCTGCAACGTCCTGGTGAGA | |
| | | GCCCCAAGCTCCTTATATACCTGGTATCAAAACTGGATTCTGGGGTTCCAGACCGTTTCACTGGGAGCGGG | |
| | | AGCGGCACAGACTTTACCCTCAAGATTTCACGGGTAGAAGCTGAAGACCTGGGAGTGTATTACTGCCTTCA | |
| | | AGCCACACATTTTCCTCAAACATTTGGGGGTGGTACTAAGCTGGAAATTAAG | |
| 607 | VH | CAAGTTCAGTTGCAGCTTCCTGGAGCTGAGTTGGTTCGGCCAGGTACATCAGTTAAAGTAAGCTGCAAAGC | 399 |
| | | AAGCGACTACACCTTCACCACATATTGGATGCACTGGGTCAAACAGCGGCCTGGACAGGGGCTGGACTGGA | |
| | | TCGGGAGGATACATCCTAGCGATTCTGATACTAACTACAATCAGAATTTCAAAGGTAAAGCCACACTCACT | |
| | | GTGGACAAATCCTCTTCAACCGCTTACATGCACTTGTCATCCTTGACATCCGAGGACTCAGCAGTTTATTA | |
| | | CTGCGCTACCGGTTTCAGCTTTTGGGGACAGGGTACTTTGGTGACAGTGAGCGCC | |
| 613 | VH | CAGGTTCAAGTGCAACTCCCTGGTGCCGAACTTGTGAAGCCCGGAGCCAGTGTGAAGGTTAGCTGTAAGGC | 400 |
| | | CTCTGGGTACACATTTACTTCCTACTGGATGCACTGGGTAAAAAAGCGGCCAGGACAGGGACTCGAATGGA | |
| | | TAGGACGTATTCACCCTTCCGACTCTGACACAAACTACAACCAAAACTTCAAAGGTAAAGCCACTCTCACC | |
| | | GTAGACAAATCATCATCAACCGCATACATGCTCCTCTCATCCCTGACATCAGAAGACAGTGCTGTTTATTA | |
| | | TTGCGCTACAGGGTTTAGTTTTTGGGGCCAAGGAACCTTGATTACCGTGTCCGCA | |
| | VL | GACGTGGTGATGACTCAGACACCTCTGACCCTGTCTGTAACCATTGGCCAGCCAGCCAGTATTAGTTGTAA | 401 |
| | | ATCATCTCAAAGTCTCCTCTACTCAGACGGCAAGACCTATTTGAACTGGTTGCTCCAGCGGCCAGGCGAAT | |
| | | CACCCGAGCTGCTCATTTACTTGGTCTCCAAGATGGATTCCGGTGTGCCAGATAGATTTCATGGTCACGGA | |
| | | AGTGGGACAGCCTTCACAATGAAGATTTCCCGGATGGGCGGCGGTGGATTGGGAAACTATTACTGTCTCCC | |
| | | TCGTACCTCCTTCCCTTACACTTTCGGTGGTGGGACAAAACTCGAGATAAAA | |
| 614 | VH | CAAGTGCAGTTGCAGCTCCCCGGTGCCGAACTCGTAAAACCCGGCGCAAGCGTGAAAGTTTCCTGTAAGGC | 402 |
| | | ATCCGGCTATACATTCACATCATATTGGATGCATTGGGTCAAACAGCGTCCTGGGCAGGGTCTTGAATGGA | |
| | | TTGGGCGGATACATCCATCTGACAGTGATACCAACTACAATCAAAATTTTAAAGGGAAGGCCACCCTCACA | |
| | | GTTGACAAGTCTAGTAATACAGCCTACATGCAGCTTTCTAGCCTGACTAGCGAGGATTCTGCTGTTTACTA | |
| | | CTGTGCAACCGGATTCAGTTTTTGGGGACAAGGAACTTTGGTGACAGTATCCGCC | |
| | VL | GACGTGGTGATGACCCCAACATCACTTCATTTGCTTGTTACTATAGGGCAACCCGGCTTTTTGTTCTGTAA | 403 |
| | | AAGTTCACAGAATCTCCTCTACAATGAAGGAAAAACATACTTGAAGTGGCTTTTGCCTGAGCCAGGTGCTT | |
| | | TCTCCAAGGTACTTATATACCTTGTCTTCAAGATGGGATTTGGGGTTCCTGATCGCTTCCACGGCCACGGA | |
| | | TCTGGCACCGACTTCCCTATGAAAATAAGCCGAATGGGAGGGGGCGGCCTTGGGGGCTACCTTTGCCTTCC | |
| | | CTCTACCCCCTTTCCTTATACCTTCGGCGGGGGTACTAAACTTGAAATAAAA | |
| 616 | VH | CAGATCCACTTGGTACAGTCTGGACCTGAGCTGAAGAAGCCTGGAGAGACAGTCAGGATCTCCTGCAAGGC | 404 |
| | | TTCTGGGTATACCTTCACAACCTATGGAATGAGCTGGGTGAAGCAGGCTCCAGGAAAGGCTTTAAAGTGGA | |
| | | TGGGCTGGATAAACACCTACTCTGGAGTGCCAACATATGCTGATGACTTCAAGGGACGGTTTGCCTTCTCT | |
| | | TTGGAAACCTCTGCCAGCACTGCCTATTTGCAGATCAACAACCTCAAAAATGAGGACACGGCTACATATTT | |
| | | CTGTACAAGAGAGGGATCTACTATGGTTACGAGGTACTACTTTGACTACTGGGGCCAAGGCACCACTCTCA | |
| | | CAGTCTCCTCA | |
| | VL | GACATTGTTATGACCCAAGCCGCCCCAAGCGTACCAGTTACTCCTGGCGAGAGTGTCTCCATTAGTTGTCG | 405 |
| | | GTCTTCAAAAAGTTTGCTCCACTCCAATGGGAATACTTACCTTTATTGGTTCCTTCAGCGTCCTGGTCAAT | |
| | | CTCCACAGCTGCTGATTTATCGAATGAGTAACCTGGCCTCAGGAGTCCCTGATCGCTTCAGTGGTTCAGGG | |
| | | TCCGGTACTGCCTTTACACTTAGGATCTCCAGGGTAGAAGCCGAGGATGTAGGCGTCTACCATTGTATGCA | |
| | | ACATCTCGAATCACCCTATACTTTCGGTGGAGGTACAAAACTCGAAATAAAA | |
| 617 | VH | CAAGTACAACTGCAACTCCCAGGCGCCGAGTTGGTTAAACCTGGCGCTTCAGTGAAGGTATCCTGCAAAGC | 406 |
| | | ATCTGCCTACACTTTCACATCTTACTGGATGCACTGGGTAAAACAGCGACCAGGGCAGGGACTTGAATGGA | |
| | | TTGGACGCATTCATCCTTCCGATAGCGACACTAACTATAACCAAAATTTTAAGGGGAAGGCCACCTTGACT | |
| | | GTGGATAAATCTAGCAACACAGCCTACATGCAACTCAGTTCACTGACTTCTGAGGATTCTGCCGTTTATTA | |
| | | TTGTGCCACAGGCTTCTCCTTCTGGGGGCAAGGAACCTTGGTGACCGTGTCAGCT | |
| | VL | GACATAGTAATGACTCAAAGCCACAAATTCATGTCCACCAGTGTTGGTGACCGCGTATCAATCACTTGCAA | 407 |
| | | GGCCAGTCAGGACGTATCCACAACAGTTGCATGGTATCAGCAAAAGCCAGGACAATCACCCAAACTTCTGA | |
| | | TTTACAGTGCCAGTTATCGATACACTGGGGTTCCCGACAGATTCACAGGATCAGGCAGCGGAACTGATTTT | |
| | | ACCTTCACCATTAGCTCAGTGCAAGCCGAAGATCTGGCCGTGTATTATTGTCAACAGCACTATAGTACCAG | |
| | | GCCCACCTTCGGCGGGGGAACTAAATTGGAAATAAAG | |
| 691 | VH | CAGATCCAGTTGGTACAGTCTGGACCTGAGCTGAAGAAGCCTGGAGAGACAGTCAAGATCTCCTGCAAGGC | 408 |
| | | TTCTGGGTATACCTTCACAACCTATGGAATGAGCTGGGTGAAACAGGCTCCAGGAAAGGGTTTAAAGTGGA | |
| | | TGGGCTGGATAAACACCTACTCTGGAGTGCCAACATATGCTGATGACTTCAAGGGACGGTTTGCCTTCTCT | |
| | | TTGGAAACCTCTGCCAGCACTGCCTATTTGCAGATCAACAACCTCAAAAATGAGGACACGGCTACATATTT | |
| | | CTGTGCAAGAGAGGGATCTACTATGGTTACGAGGTACTACTTTGACTACTGGGGCCAAGGCACCACTCTCA | |
| | | CAGTCTCCTCA | |
| | VL | GATATTGTGATGACCCAAGCTGCCCCCTCCGTCCCCGTCACACCCGGTGAGTCCGTGTCTATAAGCTGTCG | 409 |
| | | TAGTTCCAAGAGCTTGCTTCACTCAAATGGCAATACATACCTTTATTGGTTCCTGCAACGCCCCGGCCAGA | |
| | | GCCCACAGGTGTTGATTTATCGTATGTCAAACCTGGCCTCCGGCGTTCCCGACAGGTTTTCCGGCAGTGGA | |
| | | AGCGGGACCGCATTTACACTGCGAATATCTCGTGTTGAGGCAGAAGACGTTGGAGTCTATTACTGTATGCA | |
| | | ACACCTCGAAAGCCCATACACTTTCGGCGGTGGGACTAAGCTGGAAATTAAA | |
| 623 | VH | CAGATCCAGTTGGTTCAGTCTGGACCTGAGCTGAAGAAGCCTGGAGAGACAGTCAAGATCTCCTGCAAGGC | 410 |
| | | TTCTGGGTATACCTTCACAACCTATGGAATGAGCTGGGTGAAACAGGCTCCAGGAAAGGGTTTAAAGTGGA | |
| | | TGGGCTGGATAAACACCTACTCTGGAGTGCCAACATATGCTGATGACTTCAAGGGACGGTTTGCCTTCTCT | |
| | | TTGGAAACCTCTGCCAGCACTGCCTATTTGCAGATCAACAACCTCAAAAATGAGGACACGGCTACGTTTTT | |
| | | CTGTGCAAGAGAGGGATCTACTATGGTTACGAGGTACTACTTTGACTACTGGGGCCAAGGCACCACTCTCA | |
| | | CAGTCTCCTCA | |
| | VL | GATATTGTCATGACCCAGGCAGCCCCCAGTGTCCCCGTGACTCCTGGAGAAAGTGTTAGTATTAGCTGTCG | 411 |
| | | ATCAAGTAAATCACTTCTTCATAGTAACGGAAATACTTACTTGTATTGGTTCCTCCAAAGGCCAGGCCAGT | |
| | | CTCCACAGTTGCTCATCTATCGCATGAGTAATCTTGCTTCAGGTGTGCCTGATCGCTTCAGTGGCAGTGGA | |
| | | TCAGGTACTGCTTTCACACTCCGTATAAGTAGGGTGGAAGCCGAGGATGTCGGTGTCTACTATTGTATGCA | |
| | | GCACCTGGAGTATCCCTCAACATTTGGTGGGGGGACAAAACTGGAGATTAAG | |
| 624 | VH | CAAGTCCAGGTGCAACTGCCTGGCGCCGAACTTGTGAAACCCGGAGCCTCCGTTAAGGTCTCCTGCAAGGC | 412 |
| | | TAGTGGCTATACCTTTACATCTTATTGGATGCACTGGGTGAAAAAACGCCCAGGGCAGGGCCTCGAATGGA | |
| | | TCGGCCGCATCCACCCATCTGATAGCGACACTAACTATAACCAGAACTTTAAAGGCAAGGCTACTCTGACC | |
| | | GTTGATAAAAGCAGTTCCACTGCCTACATGCAACTGACATCCCTTACCAGTGAGGATTTCGCCGTGTACTA | |
| | | CTGCTCCACAGGGTTCTCCTTCTGGGGCCAGGGGACCCTTGTTACCGTGTCCGCA | |
| | VL | GATGTCGTTATGACCCAGACTCCATTGACTCTGTCTGTCACCATAGGACAACCCGCATCTATCTCCTGCAA | 413 |
| | | ATCATCACAGAGCTTGCTGTATTCTGACGGAAAGACATATTTGAACTGGCTGCTCCAACGGCCTGGGGAGT | |
| | | CCCCTAAACTCCTTATCTATCTCGTTTCTAAACTTGACAGTGGCGTCCCTGATCGTTTTACCGGCTCCGGG | |
| | | TCTGGCACTGATTTTACACTCAAGATCAGCCGGGTGGAAGCAGAGGATTTGGGTGTCTACTATTGTCTTCA | |
| | | GACCACTTACTTCCCATATACCTTCGGCGGCGGAACTAAATTGGAAATCAAA | |
| 1610 | VH | CAAGTTCAGTTGCACCAACCTGGTACAAGCCTCGTTAAGCCCGGTGCGAGTGTCAAACTTAGCTGCAAAGC | 414 |
| | | ATCTGGTTACAATTTTTCCAGTTATTACATGCACTGGGTTAAACAGCGGCCCGGCCAAGGACTGGAGTGGA | |
| | | TCGGAACCATCCACCCCTCAGACTCAACTACGAACTGCAATCAGAAGTTCAAGGGGAAGGCCACGCTTACC | |
| | | GTGGACAAGTCAAGTAGGACTGCTTACATGCAACTCAATAGCTTGACATTCGAGGATTCCGCGGTCTATTA | |
| | | TTGTGCGAATTTCGTCTATTGGGGACAAGGTACCAGCGTGACGGTCTCCAGC | |
| | VL | GACATTGTTATTACGCAAGACGAGCTGTCAAACCCTGTTACGAGTGGTGATTCTGTATCCATATCCTGTCG | 415 |
| | | CTCCTCAAAAAGTCTGTTGTACAAGGATGGAAAAACTTATCTGAACTGGTTTCTGCAACGGCCAGGCCAAT | |
| | | CTCCTCAATTGCTTATATACGTCGTTTCAACGAGAGCCTCAGGAGTGTCTGACAGATTTTCCGGCTCCGGC | |
| | | TCTGGGACCGATTTTACTCTCGAAATCAGCCGGGTTAAGGCCGAAGACGTTGGTGTGTATTATTGCCAACA | |
| | | GCTCGTAGAGTACCCATATACATTCGGCGGGGGCACAAAACTCGAAATAAAG | |
| 2510 | VH | CAAGTTCAGTTGCACCAACCTGGTACAAGCCTCGTTAAGCCCGGTGCGAGTGTCAAACTTAGCTGCAAAGC | 416 |
| | | ATCTGGTTACAATTTTTCCAGTTATTACATGCACTGGGTTAAACAGCGGCCCGGCCAAGGACTGGAGTGGA | |
| | | TCGGAACCATCCACCCCTCAGACTCAACTACGAACTACAATCAGAAGTTCAAGGGGAAGGCCACGCTTACC | |
| | | GTGGACAAGTCAAGTAGGACTGCTTACATGCAACTCAATAGCTTGACATTCGAGGATTCCGCGGTCTATTA | |
| | | TTGTGCGAATTTCGTCTATTGGGGACAAGGTACCAGCGTGACGGTCTCCAGC | |
| | VL | GACATTGTTATTACGCAAGACGAGCTGTCAAACCCTGTTACGAGTGGTGATTCTGTATCCATATCCTGTCG | 415 |
| | | CTCCTCAAAAAGTCTGTTGTACAAGGATGGAAAAACTTATCTGAACTGGTTTCTGCAACGGCCAGGCCAAT | |
| | | CTCCTCAATTGCTTATATACGTCGTTTCAACGAGAGCCTCAGGAGTGTCTGACAGATTTTCCGGCTCCGGC | |
| | | TCTGGGACCGATTTTACTCTCGAAATCAGCCGGGTTAAGGCCGAAGACGTTGGTGTGTATTATTGCCAACA | |
| | | GCTCGTAGAGTACCCATATACATTCGGCGGGGGCACAAAACTCGAAATAAAG | |
| 2610 | VH | CAAGTTCAGTTGCACCAACCTGGTACAAGCCTCGTTAAGCCCGGTGCGAGTGTCAAACTTAGCTGCAAAGC | 417 |
| | | ATCTGGTTACAGCTTTTCCAGTTATTACATGCACTGGGTTAAACAGCGGCCCGGCCAAGGACTGGAGTGGA | |
| | | TCGGAACCATCCACCCCTCAGACTCAACTACGAACTGCAATCAGAAGTTCAAGGGGAAGGCCACGCTTACC | |
| | | GTGGACAAGTCAAGTAGGACTGCTTACATGCAACTCAATAGCTTGACATTCGAGGATTCCGCGGTCTATTA | |
| | | TTGTGCGAATTTCGTCTATTGGGGACAAGGTACCAGCGTGACGGTCTCCAGC | |
| | VL | GACATTGTTATTACGCAAGACGAGCTGTCAAACCCTGTTACGAGTGGTGATTCTGTATCCATATCCTGTCG | 415 |
| | | CTCCTCAAAAAGTCTGTTGTACAAGGATGGAAAAACTTATCTGAACTGGTTTCTGCAACGGCCAGGCCAAT | |
| | | CTCCTCAATTGCTTATATACGTCGTTTCAACGAGAGCCTCAGGAGTGTCTGACAGATTTTCCGGCTCCGGC | |
| | | TCTGGGACCGATTTTACTCTCGAAATCAGCCGGGTTAAGGCCGAAGACGTTGGTGTGTATTATTGCCAACA | |
| | | GCTCGTAGAGTACCCATATACATTCGGCGGGGGCACAAAACTCGAAATAAAG | |
| 2710 | VH | CAAGTTCAGTTGCACCAACCTGGTACAAGCCTCGTTAAGCCCGGTGCGAGTGTCAAACTTAGCTGCAAAGC | 418 |
| | | ATCTGGTTACACCTTTTCCAGTTATTACATGCACTGGGTTAAACAGCGGCCCGGCCAAGGACTGGAGTGGA | |
| | | TCGGAACCATCCACCCCTCAGACTCAACTACGAACTGCAATCAGAAGTTCAAGGGGAAGGCCACGCTTACC | |
| | | GTGGACAAGTCAAGTAGGACTGCTTACATGCAACTCAATAGCTTGACATTCGAGGATTCCGCGGTCTATTA | |
| | | TTGTGCGAATTTCGTCTATTGGGGACAAGGTACCAGCGTGACGGTCTCCAGC | |
| | VL | GACATTGTTATTACGCAAGACGAGCTGTCAAACCCTGTTACGAGTGGTGATTCTGTATCCATATCCTGTCG | 415 |
| | | CTCCTCAAAAAGTCTGTTGTACAAGGATGGAAAAACTTATCTGAACTGGTTTCTGCAACGGCCAGGCCAAT | |
| | | CTCCTCAATTGCTTATATACGTCGTTTCAACGAGAGCCTCAGGAGTGTCTGACAGATTTTCCGGCTCCGGC | |
| | | TCTGGGACCGATTTTACTCTCGAAATCAGCCGGGTTAAGGCCGAAGACGTTGGTGTGTATTATTGCCAACA | |
| | | GCTCGTAGAGTACCCATATACATTCGGCGGGGGCACAAAACTCGAAATAAAG | |
| 2810 | VH | CAAGTTCAGTTGCACCAACCTGGTACAAGCCTCGTTAAGCCCGGTGCGAGTGTCAAACTTAGCTGCAAAGC | 419 |
| | | ATCTGGTTACAGCTTTTCCAGTTATTACATGCACTGGGTTAAACAGCGGCCCGGCCAAGGACTGGAGTGGA | |
| | | TCGGAACCATCCACCCCTCAGACTCAACTACGAACTACAATCAGAAGTTCAAGGGGAAGGCCACGCTTACC | |
| | | GTGGACAAGTCAAGTAGGACTGCTTACATGCAACTCAATAGCTTGACATTCGAGGATTCCGCGGTCTATTA | |
| | | TTGTGCGAATTTCGTCTATTGGGGACAAGGTACCAGCGTGACGGTCTCCAGC | |
| | VL | GACATTGTTATTACGCAAGACGAGCTGTCAAACCCTGTTACGAGTGGTGATTCTGTATCCATATCCTGTCG | 415 |
| | | CTCCTCAAAAAGTCTGTTGTACAAGGATGGAAAAACTTATCTGAACTGGTTTCTGCAACGGCCAGGCCAAT | |
| | | CTCCTCAATTGCTTATATACGTCGTTTCAACGAGAGCCTCAGGAGTGTCTGACAGATTTTCCGGCTCCGGC | |
| | | TCTGGGACCGATTTTACTCTCGAAATCAGCCGGGTTAAGGCCGAAGACGTTGGTGTGTATTATTGCCAACA | |
| | | GCTCGTAGAGTACCCATATACATTCGGCGGGGGCACAAAACTCGAAATAAAG | |
| 2910 | VH | CAAGTTCAGTTGCACCAACCTGGTACAAGCCTCGTTAAGCCCGGTGCGAGTGTCAAACTTAGCTGCAAAGC | 420 |
| | | ATCTGGTTACACCTTTTCCAGTTATTACATGCACTGGGTTAAACAGCGGCCCGGCCAAGGACTGGAGTGGA | |
| | | TCGGAACCATCCACCCCTCAGACTCAACTACGAACTACAATCAGAAGTTCAAGGGGAAGGCCACGCTTACC | |
| | | GTGGACAAGTCAAGTAGGACTGCTTACATGCAACTCAATAGCTTGACATTCGAGGATTCCGCGGTCTATTA | |
| | | TTGTGCGAATTTCGTCTATTGGGGACAAGGTACCAGCGTGACGGTCTCCAGC | |
| | VL | GACATTGTTATTACGCAAGACGAGCTGTCAAACCCTGTTACGAGTGGTGATTCTGTATCCATATCCTGTCG | 415 |
| | | CTCCTCAAAAAGTCTGTTGTACAAGGATGGAAAAACTTATCTGAACTGGTTTCTGCAACGGCCAGGCCAAT | |
| | | CTCCTCAATTGCTTATATACGTCGTTTCAACGAGAGCCTCAGGAGTGTCTGACAGATTTTCCGGCTCCGGC | |
| | | TCTGGGACCGATTTTACTCTCGAAATCAGCCGGGTTAAGGCCGAAGACGTTGGTGTGTATTATTGCCAACA | |
| | | GCTCGTAGAGTACCCATATACATTCGGCGGGGGCACAAAACTCGAAATAAAG | |
| 1409 | VH | GAAGTTCAATTGGTTGAGTCAGGGGGCGGTCTTGTTCAACCTAAAGGCTCCCTCAAGTTGTCCTGTGCAGC | 421 |
| | | CTCTGGATTTACGTTTAACACTTATGCTATGCACTGGGTTCGGCAAGCACCGGGGAAAGGGCTCGAGTGGG | |
| | | TGGCCCGCATTAGATCAAAATCATCCAACTATGCCACCTACTATGCCGATTCCGTGAAGGACAGATTCACA | |
| | | ATATCACGCGATGATAGCCAAAGTATGCTCTATTTGCAAATGAATAATCTTAAAACCGAAGACACAGCTAT | |
| | | GTATTATTGTGTCAGAGAGTTGAGACTTAGGTATGCTATGGATTACTGGGGCCAAGGTACTTCAGTGACCG | |
| | | TTTCATCC | |
| | VL | GATATACTGATGACCCAAACTCCACTGACTCTGTCTGTCACCATCGGTCAGCCCGCATCAATCAGTTGTAA | 422 |
| | | ATCTAGTCAGTCCCTGCTGTATACTAACGGAAAGACTTATCTGAATTGGCTTTTGCAACGGCCCGGTCAAT | |
| | | CACCCAAAAGGCTTATATACCTGGTAAGCAAGTTGGACAGTGGAGTTCCGGATCGCTTCAGTGGCTCTGGT | |
| | | AGTGGGACAGATTTTACGCTCAAAATTAGTAGGGTGGAGGCCGAGGATCTTGGCGTCTATTATTGCCTCCA | |
| | | ATCTACGCACTTTCCACTCACGTTTGGGGCCGGAACCAAACTCGAACTTAAA | |

TABLE 4
The amino acid sequences of the heavy chain variable region (VH) and light chain
variable region (VL) of antibody B-B4 are provided as follows.
CDRs, defined according to the Kabat or Chothia system, are indicated.

| | | | SEQ | | | SEQ | | | SEQ |
|---|---|---|---|---|---|---|---|---|---|
| | | | ID | | | ID | | | ID |
| Antibody | Chain | Amino Acid Sequence | NO | Chothia CDR | | NO | Kabat CDR | | NO |
| BB4 | VH | QVQLQQSGSELMMPGASVKISCKATG | 423 | HCDR1 | GYTFSNY | 425 | HCDR1 | NYWIE | 431 |
| | | YTFSNYWIEWVKQRPGHGLEWIGEIL | | HCDR2 | LPGTGR | 426 | HCDR2 | EILPGTGRTIYNEKFKG | 432 |
| | | PGTGRTIYNEKFKGKATFTADISSNT | | HCDR3 | RDYYGNFYYAMDY | 427 | HCDR3 | RDYYGNFYYAMDY | 427 |
| | | VQMQLSSLTSEDSAVYYCARRDYYGN | | | | | | | |
| | | FYYAMDYWGQGTSVTVSS | | | | | | | |
| | VL | DIQMTQSTSSLSASLGDRVTISCSAS | 424 | LCDR1 | SASQGINNYLN | 428 | LCDR1 | SASQGINNYLN | 428 |
| | | QGINNYLNWYQQKPDGTVELLIYYTS | | LCDR2 | YTSTLQS | 429 | LCDR2 | YTSTLQS | 429 |
| | | TLQSGVPSRFSGSGSGTDYSLTISNL | | LCDR3 | QQYSKLPRT | 430 | LCDR3 | QQYSKLPRT | 430 |
| | | EPEDIGTYYCQQYSKLPRTFGGGTKL | | | | | | | |
| | | EIK | | | | | | | |

TABLE 5
Nucleotide sequences of heavy chain variable regions (VHs)
and light chain variable regions (VLs) of antibody B-B4

| Antibody | Chain | Nucleotide Sequence | SEQ ID NO |
|---|---|---|---|
| BB4 | VH | CAGGTTCAGTTGCAGCAGTCTGGTTCCGAATTGATGATGCCAGGAGCTTCCGTGAAGATAAGCTGTAAG | 433 |
| | | GCCACAGGTTACACTTTCAGTAACTATTGGATAGAATGGGTAAAGCAAAGACCTGGTCACGGTTTGGAA | |
| | | TGGATCGGGGAGATACTGCCTGGTACCGGCAGAACTATCTACAACGAGAAATTTAAGGGTAAAGCCACT | |
| | | TTTACAGCAGACATATCCAGTAATACAGTTCAAATGCAGCTGTCATCACTCACCAGTGAAGATAGCGCC | |
| | | GTGTATTACTGCGCCAGGCGCGATTATTACGGCAACTTTTATTATGCTATGGATTACTGGGGCCAAGGT | |
| | | ACTTCTGTAACTGTAAGCTCC | |
| | VL | GATATACAGATGACGCAGTCTACTTCTTCCCTCTCTGCGTCCCTTGGCGACCGGGTCACAATAAGCTGT | 434 |
| | | TCTGCTTCCCAGGGTATAAATAACTACCTGAATTGGTATCAGCAAAAACCGGATGGGACGGTCGAACTC | |
| | | CTGATATATTACACATCTACACTTCAGTCTGGTGTCCCCTCTCGCTTTTCAGGTTCCGGTTCCGGCACT | |
| | | GATTATAGCCTTACAATTAGCAACCTCGAACCGGAGGACATCGGAACATATTATTGCCAGCAATATAGT | |
| | | AAACTGCCCAGGACGTTTGGCGGTGGCACCAAGTTGGAAATCAAA | |

In an embodiment, the antibody molecule comprises one, two, or three CDRs of the VH region of an antibody molecule described herein, e.g., in Table 1, using the Kabat or Chothia definitions of CDRs. In an embodiment, the antibody molecule comprises one, two, or three CDRs of the VL region of an antibody molecule described herein, e.g., in Table 1, using the Kabat or Chothia definitions of CDRs. In an embodiment, the antibody molecule comprises one or more (e.g., two or three) CDRs of the VH region and one or more (e.g., two or three) CDRs of the VL region of an antibody molecule described herein, e.g., in Table 1, using the Kabat or Chothia definitions of CDRs.

In an embodiment, the antibody molecule comprises one, two, or three HCDRs described in Table 1. In an embodiment, the antibody molecule comprises one, two, or three LCDRs described in Table 1. In an embodiment, the antibody molecule comprises one or more (e.g., two or three) HCDRs and one or more (e.g., two or three) LCDRs described in Table 1.

In an embodiment, the antibody molecule comprises one, two, three, or four frameworks of the VH region of an antibody molecule described in Table 1. In an embodiment, the antibody molecule comprises one, two, three, or four frameworks of the VL region of an antibody molecule described in Table 1. In an embodiment, the antibody molecule comprises one or more (e.g., two, three, or four) frameworks of the VH region and one or more (e.g., two, three, or four) frameworks of the VL region of an antibody molecule described in Table 1.

In an embodiment, the antibody molecule comprises a VH of an antibody molecule described herein, e.g., in Table 1. In an embodiment, the antibody molecule comprises a VL of an antibody molecule described herein, e.g., in Table 1. In an embodiment, the antibody molecule comprises a VH and a VL of an antibody molecule described herein, e.g., in Table 1.

In an embodiment, the antibody molecule comprises a VH having an amino acid sequence described in Table 1, or an amino acid sequence substantially identical thereof (e.g., differing by no more than 1, 2, 3, 4, 5, 6, 7, 8, 9, 10, 11, 12, 13, 14, or 15 amino acid residues therefrom, or at least 85, 90, 95, or 99% identical thereto). In an embodiment, the antibody molecule comprises a VL having an amino acid sequence described in Table 1, or an amino acid sequence substantially identical thereof (e.g., differing by no more than 1, 2, 3, 4, 5, 6, 7, 8, 9, 10, 11, 12, 13, 14, or 15 amino acid residues therefrom, or at least 85, 90, 95, or 99% identical thereto). In an embodiment, the antibody molecule comprises a VH having an amino acid sequence described in Table 1 (or an amino acid sequence substantially identical thereof) and a VL having an amino acid sequences described in Table 1 (or an amino acid sequence substantially identical thereof).

In an embodiment, the antibody molecule comprises a VH encoded by a nucleotide sequence described in Table 2, or a nucleotide sequence substantially identical thereof (e.g., differing by no more than 3, 6, 15, 30, or 45 nucleotides therefrom, or at least about 85%, 90%, 95%, or 99% identical thereto). In an embodiment, the antibody molecule comprises a VL encoded by a nucleotide sequence described in Table 2, or a nucleotide sequence substantially identical thereof (e.g., differing by no more than 3, 6, 15, 30, or 45 nucleotides therefrom, or at least about 85%, 90%, 95%, or 99% identical thereto). In an embodiment, the antibody molecule comprises a VH encoded by a nucleotide sequence described in Table 2 (or a nucleotide sequence substantially identical thereof) and a VL encoded by a nucleotide sequence described in Table 2 (or a nucleotide sequence substantially identical thereof).

In an embodiment, the VH comprises one, two, or all of: (i) an HCDR1 comprising an amino acid sequence of GYNFSSY (SEQ ID NO: 350); (ii) an HCDR2 comprising an amino acid sequence of HPSDST (SEQ ID NO: 351); or (iii) an HCDR3 comprising an amino acid sequence of FVY; and the VL comprises one, two, or all of: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); or (iii) an LCDR3 comprising an amino acid sequence of QQLVEYPYT (SEQ ID NO: 354). In an embodiment, the VH comprises: (i) an HCDR1 comprising an amino acid sequence of GYNFSSY (SEQ ID NO: 350); (ii) an HCDR2 comprising an amino acid sequence of HPSDST (SEQ ID NO: 351); and (iii) an HCDR3 comprising an amino acid sequence of FVY; and the VL comprises: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); and (iii) an LCDR3 comprising an amino acid sequence of QQLVEYPYT (SEQ ID NO: 354).

In an embodiment, the VH comprises one, two, or all of: (i) an HCDR1 comprising an amino acid sequence of SYYMH (SEQ ID NO: 380); (ii) an HCDR2 comprising an amino acid sequence of TIHPSDSTTNCNQKFKG (SEQ ID NO: 381); or (iii) an HCDR3 comprising an amino acid sequence of FVY; and the VL comprises one, two, or all of: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); or (iii) an LCDR3 comprising an amino acid sequence of QQLVEYPYT (SEQ ID NO: 354). In an embodiment, the VH comprises: (i) an HCDR1 comprising an amino acid sequence of SYYMH (SEQ ID NO: 380); (ii) an HCDR2 comprising an amino acid sequence of TIHPSDSTTNCNQKFKG (SEQ ID NO: 381); and (iii) an HCDR3 comprising an amino acid sequence of FVY; and the VL comprises: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); and (iii) an LCDR3 comprising an amino acid sequence of QQLVEYPYT (SEQ ID NO: 354).

In an embodiment, the VH comprises the amino acid sequence of SEQ ID NO: 291. In an embodiment, the VL comprises the amino acid sequence of SEQ ID NO: 292. In an embodiment, the VH comprises the amino acid sequence of SEQ ID NO: 291 and the VL comprises the amino acid sequence of SEQ ID NO: 292.

In an embodiment, the VH comprises one, two, or all of: (i) an HCDR1 comprising an amino acid sequence of GYNFSSY (SEQ ID NO: 350); (ii) an HCDR2 comprising an amino acid sequence of HPSDST (SEQ ID NO: 351); or (iii) an HCDR3 comprising an amino acid sequence of FVY; and the VL comprises one, two, or all of: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); or (iii) an LCDR3 comprising an amino acid sequence of QQLVEYPYT (SEQ ID NO: 354). In an embodiment, the VH comprises: (i) an HCDR1 comprising an amino acid sequence of GYNFSSY (SEQ ID NO: 350); (ii) an HCDR2 comprising an amino acid sequence of HPSDST (SEQ ID NO: 351); and (iii) an HCDR3 comprising an amino acid sequence of FVY; and the VL comprises: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); and (iii) an LCDR3 comprising an amino acid sequence of QQLVEYPYT (SEQ ID NO: 354).

In an embodiment, the VH comprises one, two, or all of: (i) an HCDR1 comprising an amino acid sequence of SYYMH (SEQ ID NO: 380); (ii) an HCDR2 comprising an amino acid sequence of TIHPSDSTTNYNQKFKG (SEQ ID NO: 382); or (iii) an HCDR3 comprising an amino acid sequence of FVY; and the VL comprises one, two, or all of: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); or (iii) an LCDR3 comprising an amino acid sequence of QQLVEYPYT (SEQ ID NO: 354). In an embodiment, the VH comprises: (i) an HCDR1 comprising an amino acid sequence of SYYMH (SEQ ID NO: 380); (ii) an HCDR2 comprising an amino acid sequence of TIHPSDSTTNYNQKFKG (SEQ ID NO: 382); and (iii) an HCDR3 comprising an amino acid sequence of FVY; and the VL comprises: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); and (iii) an LCDR3 comprising an amino acid sequence of QQLVEYPYT (SEQ ID NO: 354).

In an embodiment, the VH comprises the amino acid sequence of SEQ ID NO: 293. In an embodiment, the VL comprises the amino acid sequence of SEQ ID NO: 292. In an embodiment, the VH comprises the amino acid sequence of SEQ ID NO: 293 and the VL comprises the amino acid sequence of SEQ ID NO: 292.

In an embodiment, the VH comprises one, two, or all of: (i) an HCDR1 comprising an amino acid sequence of GYSFSSY (SEQ ID NO: 355); (ii) an HCDR2 comprising an amino acid sequence of HPSDST (SEQ ID NO: 351); or (iii) an HCDR3 comprising an amino acid sequence of FVY; and the VL comprises one, two, or all of: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); or (iii) an LCDR3 comprising an amino acid sequence of QQLVEYPYT (SEQ ID NO: 354). In an embodiment, the VH comprises: (i) an HCDR1 comprising an amino acid sequence of GYSFSSY (SEQ ID NO: 355); (ii) an HCDR2 comprising an amino acid sequence of HPSDST (SEQ ID NO: 351); and (iii) an HCDR3 comprising an amino acid sequence of FVY; and the VL comprises three light chain complementarity determining regions (LCDR1, LCDR2, and LCDR3), wherein the VL comprises: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); and (iii) an LCDR3 comprising an amino acid sequence of QQLVEYPYT (SEQ ID NO: 354).

In an embodiment, VH comprises one, two, or all of: (i) an HCDR1 comprising an amino acid sequence of SYYMH (SEQ ID NO: 380); (ii) an HCDR2 comprising an amino acid sequence of TIHPSDSTTNCNQKFKG (SEQ ID NO: 381); or (iii) an HCDR3 comprising an amino acid sequence of FVY; and the VL comprises one, two, or all of: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); or (iii) an LCDR3 comprising an amino acid sequence of QQLVEYPYT (SEQ ID NO: 354). In an embodiment, VH comprises: (i) an HCDR1 comprising an amino acid sequence of SYYMH (SEQ ID NO: 380); (ii) an HCDR2 comprising an amino acid sequence of TIHPSDSTTNCNQKFKG (SEQ ID NO: 381); and (iii) an HCDR3 comprising an amino acid sequence of FVY; and the VL comprises: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); and (iii) an LCDR3 comprising an amino acid sequence of QQLVEYPYT (SEQ ID NO: 354).

In an embodiment, the VH comprises the amino acid sequence of SEQ ID NO: 294. In an embodiment, the VL comprises the amino acid sequence of SEQ ID NO: 292. In an embodiment, the VH comprises the amino acid sequence of SEQ ID NO: 294 and the VL comprises the amino acid sequence of SEQ ID NO: 292.

In an embodiment, the VH comprises one, two, or all of: (i) an HCDR1 comprising an amino acid sequence of GYTESSY (SEQ ID NO: 356); (ii) an HCDR2 comprising an amino acid sequence of HPSDST (SEQ ID NO: 351); or (iii) an HCDR3 comprising an amino acid sequence of FVY; and the VL comprises one, two, or all of: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); or (iii) an LCDR3 comprising an amino acid sequence of QQLVEYPYT (SEQ ID NO: 354). In an embodiment, the VH comprises: (i) an HCDR1 comprising an amino acid sequence of GYTFSSY (SEQ ID NO: 356); (ii) an HCDR2 comprising an amino acid sequence of HPSDST (SEQ ID NO: 351); and (iii) an HCDR3 comprising an amino acid sequence of FVY; and the VL comprises: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); and (iii) an LCDR3 comprising an amino acid sequence of QQLVEYPYT (SEQ ID NO: 354).

In an embodiment, the VH comprises one, two, or all of: (i) an HCDR1 comprising an amino acid sequence of SYYMH (SEQ ID NO: 380); (ii) an HCDR2 comprising an amino acid sequence of TIHPSDSTTNCNQKFKG (SEQ ID NO: 381); or (iii) an HCDR3 comprising an amino acid sequence of FVY; and the VL comprises one, two, or all of: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); or (iii) an LCDR3 comprising an amino acid sequence of QQLVEYPYT (SEQ ID NO: 354). In an embodiment, the VH comprises: (i) an HCDR1 comprising an amino acid sequence of SYYMH (SEQ ID NO: 380); (ii) an HCDR2 comprising an amino acid sequence of TIHPSDSTTNCNQKFKG (SEQ ID NO: 381); and (iii) an HCDR3 comprising an amino acid sequence of FVY; and the VL comprises: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); and (iii) an LCDR3 comprising an amino acid sequence of QQLVEYPYT (SEQ ID NO: 354).

In an embodiment, the VH comprises the amino acid sequence of SEQ ID NO: 295. In an embodiment, the VL comprises the amino acid sequence of SEQ ID NO: 292. In an embodiment, the VH comprises the amino acid sequence of SEQ ID NO: 295 and the VL comprises the amino acid sequence of SEQ ID NO: 292.

In an embodiment, the VH comprises one, two, or all of: (i) an HCDR1 comprising an amino acid sequence of GYSFSSY (SEQ ID NO: 355); (ii) an HCDR2 comprising an amino acid sequence of HPSDST (SEQ ID NO: 351); or (iii) an HCDR3 comprising an amino acid sequence of FVY; and the VL comprises one, two, or all of: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); or (iii) an LCDR3 comprising an amino acid sequence of QQLVEYPYT (SEQ ID NO: 354). In an embodiment, the VH comprises: (i) an HCDR1 comprising an amino acid sequence of GYSFSSY (SEQ ID NO: 355); (ii) an HCDR2 comprising an amino acid sequence of HPSDST (SEQ ID NO: 351); and (iii) an HCDR3 comprising an amino acid sequence of FVY; and the VL comprises: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); and (iii) an LCDR3 comprising an amino acid sequence of QQLVEYPYT (SEQ ID NO: 354).

In an embodiment, the VH comprises one, two, or all of: (i) an HCDR1 comprising an amino acid sequence of SYYMH (SEQ ID NO: 380); (ii) an HCDR2 comprising an amino acid sequence of TIHPSDSTTNYNQKFKG (SEQ ID NO: 382); or (iii) an HCDR3 comprising an amino acid sequence of FVY; and the VL comprises one, two, or all of: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); or (iii) an LCDR3 comprising an amino acid sequence of QQLVEYPYT (SEQ ID NO: 354). In an embodiment, the VH comprises: (i) an HCDR1 comprising an amino acid sequence of SYYMH (SEQ ID NO: 380); (ii) an HCDR2 comprising an amino acid sequence of TIHPSDSTTNYNQKFKG (SEQ ID NO: 382); and (iii) an HCDR3 comprising an amino acid sequence of FVY; and the VL comprises: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); and (iii) an LCDR3 comprising an amino acid sequence of QQLVEYPYT (SEQ ID NO: 354).

In an embodiment, the VH comprises the amino acid sequence of SEQ ID NO: 296. In an embodiment, the VL comprises the amino acid sequence of SEQ ID NO: 292. In an embodiment, the VH comprises the amino acid sequence of SEQ ID NO: 296 and the VL comprises the amino acid sequence of SEQ ID NO: 292.

In an embodiment, the VH comprises one, two, or all of: (i) an HCDR1 comprising an amino acid sequence of GYTESSY (SEQ ID NO: 356); (ii) an HCDR2 comprising an amino acid sequence of HPSDST (SEQ ID NO: 351); or (iii) an HCDR3 comprising an amino acid sequence of FVY; and the VL comprises one, two, or all of: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); or (iii) an LCDR3 comprising an amino acid sequence of QQLVEYPYT (SEQ ID NO: 354). In an embodiment, the VH comprises: (i) an HCDR1 comprising an amino acid sequence of GYTFSSY (SEQ ID NO: 356); (ii) an HCDR2 comprising an amino acid sequence of HPSDST (SEQ ID NO: 351); and (iii) an HCDR3 comprising an amino acid sequence of FVY; and the VL comprises: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); and (iii) an LCDR3 comprising an amino acid sequence of QQLVEYPYT (SEQ ID NO: 354).

In an embodiment, the VH comprises one, two, or all of: (i) an HCDR1 comprising an amino acid sequence of SYYMH (SEQ ID NO: 380); (ii) an HCDR2 comprising an amino acid sequence of TIHPSDSTTNYNQKFKG (SEQ ID NO: 382); or (iii) an HCDR3 comprising an amino acid sequence of FVY; and the VL comprises one, two, or all of: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); or (iii) an LCDR3 comprising an amino acid sequence of QQLVEYPYT (SEQ ID NO: 354). In an embodiment, the VH comprises: (i) an HCDR1 comprising an amino acid sequence of SYYMH (SEQ ID NO: 380); (ii) an HCDR2 comprising an amino acid sequence of TIHPSDSTTNYNQKFKG (SEQ ID NO: 382); and (iii) an HCDR3 comprising an amino acid sequence of FVY; and the VL comprises: (i) an LCDR1 comprising an amino acid sequence of RSSKSLLYKDGKTYLN (SEQ ID NO: 352); (ii) an LCDR2 comprising an amino acid sequence of VVSTRAS (SEQ ID NO: 353); and (iii) an LCDR3 comprising an amino acid sequence of QQLVEYPYT (SEQ ID NO: 354).

In an embodiment, the VH comprises the amino acid sequence of SEQ ID NO: 297. In an embodiment, the VL comprises the amino acid sequence of SEQ ID NO: 292. In an embodiment, the VH comprises the amino acid sequence of SEQ ID NO: 297 and the VL comprises the amino acid sequence of SEQ ID NO: 292.

In an embodiment, the VH comprises one, two, or all of: (i) an HCDR1 comprising an amino acid sequence of GFTFNTY (SEQ ID NO: 357); (ii) an HCDR2 comprising an amino acid sequence of RSKSSNYA (SEQ ID NO: 358); or (iii) an HCDR3 comprising an amino acid sequence of ELRLRYAMDY (SEQ ID NO: 359); and the VL comprises one, two, or all of: (i) an LCDR1 comprising an amino acid sequence of KSSQSLLYTNGKTYLN (SEQ ID NO: 360); (ii) an LCDR2 comprising an amino acid sequence of LVSKLDS (SEQ ID NO: 304); or (iii) an LCDR3 comprising an amino acid sequence of LQSTHFPLT (SEQ ID NO: 361). In an embodiment, the VH comprises: (i) an HCDR1 comprising an amino acid sequence of GFTENTY (SEQ ID NO: 357); (ii) an HCDR2 comprising an amino acid sequence of RSKSSNYA (SEQ ID NO: 358); and (iii) an HCDR3 comprising an amino acid sequence of ELRLRYAMDY (SEQ ID NO: 359); and the VL comprises: (i) an LCDR1 comprising an amino acid sequence of KSSQSLLYTNGKTYLN (SEQ ID NO: 360); (ii) an LCDR2 comprising an amino acid sequence of LVSKLDS (SEQ ID NO: 304); and (iii) an LCDR3 comprising an amino acid sequence of LQSTHFPLT (SEQ ID NO: 361).

In an embodiment, the VH comprises one, two, or all of: (i) an HCDR1 comprising an amino acid sequence of TYAMH (SEQ ID NO: 383); (ii) an HCDR2 comprising an amino acid sequence of RIRSKSSNYATYYADSVKD (SEQ ID NO: 384); or (iii) an HCDR3 comprising an amino acid sequence of ELRLRYAMDY (SEQ ID NO: 359); and the VL comprises one, two, or all of: (i) an LCDR1 comprising an amino acid sequence of KSSQSLLYTNGKTYLN (SEQ ID NO: 360); (ii) an LCDR2 comprising an amino acid sequence of LVSKLDS (SEQ ID NO: 304); or (iii) an LCDR3 comprising an amino acid sequence of LQSTHFPLT (SEQ ID NO: 361). In an embodiment, the VH comprises: (i) an HCDR1 comprising an amino acid sequence of TYAMH (SEQ ID NO: 383); (ii) an HCDR2 comprising an amino acid sequence of RIRSKSSNYATYYADSVKD (SEQ ID NO: 384); and (iii) an HCDR3 comprising an amino acid sequence of ELRLRYAMDY (SEQ ID NO: 359); and the VL comprises: (i) an LCDR1 comprising an amino acid sequence of KSSQSLLYTNGKTYLN (SEQ ID NO: 360); (ii) an LCDR2 comprising an amino acid sequence of LVSKLDS (SEQ ID NO: 304); and (iii) an LCDR3 comprising an amino acid sequence of LQSTHFPLT (SEQ ID NO: 361).

In an embodiment, the VH comprises the amino acid sequence of SEQ ID NO: 291. In an embodiment, the VL comprises the amino acid sequence of SEQ ID NO: 298. In an embodiment, the VH comprises the amino acid sequence of SEQ ID NO: 298 and the VL comprises the amino acid sequence of SEQ ID NO: 292.

In an embodiment, the anti-CD138 antibody molecule comprises:
(a) a heavy chain variable region (VH), wherein the VH comprises three heavy chain complementarity determining regions (HCDR1, HCDR2, and HCDR3), wherein the VH comprises one, two, or all of: (i) an HCDR1 comprising an amino acid sequence of G-F/Y-S/T-F-T/I-A/T/S/R/T/D-H/Y/F; (ii) an HCDR2 comprising an amino acid sequence of D/H/Y/N-P-N/S/Y-T/D/S/Y-G/S-S/A/V; or (iii) an HCDR3 comprising an amino acid sequence of N/S/E-W/Y/G-H/X-D/X-Y/X-T/Y/X-D/E/A/X-G/F/M/X-P/A/L/D-Y/H (X=absent); and
(b) a light chain variable region (VL), wherein the VL comprises three light chain complementarity determining regions (LCDR1, LCDR2, and LCDR3), wherein the VL comprises one, two, or all of: (i) an LCDR1 comprising an amino acid sequence of K-S-S-Q/H-S-L-L-D/H/Y-G/S/T-D/N-G-K/E-T-Y-L-N(SEQ ID NO: 435); (ii) an LCDR2 comprising an amino acid sequence of L-V-S-K/N-L-D-S(SEQ ID NO: 436); or (iii) an LCDR3 comprising an amino acid sequence of W/L-Q-G/S-T-H-F-P-R/Q-T (SEQ ID NO: 437).

In an embodiment, the anti-CD138 antibody molecule comprises:
(a) a heavy chain variable region (VH), wherein the VH comprises three heavy chain complementarity determining regions (HCDR1, HCDR2, and HCDR3), wherein the VH comprises one, two, or all of: (i) an HCDR1 comprising an amino acid sequence of A/T/S/R/D/N-H/Y/F-H/W/N/G-M-H/N/S; (ii) an HCDR2 comprising an amino acid sequence of E/R/N/W-I-D/H/Y/N-P/T-N/S/Y-T/D/S/Y-G/S-S/A/V/D/Y-T/S/P-T/Q/N/G-Y-N/D/T/A-Q/E/D-K/R/N/D-F-R/K/E-A/T/N/S/G; or (iii) an HCDR3 comprising an amino acid sequence of N/S/E-W/Y/G-H/X-D/X-Y/X-T/Y/X-D/E/A/X-G/F/M/X-P/A/L/D-Y/H (X=absent); and
(b) a light chain variable region (VL), wherein the VL comprises three light chain complementarity determining regions (LCDR1, LCDR2, and LCDR3), wherein the VL comprises one, two, or all of: (i) an LCDR1 comprising an amino acid sequence of K-S-S-Q/H-S-L-L-D/H/Y-G/S/T-D/N-G-K/E-T-Y-L-N (SEQ ID NO: 435); (ii) an LCDR2 comprising an amino acid sequence of L-V-S-K/N-L-D-S(SEQ ID NO: 436); or (iii) an LCDR3 comprising an amino acid sequence of W/L-Q-G/S-T-H-F-P-R/Q-T (SEQ ID NO: 437).

In an embodiment, the antibody molecule comprises: (a) a VH comprising: (i) an HCDR1 comprising the amino acid sequence of the HCDR1 of an anti-CD138 antibody described herein, e.g., chosen from antibodies CD001, CD002, CD003, CD004, CD005, CD006, 602, 603, 604, 607, 613, 614, 617, 624, 632, 616, 619, or 623; (ii) an HCDR2 comprising the amino acid sequence of the HCDR2 of the anti-CD138 antibody; and (iii) an HCDR3 comprising the amino acid sequence of the HCDR3 of the anti-CD138 antibody, and (b) a VL comprising: (i) an LCDR1 comprising the amino acid sequence of the LCDR1 of the anti-CD138 antibody; (ii) an LCDR2 comprising the amino acid sequence of the LCDR2 of the anti-CD138 antibody; and (iii) an LCDR3 comprising the amino acid sequence of the LCDR3 of the anti-CD138 antibody.

In an embodiment the anti-CD138 antibody molecule comprises:
(a) a heavy chain variable region (VH), wherein the VH comprises three heavy chain complementarity determining regions (HCDR1, HCDR2, and HCDR3), wherein the VH comprises three heavy chain complementarity determining regions (HCDR1, HCDR2, and HCDR3), wherein the VH comprises one, two, or all of: (i) an HCDR1 comprising an amino acid sequence of G-Y-N/S/T-F-S-S-Y (SEQ ID NO: 438); (ii) an HCDR2 comprising an amino acid sequence of H-P-S-D-S-T (SEQ ID NO: 351); or (iii) an HCDR3 comprising an amino acid sequence of F-V-Y; and
(b) a light chain variable region (VL), wherein the VL comprises three light chain complementarity determining regions (LCDR1, LCDR2, and LCDR3), wherein the VL comprises one, two, or all of: (i) an LCDR1 comprising an amino acid sequence of R-S-S-K-S-L-L-Y-K-D-G-K-T-Y-L-N (SEQ ID NO: 352); (ii) an LCDR2 comprising an amino acid sequence of V-V-S-T-R-A-S(SEQ ID NO: 353); or (iii) an LCDR3 comprising an amino acid sequence of Q-Q-L-V-E-Y-P-Y-T (SEQ ID NO: 354).

In an embodiment, the HCDR1 comprises an amino acid sequence chosen from any of SEQ ID NOS: 350, 355, or 356, the HCDR2 comprises the amino acid sequence of SEQ ID NO: 351, and the HCDR3 comprises the amino acid sequence of F-V-Y. In an embodiment, the LCDR1 comprises the amino acid sequence of SEQ ID NO: 352; the LCDR2 comprises the amino acid sequence of SEQ ID NO: 353; and the LCDR3 comprises the amino acid sequence of SEQ ID NO: 354.

In an embodiment, the anti-CD138 antibody molecule comprises:
(a) a heavy chain variable region (VH), wherein the VH comprises three heavy chain complementarity determining regions (HCDR1, HCDR2, and HCDR3), wherein the VH comprises three heavy chain complementarity determining regions (HCDR1, HCDR2, and HCDR3), wherein the VH comprises one, two, or all of: (i) an HCDR1 comprising an amino acid sequence of S-Y-Y-M-H (SEQ ID NO: 380); (ii) an HCDR2 comprising an amino acid sequence of T-I-H-P-S-D-S-T-T-N-C/Y-N-Q-K-F-K-G (SEQ ID NO: 439); or (iii) an HCDR3 comprising an amino acid sequence of F-V-Y; and
(b) a light chain variable region (VL), wherein the VL comprises three light chain complementarity determining regions (LCDR1, LCDR2, and LCDR3), wherein the VL comprises one, two, or all of: (i) an LCDR1 comprising an amino acid sequence of R-S-S-K-S-L-L-Y-K-D-G-K-T-Y-L-N (SEQ ID NO: 352); (ii) an LCDR2 comprising an amino acid sequence of V-V-S-T-R-A-S(SEQ ID NO: 353); or (iii) an LCDR3 comprising an amino acid sequence of Q-Q-L-V-E-Y-P-Y-T (SEQ ID NO: 354).

In an embodiment, the HCDR1 comprises an amino acid sequence chosen from any of SEQ ID NO: 380, the HCDR2 comprises the amino acid sequence of SEQ ID NO: 381 or 382, and the HCDR3 comprises the amino acid sequence of F-V-Y. In an embodiment, the LCDR1 comprises the amino acid sequence of SEQ ID NO: 352; the LCDR2 comprises the amino acid sequence of SEQ ID NO: 353; and the LCDR3 comprises the amino acid sequence of SEQ ID NO: 354.

In an embodiment, the antibody molecule comprises: (a) a VH comprising: (i) an HCDR1 comprising the amino acid sequence of the HCDR1 of an anti-CD138 antibody described herein, e.g., chosen from antibodies 1610, 2510, 2610, 2710, 2810, 2910, or 1409; (ii) an HCDR2 comprising the amino acid sequence of the HCDR2 of the anti-CD138 antibody; and (iii) an HCDR3 comprising the amino acid sequence of the HCDR3 of the anti-CD138 antibody, and (b) a VL comprising: (i) an LCDR1 comprising the amino acid sequence of the LCDR1 of the anti-CD138 antibody; (ii) an LCDR2 comprising the amino acid sequence of the LCDR2 of the anti-CD138 antibody; and (iii) an LCDR3 comprising the amino acid sequence of the LCDR3 of the anti-CD138 antibody.

In an embodiment, the VH comprises the amino acid sequence of the VH of the anti-CD138 antibody and the VL comprises the amino acid sequence of the VL of the anti-CD138 antibody.

In an embodiment, the antibody molecule comprises two VHs and two VLs.

In an embodiment, the antibody molecule is a synthetic antibody molecule. In an embodiment, the antibody molecule is an isolated antibody molecule. In an embodiment, the antibody molecule is a humanized antibody molecule. In an embodiment, the antibody molecule comprises one or more framework regions derived from human framework germline sequence.

In an embodiment, the antibody molecule comprises a VH region comprising one or more mutations relative to an anti-CD138 antibody described herein (e.g., antibody CD001, CD002, CD003, CD004, CD005, CD006, 602, 603, 604, 607, 613, 614, 617, 624, 632, 616, 619, 623, 1610, 2510, 2610, 2710, 2810, 2910, or 1409). In an embodiment, the mutations comprise one or more substitutions relative to the VH sequence of antibody 1610. In an embodiment, the substitution is C60Y. In an embodiment, the substitution is N28S. In an embodiment, the substitution is N28T. In an embodiment, the substitutions are N28S and C60Y. In an embodiment, the substitutions are N28T and C60Y. In an embodiment, the mutated antibody molecule is expressed in transiently transfected HEK293 cells at levels equal to or greater than antibody 1610.

In an embodiment, the antibody molecule binds to the extracellular domain of CD138. In an embodiment, the antibody molecule binds to an extracellular region of CD138 proximal to the transmembrane domain. In an embodiment, the antibody molecule is capable of binding to one or more (e.g., two, three, or all) of the following peptides: a peptide comprising the amino acid sequence of ENTAVVAVEPDRRNQSPVDQGATGASQGLLDRKEVLG (SEQ ID NO: 440), a peptide comprising the amino acid sequence of TAVVAVEPDRRNQSPVDQGATGASQ (SEQ ID NO: 441), a peptide comprising the amino acid sequence of ENTAVVAVEPDRRNQSPVDQGATG (SEQ ID NO: 442), or a peptide comprising the amino acid sequence of ENTAVVAVEPDRRNQ (SEQ ID NO: 443). In an embodiment, the antibody molecule is capable of binding to one or more (e.g., two or all) of the following peptides: a peptide comprising the amino acid sequence of ENTAVVAVEPDRRNQSPVDQGATGASQGLLDRKEVLG (SEQ ID NO: 440), a peptide comprising the amino acid sequence of RNQSPVDQGATGASQGLLDRKEVLG (SEQ ID NO: 444), or a peptide comprising the amino acid sequence of ENTAVVAVEPDRRNQ (SEQ ID NO: 443).

In an embodiment, the antibody molecule further binds to an extracellular region of CD138 distal to the transmembrane domain, e.g., a region corresponding to or proximal to the integrin binding domain (IBD) of CD138. In an embodiment, the antibody molecule is capable of binding to one or both the following peptides: a peptide comprising the amino acid sequence of ASTSTLPAGEGPKEGEAVVLPEVEPGLTAREQEA (SEQ ID NO: 10) or a peptide comprising the amino acid sequence of GEAVVLPEVEPGLTA (SEQ ID NO: 445).

In an embodiment, the antibody molecule is a synthetic antibody molecule. In an embodiment, the antibody molecule is an isolated antibody molecule. In an embodiment, the antibody molecule is a humanized antibody molecule. In an embodiment, the antibody molecule comprises one or more framework regions derived from human framework germline sequence.

In an embodiment, the antibody molecule is an IgG antibody. In an embodiment, the antibody molecule comprises a heavy chain constant region of IgG chosen from IgG1, IgG2, IgG3, or IgG4. In an embodiment, the antibody molecule comprises a light chain constant region of kappa or lambda light chain.

In an embodiment, the antibody molecule comprises an Fc region comprising one or more mutations to increase the binding affinity to neonatal receptor FcRn and/or the half-life of the antibody molecule. In an embodiment, the antibody molecule comprises an Fc region comprising one or more mutations described herein, e.g., to increase one or more of half-life, ADCC, CDC, or ADCP.

In an embodiment, the antibody molecule is an IgG antibody. In an embodiment, the antibody molecule comprises a heavy chain constant region of IgG chosen from IgG1, IgG2, IgG3, or IgG4. In an embodiment, the antibody molecule comprises a light chain constant region of kappa or lambda light chain.

In an embodiment, the antibody molecule comprises an Fe region comprising one or more mutations to increase the binding affinity to neonatal receptor FcRn and/or the half-life of the antibody molecule. In an embodiment, the antibody molecule comprises an Fc region comprising one or more mutations described herein, e.g., to increase one or more of half-life, ADCC, CDC, or ADCP.

In an embodiment, the antibody molecule further comprises a heavy chain constant region. In an embodiment, the heavy chain constant region is an IgG1 constant region or a functional portion thereof. In another embodiment, the heavy chain constant region is an IgG2 constant region or a functional portion thereof. In an embodiment, the antibody molecule further comprises a light chain constant region. In an embodiment, the antibody molecule further comprises a heavy chain constant region and a light chain constant region. In an embodiment, the antibody molecule comprises a heavy chain constant region, a light chain constant region, and heavy and light chain variable regions of an antibody molecule described in Table 1. In certain embodiments, the antibody molecule comprises a heavy chain constant region, a light chain constant region, and variable regions that comprise one, two, three, four, five, or six CDRs of an antibody molecule described in Table 1.

Exemplary heavy chain constant regions are described below.

IgG1 HC constant region:
(SEQ ID NO: 446)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK
IgG2 HC constant region:
(SEQ ID NO: 447)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK

In an embodiment, the antibody molecule is a multivalent (e.g., bivalent, trivalent, or tetravalent) antibody molecule. In an embodiment, the antibody molecule binds to two or more (e.g., three or four) different regions in CD138. For example, the antibody molecule can comprise two or more sets of identical, or substantially identical, VH-VL pairs, wherein each VH-VL pair binds to two or more different regions in CD138. As another example, the antibody molecule can comprise two or more sets of different VH-VL pairs, wherein each VH-VL pair binds to a different region in CD138.

In an embodiment, the antibody molecule is a multispecific (e.g., bispecific, trispecific, or tetraspecific) antibody molecule. In an embodiment, the antibody molecule has a first binding specificity to CD138 and a second binding specificity other than CD138. For example, the antibody molecule can comprise two or more sets of identical, or substantially identical, VH-VL pairs, wherein each VH-VL pair has both the first binding specificity and the second binding specificity. As another example, the antibody molecule can comprise two or more sets of different VH-VL pairs, wherein each VH-VL pair has a different binding specificity.

### Antibody Molecule-Drug Conjugates

As used herein, the term “antibody molecule-drug conjugate” or ADC refers to an antibody molecule that is coupled to a non-antibody moiety, e.g., a therapeutic agent or label, e.g., a cytotoxic agent. The antibody molecule can be coupled to the non-antibody moiety directly, or indirectly, e.g., through a linker.

In an embodiment, the antibody molecule is coupled to the non-antibody moiety by a covalent bond. In an embodiment, the antibody molecule is coupled to the non-antibody moiety by a peptide bond. In an embodiment, the antibody molecule is coupled to the non-antibody moiety by a non-peptide bond. In an embodiment, the antibody molecule is not coupled to the non-antibody moiety by a non-peptide bond. In an embodiment, a non-antibody moiety is also referred to as a “payload.”

In an embodiment, the non-antibody moiety is coupled to the backbone of the antibody molecule. In another embodiment, the non-antibody moiety is coupled to a side chain of the antibody molecule. In an embodiment, two or more (e.g., three, four, five, six, seven, eight, or more) non-antibody moieties are coupled to the antibody molecule.

In an embodiment, the ADC comprises an antibody molecule that binds to CD138, e.g., an anti-CD138 antibody molecule described herein.

In an embodiment, the ADC comprises one, two, or three CDRs of the VH region of an antibody molecule described in Table 1 (e.g., any of antibodies CD001, CD002, CD003, CD004, CD005, CD006, 602, 603, 604, 607, 613, 614, 617, 624, 632, 616, 619, 623, 1610, 2510, 2610, 2710, 2810, 2910, or 1409), using the Kabat or Chothia definitions of CDRs. In an embodiment, the ADC comprises one, two, or three CDRs of the VL region of an antibody molecule described in Table 1 (e.g., any of antibodies CD001, CD002, CD003, CD004, CD005, CD006, 602, 603, 604, 607, 613, 614, 617, 624, 632, 616, 619, 623, 1610, 2510, 2610, 2710, 2810, 2910, or 1409), using the Kabat or Chothia definitions of CDRs. In an embodiment, the ADC comprises one or more (e.g., two or three) CDRs of the VH region and/or one or more (e.g., two or three) CDRs of the VL region of an antibody molecule described in Table 1 (e.g., any of antibodies CD001, CD002, CD003, CD004, CD005, CD006, 602, 603, 604, 607, 613, 614, 617, 624, 632, 616, 619, 623, 1610, 2510, 2610, 2710, 2810, 2910, or 1409), using the Kabat or Chothia definitions of CDRs.

In an embodiment, the ADC comprises one, two, or three VH CDRs described in Table 1. In an embodiment, the ADC comprises one, two, or three VL CDRs described in Table 1. In an embodiment, the ADC comprises one or more (e.g., two or three) VH CDRs and/or one or more (e.g., two or three) VL CDRs described in Table 1.

In an embodiment, the ADC comprises one, two, three, or four frameworks of the VH region of an antibody molecule described in Table 1 (e.g., any of antibodies CD001, CD002, CD003, CD004, CD005, CD006, 602, 603, 604, 607, 613, 614, 617, 624, 632, 616, 619, 623, 1610, 2510, 2610, 2710, 2810, 2910, or 1409). In an embodiment, the ADC comprises one, two, three, or four frameworks of the VL region of an antibody molecule described in Table 1 (e.g., any of antibodies CD001, CD002, CD003, CD004, CD005, CD006, 602, 603, 604, 607, 613, 614, 617, 624, 632, 616, 619, 623, 1610, 2510, 2610, 2710, 2810, 2910, or 1409). In an embodiment, the ADC comprises one or more (e.g., two, three, or four) frameworks of the VH region and/or one or more (e.g., two, three, or four) frameworks of the VL region of an antibody molecule described in Table 1 (e.g., any of antibodies CD001, CD002, CD003, CD004, CD005, CD006, 602, 603, 604, 607, 613, 614, 617, 624, 632, 616, 619, 623, 1610, 2510, 2610, 2710, 2810, 2910, or 1409).

In an embodiment, the ADC comprises a heavy chain variable region of an antibody molecule described in Table 1 (e.g., any of antibodies CD001, CD002, CD003, CD004, CD005, CD006, 602, 603, 604, 607, 613, 614, 617, 624, 632, 616, 619, 623, 1610, 2510, 2610, 2710, 2810, 2910, or 1409). In an embodiment, the ADC comprises a light chain variable region of an antibody molecule described in Table 1 (e.g., any of antibodies CD001, CD002, CD003, CD004, CD005, CD006, 602, 603, 604, 607, 613, 614, 617, 624, 632, 616, 619, 623, 1610, 2510, 2610, 2710, 2810, 2910, or 1409). In an embodiment, the ADC comprises a heavy chain variable region and a light chain variable region of an antibody molecule described in Table 1 (e.g., any of antibodies CD001, CD002, CD003, CD004, CD005, CD006, 602, 603, 604, 607, 613, 614, 617, 624, 632, 616, 619, 623, 1610, 2510, 2610, 2710, 2810, 2910, or 1409).

In an embodiment, the ADC comprises a heavy chain variable region having an amino acid sequence described in Table 1. In an embodiment, the ADC comprises a light chain variable region having an amino acid sequence described in Table 1. In an embodiment, the ADC comprises a heavy chain variable region having an amino acid sequence described in Table 2 and a light chain variable region having an amino acid sequence described in Table 1.

In an embodiment, the antibody molecule comprises a heavy chain variable region encoded by a nucleotide sequence described in Table 2. In an embodiment, the antibody molecule comprises a light chain variable region encoded by a nucleotide sequence described in Table 2. In an embodiment, the antibody molecule comprises a heavy chain variable region encoded by a nucleotide sequence described in Table 2 and a light chain variable region encoded by a nucleotide sequence described in Table 2.

In an embodiment, the ADC comprises a heavy chain constant region. In an embodiment, the ADC comprises a light chain constant region. In an embodiment, the ADC comprises a heavy chain constant region and a light chain constant region. In an embodiment, the ADC comprises a heavy chain constant region, a light chain constant region, and heavy and light chain variable regions of an antibody molecule described in Table 1. In certain embodiments, the ADC comprises a heavy chain constant region, a light chain constant region, and variable regions that comprise one, two, three, four, five, or six CDRs of antibody molecule described in Table 1.

In an embodiment, the non-antibody molecule comprises a cytotoxic agent (e.g., any cytotoxic agent that is active against a cancer). In an embodiment, the cytotoxic agent is chosen from a tubulin polymerase inhibitor (e.g., an auristatin), an agent associated with tubulin depolymerization (e.g., a maytansine), an agent associated with DNA cleavage (e.g., a calicheamicin), a DNA minor groove alkylating agent (e.g., a duocarymycin), a DNA minor groove cross-linker (e.g., a PBD dimers), or an RNA polymerase II inhibitor (e.g., α-amanitin).

In an embodiment, the cytotoxic agent is α-amanitin. α-amanitin is a bicyclic octapeptide which belongs to a large group of protoplasmic mushroom toxins known as amatoxins. α-Amanitin binds to the bridging helix of RNA polymerase II inhibiting the translocation of RNA and DNA needed to empty the site for the next round of synthesis, thereby reducing the rate of transcription. α-amanitin and its use in ADCs are described, e.g., in Moldenhauer et al. J Natl Cancer Inst. 2012; 104(8): 622-634. The structure of α-amanitin is as follows:

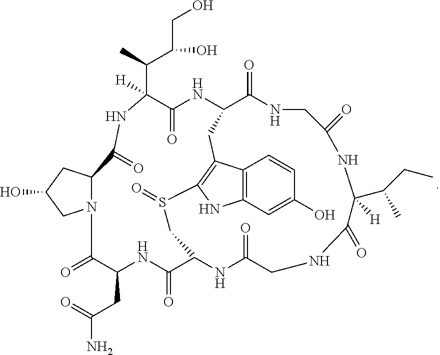

In an embodiment, the cytotoxic agent is a cryptophycin analog. The cryptophycins are a group of cyanobacterial depsipeptides with a remarkable biological activity against multi-drug-resistant (MDR) cancer cells. Cryptophycins deplete microtubules through interaction with tubulin, thereby preventing cell division. They are capable of inducing apoptosis, possibly through other mechanisms in addition to that mediated by microtubule inhibition. Cryptophycin, analogues, and their uses in ADCs are described, e.g., in Shih & Teicher. Curr Pharm Des. 2001; 7(13): 1259-1276; Eggen & Georg. Med Res Rev. 2002; 22(2): 85-101. The structure of a cryptophycin analog is as follows:

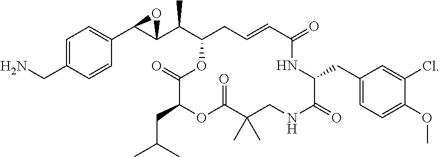 [SMILES: COc1ccc(C[C@H]2NC(=O)/C=C/C[C@@H]([C@H](C)[C@H]3O[C@@H]3c3ccc(CN)cc3)OC(=O)[C@H](CC(C)C)OC(=O)C(C)(C)CNC2=O)cc1Cl]

In an embodiment, the cytotoxic agent is calicheamicin (also known as LL-E33288). Calicheamicin contacts DNA and causes the Bergman cyclization, which results in cleaving the DNA and thus destroying cells. Calicheamicin and its use in ADCs is described, e.g., in Maiese et al. J Antibiot (Tokyo). 1989; 42(4): 558-563; Watanabe et al. Chem Biol. 2002; 9(2): 245-251; Ricart & Tolcher. Nat Clin Pract Oncol. 2007; 4: 245-255. The structure of calicheamicin is as follows.

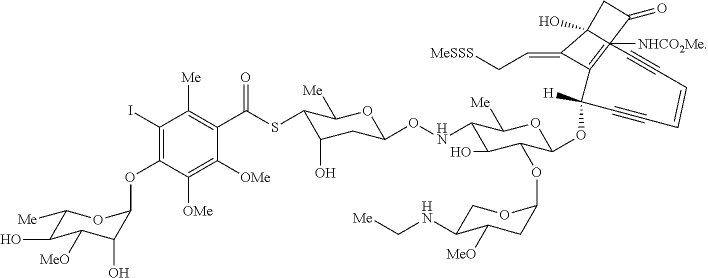

In an embodiment, the cytotoxic agent is centanamycin. Centanamycin is also known as ML-970, AS-I-145, NSC 716970, or N-[4-Amino-1-(2-chloroethyl)-2-naphthyl]-5,6,7-trimethoxy-1H-indole-2-carboxamide). Centanamycin binds the A-T-rich DNA minor groove and alkylates DNA. Centanamycin and its use in ADCs is described, e.g., in Rayburn et al. Cancer Chemother Pharmacol. 2012; 69(6): 1423-31.

In an embodiment, the cytotoxic agent is a dolastatin. In an embodiment, the dolastatin is dolastatin 10 or dolastatin 15. Dolastatins noncompetitively inhibit binding of vincristine to tubulin at the vinca/peptide region). Analogues of dolastatins include, e.g., symplostatin 1, symplostatin 3, and auristatin. Dolastatins, analogues, and their uses are described, e.g., in Amador et al. Annals of Oncology. 2003; 14: 1607-1615; Kijjoa & Sawangwong. Mar Drugs. 2004; 2(2): 73-82; Luesch et al. J Nat Prod. 2001; 64(7): 907-910; Luesch et al. J Nat Prod. 2002; 65(1): 16-20. The structure of dolastatin 10 is as follows:

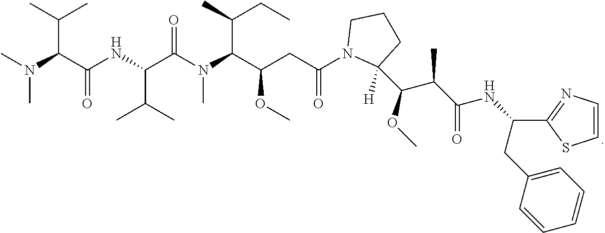 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](C(C)C)N(C)C)C(C)C]

The structure of dolastatin 15 is as follows:

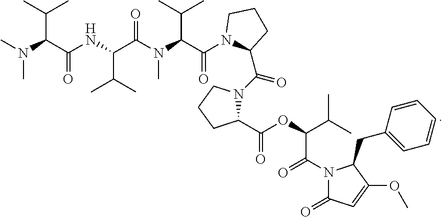 [SMILES: COC1=CC(=O)N(C(=O)[C@@H](OC(=O)[C@@H]2CCCN2C(=O)[C@@H]2CCCN2C(=O)[C@H](C(C)C)N(C)C(=O)[C@@H](NC(=O)[C@H](C(C)C)N(C)C)C(C)C)C(C)C)[C@H]1Cc1ccccc1]

In an embodiment, the cytotoxic agent is a duocarmycin analogue. Duocarmycin analogues are DNA minor groove, AT-sequence selective, and adenine-N3 alkylating agents. Duocarmycin, analogues, and their uses in ADCs are described, e.g., in Tietze & Krewer. Chem Biol Drug Des. 2009; 74(3):205-211; Cacciari et al. Expert Opinion on Therapeutic Patents. 2000; 10 (12): 1853-1871; Tercel et al. Angew Chem Int Ed Engl. 2013; 52(21): 5442-5446. Exemplary duocarmycin and analogues include, e.g., duocarmycin A, duocarmycin B1, duocarmycin B2, duocarmycin C1, duocarmycin C2, duocarmycin D, duocarmycin SA, and CC-1065. The structure of duocarymycin A is as follows:

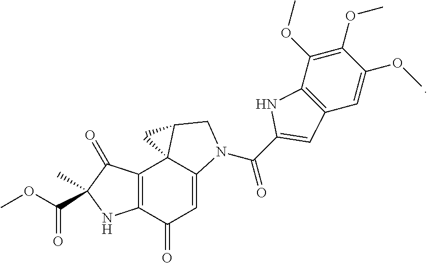

In an embodiment, the cytotoxic agent is maytansine. Maytansine, a benzoansamacrolide, is a highly potent microtubule-targeted compound that induces mitotic arrest and kills tumor cells at subnanomolar concentrations. Maytansine and its analogs (maytansinoids DM1 and DM4) are potent microtubule-targeted compounds that inhibit proliferation of cells at mitosis. Maytansine is described, e.g., in Lopus et al. Mol Cancer Ther. 2010; 9(10): 2689-2699; Widdison et al. J Med Chem. 2006; 49(14): 4392-4408; Liu et al. J Mass Spectrom. 2005; 40(3): 389-399; Tassone et al. Cancer Res. 2004; 64(13): 4629-4636; Sawada et al. Bioconjug Chem. 1993; 4(4):284-289. The structure of maytansine is as follows:

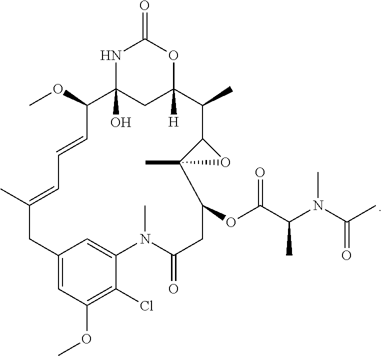

In an embodiment, the cytotoxic agent is monomethyl auristatin E (MMAE, vedotin). MMAE is a highly potent antimitotic agent that inhibits cell division by blocking the polymerization of tubulin. MMAE and its use in ADCs are described, e.g., in Francisco et al. Blood. 2003; 102(4): 1458-1465; Junutula et al. Nat Biotechnol. 2008; 26(8):925-932; Asundi et al. Clin Cancer Res. 2011; 17(5): 965-975; Younes et al. J Clin Oncol. 2012; 30(18):2183-2189; Pettit et al. Anticancer Drug Des. 1995; 10(7): 529-544; Doronina et al. Nat Biotechnol. 2003; 21(7): 778-784. The structure of MMAE is as follows:

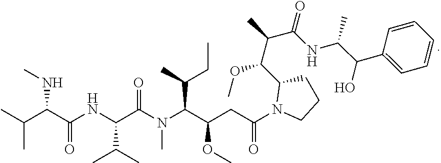 [SMILES: CC[C@H](C)[C@@H]([C@@H](CC(=O)N1CCC[C@H]1[C@H](OC)[C@@H](C)C(=O)N[C@H](C)C(O)c1ccccc1)OC)N(C)C(=O)[C@@H](NC(=O)[C@@H](NC)C(C)C)C(C)C]

In an embodiment, the cytotoxic agent is monomethyl auristatin F (MMAF). MMAF is an antitubulin agent that inhibits cell division by blocking the polymerization of tubulin. It is an auristatin derivative with a charged C-terminal phenylalanine that attenuates its cytotoxic activity compared to its uncharged counterpart, monomethyl auristatin E (MMAE). MMAF can induce potent antitumor effects when conjugated via protease cleavable linkers to a monoclonal antibody targeting internalizing, tumor-specific cell surface antigens. For example, the linker to the monoclonal antibody is stable in extracellular fluid, but can be cleaved by cathepsin once the conjugate has entered a tumor cell, thus activating the anti-mitotic mechanism. MMAF and its use in ADCs are described, e.g., in Smith et al. Mol Cancer Ther. 2006 5; 1474-1482; Doronina et al., Bioconjug Chem. 2006; 17(1): 114-24; Oflazoglu et al. Clin Cancer Res. 2008; 14(19): 6171-6180; Nilsson et al. Cancer. 2010; 116(4 Suppl): 1033-1042. The structure of MMAF is as follows:

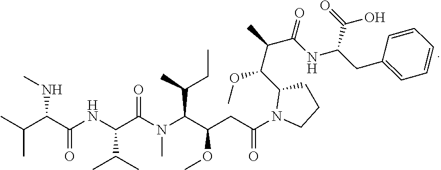 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

In an embodiment, the cytotoxic agent is a pyrrolobenzodiazepine (PBD). PBDs are a class of sequence-selective DNA minor-groove binding crosslinking agents. The mechanism of action of the PBDs is associated with their ability to form an adduct in the minor groove, thus interfering with DNA processing. Exemplary agents that belong to the pyrrolobenzodiazepine antibiotic group include, but are not limited to, anthramycin, abbeymycin, chicamycin, DC-81, mazethramycin, neothramycin A, neothramycin B, porothramycin, prothracarcin, sibanomicin (DC-102), sibiromycin, and tomamycin. PBDs and their use in ADCs are described, e.g., in Antonow & Thurston DE. Chem Rev. 2011; 111: 2815-2864; Cipolla et al. Anticancer Agents Med Chem. 2009; 9: 1-31; Gerratana. Med Res Rev. 2012; 32: 254-293; Li et al. Appl Environ Microbiol. 2009; 75(9):2869-2878; Rahman et al. Org. Biomol. Chem. 2011; 9: 1632-1641; Saunders et al. Sci Transl Med. 2015; 7(302): 302ra136; Hu et al. Chem Biol. 2007; 14(6):691-701. The structure of PBD is as follows:

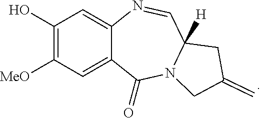 [SMILES: C=C1C[C@H]2C=Nc3cc(O)c(OC)cc3C(=O)N2C1]

In an embodiment, the ADC further comprises a linker, e.g., a linker that couples an antibody molecule to a non-antibody moiety. In an embodiment, the linker comprises a hydrazone, a disulfide bond, a peptide, or a thioether bond.

In an embodiment, the linker is a non-cleavable linker. Exemplary non-cleavable linkers include, e.g., a non-cleavable thioether linker (e.g., N-succinimidyl-4-(N-maleimidomethyl)cyclohexane-1-carboxylate (SMCC)) or a non-cleavable maleimidocaproyl linker.

In an embodiment, the liner is a cleavable linker. In an embodiment, the cleavable linker is a chemically labile linker, e.g., an acid-cleavable linker (e.g., an acid-cleavable hydrazone) or a reducible linker (e.g., a disulfide linker). In an embodiment, the cleavable linker is an enzyme cleavable linker, e.g., a peptide-based linker (e.g., a dipeptide linker (e.g., a valine-citrulline (Val-Cit) linker or a phenylalanine-lysine (Phe-Lys) dipeptide linker)) or a B-glucuronide linker. Other linkers and their use in ADCs are described, e.g., in Lu et al. Int J Mol Sci. 2016; 17(4): 561, the content of which is incorporated by reference in its entirety.

In an embodiment, the linker is a poly(ethylene glycol) (PEG) linker.

### Animal Models

The antibody molecules described herein can be evaluated in vivo, e.g., using various animal models. For example, an animal model can be used to test the efficacy of an antibody molecule described herein in inhibiting CD138 and/or in treating or preventing a disorder described herein, e.g., a myeloma (e.g., multiple myeloma). Animal models can also be used, e.g., to investigate for side effects, measure concentrations of antibody molecules in situ, demonstrate correlations between a CD138 function and a disorder described herein, e.g., a myeloma (e.g., multiple myeloma). Exemplary types of animals that can be used to evaluate the antibody molecules described herein include, but are not limited to, mice, rats, rabbits, guinea pigs, and monkeys.

Exemplary animal models for myelomas (e.g., multiple myeloma) that can be used for evaluating an antibody molecule described herein include, but are not limited to, immunocompetent murine models, e.g., 5TMM (5T Radl), 5T2, 5T33, and STGMA models (Radl et al. Am J Pathol. 1988; 132: 593-597); immunocompromised murine models, e.g., RAG-2 model (Fowler et al. Dis Model Mech. 2009; 2: 604-611), xenograft murine myeloma models, e.g., SCID and NOD/SCID models (Huang et al. Cancer Res. 1993; 53: 1392-1396; Tsunenari et al. Blood. 1997; 90: 2437-2444; Torcia et al. Exp Hematol. 1996; 24: 868-874; Hjorth-Hansen et al. J Bone Miner Res. 1999; 14: 256-263); SCID-Hu and SCID-Rab models (Urashima et al. Blood. 1997; 90: 754-765; Yaccoby et al. Blood. 1998; 92: 2908-2913; Yata & Yaccoby. Leukemia. 2004; 18: 1891-1897); genetically engineered models, e.g., IL-6- and MYC-driven models (Kovalchuk et al. Proc Natl Acad Sci USA. 2002; 99: 1509-1514; Adams et al. Nature. 1985; 318: 533-538; Chesi et al. Blood. 2012; 120: 376-385): Eu-xbp-1s model (Carrasco et al. Cancer Cell. 2007; 11(4):349-360); L-GP130 model (Dechow et al. J Clin Invest. 2014; 124(12): 5263-5274).

Various murine and human myeloma cell lines and primary human myeloma cells can be used in preclinical in vivo models. Exemplary murine and human myeloma cell lines that can be used for engraftment include, but are not limited to, 5T myeloma cells (Radl et al. Am J Pathol. 1988; 132: 593-597), human lymphoblastoid ARH-77 cells (Huang et al. Cancer Res. 1993; 53(6): 1392-1396), the human JJN3 myeloma cell line (Hjorth-Hansen et al. J Bone Miner Res. 1999; 14(2): 256-263), and IL-6-dependent myeloma cell lines (Tsunenari et al. Blood. 1997; 90(6):2437-2444). A desired cell line can be selected based on, e.g., the pace of tumor engraftment, characteristics of the particular tumor type (e.g., propensity to develop lytic bone lesions), or the type of monoclonal protein that is produced.

Other animal models for myelomas (e.g., multiple myeloma) are described, e.g., in Lwin et al. Bonekey Rep. 2016; 5: 772; Libouban et al. Morphologie. 2015; 99(325): 63-72; Campbell et al. Curr Protoc Pharmacol. 2008; Chapter 14: Unit 14.9.

### Pharmaceutical Compositions and Kits

In some aspects, this disclosure provides compositions, e.g., pharmaceutically acceptable compositions, which include an anti-CD138 antibody molecule described herein (e.g., a humanized antibody molecule described herein), formulated together with a pharmaceutically acceptable carrier.

As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion). In certain embodiments, less than about 5%, e.g., less than about 4%, 3%, 2%, or 1% of the antibody molecules in the pharmaceutical composition are present as aggregates. In other embodiments, at least about 95%, e.g., at least about 96%, 97%, 98%, 98.5%, 99%, 99.5%, 99.8%, or more of the antibody molecules in the pharmaceutical composition are present as monomers. In some embodiments, the level of aggregates or monomers is determined by chromatography, e.g., high performance size exclusion chromatography (HP-SEC).

The compositions set out herein may be in a variety of forms. These include, for example, liquid, semi-solid and solid dosage forms, such as liquid solutions (e.g., injectable and infusible solutions), dispersions or suspensions, liposomes, and suppositories. A suitable form depends on the intended mode of administration and therapeutic application. Typical suitable compositions are in the form of injectable or infusible solutions. One suitable mode of administration is parenteral (e.g., intravenous, subcutaneous, intraperitoneal, intramuscular). In some embodiments, the antibody molecule is administered by intravenous infusion or injection. In certain embodiments, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibody molecules described herein can be administered by a variety of methods. Several are known in the art, and for many therapeutic, prophylactic, or diagnostic applications, an appropriate route/mode of administration is intravenous injection or infusion. For example, the antibody molecules can be administered by intravenous infusion at a rate of less than 10 mg/min; preferably less than or equal to 5 mg/min to reach a dose of about 1 to 100 mg/m², preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody molecule can be orally administered, for example, with an inert diluent or an assimilable edible carrier. The antibody molecule (and other ingredients, if desired) may also be enclosed in a hard or soft shell gelatin capsule, compressed into tablets, or incorporated directly into the subject's diet. For oral therapeutic administration, the antibody molecule may be incorporated with excipients and used in the form of ingestible tablets, buccal tablets, troches, capsules, elixirs, suspensions, syrups, wafers, and the like. To administer an antibody molecule by other than parenteral administration, it may be necessary to coat the compound with, or co-administer the compound with, a material to prevent its inactivation. Therapeutic, prophylactic, or diagnostic compositions can also be administered with medical devices, and several are known in the art.

Dosage regimens are adjusted to provide the desired response (e.g., a therapeutic, prophylactic, or diagnostic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the antibody molecule and the particular therapeutic, prophylactic, or diagnostic effect to be achieved, and (b) the limitations inherent in the art of compounding such an antibody molecule for the treatment of sensitivity in individuals.

An exemplary, non-limiting range for a therapeutically, prophylactically, or diagnostically effective amount of an antibody molecule is about 0.1-50 mg/kg body weight of a subject, e.g., about 0.1-30 mg/kg. e.g., about 1-30, 1-15, 1-10, 1-5, 5-10, or 1-3 mg/kg, e.g., about 1, 2, 3, 4, 5, 6, 7, 8, 9, 10, 15, 20, 30, 40, or 50 mg/kg. The antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², e.g., about 10 mg/m2. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed compositions.

The pharmaceutical compositions herein may include a “therapeutically effective amount,” “prophylactically effective amount.” or “diagnostically effectively amount” of an antibody molecule described herein.

A “therapeutically effective amount” refers to an amount effective, at dosages and for periods of time necessary, to achieve the desired therapeutic result. A therapeutically effective amount of the antibody molecule may vary according to factors such as the disease state, age, sex, and weight of the individual, and the ability of the antibody or antibody portion to elicit a desired response in the individual. A therapeutically effective amount is also one in which any toxic or detrimental effect of the antibody molecule is outweighed by the therapeutically beneficial effects. A “therapeutically effective dosage” typically inhibits a measurable parameter by at least about 20%, e.g., by at least about 40%, by at least about 60%, or by at least about 80% relative to untreated subjects. The measurable parameter may be, e.g., hematuria, colored urine, foamy urine, pain, swelling (edema) in the hands and feet, or high blood pressure. The ability of an antibody molecule to inhibit a measurable parameter can be evaluated in an animal model system predictive of efficacy in treating or preventing a myeloma. Alternatively, this property of a composition can be evaluated by examining the ability of the antibody molecule to inhibit CD138, e.g., by an in vitro assay.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

A “diagnostically effective amount” refers to an amount effective, at dosages and for periods of time necessary, to achieve the desired diagnostic result. Typically, a diagnostically effective amount is one in which a disorder, e.g., a disorder described herein, e.g., A myeloma, can be diagnosed in vitro, ex vivo, or in vivo.

Also within this disclosure is a kit that comprises an antibody molecule, described herein. The kit can include one or more other elements including: instructions for use; other reagents, e.g., a label, a therapeutic agent, or an agent useful for chelating, or otherwise coupling, an antibody molecule to a label or therapeutic agent, or a radioprotective composition; devices or other materials for preparing the antibody molecule for administration; pharmaceutically acceptable carriers; and devices or other materials for administration to a subject.

### Nucleic Acids

The present disclosure also features nucleic acids comprising nucleotide sequences that encode the anti-CD138 antibody molecules (e.g., heavy and light chain variable regions and CDRs of the antibody molecules), as described herein.

For example, the present disclosure features a first and second nucleic acid encoding heavy and light chain variable regions, respectively, of an antibody molecule chosen from one or more of the antibody molecules disclosed herein, e.g., an antibody molecule of Table 2, or a portion of an antibody molecule, e.g., the variable regions of Table 2. The nucleic acid can comprise a nucleotide sequence encoding any one of the amino acid sequences in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein).

In certain embodiments, the nucleic acid can comprise a nucleotide sequence encoding at least one, two, or three CDRs from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In some embodiments, the nucleic acid can comprise a nucleotide sequence encoding at least one, two, or three CDRs from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In some embodiments, the nucleic acid can comprise a nucleotide sequence encoding at least one, two, three, four, five, or six CDRs from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs from a heavy chain variable region having the nucleotide sequence as set forth in Table 2, a sequence substantially homologous thereto (e.g., a sequence at least about 85%, 90%, 95%, 99% or more identical thereto, and/or capable of hybridizing under the stringency conditions described herein). In some embodiments, the nucleic acid can comprise a nucleotide sequence encoding at least one, two, or three CDRs from a light chain variable region having the nucleotide sequence as set forth in Table 2, or a sequence substantially homologous thereto (e.g., a sequence at least about 85%, 90%, 95%, 99% or more identical thereto, and/or capable of hybridizing under the stringency conditions described herein). In certain embodiments, the nucleic acid can comprise a nucleotide sequence encoding at least one, two, three, four, five, or six CDRs from heavy and light chain variable regions having the nucleotide sequence as set forth in Table 2, or a sequence substantially homologous thereto (e.g., a sequence at least about 85%, 90%, 95%, 99% or more identical thereto, and/or capable of hybridizing under the stringency conditions described herein).

In certain embodiments, the nucleic acid comprises a nucleotide sequence as set forth in Table 2 or a sequence substantially homologous thereto (e.g., a sequence at least about 85%, 90%, 95%, 99% or more identical thereto, and/or capable of hybridizing under the stringency conditions described herein). In some embodiments, the nucleic acid comprises a portion of a nucleotide sequence as set forth in Table 2 or a sequence substantially homologous thereto (e.g., a sequence at least about 85%, 90%, 95%, 99% or more identical thereto, and/or capable of hybridizing under the stringency conditions described herein). The portion may encode, for example, a variable region (e.g., VH or VL); one, two, or three or more CDRs; or one, two, three, or four or more framework regions.

The nucleic acids disclosed herein include deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

In some aspects, the application features host cells and vectors containing the nucleic acids described herein. The nucleic acids may be present in a single vector or separate vectors present in the same host cell or separate host cell, as described in more detail below.

### Vectors

Further provided herein are vectors that comprise nucleotide sequences encoding an anti-CD138 antibody molecule described herein.

In an embodiment, the vector comprises a nucleotide encoding an antibody molecule described herein, e.g., as described in Table 1. In another embodiment, the vector comprises a nucleotide sequence described herein, e.g., in Table 2. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

The present disclosure also provides cells (e.g., host cells) comprising a nucleic acid encoding an anti-CD138 antibody molecule as described herein. For example, the host cells may comprise a nucleic acid molecule having a nucleotide sequence described in Table 2, a sequence substantially homologous thereto (e.g., a sequence at least about 85%, 90%, 95%, 99% or more identical thereto, and/or capable of hybridizing under the stringency conditions described herein), or a portion of one of said nucleic acids. Additionally, the host cells may comprise a nucleic acid molecule encoding an amino acid sequence of Table 1, a sequence substantially homologous thereto (e.g., a sequence at least about 80%, 85%, 90%, 95%, 99% or more identical thereto), or a portion of one of said sequences.

In some embodiments, the host cells are genetically engineered to comprise nucleic acids encoding the antibody molecule described herein.

In certain embodiments, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells. In an embodiment, the cell (e.g., host cell) is an isolated cell.

### Uses of Antibody Molecules

The anti-CD138 antibody molecules disclosed herein, as well as the pharmaceutical compositions disclosed herein, have in vitro, ex vivo, and in vivo therapeutic, prophylactic, and/or diagnostic utilities.

In an embodiment, the antibody molecule causes (e.g., induces or increases) an effector function on a cell expressing CD138. For example, the antibody molecules can be administered to a subject, e.g., a human subject, to cause an antibody-dependent cellular cytotoxicity activity on a diseased cell (e.g., a cancer cell or a precancerous cell) that it binds to. In an embodiment, the antibody molecule causes a complement-dependent cytotoxicity activity on a cell expressing CD138. In an embodiment, the antibody molecule reduces (e.g., inhibits, blocks, or neutralizes) one or more biological activities of a cell expressing CD138. In an embodiment, the antibody molecule inhibits the action of a protease on a membrane-bound CD138, e.g., to reduce shedding of CD138. For example, these antibodies molecules can be administered to cells in culture, in vitro or ex vivo, or to a subject, e.g., a human subject, e.g., in vivo, to reduce (e.g., inhibits, blocks, or neutralizes) one or more biological activities of the cell.

Accordingly, in an aspect, the disclosure provides a method of treating, preventing, or diagnosing a disorder, e.g., a disorder described herein (e.g., multiple myeloma), in a subject, comprising administering to the subject an anti-CD138 antibody molecule described herein, such that the disorder is treated, prevented, or diagnosed. For example, the disclosure provides a method comprising contacting the antibody molecule described herein with cells in culture, e.g. in vitro or ex vivo, or administering the antibody molecule described herein to a subject, e.g., in vivo, to treat, prevent, or diagnose a disorder, e.g., a disorder associated with CD138 (e.g., multiple myeloma).

As used herein, the term “subject” is intended to include human and non-human animals. In some embodiments, the subject is a human subject, e.g., a human patient having a disorder described herein (e.g., multiple myeloma), or at risk of having a disorder described herein (e.g., multiple myeloma). The term “non-human animals” includes mammals and non-mammals, such as non-human primates. In some embodiments, the subject is a human. The methods and compositions described herein are suitable for treating human patients a disorder described herein (e.g., multiple myeloma). Patients having a disorder described herein include, e.g., those who have developed a disorder described herein but are (at least temporarily) asymptomatic, patients who have exhibited a symptom of a disorder described herein, and patients having a disorder related to or associated with a disorder described herein.

### Methods of Treating or Preventing Disorders

The antibody molecules described herein can be used to treat or prevent disorders associated with CD138 or symptoms thereof.

Exemplary disorders or conditions that can be associated with CD138 include, but are not limited to cancer (e.g., hematological cancer (e.g., a myeloma, e.g., multiple myeloma) or solid tumors, and precancerous conditions (e.g., smoldering myeloma or monoclonal gammopathy of undetermined significance (MGUS)). In an embodiment, the disorder is associated with aberrant expression of CD138. In an embodiment, the antibody molecule is used to treat a subject having a disorder described herein, or is at risk of developing a disorder described herein. In an embodiment, the antibody molecule is used to reduce progression of the disorder, e.g., to reduce progression of a precancerous condition to cancer.

The antibody molecules described herein are typically administered at a frequency that keeps a therapeutically effective level of antibody molecules in the patient's system until the patient recovers. For example, the antibody molecules may be administered at a frequency that achieves a serum concentration sufficient for at least about 1, 2, 5, 10, 20, 30, or 40 antibody molecules to bind each CD138 molecule. In an embodiment, the antibody molecules are administered every 1, 2, 3, 4, 5, 6, or 7 days, every 1, 2, 3, 4, 5, or 6 weeks, or every 1, 2, 3, 4, 5, or 6 months.

Methods of administering various antibody molecules are known in the art and are described below. Suitable dosages of the antibody molecules used will depend on the age and weight of the subject and the particular drug used.

In an embodiment, the antibody molecule is administered to the subject (e.g., a human subject) intravenously. In an embodiment, the antibody molecule is administered to the subject at a dose between 0.1 mg/kg and 50 mg/kg, e.g., between 0.2 mg/kg and 25 mg/kg, between 0.5 mg/kg and 10 mg/kg, between 0.5 mg/kg and 5 mg/kg, between 0.5 mg/kg and 3 mg/kg, between 0.5 mg/kg and 2.5 mg/kg, between 0.5 mg/kg and 2 mg/kg, between 0.5 mg/kg and 1.5 mg/kg, between 0.5 mg/kg and 1 mg/kg, between 1 mg/kg and 1.5 mg/kg, between 1 mg/kg and 2 mg/kg, between 1 mg/kg and 2.5 mg/kg, between 1 mg/kg and 3 mg/kg, between 1 mg/kg and 2.5 mg/kg, or between 1 mg/kg and 5 mg/kg. In an embodiment, the antibody molecule is administered to the subject at a fixed dose between 10 mg and 1000 mg, e.g., between 10 mg and 500 mg, between 10 mg and 250 mg, between 10 mg and 150 mg, between 10 mg and 100 mg, between 10 mg and 50 mg, between 250 mg and 500 mg, between 150 mg and 500 mg, between 100 mg and 500 mg, between 50 mg and 500 mg, between 25 mg and 250 mg, between 50 mg and 150 mg, between 50 mg and 100 mg, between 100 mg and 150 mg, between 100 mg and 200 mg, or between 150 mg and 250 mg. In an embodiment, the antibody molecule is administered once a week, twice a week, once every two weeks, once every three weeks, once every four weeks, once every eight weeks, once a month, once every two months, or once every three months. In an embodiment, the antibody molecule is administered between 0.5 mg/kg and 3 mg/kg or between 50 mg and 150 mg, once a week, twice a week, once every two weeks, or once every four weeks.

The antibody molecules can be used by themselves or conjugated to a second agent, e.g., a bacterial agent, toxin, or protein, e.g., a second anti-CD138 antibody molecule. This method includes: administering the antibody molecule, alone or conjugated to a second agent, to a subject requiring such treatment. The antibody molecules can be used to deliver a variety of therapeutic agents, e.g., a toxin, or mixtures thereof.

### Cancer

The anti-CD138 antibody molecules described herein can be used to treat or prevent a cancer or a precancerous condition.

CD138 expression is dysregulated in many cancers, e.g., prostate cancer, breast cancer, pancreatic cancer, ovarian cancer, colon cancer, lung cancer, and myeloma (Kiviniemi et al. APMIS. 2004; 112(2): 89-97; Lendorf et al. J Histochem Cytochem. 2011; 59(6): 615-629; Juuti et al. Oncology. 2005; 68(2-3): 97-106; Kusumoto et al. Oncol Rep. 2010; 23(4): 917-25; Hashimoto et al. BMC Cancer. 2008; 8: 185; Joensuu et al. Cancer Res. 2002; 62(18):5210-5217; Seidel et al. Blood. 2000; 95(2): 388-392). CD138 can modulate several key processes of tumorigenesis, e.g., cancer cell proliferation, apoptosis, and angiogenesis (Teng et al. Matrix Biol. 2012; 31(1): 3-16). The molecular and clinical profiles of CD138 in solid and hematological cancers are described, e.g., in Akl et al. Oncotarget. 2015; 6(30):28693-28715.

CD138 can affect tumorigenesis by regulating mediators of tumor cell survival and proliferation (e.g., oncogenes or growth factors). For example, Sdc1−/−mice were protected against Wnt-1 induced mammary tumorigenesis (Alexander et al. Nat Genet. 2000; 25(3): 329-32). Hepatocyte growth factor (HGF) binds to CD138 on myeloma cells (Derksen et al. Blood. 2002; 99(4): 1405-1410). The interaction of HGF with CD138 potentiated Met signaling, which is involved in the growth, survival, and spread of a number of cancers (Birchmeier et al. Nat Rev Mol Cell Biol. 2003; 4(12): 915-925; Derksen et al. Blood. 2002; 99(4):1405-1410). CD138 expression is elevated in the reactive stroma of breast carcinoma tissue (Stanley et al. Am J Clin Pathol. 1999; 112(3): 377-383). MEFs expressing CD138 enhanced the growth of breast cancer cell lines in co-culture and promoted breast carcinoma progression in vivo (Maeda et al. Cancer Res. 2004; 64(2):612-621).

CD138 can regulate tumor cell apoptosis. Knock-down of CD138 in myeloma cells induced growth arrest and apoptosis (Khotskaya et al. J Biol Chem. 2009; 284(38): 26085-26095). Recombinant CD138 ectodomains induced apoptosis in MCF-7 breast cancer cells and cultured human prostate cancer cells (Sun et al. Cancer Res. 2008; 68(8):2912-2919; Hu et al. Neoplasia. 2010; 12(10): 826-836).

CD138 can bind to pro-angiogenic factors (e.g., FGF-2 and VEGF) and present these factors to their respective receptors on endothelial cells to initiate endothelial invasion and budding (Teng et al. Matrix Biol. 2012; 31(1): 3-16). Increased CD138 expression in stromal fibroblasts was observed in several carcinomas, such as those of the breast, stomach, and thyroid (Stanley et al. Am J Clin Pathol. 1999; 112(3): 377-383; Wiksten et al. Int J Cancer. 2001; 95(1): 1-6; Barbareschi et al. Cancer. 2003; 98(3): 474-483). In a xenograft model of human breast carcinoma cells and CD138-transfected fibroblasts implantation into mice, stromal CD138 expression was associated with significantly elevated microvessel density and larger vessel area (Maeda et al. Oncogene. 2006; 25(9): 1408-1412).

Exemplary cancers that can be treated or prevented by the antibody molecules described herein include, but are not limited to, acute lymphoblastic leukemia (ALL), acute myeloid leukemia (AML), adrenocortical carcinoma, Kaposi sarcoma, an AIDS-related lymphoma, primary central nervous system (CNS) lymphoma, anal cancer, appendix cancer, astrocytoma, atypical teratoid/rhabdoid tumor, basal cell carcinoma, bile duct cancer, bladder cancer, bone cancer (e.g., Ewing sarcoma or osteosarcoma and malignant fibrous histiocytoma), brain tumor (e.g., astrocytomas, brain stem glioma, central nervous system atypical teratoid/rhabdoid tumor, central nervous system embryonal tumor, central nervous system germ cell tumor, craniopharyngioma, or ependymoma), breast cancer, bronchial tumor, Burkitt lymphoma, carcinoid tumor (e.g., gastrointestinal carcinoid tumor), cardiac (heart) tumor, embryonal tumor, germ cell tumor, lymphoma, cervical cancer, cholangiocarcinoma, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma in situ (DCIS), endometrial cancer, ependymoma, esophageal cancer, esthesioneuroblastoma, Ewing sarcoma, extracranial germ cell tumor, extragonadal germ cell tumor, eye cancer (e.g., intraocular melanoma or retinoblastoma), fallopian tube cancer, fibrous histiocytoma of bone, osteosarcoma, gallbladder cancer, gastric (stomach) cancer, gastrointestinal carcinoid tumor, gastrointestinal stromal tumors (GIST), germ cell tumor (e.g., central nervous system tumor, extracranial tumor, extragonadal tumor, ovarian cancer, or testicular cancer), gestational trophoblastic disease, glioma, hairy cell leukemia, head and neck cancer, hepatocellular (liver) cancer, Hodgkin lymphoma, hypopharyngeal cancer, intraocular melanoma, islet cell tumor, pancreatic neuroendocrine tumor, Kaposi sarcoma, kidney cancer (e.g., renal cell cancer or Wilms tumor), Langerhans cell histiocytosis (LCH), laryngeal cancer, leukemia (e.g., acute lymphoblastic leukemia (ALL), acute myeloid leukemia (AML), chronic lymphocytic leukemia (CLL), chronic myclogenous leukemia (CML), or hairy cell leukemia), lip and oral cavity cancer, liver cancer, lung cancer (e.g., non-small cell lung cancer (NSCLC) or small cell lung cancer), lymphoma (e.g., aids-related, Burkitt lymphoma, cutaneous T-cell lymphoma, Hodgkin lymphoma, non-Hodgkin lymphoma, or primary central nervous system (CNS) lymphoma), Waldenström macroglobulinemia, male breast cancer, malignant fibrous histiocytoma of bone and osteosarcoma, melanoma (e.g., intraocular (eye) melanoma), Merkel cell carcinoma, mesothelioma, metastatic squamous neck cancer, midline tract carcinoma, mouth cancer, multiple endocrine neoplasia syndrome, multiple myeloma/plasma cell neoplasm, mycosis fungoides, myelodysplastic syndrome, myelodysplastic/myeloproliferative neoplasm, chronic myeloproliferative neoplasm, nasal cavity and paranasal sinus cancer, nasopharyngeal cancer, neuroblastoma, oral cancer, lip and oral cavity cancer, oropharyngeal cancer, osteosarcoma and malignant fibrous histiocytoma of bone, ovarian cancer (e.g., epithelial ovarian cancer or germ cell ovarian tumor), pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, pleuropulmonary blastoma, peritoneal cancer, prostate cancer, rectal cancer, retinoblastoma, rhabdomyosarcoma, salivary gland cancer, sarcoma (e.g., Ewing sarcoma, Kaposi sarcoma, osteosarcoma, rhabdomyosarcoma, soft tissue sarcoma, or uterine sarcoma), Sézary syndrome, skin cancer (e.g., melanoma, Merkel cell carcinoma, or nonmelanoma skin cancer), small intestine cancer, squamous cell carcinoma, testicular cancer, throat cancer, thymoma and thymic carcinoma, thyroid cancer, transitional cell cancer of the renal pelvis and ureter, urethral cancer, endometrial uterine cancer, vaginal cancer, vulvar cancer, or a metastatic lesion thereof.

In an embodiment, the cancer is a hematological cancer, e.g., a myeloma, lymphoma, or leukemia. In an embodiment, the cancer is a myeloma. In an embodiment, the cancer is a multiple myeloma.

In another embodiment, the cancer is a solid tumor. In an embodiment, the cancer is a cervical cancer (e.g., a cervical squamous cell carcinoma or an endocervical adenocarcinoma), a uterine cancer (e.g., a uterine corpus endometrioid carcinoma), a brain cancer (e.g., a glioblastoma), a lung cancer (e.g., a lung squamous cell carcinoma), or a breast cancer (e.g., a breast invasive carcinoma).

In an embodiment, the cancer is chosen from a bladder cancer, a breast cancer, a cervical cancer, a colorectal cancer, an endometrial cancer, a gallbladder cancer, a gastric cancer, a glioma, a head and neck cancer, a laryngeal cancer, a liver cancer, a lung cancer, a mesothelioma, a nasopharyngeal cancer, an oral cancer, an ovarian cancer, a pancreatic cancer, a prostate cancer, or a thyroid cancer.

In an embodiment, the cancer is a bladder cancer. CD138 is expressed in bladder cancer (Kim & Park. Hum Pathol. 2014; 45: 1830-1838). In an embodiment, the bladder cancer is a urothelial carcinoma, a squamous cell carcinoma, or an adenocarcinoma. In an embodiment, the bladder cancer is a noninvasive, non-muscle-invasive, or muscle-invasive. The anti-CD138 antibody molecules described herein can be used alone or in combination with a second therapeutic agent, procedure, or modality to treat a bladder cancer. In an embodiment, the anti-CD138 antibody molecule is used in combination with a surgery (e.g., transurethral resection of bladder tumor (TURBT) or cystectomy), an intravesical therapy (e.g., an intravesical immunotherapy (e.g., Bacillus Calmette-Guerin (BCG) therapy) or an intravesical chemotherapy (e.g., mitomycin, valrubicin, docetaxel, thiotepa, or gemcitabine)), a chemotherapy (e.g., an intravesical chemotherapy or a systemic chemotherapy (e.g., cisplatin, fluorouracil (5-FU), mitomycin, gemcitabine, methotrexate, vinblastine, doxorubicin, carboplatin, paclitaxel, docetaxel, ifosfamide, or pemetrexed), a radiation therapy, or an immunotherapy (e.g., intravesical BCG, an immune checkpoint inhibitor (e.g., a PD-L1 inhibitor (e.g., atezolizumab, durvalumab, or avelumab) or a PD-1 inhibitor (e.g., nivolumab or pembrolizumab).

In an embodiment, the cancer is a breast cancer. CD138 is expressed in breast cancer (Akl et al. Oncotarget. 2015; 6(30):28693-28715; Barbareschi et al. Cancer. 2003; 98: 474-483; Lim et al. Singapore Med J. 2014; 55: 468-472; Nguyen et al. Am J Clin Pathol. 2013; 140: 468-474; Lendorf et al. J Histochem Cytochem. 2011; 59: 615-629; Gotte et al. Breast Cancer Res. 2007; 9(1):R8; Tsanou et al. J Exp Clin Cancer Res. 2004; 23(4):641-650). In an embodiment, the breast cancer is a ductal carcinoma (e.g., ductal carcinoma in situ (DCIS), or invasive ductal carcinoma (IDC) (e.g., a tubular carcinoma, a medullary carcinoma, a mucinous carcinoma, a papillary carcinoma, or a cribriform carcinoma), a lobular carcinoma (e.g., a lobular carcinoma in situ (LCIS) or an invasive lobular carcinoma (ILC)), or an inflammatory breast cancer. In an embodiment, the breast cancer is ER-positive, PR-positive, HER2-positive, or triple-negative (ER−, PR− and HER2−). The anti-CD138 antibody molecules described herein can be used alone or in combination with a second therapeutic agent, procedure, or modality to treat a bladder cancer. In an embodiment, the anti-CD138 antibody molecule is used in combination with a surgery (e.g., a breast-conserving surgery or a mastectomy), a radiation therapy, a chemotherapy (e.g., an anthracycline (e.g., doxorubicin, liposomal doxorubicin, epirubicin), a taxane (e.g., paclitaxel, albumin-bound paclitaxel (e.g., nab-paclitaxel) or docetaxel), 5-fluorouracil (5-FU), cyclophosphamide, a platinum agent (e.g., cisplatin or carboplatin), vinorelbine, capecitabine, gemcitabine, mitoxantrone, ixabepilone, or eribulin), a hormone therapy (e.g., tamoxifen, toremifene, fulvestrant, an aromatase inhibitor (e.g., letrozole, anastrozole, or exemestane), ovarian ablation (e.g., oophorectomy, a luteinizing hormone-releasing hormone (LHRH) analog, or a chemotherapy drug)), a targeted therapy (e.g., trastuzumab, pertuzumab, ado-trastuzumab emtansine, lapatinib, neratinib, a CDK4/6 inhibitor (e.g., palbociclib or ribociclib), an mTOR inhibitor (e.g., everolimus), or a combination thereof.

In an embodiment, the cancer is a cervical cancer. CD138 is expressed in cervical cancer (Akl et al. Oncotarget. 2015; 6(30):28693-28715). In an embodiment, the cervical cancer is a microinvasive cervical cancer or invasive cervical cancer, In an embodiment, the cervical cancer is a squamous cell carcinoma or an adenocarcinoma. The anti-CD138 antibody molecules described herein can be used alone or in combination with a second therapeutic agent, procedure, or modality to treat a cervical cancer. In an embodiment, the anti-CD138 antibody molecule is used in combination with a surgery (e.g., a cryosurgery, a laser surgery, a conization, a simple hysterectomy, a radical hysterectomy, a trachelectomy, or a pelvic exenteration), a radiation therapy, a chemotherapy (e.g., cisplatin, carboplatin, paclitaxel, topotecan, gemcitabine, docetaxel, ifosfamide, 5-fluorouracil (5-FU), irinotecan, or mitomycin), a targeted therapy (e.g., an angiogenesis inhibitor (e.g., bevacizumab)), or a combination thereof.

In an embodiment, the cancer is an endometrial cancer. CD138 is expressed in endometrial cancer (Hasengaowa et al. Ann Oncol. 2005; 16:1109-1115). In an embodiment, the endometrial cancer is an endometrioid carcinoma, a serous carcinoma, a clear cell carcinoma, a mucinous carcinoma, a mixed or undifferentiated carcinoma, a squamous cell carcinoma, a transitional cell carcinoma, or an endometrial stromal sarcoma. The anti-CD138 antibody molecules described herein can be used alone or in combination with a second therapeutic agent, procedure, or modality to treat an endometrial cancer. In an embodiment, the anti-CD138 antibody molecule is used in combination with a surgery, a radiation therapy, a hormone therapy (e.g., a progestin (e.g., medroxyprogesterone acetate) or megestrol acetate), tamoxifen, a luteinizing hormone-releasing hormone (LHRH) agonist (e.g., goserelin or leuprolide), an aromatase inhibitor (e.g., letrozole, anastrozole, or exemestane), a chemotherapy (e.g., paclitaxel, carboplatin, doxorubicin, liposomal doxorubicin, or cisplatin), or a combination thereof.

In an embodiment, the cancer is a gallbladder cancer. CD138 is overexpressed in gallbladder cancer (Roh et al. Eur Surg Res. 2008; 41(2): 245-250). In an embodiment, the gallbladder cancer is an adenocarcinoma or a papillary adenocarcinoma. The anti-CD138 antibody molecules described herein can be used alone or in combination with a second therapeutic agent, procedure, or modality to treat a gallbladder cancer. In an embodiment, the anti-CD138 antibody molecule is used in combination with a surgery, a radiation therapy, a chemotherapy (e.g., gemcitabine, cisplatin, 5-fluorouracil (5-FU), capecitabine, or oxaliplatin), or a palliative therapy (e.g., a biliary stent, a biliary catheter, a biliary bypass, an alcohol injection, a pain medicine, or a combination thereof.

In an embodiment, the cancer is a gastric cancer. Strong stromal CD138 expression is associated with gastric cancer (Wiksten et al. Int J Cancer. 2001; 95(1):1-6). In an embodiment, the gastric cancer is an adenocarcinoma (ACA). The anti-CD138 antibody molecules described herein can be used alone or in combination with a second therapeutic agent, procedure, or modality to treat a gastric cancer. In an embodiment, the anti-CD138 antibody molecule is used in combination with a surgery, a chemotherapy (e.g., 5-FU (fluorouracil), capecitabine, carboplatin, cisplatin, docetaxel, epirubicin, irinotecan, oxaliplatin, or paclitaxel), or a combination thereof.

In an embodiment, the cancer is a brain cancer (e.g., a glioma). CD138 is expressed in glioma (Xu et al. Mol Biol Rep. 2012; 39(9): 8979-8985). In an embodiment, the glioma is an astrocytoma, an en ependymoma, or an oligodendroglioma. The anti-CD138 antibody molecules described herein can be used alone or in combination with a second therapeutic agent, procedure, or modality to treat a glioma. In an embodiment, the anti-CD138 antibody molecule is used in combination with a surgery, a radiation therapy, a chemotherapy (e.g., carboplatin, carmustine (BCNU), cisplatin, cyclophosphamide, etoposide, irinotecan, lomustine (CCNU), methotrexate, procarbazine, temozolomide, or vincristine), a targeted therapy (e.g., bevacizumab or everolimus), a corticosteroid (e.g., dexamethasone), an anti-seizure drug, or a hormone, or a combination thereof.

In an embodiment, the cancer is a laryngeal cancer. CD138 expression is in laryngeal cancer (Klatka et al. Otolaryngol Pol. 2004; 58: 933-940). In an embodiment, the laryngeal cancer is a squamous cell carcinoma or an adenocarcinoma. The anti-CD138 antibody molecules described herein can be used alone or in combination with a second therapeutic agent, procedure, or modality to treat a laryngeal cancer. In an embodiment, the anti-CD138 antibody molecule is used in combination with a surgery, a radiation therapy, a chemotherapy (e.g., cisplatin, carboplatin, 5-fluorouracil (5-FU), docetaxel, paclitaxel, bleomycin, methotrexate, or ifosfamide), a targeted therapy (e.g., an EGFR inhibitor (e.g., cetuximab)), or a combination thereof. In an embodiment, the cancer is a liver cancer. In an embodiment, the liver cancer is a hepatocellular carcinoma (HCC), a cholangiocarcinoma, an angiosarcoma, or a secondary liver cancer. The anti-CD138 antibody molecules described herein can be used alone or in combination with a second therapeutic agent, procedure, or modality to treat a liver cancer. In an embodiment, the anti-CD138 antibody molecule is used in combination with a surgery, tumor ablation, tumor embolization, a radiation therapy, a targeted therapy (e.g., sorafenib or regorafenib), a chemotherapy (e.g., doxorubicin, 5-fluorouracil (5-FU), or cisplatin), or a combination thereof.

In an embodiment, the cancer is a lung cancer. CD138 is expressed in lung cancer (Anttonen et al. Lung Cancer. 2001; 32:297-305). In an embodiment, the lung cancer is a non-small cell lung cancer (NSCLC) (e.g., an adenocarcinoma, a squamous cell carcinoma, a large cell carcinoma, or a large cell neuroendocrine tumor) or a small cell lung cancer (SCLC). The anti-CD138 antibody molecules described herein can be used alone or in combination with a second therapeutic agent, procedure, or modality to treat a lung cancer. In an embodiment, the anti-CD138 antibody molecule is used in combination with a surgery, radiofrequency ablation (RFA), a radiation therapy, a chemotherapy (cisplatin, carboplatin, paclitaxel, albumin-bound paclitaxel (nab-paclitaxel), docetaxel, gemcitabine, vinorelbine, irinotecan, etoposide, vinblastine, or pemetrexed), a targeted therapy (an angiogenesis inhibitor (e.g., bevacizumab or ramucirumab), an EGFR inhibitor (e.g., erlotinib, afatinib, gefitinib, osimertinib, or necitumumab), an ALK inhibitor (e.g., crizotinib, ceritinib, alectinib, or brigatinib), a BRAF inhibitor (e.g., dabrafenib or trametinib), an immunotherapy (e.g., a PD-1 inhibitor (e.g., nivolumab or pembrolizumab) or a PD-L1 inhibitor (e.g., atezolizumab), or a combination thereof, e.g., to treat a non-small cell lung cancer. In an embodiment, the anti-CD138 antibody molecule is used in combination with a surgery, a radiation therapy, a chemotherapy (cisplatin, etoposide, carboplatin, or irinotecan), or a combination thereof, e.g., to treat a small cell lung cancer.

In an embodiment, the cancer is a mesothelioma. CD138 is expressed in mesothelioma (Kumar-singh et al. J Pathol. 1998; 186:300-305). In an embodiment, the mesothelioma is an epithelioid mesothelioma, a sarcomatoid mesothelioma, or abiphasic mesothelioma. In an embodiment, the mesothelioma is a pleural mesothelioma, a peritoneal mesothelioma, or a pericardial mesothelioma. The anti-CD138 antibody molecules described herein can be used alone or in combination with a second therapeutic agent, procedure, or modality to treat a mesothelioma. In an embodiment, the anti-CD138 antibody molecule is used in combination with a surgery, a radiation therapy, a chemotherapy (e.g., pemetrexed, cisplatin, carboplatin, gemcitabine, methotrexate, vinorelbine, mitomycin, or doxorubicin), or a combination thereof.

In an embodiment, the cancer is a nasopharyngeal cancer. CD138 is expressed in nasopharyngeal cancer (Kim et al. Head Neck. 2011; 33:1458-1466). In an embodiment, the nasopharyngeal cancer is a keratinizing squamous cell carcinoma, a non-keratinizing differentiated carcinoma, or an undifferentiated carcinoma. The anti-CD138 antibody molecules described herein can be used alone or in combination with a second therapeutic agent, procedure, or modality to treat a nasopharyngeal cancer. In an embodiment, the anti-CD138 antibody molecule is used in combination with a surgery, a radiation therapy, a chemotherapy (e.g., carboplatin, doxorubicin, epirubicin, paclitaxel, docetaxel, gemcitabine, bleomycin, or methotrexate), a targeted therapy (e.g., cetuximab), or a combination thereof.

In an embodiment, the cancer is a nasopharyngeal cancer. CD138 is expressed in oral cancer (Al-Otaibi et al. J Oral Pathol Med. 2013; 42: 186-193). In an embodiment, the oral cancer is a squamous cell carcinoma, a verrucous carcinoma, or a minor salivary gland carcinoma. The anti-CD138 antibody molecules described herein can be used alone or in combination with a second therapeutic agent, procedure, or modality to treat an oral cancer. In an embodiment, the anti-CD138 antibody molecule is used in combination with a surgery, a radiation therapy, a chemotherapy (e.g., cisplatin, carboplatin, 5-fluorouracil (5-FU), paclitaxel, docetaxel, methotrexate, ifosfamide, or bleomycin), a targeted therapy (e.g., cetuximab), or a combination thereof.

In an embodiment, the cancer is an ovarian cancer. CD138 is expressed in ovarian cancer (Kusumoto et al. Oncol Rep. 2010; 23: 917-925; Davies et al. Clin Cancer Res. 2004; 10: 5178-5186). In an embodiment, the ovarian cancer is an epithelial cancer, a germ cell carcinoma, a stromal carcinoma, or a small cell carcinoma. The anti-CD138 antibody molecules described herein can be used alone or in combination with a second therapeutic agent, procedure, or modality to treat an ovarian cancer. In an embodiment, the anti-CD138 antibody molecule is used in combination with a surgery, a chemotherapy (e.g., cisplatin, carboplatin, paclitaxel, albumin bound paclitaxel (nab-paclitaxel), docetaxel, altretamine, capecitabine, cyclophosphamide, etoposide, gemcitabine, ifosfamide, irinotecan, liposomal doxorubicin, melphalan, pemetrexed, topotecan, or vinorelbine), a hormone therapy (e.g., a luteinizing-hormone-releasing hormone (LHRH) agonist (e.g., goserelin or leuprolide), tamoxifen, or aromatase inhibitor (e.g., letrozole, anastrozole, or exemestane), a targeted therapy (e.g., an angiogenesis inhibitor (e.g., bevacizumab), a PARP inhibitor (e.g., olaparib, rucaparib, or niraparib), a radiation therapy, or a combination thereof.

In an embodiment, the cancer is a pancreatic cancer. CD138 is expressed in pancreatic cancer (Juuti et al. Oncology. 2005; 68: 97-106). In an embodiment, the pancreatic cancer is an exocrine tumor or an endocrine tumor. The anti-CD138 antibody molecules described herein can be used alone or in combination with a second therapeutic agent, procedure, or modality to treat a pancreatic cancer. In an embodiment, the anti-CD138 antibody molecule is used in combination with a surgery, ablation, embolization, a radiation therapy, or a chemotherapy (cemcitabine, 5-fluorouracil (5-FU), irinotecan, oxaliplatin, albumin-bound paclitaxel, capecitabine, cisplatin, paclitaxel, docetaxel, or irinotecan liposome.

In an embodiment, the cancer is a prostate cancer. CD138 is expressed in prostate cancer (Ledezma et al. Asian J Androl. 2011; 13: 476-480; Shariat et al. BJU Int. 2008; 101:232-237; Kiviniemi et al. Apmis. 2004; 112: 89-97; Zellweger et al. Prostate. 2003; 55: 20-29). In an embodiment, the prostate cancer is an adenocarcinoma, a transitional cell (or urothelial) cancer, a squamous cell cancer, or a small cell prostate cancer. The anti-CD138 antibody molecules described herein can be used alone or in combination with a second therapeutic agent, procedure, or modality to treat a prostate cancer. In an embodiment, the anti-CD138 antibody molecule is used in combination with a surgery, a radiation therapy, a cryotherapy, a hormone therapy (e.g., orchiectomy, an LHRH agonist (e.g., leuprolide, goserelin, triptorelin, or histrelin), an LHRH antagonist (e.g., degarelix), a CYP17 inhibitor (e.g., abiraterone), an anti-androgen (e.g., flutamide, bicalutamide, nilutamide, or enzalutamide), an estrogen, or ketoconazole), a chemotherapy (e.g., docetaxel, cabazitaxel, mitoxantrone, or estramustine), a vaccine treatment (e.g., Sipuleucel-T), or a bone-directed treatment (e.g., a bisphosphonate (e.g., zoledronic acid), denosumab, a corticosteroid (e.g., prednisone or dexamethasone), an external radiation therapy, a radiopharmaceutical (e.g., Strontium-89, Samarium-153, or Radium-223), or a combination thereof.

In an embodiment, the cancer is a head and neck cancer. CD138 is expressed in head and neck cancer (Anttonen et al. Br J Cancer. 1999; 79: 558-564; Inki et al. Br J Cancer. 1994; 70: 319-323). In an embodiment, the head and neck cancer is a squamous cell carcinoma. The anti-CD138 antibody molecules described herein can be used alone or in combination with a second therapeutic agent, procedure, or modality to treat a head and neck cancer. In an embodiment, the anti-CD138 antibody molecule is used in combination with a surgery, a radiation therapy, a chemotherapy (e.g., methotrexate, bleomycin, or docetaxel), a targeted therapy (e.g., cetuximab), an immunotherapy (e.g., a PD-1 inhibitor (e.g., nivolumab or pembrolizumab)), or a combination thereof.

In an embodiment, the cancer is a thyroid cancer. CD138 is expressed in thyroid cancer (Oh & Park. J Korean Med Sci. 2006; 21: 397-405). In an embodiment, the thyroid cancer is a papillary carcinoma, a follicular carcinoma, a Hürthle cell carcinoma, a medullary thyroid carcinoma, or an anaplastic carcinoma. The anti-CD138 antibody molecules described herein can be used alone or in combination with a second therapeutic agent, procedure, or modality to treat a thyroid cancer. In an embodiment, the anti-CD138 antibody molecule is used in combination with a surgery, a radioactive iodine treatment, a thyroid hormone therapy, a radiation therapy, a chemotherapy, a targeted therapy (e.g., a kinase inhibitor (e.g., sorafenib or lenvatinib), or a combination thereof.

In an embodiment, the cancer is a chronic lymphocytic leukemia (CLL). CD138 is expressed in chronic lymphocytic leukemia cancer (Jilani et al. Int J Lab Hematol. 2009; 31:97-105). The anti-CD138 antibody molecules described herein can be used alone or in combination with a second therapeutic agent, procedure, or modality to treat a thyroid cancer. In an embodiment, the anti-CD138 antibody molecule is used in combination with a chemotherapy (e.g., a purine analog (e.g., fludarabine, pentostatin, or cladribine), an alkylating agent (e.g., chlorambucil, cyclophosphamide, or bendamustine), a corticosteroid (e.g., prednisone, methylprednisolone, or dexamethasone), doxorubicin, methotrexate, oxaliplatin, vincristine, etoposide, and cytarabine), an anti-CD20 antibody (rituximab, obinutuzumab, or ofatumumab), an anti-CD52 antibody (e.g., alemtuzumab), a targeted therapy (e.g., ibrutinib, idelalisib, or venetoclax), a stem cell transplant (SCT), or a combination thereof.

In an embodiment, the cancer is a lymphoma (e.g., a diffuse large B-cell lymphoma (DLBCL)). CD138 is expressed in DLBCL (Oh & Park. J Korean Med Sci. 2006; 21: 397-405; Bodoor et al. Asian Pac J Cancer Prev. 2012; 13: 3037-3046). The anti-CD138 antibody molecules described herein can be used alone or in combination with a second therapeutic agent, procedure, or modality to treat a DLBCL. In an embodiment, the anti-CD138 antibody molecule is used in combination with a chemotherapy (e.g., an alkylating agent (e.g., cyclophosphamide, chlorambucil, bendamustine, or ifosfamide), a corticosteroid (e.g., prednisone or dexamethasone), a platinum drug (cisplatin, carboplatin, or oxaliplatin), a purine analog (e.g., fludarabine, pentostatin, or cladribine), an anti-metabolite (e.g., cytarabine, gemcitabine, methotrexate, or pralatrexate), vincristine, doxorubicin, mitoxantrone, etoposide, or bleomycin), an immunotherapy (e.g., an anti-CD20 antibody (rituximab, obinutuzumab, or ofatumumab), an anti-CD52 antibody (e.g., alemtuzumab), an anti-CD30 antibody (e.g., brentuximab vedotin), interferon, an immunomodulating drug (e.g., thalidomide or lenalidomide), a targeted therapy (e.g., a proteasome inhibitor (e.g., bortezomib), a histone deacetylase (HDAC) inhibitor (e.g., romidepsin or belinostat), or a kinase inhibitor (e.g., ibrutinib or idelalisib)), a radiation therapy, a stem cell transplant (SCT), or a combination thereof.

In an embodiment, the cancer is a Hodgkin's lymphoma. CD138 is expressed in Hodgkin's lymphoma (Gharbaran et al. J Hematol Oncol. 2013; 6:62; Vassilakopoulos et al. Anticancer Res. 2005; 25: 4743-4746). The anti-CD138 antibody molecules described herein can be used alone or in combination with a second therapeutic agent, procedure, or modality to treat a Hodgkin's lymphoma. In an embodiment, the anti-CD138 antibody molecule is used in combination with a chemotherapy (e.g., doxorubicin, bleomycin, vinblastine, dacarbazine, etoposide, cyclophosphamide, vincristine, procarbazine, prednisone, mechlorethamine, vincristine, or vinblastine), a radiation therapy, an immunotherapy (e.g., an anti-CD30 antibody (e.g., brentuximab vedotin)), a stem cell transplant, or a combination thereof.

In an embodiment, the antibody molecule is used to treat or prevent a precancerous condition. Precancerous condition, also known as premalignant condition, potentially precancerous condition, or potentially premalignant condition, refers to a state of disordered morphology of cells that is associated with an increased risk of cancer. If left untreated, precancerous conditions may lead to cancer. In an embodiment, the premalignant lesion is morphologically atypical tissue which appears abnormal under microscopic examination, and in which cancer is more likely to occur than in its apparently normal counterpart. In an embodiment, the precancerous condition is smoldering myeloma or asymptomatic myeloma. In an embodiment, the precancerous condition is monoclonal gammopathy of undetermined significance (MGUS). Other examples of precancerous conditions include, but are not limited to, actinic keratosis, Barrett's esophagus, atrophic gastritis, ductal carcinoma in situ, dyskeratosis congenital, sideropenic dysphagia, lichen planus, oral submucous fibrosis, solar elastosis, cervical dysplasia, leukoplakia, and erythroplakia.

### Multiple Myeloma

The antibody molecule described herein can be used to treat or prevent multiple myeloma.

Multiple myeloma, also known as plasma cell myeloma, is a cancer of plasma cells, which are normally responsible for producing antibodies (Raab et al. Lancet. 2009; 374(9686): 324-39).

Signs or symptoms of multiple myeloma include, e.g., bone pain, anemia (e.g., normocytic and/or normochromic anemia), kidney failure (e.g., acute or chronical kidney failure), infection (e.g., pneumonias or pyelonephritis), a neurological symptom (e.g., weakness, confusion, fatigue, headache, visual change, retinopathy, radicular pain, loss of bowel or bladder control, carpal tunnel syndrome, or paraplegia).

Risk factors for multiple myeloma include, e.g., smoldering myeloma (also known as asymptomatic myeloma), monoclonal gammopathy of undetermined significance (MGUS), obesity, or familial predisposition. In an embodiment, the anti-CD138 antibody molecules described herein can be used to reduce (e.g., prevent) the progression of smoldering myeloma or MGUS to multiple myeloma.

Diagnostic criteria for symptomatic myeloma, asymptomatic myeloma and MGUS are described, e.g., in Kyle & Rajkumar Leukemia. 2009; 23(1): 3-9.

Diagnostic criteria for symptomatic myeloma (all three criteria must be met) include, e.g., clonal plasma cells >10% on bone marrow biopsy or (in any quantity) in a biopsy from other tissues (plasmacytoma), a monoclonal protein (Myeloma protein) in either serum or urine (except in cases of true non-secretory myeloma), and evidence of end-organ damage felt related to the plasma cell disorder (related organ or tissue impairment, commonly referred to by the acronym “CRAB”): hypercalcemia (corrected calcium >2.75 mmol/l, >11 mg/dL), renal insufficiency attributable to myeloma, anemia (hemoglobin <10 g/dl), bone lesions (lytic lesions or osteoporosis with compression fractures). Diagnostic criteria for asymptomatic/smoldering myeloma include, e.g., serum M protein >30 g/l (3 g/dL) and/or clonal plasma cells >10% on bone marrow biopsy and no myeloma-related organ or tissue impairment). Diagnostic criteria for monoclonal gammopathy of undetermined significance (MGUS) include, e.g., serum paraprotein <30 g/l (3 g/dL) and clonal plasma cells <10% on bone marrow biopsy and no myeloma-related organ or tissue impairment or a related B-cell lymphoproliferative disorder

Related conditions include, e.g., solitary plasmacytoma, plasma cell dyscrasia (e.g., AL amyloidosis), and peripheral neuropathy, organomegaly, endocrinopathy, monoclonal plasma cell disorder, and skin changes.

The International Staging System (ISS) for myeloma is described, e.g., in Greipp et al. J Clin Oncol. 2005; 23(15): 3412-20. For example, the ISS includes the following; Stage I: β2 microglobulin (β2M)<3.5 mg/L, albumin ≥3.5 g/dL; Stage II: β2M <3.5 mg/L and albumin <3.5 g/dL: or ß2M 3.5-5.5 mg/L irrespective of the serum albumin; Stage III: β2M ≥5.5 mg/L.

The ISS can be used along with the Durie-Salmon Staging System. The Durie-Salmon Staging System is described, e.g., in Durie & Salmon Cancer. 1975; 36(3):842-54. For example, the Durie-Salmon Staging System include the following; Stage I (all of Hb >10 g/dL, normal calcium, skeletal survey: normal or single plasmacytoma or osteoporosis, serum paraprotein level <5 g/dL if IgG. <3 g/dL if IgA, urinary light chain excretion <4 g/24 h); Stage II (fulfilling the criteria of neither I nor III); Stage III (one or more of Hb<8.5 g/dL, high calcium >12 mg/dL, skeletal survey: three or more lytic bone lesions, serum paraprotein >7 g/dL if IgG. >5 g/dL if IgA, urinary light chain excretion >12 g/24 h). Stages I, II, and III of the Durie-Salmon Staging System can be divided into A or B depending on serum creatinine: A: serum creatinine <2 mg/dL (<177 μmol/L): B: serum creatinine >2 mg/dl (>177 μmol/L).

Other treatments for multiple myeloma that can be used in combination with an anti-CD138 antibody molecule described herein include, e.g., a protease inhibitor (e.g., bortezomib (VELCADE®), carfilzomib (KYPROLIS®), or ixazomib (NINLARO®)), an immunomodulating agent (e.g., thalidomide (THALOMID®), lenalidomide (REVLIMID®), or pomalidomide (POMALYST®)), a chemotherapy (e.g., melphalan, vincristine (ONCOVIN®), cyclophosphamide, etoposide, doxorubicin (ADRIAMYCIN®), liposomal doxorubicin (DOXIL®), or bendamustine (TREANDA®)), a corticosteroid (e.g., prednisone or dexamethasone), a histone deacetylase (HDAC) inhibitor (e.g., panobinostat (FARYDAK®), an anti-CD38 antibody (e.g., daratumumab (DARZALEX®)), an anti-SLAMF7 antibody (e.g., clotuzumab (EMPLICITI®)), an interferon, or a bone marrow transplantation (e.g., autologous stem cell transplantation (ASCT) or allogeneic stem cell transplantation), a bisphosphonate (e.g., pamidronate (AREDIA®) and zoledronic acid (ZOMETA®), a radiation therapy, a surgery, an intravenous immunoglobulin (IVIG), a treatment for low blood cell count (e.g., erythropoietin (PROCRIT®) or darbepoietin (ARANESP®), plasmapheresis, or a combination thereof.

Exemplary combination therapies that can be used in combination with an anti-CD138 antibody molecule described herein for treating multiple myeloma include, but are not limited to, melphalan and prednisone (MP), with or without thalidomide or bortezomib; vincristine, doxorubicin (ADRIAMYCIN®), and dexamethasone (VAD); thalidomide (or lenalidomide) and dexamethasone; bortezomib, doxorubicin, and dexamethasone; bortezomib, dexamethasone, and thalidomide (or lenalidomide); liposomal doxorubicin, vincristine, and dexamethasone; carfilzomib, lenalidomide, and dexamethasone; dexamethasone, cyclophosphamide, etoposide, and cisplatin (DCEP); dexamethasone, thalidomide, cisplatin, doxorubicin, cyclophosphamide, and etoposide (DT-PACE), with or without bortezomib; panobinostat, bortezomib, and dexamethasone; ixazomib, lenalidomide; and dexamethasone, and elotuzumab, lenalidomide, and dexamethasone.

### Combination Therapies

The antibody molecules described herein can be used in combination with other therapies. For example, the combination therapy can include an antibody molecule co-formulated with, and/or co-administered with, one or more additional therapeutic agents, e.g., one or more additional therapeutic agents described herein. In other embodiments, the antibody molecules are administered in combination with other therapeutic treatment modalities, e.g., other therapeutic treatment modalities described herein. Such combination therapies may advantageously utilize lower dosages of the administered therapeutic agents, thus avoiding possible toxicities or complications associated with the various monotherapies.

Administered “in combination”, as used herein, means that two (or more) different treatments are delivered to the subject before, or during the course of the subject's affliction with a disorder. In an embodiment, two or more treatments are delivered prophylactically, e.g., before the subject has the disorder or is diagnosed with the disorder. In another embodiment, the two or more treatments are delivered after the subject has developed or diagnosed with the disorder. In some embodiments, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, the additional agent is a second antibody molecule, e.g., an antibody molecule different from a first antibody molecule. Exemplary antibody molecules that can be used in combination include, but are not limited to, any combination of the antibody molecules listed in Table 1.

In an embodiment, the antibody molecule is administered in combination with a second therapy to treat or prevent a myeloma, e.g., multiple myeloma.

In an embodiment, the antibody molecule is administered in combination with a protease inhibitor. Exemplary protease inhibitors include, e.g., bortezomib (VELCADE®), carfilzomib (KYPROLIS®), and ixazomib (NINLARO®).

In an embodiment, the antibody molecule is administered in combination with an immunomodulating agent. Exemplary immunomodulating agents include, e.g., thalidomide (THALOMID®), lenalidomide (REVLIMID®), and pomalidomide (POMALYST®).

In an embodiment, the antibody molecule is administered in combination with a chemotherapeutic agent. Exemplary chemotherapeutic agents include, e.g., melphalan, vincristine (ONCOVIN®), cyclophosphamide, etoposide, doxorubicin (ADRIAMYCIN®), liposomal doxorubicin (DOXIL®), and bendamustine (TREANDA®).

In an embodiment, the antibody molecule is administered in combination with a corticosteroid, e.g., prednisone and dexamethasone.

In an embodiment, the antibody molecule is administered in combination with a histone deacetylase (HDAC) inhibitor, e.g., panobinostat (FARYDAK®).

In an embodiment, the antibody molecule is administered in combination with an anti-CD38 antibody, e.g., daratumumab (DARZALEX®).

In an embodiment, the antibody molecule is administered in combination with an anti-SLAMF7 antibody, e.g., elotuzumab (EMPLICITI®).

In an embodiment, the antibody molecule is administered in combination with an interferon.

In an embodiment, the antibody molecule is administered in combination with bone marrow transplantation (e.g., autologous stem cell transplantation (ASCT) or allogeneic stem cell transplantation).

In an embodiment, the antibody molecule is administered in combination with a bisphosphonate, e.g., pamidronate (AREDIA®) or zoledronic acid (ZOMETA®).

In an embodiment, the antibody molecule is administered in combination with a radiation therapy.

In an embodiment, the antibody molecule is administered in combination with a surgery.

In an embodiment, the antibody molecule is administered in combination with an intravenous immunoglobulin (IVIG).

In an embodiment, the antibody molecule is administered in combination with a treatment for low blood cell count, e.g., erythropoietin (PROCRIT®) or darbepoietin (ARANESP®).

In an embodiment, the antibody molecule is administered in combination with plasmapheresis.

In an embodiment, the antibody molecule is administered in combination with melphalan and prednisone (MP), with or without thalidomide or bortezomib.

In an embodiment, the antibody molecule is administered in combination with vincristine, doxorubicin (ADRIAMYCIN®), and dexamethasone (VAD).

In an embodiment, the antibody molecule is administered in combination with thalidomide (or lenalidomide) and dexamethasone.

In an embodiment, the antibody molecule is administered in combination with bortezomib, doxorubicin, and dexamethasone.

In an embodiment, the antibody molecule is administered in combination with bortezomib, dexamethasone, and thalidomide (or lenalidomide).

In an embodiment, the antibody molecule is administered in combination with liposomal doxorubicin, vincristine, and dexamethasone;

In an embodiment, the antibody molecule is administered in combination with carfilzomib, lenalidomide, and dexamethasone.

In an embodiment, the antibody molecule is administered in combination with dexamethasone, cyclophosphamide, etoposide, and cisplatin (DCEP).

In an embodiment, the antibody molecule is administered in combination with dexamethasone, thalidomide, cisplatin, doxorubicin, cyclophosphamide, and etoposide (DT-PACE), with or without bortezomib.

In an embodiment, the antibody molecule is administered in combination with panobinostat, bortezomib, and dexamethasone.

In an embodiment, the antibody molecule is administered in combination with ixazomib, lenalidomide, and dexamethasone.

In an embodiment, the antibody molecule is administered in combination with elotuzumab, lenalidomide, and dexamethasone.

In an embodiment, the antibody molecule is administered in combination with a second agent that targets the CD138 pathway. Exemplary agents that target the CD138 pathway include, e.g., an agent that targets the extracellular domain of CD138 (e.g., synstatin, BT-062-DM4 (indatuximab ravtansine), B-B4 conjugated to ¹³¹I, OC-46F2, or GLVGLIFAV (SEQ ID NO: 448)), an agent that targets shed CD138 (e.g., NSC 405020, BB-94, PI-88, PG545, M402, SST00001, or Pentraxin-3), and an agent that targets genetic expression of CD138 (e.g., an all-trans retinoic acid, nimesulide, zoledronic acid, or imatinib). Other agents that target the CD138 pathway are described, e.g., Akl et al. Oncotarget. 2015; 6(30):28693-28715, the content of which in incorporated by reference in its entirety.

In an embodiment, the antibody molecule is administered in combination with lenalidomide and/or dexamethasone, e.g., to treat a multiple myeloma (e.g., a relapsed multiple myeloma).

In an embodiment, the antibody molecule is administered in combination with an FGFR2 antagonist (e.g., an anti-FGFR2 antibody, e.g., FPA144) to treat a solid tumor (e.g., an advanced solid tumor).

In an embodiment, the antibody molecule is administered in combination with a αᵥβ₃ inhibitor (e.g., an ADC against integrin αᵥβ₃, e.g., brentuximab vedotin), e.g., to treat Hodgkin lymphoma (e.g., relapsed or refractory Hodgkin lymphoma).

In an embodiment, the antibody molecule is administered in combination with a heparin or heparanase inhibitor (e.g., roneparstat (SST0001)), e.g., to treat a multiple myeloma (e.g., an advanced multiple myeloma).

In an embodiment, the antibody molecule is administered in combination with a VEGFR inhibitor (e.g., bevacizumab or cediranib), e.g., to treat a cancer (e.g., an advanced cancer).

In an embodiment, the antibody molecule is administered in combination with a Wnt signaling pathway inhibitor (e.g., ipafricept (OMP-54F28)), e.g., to treat a solid tumor.

In an embodiment, the antibody molecule is administered in combination with an FAK inhibitor (e.g., defactinib (VS-6063) or GSK2256098), e.g., to treat a solid tumor, e.g., a lung cancer (e.g., a non-small cell lung cancer, e.g., with a KRAS mutation).

In an embodiment, the antibody molecule is administered in combination with a glysoaminoglycan or heparanase inhibitor (e.g., necuparanib (M402)), optionally, further in combination with a chemotherapeutic agent (e.g., nab-paclitaxel or gemcitabine), e.g., to treat a pancreatic cancer (e.g., a metastatic pancreatic cancer).

In an embodiment, the antibody molecule is administered in combination with a mannose oligosaccharide, or a FGF, heparanase, and/or VEGF inhibitor (e.g., muparfostat (PI-88)), e.g., to treat a cancer (e.g., a melanoma).

In an embodiment, the antibody molecule is administered in combination with a chemically modified heparin sulfate/heparanase inhibitor (e.g., PG545), e.g., to treat a solid tumor (e.g., an advanced solid tumor).

In an embodiment, the antibody molecule is administered in combination with an amino acid or matrix metalloprotease inhibitor (e.g., intrapleural batimastat (BB-94)), e.g., to treat a malignant pleural effusion.

In an embodiment, the antibody molecule is administered in combination with a chimeric anti-CD138 antigen receptor-modified T cells, e.g., to treat a multiple myeloma (e.g., a relapsed and/or refractory multiple myeloma). Exemplary therapies that can be used in combination with an antibody molecule or composition described herein to treat or prevent other disorders are also described in the section of “Methods of Treating or Preventing Disorders” herein.

### Methods of Diagnosis

In some aspects, the present disclosure provides a diagnostic method for detecting the presence of CD138 in vitro (e.g., in a biological sample, such as a biopsy or blood sample) or in vivo (e.g., in vivo imaging in a subject). The method includes: (i) contacting the sample with an anti-CD138 antibody molecule described herein, or administering to the subject, the antibody molecule; (optionally) (ii) contacting a reference sample, e.g., a control sample (e.g., a control biological sample, such as a biopsy or blood sample) or a control subject with an antibody molecule described herein; and (iii) detecting formation of a complex between the antibody molecule and CD138 in the sample or subject, or the control sample or subject, wherein a change, e.g., a statistically significant change, in the formation of the complex in the sample or subject relative to the control sample or subject is indicative of the presence of CD138 in the sample. The antibody molecule can be directly or indirectly labeled with a detectable substance to facilitate detection of the bound or unbound antibody molecule. Suitable detectable substances include various enzymes, prosthetic groups, fluorescent materials, luminescent materials and radioactive materials, as described above and described in more detail below.

The term “sample,” as it refers to samples used for detecting a polypeptide (e.g., CD138) or a nucleic acid encoding the polypeptide includes, but is not limited to, cells, cell lysates, proteins or membrane extracts of cells, body fluids such as blood, or tissue samples such as biopsies.

Complex formation between the antibody molecule, and CD138, can be detected by measuring or visualizing either the antibody molecule bound to CD138 or unbound antibody molecule. Any suitable detection assays can be used, and conventional detection assays include an enzyme-linked immunosorbent assays (ELISA), a radioimmunoassay (RIA) or tissue immunohistochemistry. Alternative to labeling the antibody molecule, the presence of CD138 can be assayed in a sample by a competition immunoassay utilizing standards labeled with a detectable substance and an unlabeled antibody molecule. In this assay, the biological sample, the labeled standards and the antibody molecule are combined and the amount of labeled standard bound to the unlabeled binding molecule is determined. The amount of CD138 in the sample is inversely proportional to the amount of labeled standard bound to the antibody molecule.

The anti-CD138 antibody molecules described herein can be used to diagnose disorders that can be treated or prevented by the anti-CD138 antibody molecules described herein. The detection or diagnostic methods described herein can be used in combination with other methods described herein to treat or prevent a disorder described herein.

The present disclosure also includes any of the following numbered paragraphs:

1. An anti-CD138 antibody molecule, which:
(i) binds, or substantially binds, to CD138 in an extracellular region proximal to the transmembrane domain of CD138; and
(ii) causes an antibody-dependent cellular cytotoxicity (ADCC) activity on a cell expressing CD138.

2. The antibody molecule of paragraph 1, wherein the C-terminus of the extracellular region proximal to the transmembrane domain is within 70, 65, 60, 55, 50, 45, 40, 35, 30, 25, 20, 15, 10, or 5 amino acids from the N-terminus of the transmembrane domain.

3. The antibody molecule of paragraph 1 or 2, wherein the N-terminus of the extracellular region proximal to the transmembrane domain is within 75, 70, 65, 60, 55, 50, 45, 40, 35, 30, 25, 20, 15, 10, or 5 amino acids from the N-terminus of the transmembrane domain.

4. The antibody molecule of any of paragraphs 1-3, which binds to an epitope on CD138 comprising five or more (e.g., 6, 7, 8, 9, 10, 11, 12, 13, 14, 15, 20, 25, 30, 35, 40, 45, 50, 55, 60, 65, 70, 75, or more) consecutive amino acid residues in the extracellular region.

5. The antibody molecule of any of paragraphs 1-4, wherein the extracellular region proximal to the transmembrane domain comprises, or consists of, amino acids 210-250 or 220-245 of any of SEQ ID NOS: 1-3 or 450.

6. The antibody molecule of any of paragraphs 1-5, which binds to an Fc receptor (FcR) (e.g., one or more of FcγRI, FcγRIIa, FcγRIIb, FcγRIIc, FcγRIIIa, or FcγRIIIb) on the surface of an immune cell (e.g., a natural killer (NK) cell, a macrophage, a monocyte, or an eosinophil).

7. The antibody molecule of any of paragraphs 1-5, wherein the cell expressing CD138 is a cancer cell or precancerous cell.

8. The antibody molecule of paragraph 7, wherein the cancer or precancerous cell is a myeloma cell.

9. The antibody molecule of any of paragraphs 1-8, which does not bind, or binds with low affinity, to an extracellular region of CD138 distant from the transmembrane domain.

10. The antibody molecule of any of paragraphs 1-9, which does not bind to an epitope on CD138 comprising five or more (e.g., 6, 7, 8, 9, 10, 11, 12, 13, 14, 15, 20, 25, 30, 35, or more) consecutive amino acid residues in an extracellular region distant from the transmembrane domain.

11. The antibody molecule of any of paragraphs 1-8, which binds, or substantially binds, an epitope on CD138 comprising five or more (e.g., 6, 7, 8, 9, 10, 11, 12, 13, 14, 15, 20, 25, 30, 35, or more) consecutive amino acid residues in an extracellular region distant from the transmembrane domain.

12. The antibody molecule of any of paragraphs 9-11, wherein the C-terminus of the extracellular region distant from the transmembrane domain is at least 100, 110, 120, 130, 140, 150, 160, 170, 180, 190, or 200 amino acids away from the N-terminus of the transmembrane domain.

13. The antibody molecule of any of paragraphs 9-12, wherein the extracellular region distant from the transmembrane domain comprises amino acids 23-50, 51-95, 88-121, or 111-150 of any of SEQ ID NOS: 1-3 or 450.

14. The antibody molecule of any of paragraphs 1-13, which does not bind, or binds with low affinity, to the integrin binding domain (IBD) of CD138, a region N-terminal to the IBD of CD138, or both.

15. The antibody molecule of any of paragraphs 1-13, which binds to the IBD of CD138, a region N-terminal to the IBD of CD138, or both.

16. The antibody molecule of any of paragraphs 1-15, which binds to CD138 with a disassociation constant (K_{D}) of less than about 100, 90, 80, 70, 60, 50, 40, 30, 20, 10, 8, 6, 4, 3, 2, 1, 0.5, 0.2, 0.1, 0.05, 0.01, 0.005, or 0.001 nM, or about 10-0.001, 10-0.01, 5-0.01, 3-0.05, or 1-0.1 nM.

17. The antibody molecule of any of paragraphs 1-16, wherein the binding affinity of the antibody molecule to a membrane-bound CD138 is at least 2, 3, 4, 5, 6, 7, 8, 9, 10, 20, 50, 100, 200, or 500-fold higher than the binding affinity to a soluble CD138.

18. The antibody molecule of any of paragraphs 1-17, which binds to a membrane-bound CD138 with a K_{D} less than about 100, 90, 80, 70, 60, 50, 40, 30, 20, 10, 8, 6, 4, 3, 2, 1, 0.5, 0.2, 0.1, 0.05, 0.01, 0.005, or 0.001 nM, or about 10-0.001, 10-0.01, 5-0.01, 3-0.05, or 1-0.1 nM.

19. The antibody molecule of any of paragraphs 1-18, which binds to a soluble CD138 with a K_{D} less than about 100, 90, 80, 70, 60, 50, 40, 30, 20, 10, 8, 6, 4, 3, 2, 1, 0.5, 0.2, 0.1, 0.05, 0.01, 0.005, or 0.001 nM, or about 10-0.001, 10-0.01, 5-0.01, 3-0.05, or 1-0.1 nM, or with a K_{D} of more than about 100, 200, 300, 400, or 500 nM.

20. The antibody molecule of any of paragraphs 1-19, which binds to a membrane-bound CD138 preferably over a soluble CD138, e.g., the binding affinity to a membrane-bound CD138 is at least 2, 3, 4, 5, 6, 7, 8, 9, or 10-fold higher than the binding affinity to a soluble CD138; or binds with similar affinity to a membrane-bound CD138 and a soluble CD138, e.g., the binding affinity to a membrane-bound CD138 is less than about 10%, 20%, 30%, 40%, 50%, 60%, 70%, 80%, 90%, or 100% higher than the binding affinity to a soluble CD138.

21. The antibody molecule of any of paragraphs 1-20, which binds to C1q and causes a complement-dependent cytotoxicity (CDC) activity on a cell expressing CD138.

22. The antibody molecule of any of paragraphs 1-21, which reduces (e.g., inhibits, blocks, or neutralizes) one or more biological activities of a cell expressing CD138 in vitro, ex vivo, or in vivo.

23. The antibody molecule of any of paragraphs 1-22, which mediates homotypic adhesion of one or more CD138-expressing cells.

24. The antibody molecule of any of paragraphs 1-23, which inhibits the action of a protease on a membrane-bound CD138, e.g., to reduce shedding of CD138.

25. The antibody molecule of any of paragraphs 1-24, which reduces (e.g., inhibits) proliferation of a cancer or precancerous cell expressing CD138.

26. The antibody molecule of any of paragraphs 1-25, comprising one or more (e.g., two or three) heavy chain CDRs and/or one or more (e.g., two or three) light chain CDRs of an anti-CD138 monoclonal antibody described herein.

27. The antibody molecule of any of paragraphs 1-26, comprising a heavy chain variable region (VH) and/or light chain variable region (VL) of an anti-CD138 monoclonal antibody described herein.

28. The antibody molecule of any of paragraphs 1-27, comprising an Fc region.

29. An anti-CD138 antibody molecule, which binds, or substantially binds, to an epitope on CD138 comprising four or more (e.g., 5, 6, 7, 8, 9, 10, 11, 12, 13, 14, 15, 20, 25, 30, 35, 40, 45, 50, 55, 60, 65, 70, 75, or more) consecutive amino acid residues in an extracellular region proximal to the transmembrane domain of CD138.

30. The antibody molecule of paragraph 29, wherein the C-terminus of the extracellular region proximal to the transmembrane domain is within 70, 65, 60, 55, 50, 45, 40, 35, 30, 25, 20, 15, 10, or 5 amino acids from the N-terminus of the transmembrane domain.

31. The antibody molecule of paragraph 29 or 30, wherein the N-terminus of the extracellular region proximal to the transmembrane domain is within 75, 70, 65, 60, 55, 50, 45, 40, 35, 30, 25, 20, 15, 10, or 5 amino acids from the N-terminus of the transmembrane domain.

32. The antibody molecule of any of paragraphs 29-31, wherein the extracellular region proximal to the transmembrane domain comprises, or consists of, amino acids 176-250 of any of SEQ ID NOS: 1-3 or 450.

33. The antibody molecule of any of paragraphs 29-32, which does not bind, or binds with low affinity, to an extracellular region of CD138 distant from the transmembrane domain.

34. The antibody molecule of any of paragraphs 29-33, wherein the epitope does not comprise five or more (e.g., 6, 7, 8, 9, 10, 11, 12, 13, 14, 15, 20, 25, 30, 35, or more) consecutive amino acid residues in an extracellular region distant from the transmembrane domain.

35. The antibody molecule of paragraph 33 or 34, wherein the C-terminus of the extracellular region distant from the transmembrane domain is at least 100, 110, 120, 130, 140, 150, 160, 170, 180, 190, or 200 amino acids away from the N-terminus of the transmembrane domain.

36. The antibody molecule of any of paragraphs 33-35, wherein the extracellular region distant from the transmembrane domain comprises amino acids 23-50, 51-95, 88-121, or 111-150 of any of SEQ ID NOS: 1-3 or 450.

37. The antibody molecule of any of paragraphs 33-36, which does not bind, or binds with low affinity, to the integrin binding domain (IBD) of CD138, a region N-terminal to the IBD of CD138, or both.

38. The antibody molecule of any of paragraphs 33-36, which binds to the IBD of CD138, a region N-terminal to the IBD of CD138, or both.

39. An anti-CD138 antibody molecule, which binds, or substantially binds, to an epitope on CD138 comprising four or more (e.g., 5, 6, 7, 8, 9, 10, 11, 12, 13, 14, 15, 20, 25, 30, 35, 40, 45, 50, 55, 60, 65, 70, 75, or more) consecutive amino acid residues in an extracellular region distant to the transmembrane domain of CD138, wherein the epitope does not consist of amino acid residues 107-111 of any of SEQ ID NOS: 1-3 or 450.

40. The antibody molecule of paragraph 39, wherein the epitope does not comprise amino acids 107-111 of any of SEQ ID NOS: 1-3 or 450.

41. The antibody molecule of paragraph 39 or 40, wherein the C-terminus of the extracellular region distant from the transmembrane domain is at least 100, 110, 120, 130, 140, 150, 160, 170, 180, 190, or 200 amino acids away from the N-terminus of the transmembrane domain.

42. The antibody molecule of any of paragraphs 39-41, wherein the extracellular region distant to the transmembrane domain comprises, or consists of, amino acids 88-121 of any of SEQ ID NOS: 1-3 or 450.

43. An anti-CD138 antibody molecule, which binds, or substantially binds, to an epitope on CD138 comprising four or more (e.g., 5, 6, 7, 8, 9, 10, 11, 12, 13, 14, 15, 20, 25, 30, 35, 40, 45, 50, 55, 60, 65, 70, 75, or more) consecutive amino acid residues in an extracellular region proximal to the transmembrane domain of CD138; and four or more (e.g., 5, 6, 7, 8, 9, 10, 11, 12, 13, 14, 15, 20, 25, 30, 35, 40, 45, 50, 55, 60, 65, 70, 75, or more) consecutive amino acid residues in an extracellular region distant to the transmembrane domain of CD138.

44. The antibody molecule of paragraph 43, wherein the C-terminus of the extracellular region proximal to the transmembrane domain is within 70, 65, 60, 55, 50, 45, 40, 35, 30, 25, 20, 15, 10, or 5 amino acids from the N-terminus of the transmembrane domain.

45. The antibody molecule of paragraph 43 or 44, wherein the N-terminus of the extracellular region proximal to the transmembrane domain is within 75, 70, 65, 60, 55, 50, 45, 40, 35, 30, 25, 20, 15, 10, or 5 amino acids from the N-terminus of the transmembrane domain.

46. The antibody molecule of any of paragraphs 43-45, wherein the extracellular region proximal to the transmembrane domain comprises, or consists of, amino acids 176-250 or amino acids 210-250 of any of SEQ ID NOS: 1-3 or 450.

47. The antibody molecule of any of paragraphs 43-46, wherein the C-terminus of the extracellular region distant from the transmembrane domain is at least 100, 110, 120, 130, 140, 150, 160, 170, 180, 190, or 200 amino acids away from the N-terminus of the transmembrane domain.

48. The antibody molecule of any of paragraphs 43-47, wherein the extracellular region distant from the transmembrane domain comprises, or consists of, amino acids 23-50, 51-95, 88-121, or 111-150 of any of SEQ ID NOS: 1-3 or 450.

49. The antibody molecule of any of paragraphs 43-48, wherein the extracellular region distant to the transmembrane domain comprises, or consists of, amino acids 88-121 of any of SEQ ID NOS: 1-3 or 450.

50. The antibody molecule of any of paragraphs 43-49, which does not bind, or binds with low affinity, to the integrin binding domain (IBD) of CD138.

51. The antibody molecule of any of paragraphs 43-50, which does not bind, or binds with low affinity, to a region N-terminal to the IBD of CD138.

52. The antibody molecule of paragraph 51, wherein the epitope does not comprise amino acids 107-111 of any of SEQ ID NOS: 1-3 or 450.

53. The antibody molecule of any of paragraphs 43-49, which binds to the IBD of CD138.

54. The antibody molecule of any of paragraphs 43-50, which binds to a region N-terminal to the IBD of CD138.

55. The antibody molecule of paragraph 54, wherein the epitope comprises amino acids 107-111 of any of SEQ ID NOS: 1-3 or 450.

56. The antibody molecule of any of paragraphs 29-55, which binds to an Fc receptor (FcR) (e.g., one or more of FcγRI, FcγRIIa, FcγRIIb, FcγRIIc, FcγRIIIa, or FcγRIIIb) on the surface of an immune cell (e.g., a natural killer (NK) cell, a macrophage, a monocyte, or an eosinophil).

57. The antibody molecule of any of paragraphs 29-56, which is capable of causing an ADCC activity on a cell expressing CD138.

58. The antibody molecule of paragraph 57, wherein the cell expressing CD138 is a cancer cell or precancerous cell.

59. The antibody molecule of paragraph 58, wherein the cancer or precancerous cell is a myeloma cell.

60. The antibody molecule of any of paragraphs 29-59, which binds to CD138 with a disassociation constant (K_{D}) of less than about 100, 90, 80, 70, 60, 50, 40, 30, 20, 10, 8, 6, 4, 3, 2, 1, 0.5, 0.2, 0.1, 0.05, 0.01, 0.005, or 0.001 nM, or about 10-0.001, 10-0.01, 5-0.01, 3-0.05, or 1-0.1 nM.

61. The antibody molecule of any of paragraphs 29-60, wherein the binding affinity of the antibody molecule to a membrane-bound CD138 is at least 2, 3, 4, 5, 6, 7, 8, 9, 10, 20, 50, 100, 200, or 500-fold higher than the binding affinity to a soluble CD138; or binds with similar affinity to a membrane-bound CD138 and a soluble CD138, e.g., the binding affinity to a membrane-bound CD138 is less than about 10%, 20%, 30%, 40%, 50%, 60%, 70%, 80%, 90%, or 100% higher than the binding affinity to a soluble CD138.

62. The antibody molecule of any of paragraphs 29-61, which binds to a membrane-bound CD138 with a K_{D} less than about 100, 90, 80, 70, 60, 50, 40, 30, 20, 10, 8, 6, 4, 3, 2, 1, 0.5, 0.2, 0.1, 0.05, 0.01, 0.005, or 0.001 nM, or about 10-0.001, 10-0.01, 5-0.01, 3-0.05, or 1-0.1 nM.

63. The antibody molecule of any of paragraphs 29-62, which binds to a soluble CD138 with a K_{D} of less than about 100, 90, 80, 70, 60, 50, 40, 30, 20, 10, 8, 6, 4, 3, 2, 1, 0.5, 0.2, 0.1, 0.05, 0.01, 0.005, or 0.001 nM, or about 10-0.001, 10-0.01, 5-0.01, 3-0.05, or 1-0.1 nM, or greater than about 100, 200, 300, 400, or 500 nM.

64. The antibody molecule of any of paragraphs 29-63, which binds to a membrane-bound CD138 preferably over a soluble CD138, e.g., the binding affinity to a membrane-bound CD138 is at least 2, 3, 4, 5, 6, 7, 8, 9, or 10-fold higher than the binding affinity to a soluble CD138.

65. The antibody molecule of any of paragraphs 29-63, which binds to Clq and causes a complement-dependent cytotoxicity (CDC) activity on a cell expressing CD138.

66. The antibody molecule of any of paragraphs 29-65, which reduces (e.g., inhibits, blocks, or neutralizes) one or more biological activities of a cell expressing CD138 in vitro, ex vivo, or in vivo.

67. The antibody molecule of any of paragraphs 29-66, which mediates homotypic adhesion of one or more CD138-expressing cells.

68. The antibody molecule of any of paragraphs 29-67, which inhibits the action of a protease on a membrane-bound CD138, e.g., to reduce shedding of CD138.

69. The antibody molecule of any of paragraphs 29-68, which reduces (e.g., inhibits) proliferation of a cancer or precancerous cell expressing CD138.

70. The antibody molecule of any of paragraphs 29-69, comprising one or more (e.g., two or three) heavy chain CDRs and/or one or more (e.g., two or three) light chain CDRs of an anti-CD138 monoclonal antibody described herein.

71. The antibody molecule of any of paragraphs 29-70, comprising a heavy chain variable region (VH) and/or light chain variable region (VL) of an anti-CD138 monoclonal antibody described herein.

72. The antibody molecule of any of paragraphs 29-71, comprising an Fc region.

73. An anti-CD138 antibody molecule comprising one or both of:
(a) a heavy chain variable region (VH), wherein the VH comprises three heavy chain complementarity determining regions (HCDR1, HCDR2, and HCDR3), wherein the VH comprises one, two, or all of the following:
   (i) an HCDR1 comprising an amino acid sequence that differs by no more than 1, 2, or 3 amino acid residues from, or has at least 85, 90, 95, 99 or 100% homology with, the amino acid sequence of the HCDR1 of an anti-CD138 monoclonal antibody described herein (e.g., any of antibodies CD001, CD002, CD003, CD004, CD005, CD006, 602, 603, 604, 607, 613, 614, 617, 624, 632, 616, 619, 623, 1610, 2510, 2610, 2710, 2810, 2910, or 1409);
   (ii) an HCDR2 comprising an amino acid sequence that differs by no more than 1, 2, or 3 amino acid residues from, or has at least 85, 90, 95, 99 or 100% homology with, the amino acid sequence of the HCDR2 of the anti-CD138 antibody; or
   (iii) an HCDR3 comprising an amino acid sequence that differs by no more than 1, 2, or 3 amino acid residues from, or has at least 85, 90, 95, 99 or 100% homology with, the amino acid sequence of the HCDR3 of the anti-CD138 antibody; or
(b) a light chain variable region (VL), wherein the VL comprises three light chain complementarity determining regions (LCDR1, LCDR2, and LCDR3), wherein the VL comprises one, two, or all of the following:
   (i) an LCDR1 comprising an amino acid sequence that differs by no more than 1, 2, or 3 amino acid residues from, or has at least 85, 90, 95, 99 or 100% homology with, the amino acid sequence of the LCDR1 of the anti-CD138 antibody;
   (ii) an LCDR2 comprising an amino acid sequence that differs by no more than 1, 2, or 3 amino acid residues from, or has at least 85, 90, 95, 99 or 100% homology with, the amino acid sequence of the LCDR2 of the anti-CD138 antibody; or
   (iii) an LCDR3 comprising an amino acid sequence that differs by no more than 1, 2, or 3 amino acid residues from, or has at least 85, 90, 95, 99 or 100% homology with, the amino acid sequence of the LCDR3 of the anti-CD138 antibody.

74. The antibody molecule of paragraph 73, wherein the VH comprises:
(i) an HCDR1 comprising an amino acid sequence that differs by no more than 1, 2, or 3 amino acid residues from, or has at least 85, 90, 95, 99 or 100% homology with, the amino acid sequence of the HCDR1 of the anti-CD138 antibody;
(ii) an HCDR2 comprising an amino acid sequence that differs by no more than 1, 2, or 3 amino acid residues from, or has at least 85, 90, 95, 99 or 100% homology with, the amino acid sequence of the HCDR2 of the anti-CD138 antibody; and
(iii) an HCDR3 comprising an amino acid sequence that differs by no more than 1, 2, or 3 amino acid residues from, or has at least 85, 90, 95, 99 or 100% homology with, the amino acid sequence of the HCDR3 of the anti-CD138 antibody.

75. The antibody molecule of paragraph 73 or 74, wherein the VH comprises: (i) an HCDR1 comprising the amino acid sequence of the HCDR1 of the anti-CD138 antibody; (ii) an HCDR2 comprising the amino acid sequence of the HCDR2 of the anti-CD138 antibody; and (iii) an HCDR3 comprising the amino acid sequence of the HCDR3 of the anti-CD138 antibody.

76. The antibody molecule of any of paragraphs 73-75, wherein the VL comprises:
(i) an LCDR1 comprising an amino acid sequence that differs by no more than 1, 2, or 3 amino acid residues from, or has at least 85, 90, 95, 99 or 100% homology with, the amino acid sequence of the LCDR1 of the anti-CD138 antibody;
(ii) an LCDR2 comprising an amino acid sequence that differs by no more than 1, 2, or 3 amino acid residues from, or has at least 85, 90, 95, 99 or 100% homology with, the amino acid sequence of the LCDR2 of the anti-CD138 antibody; and
(iii) an LCDR3 comprising an amino acid sequence that differs by no more than 1, 2, or 3 amino acid residues from, or has at least 85, 90, 95, 99 or 100% homology with, the amino acid sequence of the LCDR3 of the anti-CD138 antibody.

77. The antibody molecule of any of paragraphs 73-76, wherein the VL comprises: (i) an LCDR1 comprising the amino acid sequence of the LCDR1 of the anti-CD138 antibody; (ii) an LCDR2 comprising the amino acid sequence of the LCDR2 of the anti-CD138 antibody; and (iii) an LCDR3 comprising the amino acid sequence of the LCDR3 of the anti-CD138 antibody.

78. The antibody molecule of any of paragraphs 73-77, comprising:
(a) a VH comprising:
   (i) an HCDR1 comprising an amino acid sequence that differs by no more than 1, 2, or 3 amino acid residues from, or has at least 85, 90, 95, 99 or 100% homology with, the amino acid sequence of the HCDR1 of the anti-CD138 antibody;
   (ii) an HCDR2 comprising an amino acid sequence that differs by no more than 1, 2, or 3 amino acid residues from, or has at least 85, 90, 95, 99 or 100% homology with, the amino acid sequence of the HCDR2 of the anti-CD138 antibody; and
   (iii) an HCDR3 comprising an amino acid sequence that differs by no more than 1, 2, or 3 amino acid residues from, or has at least 85, 90, 95, 99 or 100% homology with, the amino acid sequence of the HCDR3 of the anti-CD138 antibody, and
(b) a VL comprising:
   (i) an LCDR1 comprising an amino acid sequence that differs by no more than 1, 2, or 3 amino acid residues from, or has at least 85, 90, 95, 99 or 100% homology with, the amino acid sequence of the LCDR1 of the anti-CD138 antibody;
   (ii) an LCDR2 comprising an amino acid sequence that differs by no more than 1, 2, or 3 amino acid residues from, or has at least 85, 90, 95, 99 or 100% homology with, the amino acid sequence of the LCDR2 of the anti-CD138 antibody; and
   (iii) an LCDR3 comprising an amino acid sequence that differs by no more than 1, 2, or 3 amino acid residues from, or has at least 85, 90, 95, 99 or 100% homology with, the amino acid sequence of the LCDR3 of the anti-CD138 antibody.

79. The antibody molecule of any of paragraphs 73-78, comprising:
(a) a VH comprising: (i) an HCDR1 comprising the amino acid sequence of the HCDR1 of the anti-CD138 antibody; (ii) an HCDR2 comprising the amino acid sequence of the HCDR2 of the anti-CD138 antibody; and (iii) an HCDR3 comprising the amino acid sequence of the HCDR3 of the anti-CD138 antibody, and
(b) a VL comprising: (i) an LCDR1 comprising the amino acid sequence of the LCDR1 of the anti-CD138 antibody; (ii) an LCDR2 comprising the amino acid sequence of the LCDR2 of the anti-CD138 antibody; and (iii) an LCDR3 comprising the amino acid sequence of the LCDR3 of the anti-CD138 antibody.

80. The antibody molecule of any of paragraphs 73-79, wherein the VH comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VH of the anti-CD138 antibody.

81. The antibody molecule of any of paragraphs 73-80, wherein the VH comprises the amino acid sequence of the VH of the anti-CD138 antibody.

82. The antibody molecule of any of paragraphs 73-81, wherein the VL comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VL of the anti-CD138 antibody.

83. The antibody molecule of any of paragraphs 73-82, wherein the VL comprises the amino acid sequence of the VL of the anti-CD138 antibody.

84. The antibody molecule of any of paragraphs 73-83, wherein:
(a) the VH comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VH of the anti-CD138 antibody; and
(b) the VL comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VH of the anti-CD138 antibody.

85. The antibody molecule of any of paragraphs 73-84, wherein the VH comprises the amino acid sequence of the VH of the anti-CD138 antibody and the VL comprises the amino acid sequence of the VL of the anti-CD138 antibody.

86. The antibody molecule of any of paragraphs 73-85, comprising an Fc region.

87. An anti-CD138 antibody molecule comprises:
(I) (a) a heavy chain variable region (VH), wherein the VH comprises three heavy chain complementarity determining regions (HCDR1, HCDR2, and HCDR3), wherein the VH comprises three heavy chain complementarity determining regions (HCDR1, HCDR2, and HCDR3), wherein the VH comprises: (i) an HCDR1 comprising an amino acid sequence of G-Y-N/S/T-F-S-S-Y (SEQ ID NO: 438); (ii) an HCDR2 comprising an amino acid sequence of H-P-S-D-S-T (SEQ ID NO: 351); or (iii) an HCDR3 comprising an amino acid sequence of F-V-Y; and (b) a light chain variable region (VL), wherein the VL comprises three light chain complementarity determining regions (LCDR1, LCDR2, and LCDR3), wherein the VL comprises one, two, or all of: (i) an LCDR1 comprising an amino acid sequence of R-S-S-K-S-L-L-Y-K-D-G-K-T-Y-L-N(SEQ ID NO: 352); (ii) an LCDR2 comprising an amino acid sequence of V-V-S-T-R-A-S(SEQ ID NO: 353); or (iii) an LCDR3 comprising an amino acid sequence of Q-Q-L-V-E-Y-P-Y-T (SEQ ID NO: 354); or
(II) (a) a heavy chain variable region (VH), wherein the VH comprises three heavy chain complementarity determining regions (HCDR1, HCDR2, and HCDR3), wherein the VH comprises three heavy chain complementarity determining regions (HCDR1, HCDR2, and HCDR3), wherein the VH comprises: (i) an HCDR1 comprising an amino acid sequence of S-Y-Y-M-H (SEQ ID NO: 380); (ii) an HCDR2 comprising an amino acid sequence of T-I-H-P-S-D-S-T-T-N-C/Y-N-Q-K-F-K-G (SEQ ID NO: 439); or (iii) an HCDR3 comprising an amino acid sequence of F-V-Y; and (b) a light chain variable region (VL), wherein the VL comprises three light chain complementarity determining regions (LCDR1, LCDR2, and LCDR3), wherein the VL comprises one, two, or all of: (i) an LCDR1 comprising an amino acid sequence of R-S-S-K-S-L-L-Y-K-D-G-K-T-Y-L-N(SEQ ID NO: 352); (ii) an LCDR2 comprising an amino acid sequence of V-V-S-T-R-A-S(SEQ ID NO: 353); or (iii) an LCDR3 comprising an amino acid sequence of Q-Q-L-V-E-Y-P-Y-T (SEQ ID NO: 354).

88. The antibody molecule of any of paragraphs 1-87, comprising two VHs and two VLs.

89. The antibody molecule of any of paragraphs 1-88, which is a synthetic antibody molecule or an isolated antibody molecule.

90. The antibody molecule of any of paragraphs 1-89, which is a monovalent antibody molecule, a multivalent (e.g., bivalent, trivalent, or tetravalent) antibody molecule, a monospecific molecule, or a multispecific (e.g., bispecific, trispecific, or tetraspecific) antibody molecule.

91. The antibody molecule of any of paragraphs 1-90, which is a humanized antibody molecule.

92. The antibody molecule of any of paragraphs 1-91, comprising one or more framework regions derived from human framework germline sequence.

93. The antibody molecule of any of paragraphs 1-92, which is an IgG antibody.

94. The antibody molecule of any of paragraphs 1-93, comprising a heavy chain constant region of IgG chosen from IgG1, IgG2, IgG3, or IgG4.

95. The antibody molecule of any of paragraphs 1-94, comprising a light chain constant region of kappa or lambda light chain.

96. The antibody molecule of any of paragraphs 1-95, comprising an Fc region comprising one or more mutations to increase the binding affinity to neonatal receptor FcRn and/or the half-life of the antibody molecule.

97. The antibody molecule of any of paragraphs 1-96, comprising an Fc region comprising one or more mutations described herein, e.g., to increase one or more of half-life, ADCC, CDC, or ADCP.

98. An antibody molecule, which competes for binding to CD138 with an anti-CD138 antibody molecule described herein, e.g., an anti-CD138 monoclonal antibody described herein (e.g., any of antibodies CD001, CD002, CD003, CD004, CD005, CD006, 602, 603, 604, 607, 613, 614, 617, 624, 632, 616, 619, 623, 1610, 2510, 2610, 2710, 2810, 2910, or 1409).

99. An antibody molecule, which binds, or substantially binds, to an epitope that completely or partially overlaps with the epitope of an anti-CD138 antibody molecule described herein, e.g., an anti-CD138 monoclonal antibody described herein (e.g., any of antibodies CD001, CD002, CD003, CD004, CD005, CD006, 602, 603, 604, 607, 613, 614, 617, 624, 632, 616, 619, 623, 1610, 2510, 2610, 2710, 2810, 2910, or 1409).

100. An antibody-molecule drug conjugate (ADC) comprising an antibody molecule of any of paragraphs 1-99, optionally comprising a cytotoxic agent, further optionally comprising a linker.

101. A composition comprising an antibody molecule of any of paragraphs 1-99, or an ADC of paragraph 100, optionally, wherein the composition is a pharmaceutical composition.

102. The composition of paragraph 101, further comprising a pharmaceutically acceptable carrier. 103. A nucleic acid molecule encoding a heavy chain variable region (VH), a light chain variable region (VL), or both, of an antibody molecule of any of paragraphs 1-99.

104. A vector comprising a nucleic acid molecule of paragraph 103.

105. A cell comprising a nucleic acid molecule of paragraph 103 or a vector of paragraph 104, optionally, wherein the cell is an isolated cell.

106. A kit comprising an antibody molecule of any of paragraphs 1-99, an ADC of paragraph 100, or a composition of paragraph 101 or 102, and instructions to use of the antibody molecule or composition.

107. A container comprising an antibody molecule of any of paragraphs 1-99, an ADC of paragraph 100, or a composition of paragraph 101 or 102.

108. A method of producing an anti-CD138 antibody molecule, the method comprising culturing a cell of paragraph 105 under conditions that allow production of an antibody molecule, thereby producing the antibody molecule.

109. The method of paragraph 108, further comprising isolating or purifying the antibody molecule.

110. An antibody molecule of any of paragraphs 1-99, an ADC of paragraph 100, or a composition of paragraph 101 or 102, for use in a method of treating a cancer in a subject.

111. The antibody molecule, ADC, or composition for use of paragraph 110, wherein the cancer is a hematological cancer.

112. The antibody molecule, ADC, or composition for use of paragraph 110 or 111, wherein the cancer is a multiple myeloma.

113. The antibody molecule, ADC, or composition for use of paragraph 110, wherein the cancer is a solid tumor, e.g., a solid tumor described herein.

114. The antibody molecule, ADC, or composition for use of any of paragraphs 110-113, wherein the antibody molecule, ADC, or composition is administered to the subject intravenously.

115. The antibody molecule, ADC, or composition for use of any of paragraphs 110-114, wherein the antibody molecule, ADC, or composition is administered to the subject at a dose between 0.1 mg/kg and 50 mg/kg, between 0.2 mg/kg and 25 mg/kg, between 0.5 mg/kg and 10 mg/kg, between 0.5 mg/kg and 5 mg/kg, between 0.5 mg/kg and 3 mg/kg, between 0.5 mg/kg and 2.5 mg/kg, between 0.5 mg/kg and 2 mg/kg, between 0.5 mg/kg and 1.5 mg/kg, between 0.5 mg/kg and 1 mg/kg, between 1 mg/kg and 1.5 mg/kg, between 1 mg/kg and 2 mg/kg, between 1 mg/kg and 2.5 mg/kg, between 1 mg/kg and 3 mg/kg, between 1 mg/kg and 2.5 mg/kg, or between 1 mg/kg and 5 mg/kg.

116. The antibody molecule, ADC, or composition for use of any of paragraphs 110-115, wherein the antibody molecule, ADC, or composition is administered to the subject at a fixed dose between 10 mg and 1000 mg, between 10 mg and 500 mg, between 10 mg and 250 mg, between 10 mg and 150 mg, between 10 mg and 100 mg, between 10 mg and 50 mg, between 250 mg and 500 mg, between 150 mg and 500 mg, between 100 mg and 500 mg, between 50 mg and 500 mg, between 25 mg and 250 mg, between 50 mg and 150 mg, between 50 mg and 100 mg, between 100 mg and 150 mg, between 100 mg and 200 mg, or between 150 mg and 250 mg.

117. The antibody molecule, ADC, or composition for use of any of paragraphs 110-116, wherein the antibody molecule, ADC, or composition is administered once a week, twice a week, once every two weeks, once every three weeks, or once every four weeks.

118. The antibody molecule, ADC, or composition for use of any of paragraphs 110-117, further comprising determining the level of CD138 in a sample from the subject.

119. The antibody molecule, ADC, or composition for use of any of paragraphs 110-118, further comprising administering to the subject a second therapy for cancer.

120. An antibody molecule of any of paragraphs 1-99, an ADC of paragraph 100, or a composition of paragraph 101 or 102, for use in a method of treating a precancerous condition or preventing a cancer.

121. The antibody molecule, ADC, or composition for use of paragraph 120, wherein the precancerous condition is smoldering myeloma or monoclonal gammopathy of undetermined significance (MGUS).

122. The antibody molecule, ADC, or composition for use of paragraph 120, wherein the cancer is multiple myeloma.

123. A method of causing an ADCC activity, the method comprising contacting a cell or subject an antibody molecule of any of paragraphs 1-99, an ADC of paragraph 100, or a composition of paragraph 101 or 102, thereby causing the ADCC activity.

124. A method of treating a cancer, the method comprising administering to a subject in need thereof an effective amount of an antibody molecule of any of paragraphs 1-99, an ADC of paragraph 100, or a composition of paragraph 101 or 102, thereby treating the cancer.

125. A method of treating a precancerous condition or preventing a cancer, the method comprising administering to a subject in need thereof an effective amount of an antibody molecule of any of paragraphs 1-99, an ADC of paragraph 100, or a composition of paragraph 101 or 102, thereby treating the precancerous condition or preventing the cancer.

126. A method of detecting an anti-CD138 molecule, the method comprising contacting a cell or a subject with an antibody molecule of any of paragraphs 1-99, thereby detecting the CD138 molecule.

127. The method of paragraph 126, wherein the antibody molecule is coupled with a detectable label.

128. The method of paragraph 126 or 127, wherein the CD138 molecule is detected in vitro, ex vivo, or in vivo.

### EXAMPLES

### Example 1: Mouse Immunizations

CD-1 IGS (outbred stock) mice (Charles River Laboratories), female (20-25 g weight), 5-6 weeks old were immunized intravenously (i.v.) with 50 μg of plasmid encoding human CD138 (PCDNA3.1-hCD138) vector on day 0, 14 and 28. A second group of mice were immunized intraperitoneally with rCD138 (Sino Biological, Inc.)+Sigma adjuvant (1:1) or Peptide 6+Sigma adjuvant (1:1) on day 0 and boosted with the same on day 14 and day 30. Following 3 rounds of DNA or protein/peptide immunization, the serum titers of anti-CD138 antibodies were detected by indirect ELISA using recombinant CD138 (R&D Systems). The titer of peptide-6 binding antibody was also evaluated by ELISA using Peptide-6. In brief, 200 ng of rCD138 or Peptide-6 in PBS were coated on Maxisorp 96-well flat bottom plates (NUNC #439454), overnight at 4° C. Coated plates were blocked in 1× blocking buffer containing 5% BLOTTO™ in PBS and 0.05% Tween-20 (PBST) for 1 hour at room temperature. All subsequent incubation steps were followed out with an intervening 3× wash step in PBST. Anti-CD138 (or anti-peptide-6) antibody titers were determined from a fold-dilution of mouse sera (in PBS) initially starting at 1:50 and followed by incubation of a 1:5000-1:10000 HRP conjugated goat anti-mouse IgG secondary antibody (Jackson ImmunoResearch Laboratories) for 1 hour at room temperature. Anti-CD138 (or anti-peptide-6) immunoglobulin reactivity was visualized using 100 μl/well of freshly prepared TMB substrate (KPL). Colorimetric development was carried out for up to 10 minutes at room temperature before quenching enzymatic reaction by the addition of 100 μl of 1N sulfuric acid and quantification by absorbance at 450 nm. Mice with strong seropositive titers against primary immunogen (human CD138) were boosted with 5-10 μg of rCD138 or Peptide-6 by tail vein injection three days prior to sacrifice, removal of spleen and isolation of splenoctye fusions. Select mice received two additional intraperitoneal (i.p.) immunizations with peptide-6 mixed with Sigma adjuvant prior to the Peptide-6 tail vein boost. Mice with preferable species cross-reactivity from serum profiling were noted.

### Example 2: Hybridoma Development

P3X63Ag8.653 plasmacytomas (ATCC #CRL-1580), herein referred to as P3X cells, were used as source of fusion partner myelomas. Splenically-derived B cell clones were immortalized using published methods with modification. In brief, P3X cells were cultured at least 1 week prior to use and maintained in log phase to achieve a target cell density of between 6×10⁵ and 1.2×10⁶ cells/mL and 95% viability the day prior to subsequently performing the splenic fusion. Spleen cells were isolated from 2-3 mice per immunization arm following euthanization and cardiac puncture and collected into DMEM+1% antibiotic (penicillin/streptomycin), followed by gently washing centrifugation (2×) to pellet tissue debris and clarify suspended splenocytes. Splenocytes were then pelleted by centrifugation for 10 min at 400×g at 4° C., and red blood cells lysed at room temperature for 5 minutes following gentle resuspension of cell pellet in 1× red blood cell lysis buffer. Splenocytes were collected by centrifugation (2×) following dilution with ice cold DMEM. P3X cells were also washed 3× in DMEM prior to fusion.

Mouse splenocytes were fused with P3X cells in fusion medium (50% PEG 1450, Sigma Aldrich) at a 3:1 ratio in accordance with established methods. In brief, pre-warmed PEG was added gradually to pelleted mixture of splenocytes and P3X cells (37° C., with gentle resuspension) followed by gradual addition of pre-warmed DMEM. Fused cells were collected by low speed centrifugation and resuspended in hybridoma selective media (hypoxanthine-aminopterin-thymidine, Sigma Aldrich) followed by incubation at 37° C. for 30 minutes. Fused cells sere plated in a 96 well plate at a density of approximately 2.0×10⁶ spleen cells per plate (20,000 cells per well). Hybridoma supernatants were screened for CD138 binding by ELISA on day 10-14 post-fusion as described. In brief, supernatants from conditioned media were quantified for total IgG by bioinferometry using AMC anti mouse IgG quantification kit (Pall Biosciences). Supernatants from hybridoma conditioned media were normalized to 10 μg/mL when possible and assayed for binding to CD138 or Peptide-6 by ELISA. Positive hybridomas were selected for culture scale up, antibody purification and further characterization as described.

CD138 and Peptide-6 positive hybridomas were screened for receptor blocking activity by ELISA. In brief, Recombinant CD138 (10 μg/ml) or Peptide-6 (20 μg/ml) in 1×PBS (pH 7.4) were coated on to Maxisorp 96-well flat bottom plates overnight at 4° C. Plates were washed with PBS+0.05% Tween20 (PBST) and blocked with 5% BLOTTO™. Mouse sera or anti-CD138 antibodies were diluted in PBST and incubated for 2 hr at room temperature. The unbound antibody/sera was washed away post incubation by 3×wash with PBST. Detection of CD138 or Peptide-6 antibody was quantified using goat anti-mouse HRP secondary antibody conjugated with HRP (Sigma Aldrich) used at 1:5000 dilution followed by colorimetric development using 100 μl/well of freshly prepared TMB substrate (KPL) carried out for up to 30 minutes at room temperature before quenching enzymatic reaction by the addition of 1N sulfuric acid. ELISA signal was quantified by absorbance at 450 nm. ELISA data was analyzed by non-linear regression. IC₅₀ values were calculated based on a 4-parameter fit of antibody titration curves.

### Example 3: Determination and Molecular Cloning of Anti-CD138 Immunoglobulin Sequences

VH and VL gene sequences of mouse antibodies derived from hybridoma screening were initially determined by reverse transcriptase PCR of B cell RNA using a pool of pre-defined set of mouse Ig sequence-specific primers of varying degeneracy. 5′ Primer design for VH sequencing was based on a comprehensive analysis of the mouse immunoglobulin database with corresponding alignment to variable leader sequences. From this analysis, VH leader sequences were clustered (or binned based on sequence relatedness and representation of germline “families”): a unique set of primers, each predicted to anneal more specifically to these binned VH sequence families were designed and used as a cocktail in the RT-PCR reaction. 3′ primers were designed to anneal in the constant region of the heavy chain and corresponded to unique sequences in CHI that define the four known mouse IgG constant regions (IgG1, IgG2a, IgG2b and IgG3). IgM related VH sequences were amplified as above but with substitution of an IgM isotype 3′ primer. Similarly, a so-called “pooled primer” RT-PCR approach was used to amplify the corresponding VL sequences from mouse hybridoma RNA. A systematic query of all known mouse VL leader sequences was likewise performed. As kappa and lambda light chains share neither the constant region nor variable region sequences, separate primer sets (kappa vs. lambda specific) were designed. 3′ primers were designed based on isotype specific light chain constant region sequence (kappa vs. lambda) in a manner analogous to the one described above for heavy chain sequences.

RT-PCR amplification of hybridoma gene sequences from B cell RNA was completed using otherwise established methods. In brief, RNA was extracted from 0.5-2×10⁶ cells using the RNeasy kit (Life Technologies) as per manufacturer's instructions. Cell lysis was facilitated using QIAshredder or related method for initial nucleic acid extraction. Purified RNA was quantified by UV absorbance. cDNA synthesis and subsequent PCR amplification (using Platinum Taq polymerase and primer mixes described above) were completed in tandem using Superscript III One Step RT-PCR kit (Life Technologies). PCR amplicons were purified using QIAquick PCR clean up kit (Life Technologies) and quantified by UV absorbance at 260 and 280 nm using a Nanodrop spectrophotometer. PCR products were also analyzed by agarose gel electrophoresis to confirm predicted size and gel purified as needed. VH and VL gene sequences were determined by directly sequencing of PCR products using nested primers. Ambiguous sequence data was followed by re-amplification of cell RNA by RT PCR as described above but with modification to protocol and using a subset of smaller pooled primer sets; if necessary PCR products were cloned by TA cloning into an intermediate vector) and transformed into chemically competent TOP 10 (Life Technologies) or DH5a (New England Biolabs) as per the manufacturers protocols.

DNA sequence data was analyzed using publically available databases (e.g., International Immunogenetics Information system (IMGT), VBase, or NCBI Ig-Blast) to evaluate germline usage, identify CDR sequences and assign putative isotype when possible. gBlocks based on the identified VH and VL sequences were ordered (IDT DNA) and sub-cloned into pcDNA3.1 vectors containing osteonectin leader sequence and human IgG1k heavy chain or light chain constant regions.

### Example 4: Purification of Anti-CD138 Antibodies

CD138 positive hybridoma clones were cultured at sequentially higher scale from 96 well plates to 24 well plates and subsequently to T150 flasks (20 mL culture volume). Prior to purification, cells were transferred out of HAT selective media into pre-defined, low Ig media. Supernatants were harvested 3-5 days after media transfer and clarified by centrifugation, followed by sterile filtration through a 0.22 μm PES membranes (Corning). IgG titers were confirmed by Bioinferometry as described. Supernatants were diluted 1:1 with 2× Protein G binding buffer (1M glycine, 2M NaCl, pH 9.0). Antibodies were purified by Protein G affinity chromatography using 1 mL Protein G HiTrap columns (GE Health Care) at a flow rate of 1 ml/min and as per the manufacturer's recommendations. IgG was eluted from the protein G column by lowering pH using 0.1M glycine buffer, pH 2.8 followed by immediate neutralization using 2M TRIS. pH 8.5. Purified antibodies were reformulated by dialysis in 1×PBS. pH 7.4 followed by concentration by ultrafiltration using an Ultra-30 AMICON 30 KD MWCO filtration unit. Final antibody concentration was determined spectrophotometrically by NanoDrop using a generalized extinction coefficient for murine antibodies (IgG1). Antibody purity and integrity was confirmed by SDS-PAGE under both reducing and non-reducing conditions.

### Example 5: Recombinant Expression and Purification of Antibodies

Co-expression of the heavy and light chain vectors was performed by transient transfection in Expi293 cells using the Expi293 transfection kit (Thermo Fisher catalogue #A14524) following the manufacturer's protocol. The heavy and light chain vectors were co-transfected at a 1:2 ratio. Supernatant was harvested 5 to 7 days post transfection for protein A purification. Antibody titer was quantified by bioinferometry using Protein A-immobilized biosensors (Pall Biosensors). Recombinant antibodies were purified from culture supernatant following clarification by low speed centrifugation and sterile filtration through 0.22 μm PES membranes. Antibodies were purified from cell culture supernatant using 1 mL columns packed with mAb select sure protein A resin (GE catalogue #17543801) using the AKTA purifier 10 FPLC system. Briefly, sterile filtered cell culture supernatant was loaded onto the columns at a flow rate of 2 mL/minute. Columns were washed with 10 column volumes of PBSN (1×PBS with 0.05% sodium azide). Antibodies were eluted with 10 column volumes of elution buffer (100 mM glycine pH 2.5) and neutralized by addition 17.5% v/v of neutralization buffer (1M Tris, 1M NaCl, pH 8.0) and collated in 1 mL fractions. The chromatogram for absorbance at 280 nm was used to identify elution fractions containing the antibody. All antibodies were then dialyzed into 1×PBS using 10,000 dalton molecular weight cut-off cassette (Thermo Fisher catalogue #66380).

### Example 6: Characterization of Anti-CD138 Antibodies

Binding of anti-CD138 antibodies to CD138 was tested by flow cytometry binding assay. Multiple myeloma cell lines RPMI 8226 (ATCC) and U266 (ATCC) were grown in RPMI1640 with 10% FBS. On the day of experiment, 0.25×10⁶ cells were washed with FACS buffer (PBS+0.5% BSA) and incubated with dilution series of anti-CD138 antibodies (starting 10 μg/ml) or hybridoma supernatants (starting with undiluted supernatant) for 30 min at 4° C. followed by incubation with goat-anti-human/mouse conjugated APC antibodies (BioLegend) for 30 min at 4° C. Fluorescence was detected using flow cytometer.

Antibody dependent cellular cytotoxicity (ADCC) assays were performed using the ADCC Reporter Bioassay from Promega (catalogue #G7014) following the manufacturer's protocol. Purified anti-CD138 antibodies or hybridoma supernatants were assessed for their ADCC activity on U266 myeloma cells in low IgG growth media. Briefly, in a 96 well white bottom plate Anti-CD138 antibodies were mixed with U266 cells at different concentration followed by Jurkat T cells were added at a ratio of 10:1 effector to target ratio and incubated at 37° C. for 16 hr. The Jurkat T cell used the assays express human/mouse CD16 (Promega effector cells). Bio Glo (Luciferin from Promega) was added to all wells and luminescence was analyzed by spectrophotometer. The values of antibody concentration (x-axis) and fold induction of the luminescent reporter gene (y-axis) were fit to a 4-parameter logistic regression (4PL) curve. The curve fit was then used to determine the EC50 (the midpoint of the 4PL) and the maximum induction for each Fc variant.

Anti-CD138 antibodies were tested for growth inhibition properties using WST assay. U266 and RPMI8226 cells were seeded in a 96 well tissue culture plates at a density of 5000 cells/well. Purified anti-CD138 antibodies were diluted in low serum media at different concentrations and incubated at 37° C. After 3-5 days cell Proliferation reagent WST-1 was added at 1:10 final volume and incubated up to 4 h at 37° C. Absorbance was read at 440 nm using spectrophotometer.

### Example 7: Identification of Anti-CD138 Antibodies that Bind to Desired Epitopes

Multiple antibodies that bind to desired epitope were identified. The peptides used to identify the antibodies are described in FIG. 2. Representative examples are shown in Table 6 below.

TABLE 6
Exemplary Anti-CD138 Antibodies and Their Binding to CD138

| | rCD138 | RPMI 8226 | U266 | Pep1/Pep2 | Pep3 | Pep4 | Pep5 | Pep6 |
|---|---|---|---|---|---|---|---|---|
| mAb | (ELISA | (% cells | (% cells | (ELISA | (ELISA | (ELISA | (ELISA | (ELISA |
| ID | O.D.) | Positive) | Positive) | O.D.) | O.D.) | O.D.) | O.D.) | O.D.) |
| #101 | 3.032 | 11.8 | 7.6 | 0.162 | 0.138 | 0.176 | 2.817 | 0.108 |
| #102 | 2.878 | 12.9 | 6.8 | 0.109 | 0.087 | 0.129 | 2.581 | 0.078 |
| #106 | 2.861 | 89.3 | 91.3 | 0.121 | 0.095 | 0.120 | 2.834 | 0.292 |
| #110 | 2.780 | 33.1 | 58.7 | 0.123 | 0.094 | 0.125 | 0.359 | 0.083 |
| #128 | 2.815 | 65.0 | 19.2 | 0.128 | 0.138 | 2.926 | 0.084 | 0.073 |
| #135 | 2.861 | 96.8 | 98.6 | 0.120 | 0.090 | 0.115 | 2.810 | 0.111 |
| #149 | 2.879 | 95.7 | 98.4 | 0.097 | 0.089 | 0.106 | 2.792 | 0.075 |
| #150 | 2.884 | 9.8 | 12.0 | 0.104 | 0.080 | 0.154 | 2.806 | 0.086 |
| 602 | 0.574 | 87.9 | 96.8 | 0.150 | 0.058 | 0.056 | 0.059 | 1.002 |
| 603 | 0.585 | 81.4 | 95.8 | 0.075 | 0.047 | 0.051 | 0.053 | 0.863 |
| 604 | 0.610 | 82.5 | 96.0 | 0.062 | 0.058 | 0.058 | 0.067 | 0.939 |
| 607 | 0.453 | 7.6 | 69.1 | 0.062 | 0.062 | 0.074 | 0.076 | 0.746 |
| 613 | 0.486 | 77.4 | 94.8 | 0.062 | 0.056 | 0.058 | 0.053 | 0.642 |
| 614 | 0.682 | 85.3 | 96.3 | 0.147 | 0.066 | 0.069 | 0.082 | 0.925 |
| 617 | 0.581 | 43.3 | 89.4 | 0.102 | 0.084 | 0.091 | 0.066 | 0.809 |
| 624 | 1.525 | 89.3 | 96.7 | 0.680 | 0.069 | 0.069 | 0.066 | 1.682 |
| 632 | 1.503 | 43.1 | 80.9 | 0.477 | 0.062 | 0.063 | 0.068 | 1.642 |
| 616 | 1.178 | 18.3 | 6.1 | 0.069 | 0.063 | 0.065 | 0.061 | 1.618 |
| 619 | 0.882 | 85.0 | 3.7 | 0.064 | 0.067 | 0.066 | 0.066 | 1.367 |
| 623 | 0.803 | 63.8 | 7.0 | 0.098 | 0.086 | 0.082 | 0.080 | 1.674 |

### Example 8: Effect of Epitope Engagement on Effector Functions

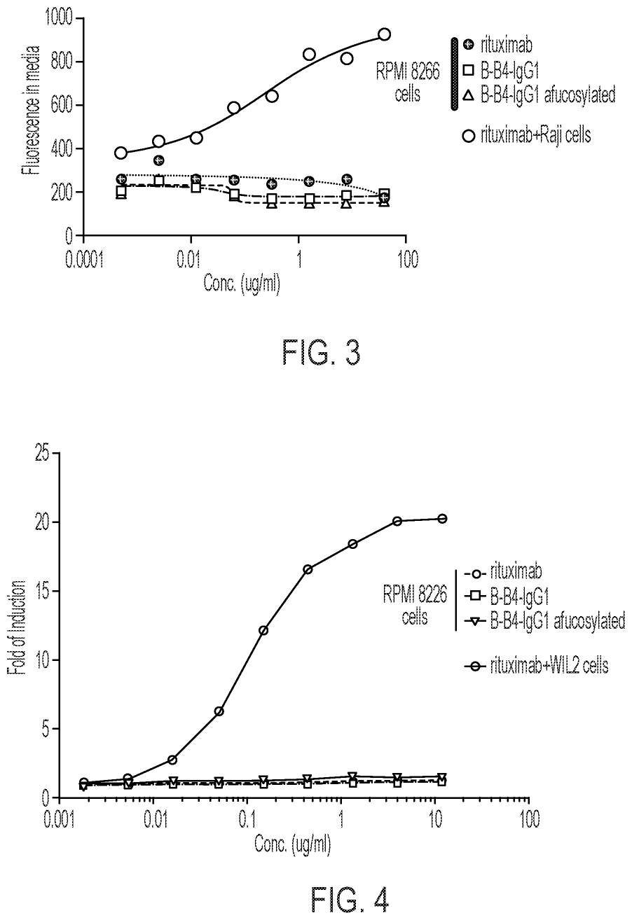

B-B4 is an anti-CD138 antibody that binds to the integrin binding domain (IBD) of CD138. The ability of B-B4 to induce complement-dependent cytotoxicity (CDC) was examined. As shown in FIG. 3, both B-B4-IgG1 and afucosylated B-B4-IgG1 did not induce CDC in human myeloma RPMI 8226 cells. Rituximab, an antibody targeting B-lymphocyte antigen CD20, did not induce CDC in PRMI 8226 cells. Rituximab induced CDC in Raji cells, which are lymphoblastoid cells with B-cell characteristics derived from a Burkitt's lymphoma.

The ability of B-B4 to induce antibody dependent cellular cytotoxicity (ADCC) was examined. As shown in FIG. 4, both B-B4-IgG1 and afucosylated B-B4-IgG1 did not induce ADCC in RPMI 8226 cells. Rituximab did not induce ADCC in PRMI 8226 cells. Rituximab induced CDC in WIL2 cells, which are human B lymphocytes.

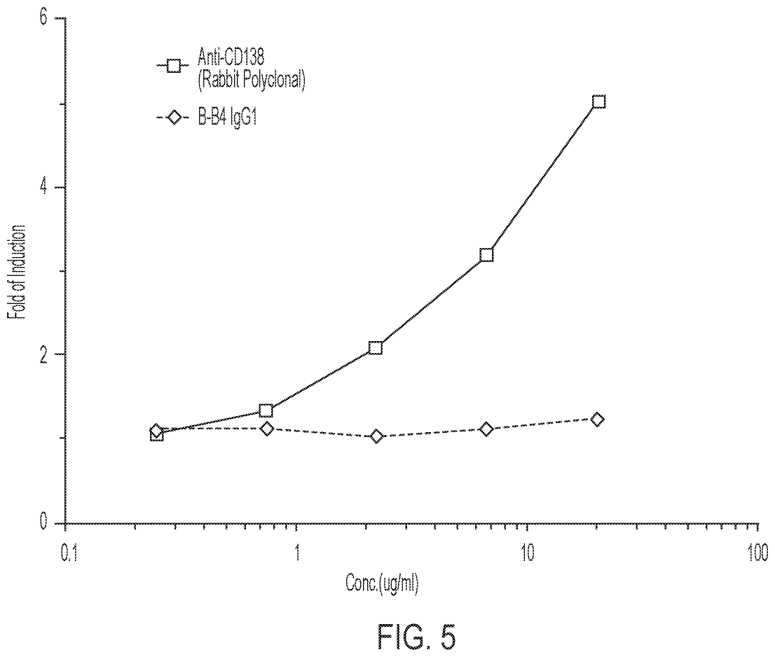

The ability of rabbit anti-CD138 polyclonal antibody to induce ADCC was examined. As shown in FIG. 5, rabbit anti-CD138 polyclonal antibody induced ADCC in human multiple myeloma U266 cells. Compared to B-B4 IgG1, the induction of ADCC was increased by up to 5-fold.

### Example 9: Role of Epitope Distance on ADCC Activity

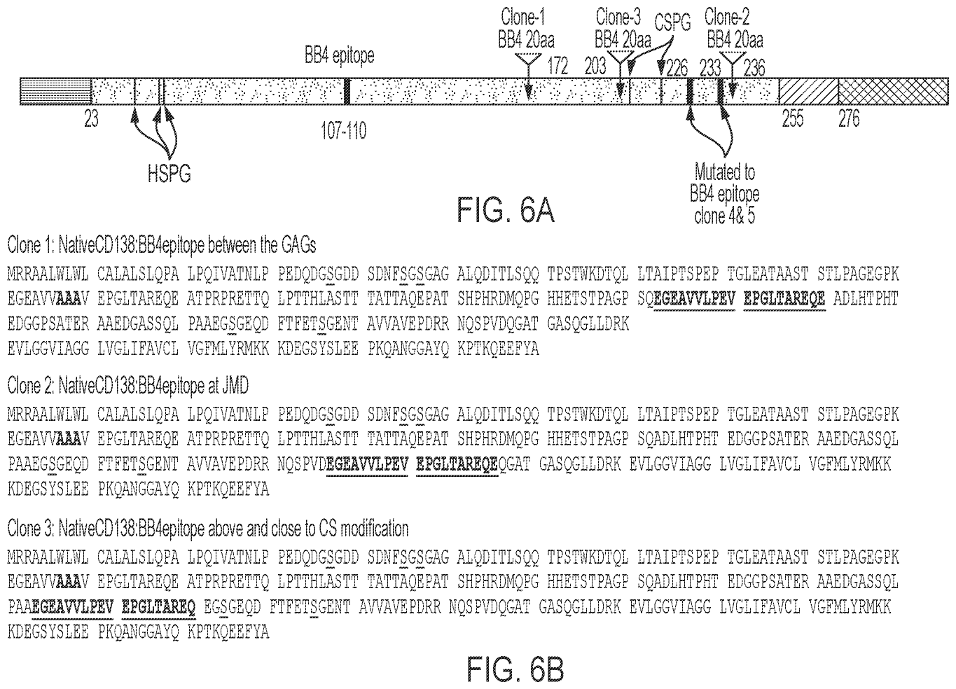

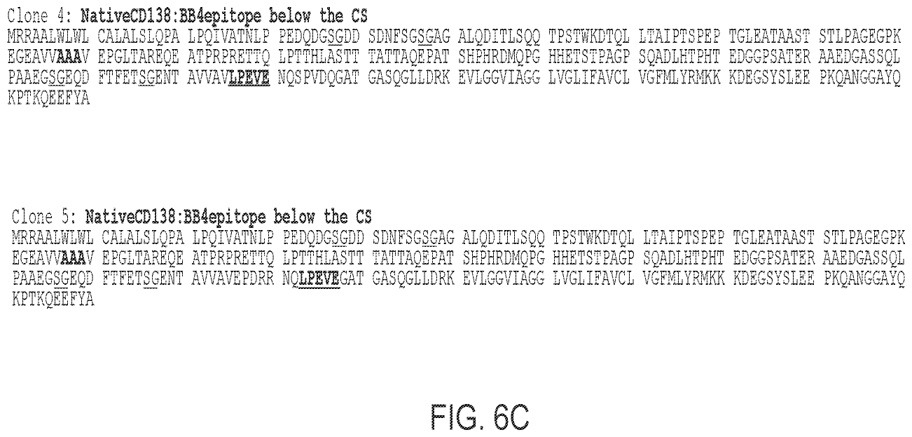

The epitope of B-B4 has been mapped to a linear peptide toward the N-terminal of CD138 (FIG. 1). As shown in FIGS. 6A-6C, CD138 constructs were designed in which the native B-B4 epitope was mutated and B-B4 epitope was introduced at midway through the ectodomain or proximal to the membrane.

In clones 1, 2, and 3, a 20-amino acid peptide (residues 101-120) around the inferred B-B4 epitope (residues 107-110) is inserted at predetermined positions of the CD138 ectodomain while removing the original B-B4 binding site by mutating its hot spot residues Leu107, Pro108, and Glu109 to Ala. In clones 1, 2, and 3, the 20-amino acid B-B4 binding peptide is inserted between residues 172 and 173, residues 236 and 237, and residues 203 and 204, respectively.

Unlike the insertion of the 20-amino acid B-B4 binding peptide as in clone 1, 2 and 3, in clones 4 and 5 only the five amino acid B-B4 binding epitope is created by mutating the original CD138 residues, and in addition to these mutations, the original B-B4 hot spot residues Leu107, Pro108, and Glu109 are mutated to Ala. In clone 4, the mutations are E226L, D228E, R229V, and R230E, while in clone 5, the mutations are S233L, V235E, D236V, and Q237E.

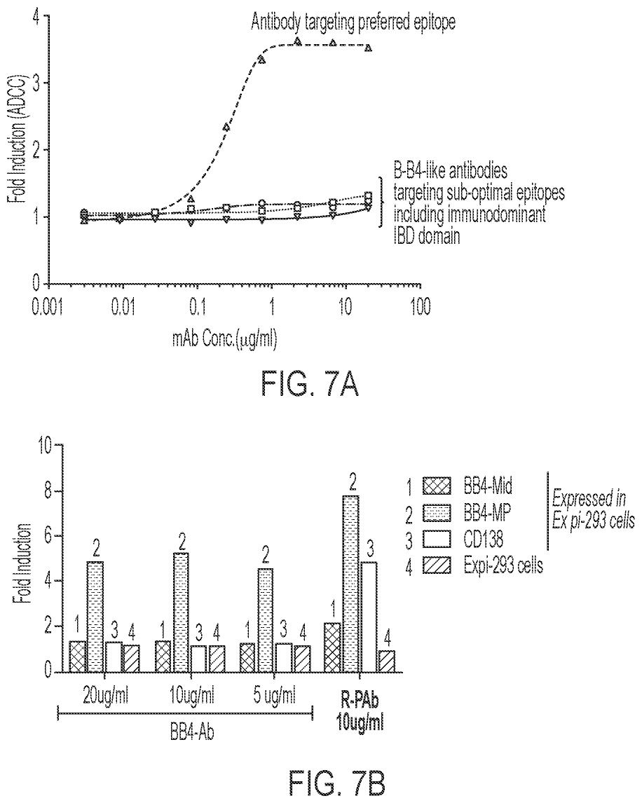

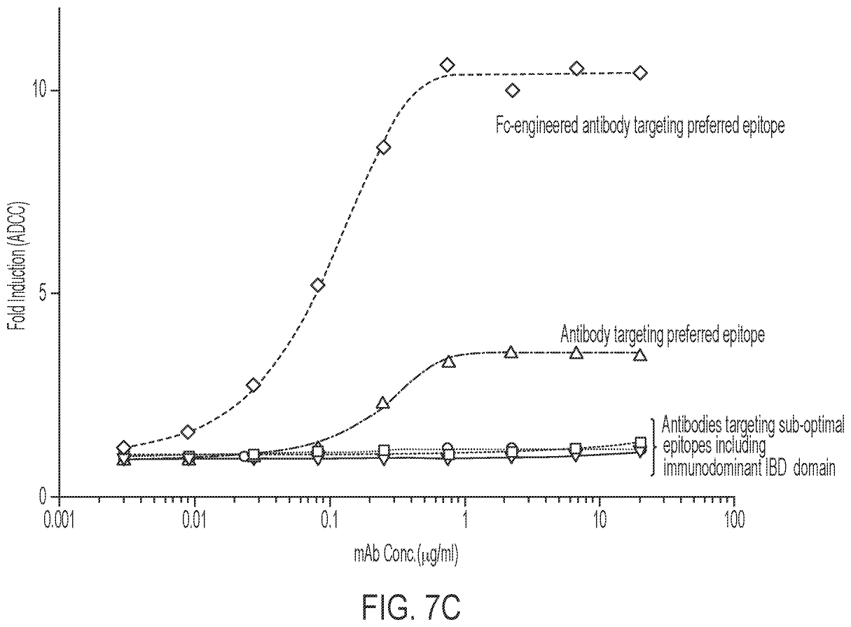

Wild-type CD138 and variants with the B-B4 epitope introduced at different sites of CD138 were recombinantly expressed on the surface of Expi293 cells. Expression was confirmed by staining with B-B4 and polyclonal anti-CD138 antibody. ADCC activity was assessed using ADCC reporter assay as described above. As shown in FIGS. 7A-7B, B-B4-like antibodies that target sub-optimal epitopes including immunodominant IBD do not elicit ADCC, and B-B4 is capable of inducing ADCC activity when the epitope is moved proximal to the cell membrane. As shown in FIG. 7C, Fe engineering further enhances ADCC.

### Example 10: Binding of Additional Anti-CD138 Monoclonal Antibodies to Soluble Human CD138 Extracellular Domain

Additional monoclonal anti-CD138 antibodies 1610 and 1409 were identified from screening of immunized mice. Briefly, total RNA from splenocytes of CD138/peptide6 immunized mice was extracted and cDNA was synthesized using SuperScript™ IV First-Strand Synthesis System. Variable regions i.e. VH and VL were amplified using mouse VH and VL specific primers. After a series of PCR reactions, VH and VL DNA with appropriate overhang sequences were amplified and VH and VL sequences were cloned into yeast expression vector pYDv6 by homologous recombination and as single chain Fragment variable (scFv) for yeast surface display. VH and VL DNA along with the linearized pYDv6 vector were transformed into EBY100 yeast cells by electroporation for surface scFv expression. Transformed yeast were grown in SDCAA media at 30° C., induced in SGCAA media at 20° C. and enriched for rCD138 binders magnetic bead capture using biotinylated CD138 and anti-biotin magnetic beads from Miltenyi Biotec. Yeast were then enriched for binding to recombinant CD138 (extracellular domain) by fluorescence-activated cell sorting (FACS) for at least 2-3 rounds to achieve >95% CD138 positive binders. Yeast were concurrently analyzed for surface scFv expression using anti-MYC antibody and binding to rCD138. Derivative yeast display libraries of CD138 positive binders were also further analyzed for binding to CD138 derived peptides, likewise biotinylated for detection by flow cytometry. After 3 rounds of enrichment by FACS, CD138 binders were plated on SDCAA plates and VH and VL genetic sequences of individual clones were genetically analyzed by direct DNA sequencing by the Sanger method. Antibody sequences were further analyzed using IMGT/V-quest. Based on this combined phenotype and genotype analyses, select VH and VL sequences were subsequently cloned and transiently expressed in HEK 293 cells as chimeric monoclonal antibodies with murine variable regions (Fab) and human IgG1 isotype IgG1. Recombinant antibodies were purified by affinity capture chromatography using protein A and characterized for binding to CD138, CD138 peptides and myeloma cell lines by methods described herein. Fc afucosylated variants of these antibodies were also produced in an engineered CHO M cell line in which fucosyltransferase 8 (FUT8) gene was ablated using Crisper-Cas based gene editing technologies commonly described in the literature.

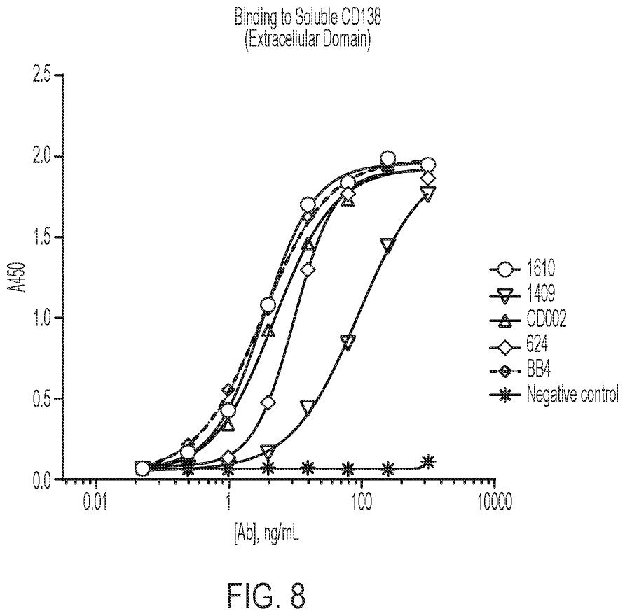

These antibodies were assessed for their capacity to bind to the soluble CD138 extracellular domain in an ELISA assay, alongside antibodies CD002 and 624 described above. Antibody B-B4 was included as a reference. Briefly, the monoclonal antibodies were tested for binding to recombinant CD138 extracellular domain consisting of amino acids 23-254 of human CD138, in four-fold serial dilutions starting at 1 μg/mL. HRP-conjugated anti-human IgG-Fc antibody (1:5000 dilution) was used for detection. As shown in FIG. 8, both antibodies 1610 and 1409 were able to bind to the CD138 extracellular domain. Antibody 1610 exhibited comparable binding to antibody CD002 and the reference antibody B-B4.

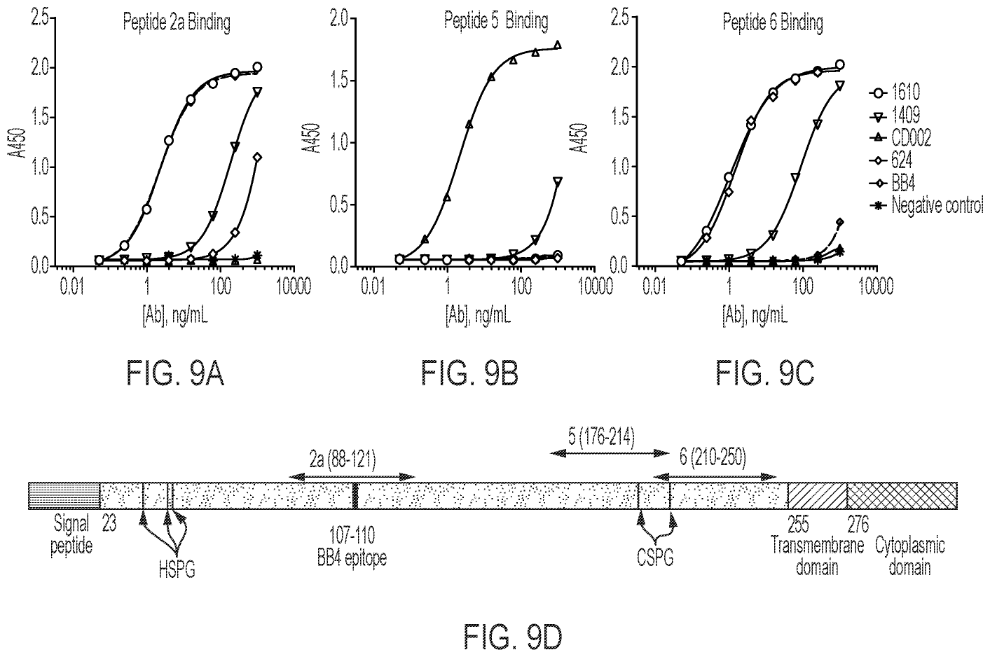

Monoclonal anti-CD138 antibodies 1610, 1409, CD002, and 624 were then tested for their capacity to bind to different regions of CD138 using peptide binding ELISA. As above, the monoclonal antibodies were tested for binding to a series of CD138 peptides in four-fold serial dilutions starting at 1 μg/mL. A set of three CD138 peptides were tested: Peptide 2a (amino acids 88-121 of human CD138) Peptide 5 (amino acids 176-214 of human CD138), and Peptide 6 (amino acids 210-250 of human CD138) (FIG. 9D). HRP-conjugated anti-human IgG-Fc antibody (1:5000 dilution) was used for detection. Antibody B-B4 was also tested as a reference. As shown in FIGS. 9A-9C, antibodies 1610 and 1409 bound to Peptides 2a and Peptide 6, while antibody 1409 also bound to a lesser degree to Peptide 5. Antibody CD002 bound selectively to Peptide 5, and antibody 624 bound selectively to Peptide 6. Reference antibody B-B4 only bound to Peptide 2a.

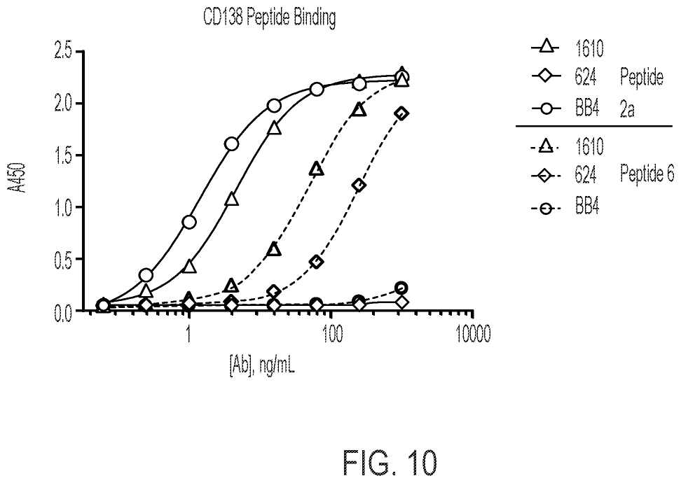

Monoclonal antibodies 1610 and 624 were further evaluated for preferential binding to Peptide 2a or Peptide 6, using the peptide binding ELISA method described above. As shown in FIG. 10, antibody 1610 bound to both Peptide 2a and Peptide 6, and showed greater affinity for Peptide 2a than for Peptide 6. Antibody 624 bound preferentially to the membrane-proximal Peptide 6. The reference antibody B-B4 bound preferentially to Peptide 2a.

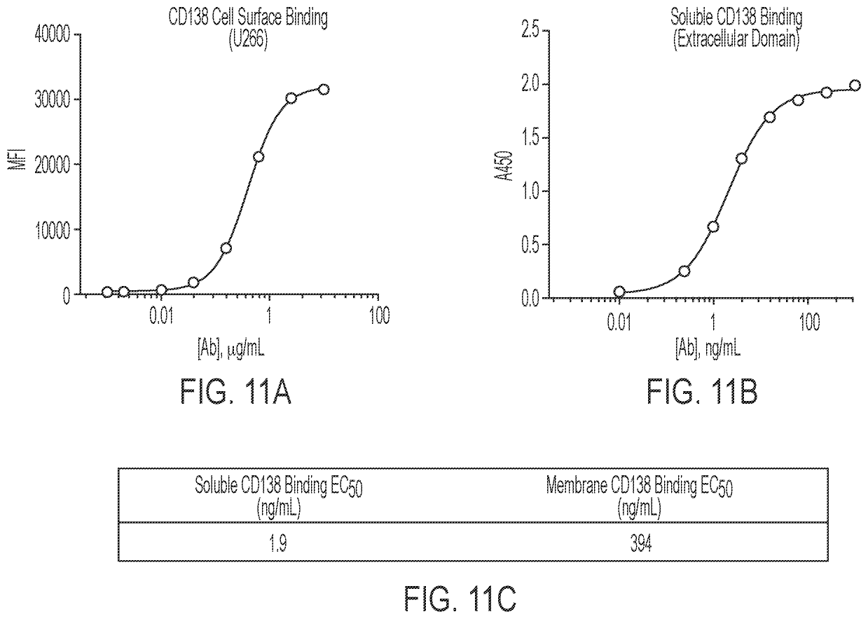

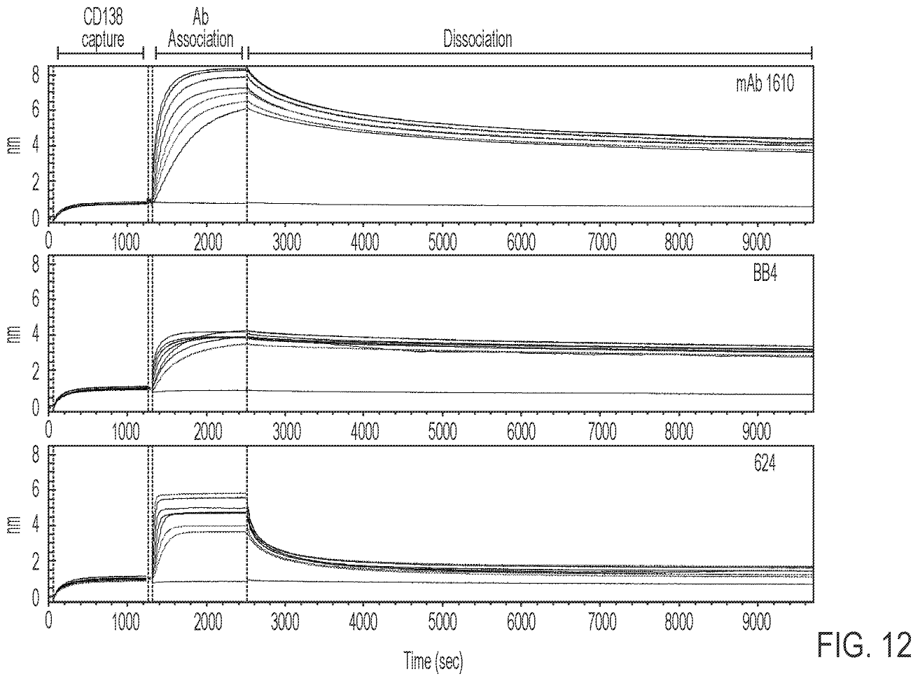

In addition, monoclonal antibody 1610 was tested for binding to soluble and cell surface forms of CD138, using the ELISA method described above and the cell binding assay described in Example 6. As shown in FIG. 11A-11C, antibody 1610 was able to bind CD138 on the surface of U266 cells in a dose-dependent manner, with a binding EC50 of 1.9 ng/mL. Antibody 1610 was also able to bind soluble CD138 in a dose-dependent manner, with a binding EC50 of 394 ng/mL.

### Example 11: Comparison of CD138 Binding Between Antibody 1610 and Reference Antibody B-B4

The binding kinetics for antibody 1610 to CD138 was tested and compared to that of reference antibody B-B4. Briefly, binding to recombinant CD138 extracellular domain was evaluated by bio-layer interferometry (Octet). Biotinylated CD138 (150 nM) was immobilized on streptavidin biosensors, and then monoclonal antibodies 1610 and B-B4 were each tested for binding at 0-300 nM. As shown in FIG. 12, antibody 1610 was found to bind to CD138 with a substantially higher binding association in comparison to reference antibody B-B4. A faster dissociation rate for antibody 1610 was observed, which may be due to a second, lower-affinity binding site. These data suggest a potentially 2:1 binding stoichiometry of antibody 1610 to CD138.

The binding kinetics of antibody 1610 for several CD138 peptides, representing two distinct regions of CD138, was also tested by bio-layer interferometry according to the methodology described above but with the use of peptides modified with biotin at their respective amino termini. As shown in FIG. 13, the peptides tested had the following amino acid sequences:

(SEQ ID NO: 10)

| | |
|---|---|
| Peptide 2A: | ASTSTLPAGEGPKEGEAVVLPEVEPGLTAREQEA |

(SEQ ID NO: 449)

| | |
|---|---|
| Peptide 2C: | GEAVVLPEVEPGLTAREQEA |

(SEQ ID NO: 440)

| | |
|---|---|
| Peptide 6B: | ENTAVVAVEPDRRNQSPVDQGATGASQGLLDRKEVLG |

(SEQ ID NO: 444)

| | |
|---|---|
| Peptide 6E: | RNQSPVDQGATGASQGLLDRKEVLG |

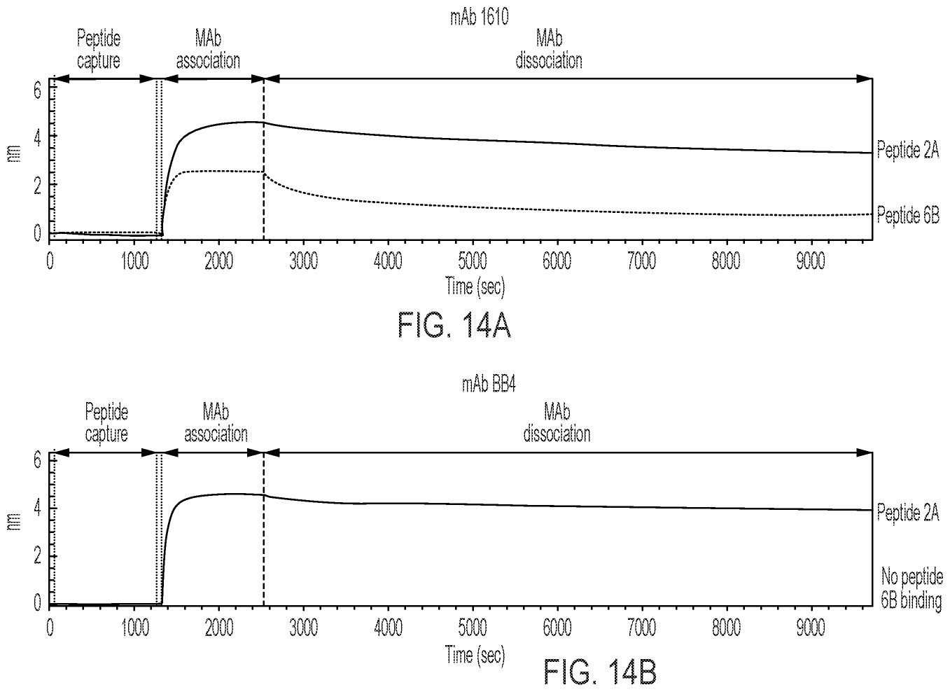

FIG. 13 shows that antibody 1610 bound to Peptides 2A and 2C with similar binding association, but of the Peptide 6 variants, only bound to Peptide 6B, and not to Peptide 6E. Comparative binding kinetics for antibodies 1610 and B-B4 for CD138 peptide fragments were also measured by bio-layer interferometry. As shown in FIG. 14A, antibody 1610 was able to bind to both peptides 2A and 6B. In contrast, antibody B-B4 only bound to Peptide 2A, not to Peptide 6B (FIG. 14B).

### Example 12: Competition for Binding to Cell Surface CD138

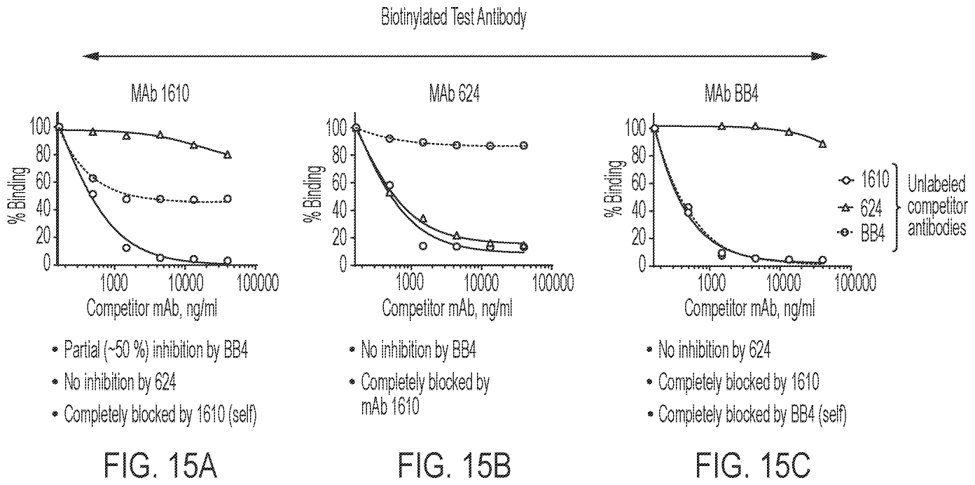

Competitive antibody binding to membrane CD138 expressed on human myeloma cell line U266 was assessed in an approach that is commonly referred to as “epitope binning.” In this example, antibody competition for binding to the cell surface antigen (CD138 here) was set between a biotinylated test antibody at a fixed concentration and varying concentrations of unlabeled, competing antibody. Antibodies 1610, B-B4, and 624 were chemically biotinylated using EZ-Link™ Sulfo-NHS-LC-Biotinylation Kit (Thermal Fisher Scientific, catalog number 21435) according to the manufacturer's instruction. In brief, recombinant monoclonal antibodies (100 microgram) were incubated overnight at 4° C. in the presence of 5-fold molar excess of the biotin reagent. Excess, unconjugated biotin was removed by buffer exchange into PBS buffer, pH 7.4 using Amicon Ultra centrifugal filters (30 kDa MWCO). For the competition analysis, serially diluted unlabeled competing antibodies were pre-mixed with a fixed level of the biotinylated test antibody. Each of the mixtures contained 0.5 μg/mL of the biotinylated antibody and a varied amount (0-40 μg/mL) of the competing antibody. U266 cells were placed in a 96 well microtiter plate at 2-5E+4 cells/well, washed once with 1×PBS and then resuspended in 100 μl of the antibody pre-mixes. Competition between unlabeled and the biotinylated versions of the same antibody was used as a positive assay control (“self-competition”). The cells were incubated in the presence of the antibody for 30 minutes at 4° C., washed, and exposed to Alexa fluor 488-tagged streptavidin for additional 30 minutes at 4° C. The cells were washed again before being evaluated for biotin-antibody binding by flow cytometry as described in Example 6. MAb 1610 showed partial (˜50%) inhibition by B-B4, no inhibition by 624, and was completely blocked by 1610 itself (FIG. 15A). MAb 624 showed no inhibition by B-B4 and was completely blocked by 1610 (FIG. 15B). MAb B-B4 showed no inhibition by 624, but was completely blocked by either 1610 or B-B4 itself (FIG. 15C).

### Example 13: Antibody 1610 Demonstrates Potent ADCC Activity in a Reporter Based Cell Assay

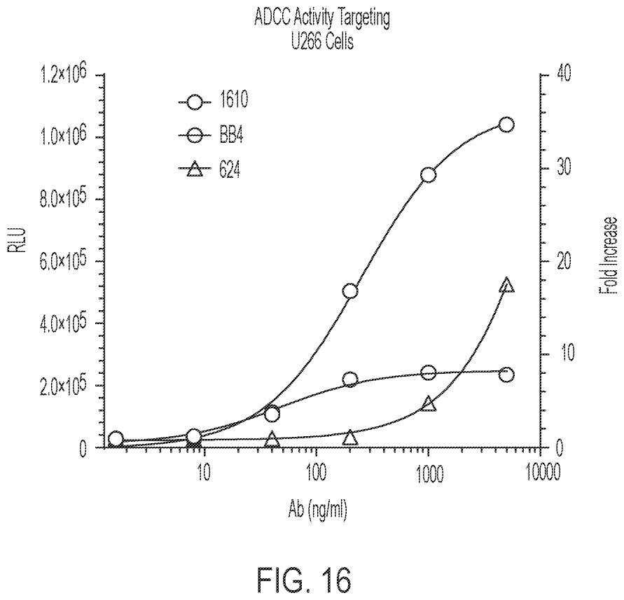

The ability of antibody 1610 to induce ADCC in its afucosylated form was tested and compared to that of antibody 624 and the reference antibody B-B4. Briefly, each of the anti-CD138 antibodies was produced in a CHO-based Fut8−/−cell line to reduce Fc fucosylation. ADCC activity induced by each antibody was measured using the ADCC Reporter Bioassay Kit (Promega), which utilizes, as effector cells, Jurkat T cells engineered to stably express the high-affinity human FcγRIIIa (V/V 158) variant and an NFAT response element driving the expression of firefly luciferase. A CD138-positive multiple myeloma cell line (U266) as the target cells. As shown in FIG. 16, afucosylation of antibody 1610 resulted in potent ADCC activity, which was not observed for antibody 624, which bound preferentially to a membrane proximal region, or for reference antibody B-B4, which bound to a region distal to the membrane proximal region. These data show that antibody 1610 binds differentially to CD138 in a manner that confers potent ADCC activity when afucosylated.

### Example 14: Generation and Characterization of Variants of Antibody 1610

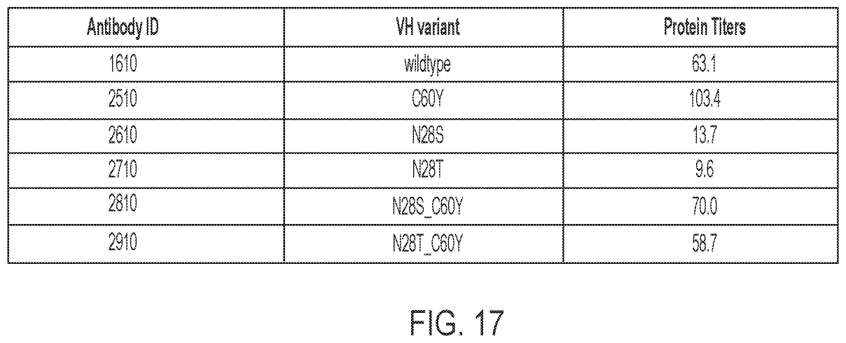

Monoclonal antibody 1610 was modified to produce a series of variants (FIG. 17). In one instance, an N-linked glycosylation site in HCDR1 of the heavy chain variable region of antibody 1610 was removed by mutating N28 to either S or T to produce antibodies 2610 and 2710, respectively. Antibodies 2610 and 2710 retained the CD138 binding and ADCC-inducing activities of the parental antibody 1610, as shown below, although the mutation resulted in lower expression levels in transiently-transfected HEK293 cells. A further mutation to antibodies 2610 and 2710, in which C60 was mutated to Y (antibodies 2810 and 2910, respectively), restored expression to levels comparable to that of antibody 1610. Without wishing to be bound by theory, it is contemplated that the C60Y mutation may also improve heavy and light chain pairing.

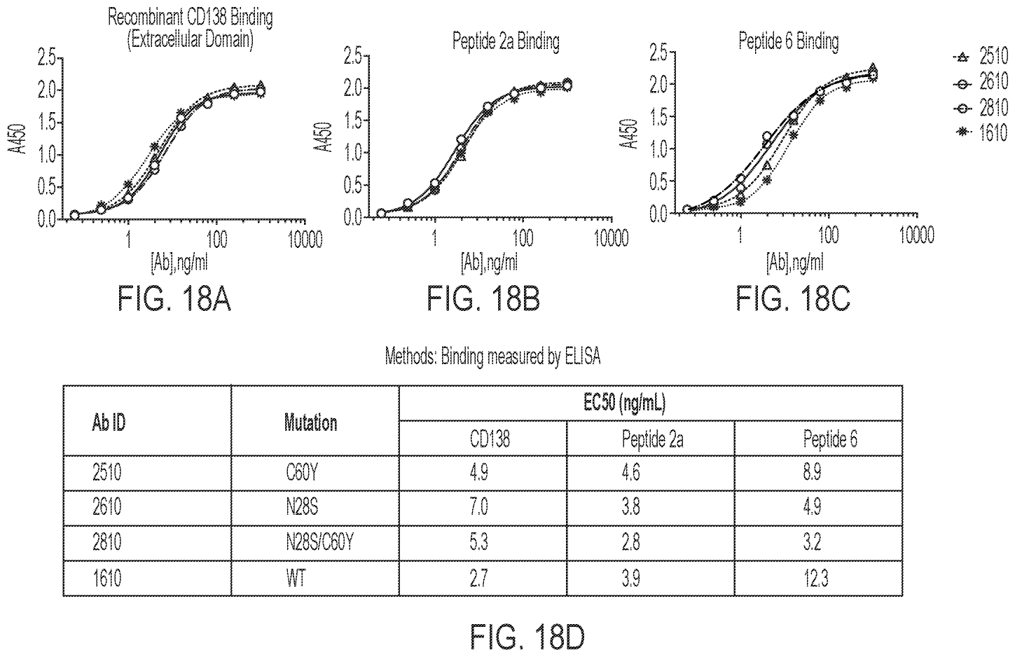

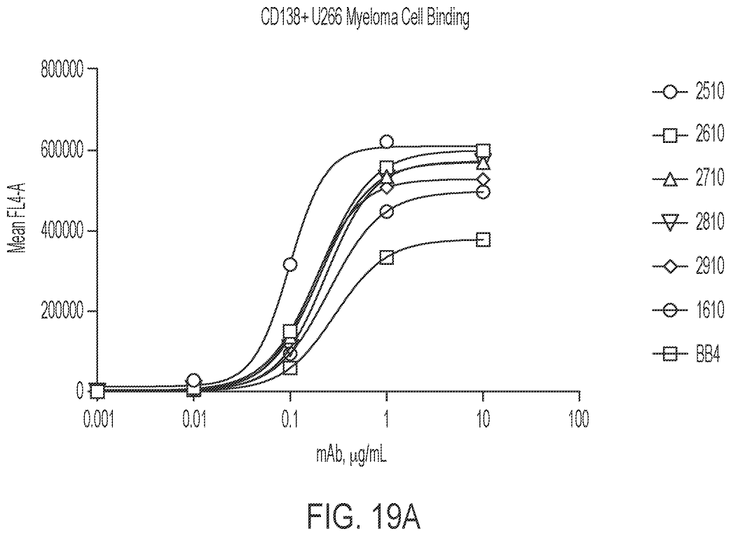

The binding properties of the antibody 1610 variants for CD138 was tested using assays as described above. In particular, antibodies 2510, 2610, and 2810 each showed similar dose-dependent binding to the extracellular domain of CD138 (FIG. 18A), Peptide 2a of CD138 (FIG. 18B), and Peptide 6 of CD138 (FIG. 18C), as shown in Table 3, when tested in ELISA assays. The EC50 values calculated for each antibody variant for CD138 extracellular domain, Peptide 2a, and Peptide 6 are shown in FIG. 18D.

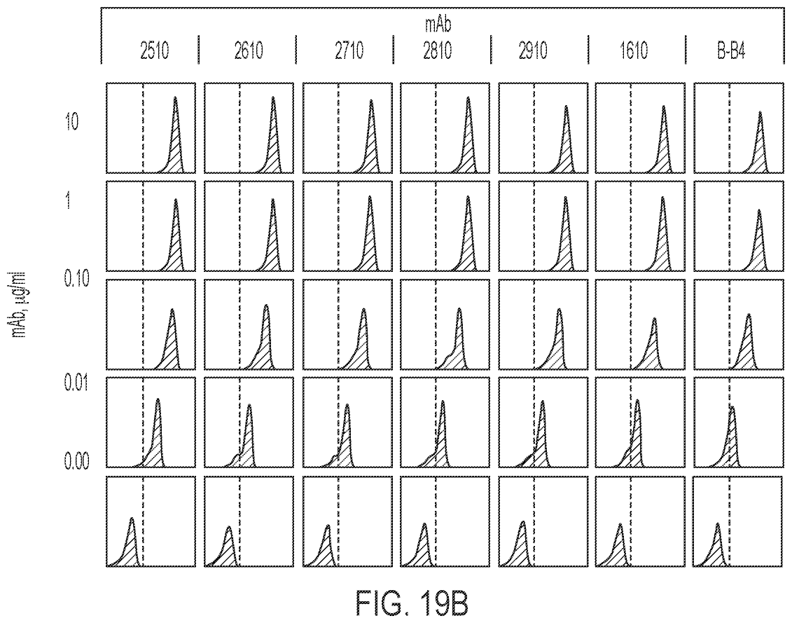

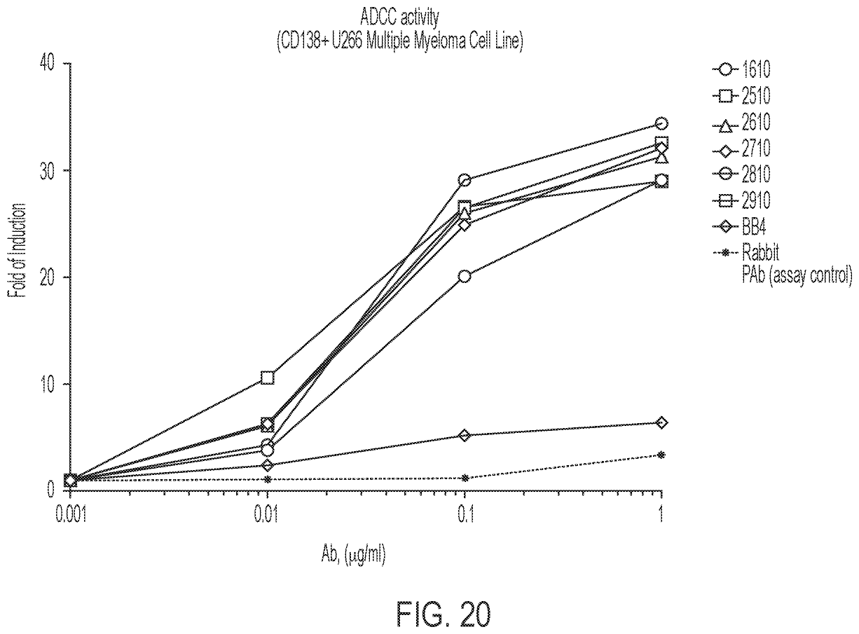

Afucosylated versions of antibody 1610 variants 2510, 2610, 2710, 2810, and 2910 were generated as described above and then tested for binding to U266 cells expressing CD138 on their cell surfaces. As shown in FIG. 19A, antibody 1610 and its variants all exhibited stronger binding to cell surface CD138 than did reference antibody B-B4. Representative flow cytometry plots for each afucosylated antibody at varying antibody concentrations are shown in FIG. 19B. The afucosylated antibody variants were then tested for capacity to induce ADCC, as described above. As shown in FIG. 20, antibody 1610 and all of its variants were capable of inducing ADCC in CD138+U266 cells in a dose-dependent manner, whereas antibody B-B4 did not substantially induce ADCC in these cells.

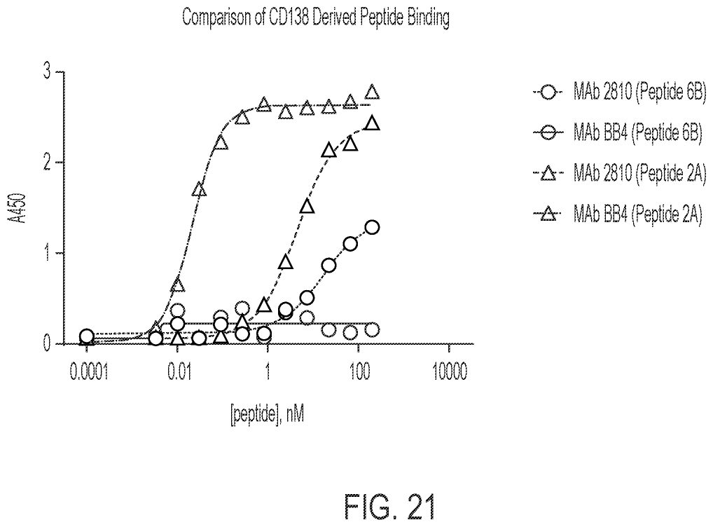

The capability of an antibody 1610 variant, antibody 2810, to bind to CD138 peptide fragments was also determined using ELISA. Briefly, antibodies 2810 or B-B4 were captured on an ELISA plate and the binding of CD138 peptides was measured at varying concentrations. As shown in FIG. 21, antibody 2810 exhibited stronger binding to Peptide 6B than did antibody B-B4, whereas antibody B-B4 bound to Peptide 2A more strongly than did antibody 2810 (although antibody 2810 did show binding to Peptide 2A). The binding kinetics of the antibody 1610 variant 2810 were compared to that of reference antibody B-B4. Briefly, biotinylated peptides (Peptides 2A, 2D, 6B, and 6F; sequences shown in FIG. 22C) were used at 50 nM and captured on streptavidin capture biosensors. Antibodies were then run over the captured peptides at concentrations of 25 nM to 6.25 nM. FIGS. 22A-22B show binding for antibodies 2810 and B-B4, respectively, at 12.5 nM. These data confirm that antibody 2810, like the parental antibody 1610, bound to two different regions of CD138, as represented by Peptides 2A and 2D (mid region) and Peptides 6B and 6F (membrane proximal region), respectively. As shown earlier, antibody B-B4 did not bind to the membrane proximal region.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1-38. (canceled)

39. A pharmaceutical composition comprising an antibody-molecule drug conjugate (ADC) and a pharmaceutically acceptable carrier, wherein the ADC comprises an anti-CD138 antibody molecule coupled to a non-antibody moiety, and wherein the antibody molecule comprises:
(a) a heavy chain variable region (VH), wherein the VH comprises three heavy chain complementarity determining regions (HCDR1, HCDR2, and HCDR3), wherein the VH comprises:
(i) an HCDR1 comprising the amino acid sequence of SEQ ID NO: 350, 355, or 356, an HCDR2 comprising the amino acid sequence of SEQ ID NO: 351, and an HCDR3 comprising the amino acid sequence of FVY; or
(ii) an HCDR1 comprising the amino acid sequence of SEQ ID NO: 380, an HCDR2 comprising the amino acid sequence of SEQ ID NO: 381 or 382, and an HCDR3 comprising the amino acid sequence of FVY; and
(b) a light chain variable region (VL), wherein the VL comprises three light chain complementarity determining regions (LCDR1, LCDR2, and LCDR3), wherein the VL comprises:
an LCDR1 comprising the amino acid sequence of SEQ ID NO: 352 or an amino acid sequence that differs by no more than 1 amino acid residue therefrom; an LCDR2 comprising the amino acid sequence of SEQ ID NO: 353; and an LCDR3 comprising the amino acid sequence of SEQ ID NO: 354.

40. The pharmaceutical composition of claim 39, wherein the VH comprises an HCDR1 comprising the amino acid sequence of SEQ ID NO: 350, 355, or 356, an HCDR2 comprising the amino acid sequence of SEQ ID NO: 351, and an HCDR3 comprising the amino acid sequence of FVY.

41. The pharmaceutical composition of claim 39, wherein the VH comprises an HCDR1 comprising the amino acid sequence of SEQ ID NO: 380, an HCDR2 comprising the amino acid sequence of SEQ ID NO: 381 or 382, and an HCDR3 comprising the amino acid sequence of FVY.

42. The pharmaceutical composition of claim 39, wherein:
(a) the VH comprises: an HCDR1 comprising the amino acid sequence of SEQ ID NO: 350, an HCDR2 comprising the amino acid sequence of SEQ ID NO: 351, and an HCDR3 comprising the amino acid sequence of FVY; and
(b) the VL comprises: an LCDR1 comprising the amino acid sequence of SEQ ID NO: 352 or an amino acid sequence that differs by no more than 1 amino acid residue therefrom, an LCDR2 comprising the amino acid sequence of SEQ ID NO: 353, and an LCDR3 comprising the amino acid sequence of SEQ ID NO: 354.

43. The pharmaceutical composition of claim 39, wherein:
(a) the VH comprises: an HCDR1 comprising the amino acid sequence of SEQ ID NO: 355, an HCDR2 comprising the amino acid sequence of SEQ ID NO: 351, and an HCDR3 comprising the amino acid sequence of FVY; and
(b) the VL comprises: an LCDR1 comprising the amino acid sequence of SEQ ID NO: 352 or an amino acid sequence that differs by no more than 1 amino acid residue therefrom, an LCDR2 comprising the amino acid sequence of SEQ ID NO: 353, and an LCDR3 comprising the amino acid sequence of SEQ ID NO: 354.

44. The pharmaceutical composition of claim 39, wherein:
(a) the VH comprises: an HCDR1 comprising an amino acid sequence comprising the amino acid sequence of SEQ ID NO: 356, an HCDR2 comprising an amino acid sequence comprising the amino acid sequence of SEQ ID NO: 351, and an HCDR3 comprising the amino acid sequence of FVY; and
(b) the VL comprises: an LCDR1 comprising the amino acid sequence of SEQ ID NO: 352 or an amino acid sequence that differs by no more than 1 amino acid residue therefrom, an LCDR2 comprising the amino acid sequence of SEQ ID NO: 353, and an LCDR3 comprising the amino acid sequence of SEQ ID NO: 354.

45. The pharmaceutical composition of claim 39, wherein:
(a) the VH comprises: an HCDR1 comprising an amino acid sequence comprising the amino acid sequence of SEQ ID NO: 380, an HCDR2 comprising an amino acid sequence comprising the amino acid sequence of SEQ ID NO: 381, and an HCDR3 comprising the amino acid sequence of FVY; and
(b) the VL comprises: an LCDR1 comprising the amino acid sequence of SEQ ID NO: 352 or an amino acid sequence that differs by no more than 1 amino acid residue therefrom, an LCDR2 comprising the amino acid sequence of SEQ ID NO: 353, and an LCDR3 comprising the amino acid sequence of SEQ ID NO: 354.

46. The pharmaceutical composition of claim 39, wherein:
(a) the VH comprises: an HCDR1 comprising an amino acid sequence comprising the amino acid sequence of SEQ ID NO: 380, an HCDR2 comprising an amino acid sequence comprising the amino acid sequence of SEQ ID NO: 382, and an HCDR3 comprising the amino acid sequence of FVY; and
(b) the VL comprises: an LCDR1 comprising the amino acid sequence of SEQ ID NO: 352 or an amino acid sequence that differs by no more than 1 amino acid residue therefrom, an LCDR2 comprising the amino acid sequence of SEQ ID NO: 353, and an LCDR3 comprising the amino acid sequence of SEQ ID NO: 354.

47. The pharmaceutical composition of claim 39, wherein the VH comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of SEQ ID NO: 293, 294, 295, 296, or 297, and/or
wherein the VL comprises the amino acid sequence of SEQ ID NO: 292, or an amino acid sequence that differs by no more than 1, 2, 3, 4, 5, 6, 7, 8, 9, 10, 11, 12, 13, 14, or 15 amino acid residues therefrom, or has at least 85, 90, 95, 99 or 100% homology therewith.

48. The pharmaceutical composition of claim 39, wherein:
(a) the VH comprises the amino acid sequence of SEQ ID NO: 293, and the VL comprises the amino acid sequence of 292;
(b) the VH comprises the amino acid sequence of SEQ ID NO: 294, and the VL comprises the amino acid sequence of 292;
(c) the VH comprises the amino acid sequence of SEQ ID NO: 295, and the VL comprises the amino acid sequence of 292;
(d) the VH comprises the amino acid sequence of SEQ ID NO: 296, and the VL comprises the amino acid sequence of 292; or
(e) the VH comprises the amino acid sequence of SEQ ID NO: 297, and the VL comprises the amino acid sequence of 292.

49. The pharmaceutical composition of claim 39, wherein the antibody molecule comprises a heavy chain constant region of IgG1 and/or a light chain constant region of kappa light chain.

50. The pharmaceutical composition of claim 39, wherein the antibody molecule is an afucosylated antibody molecule.

51. The pharmaceutical composition of claim 39, wherein the antibody molecule is a synthetic antibody molecule, a humanized antibody molecule, or an isolated antibody molecule, and/or is a monovalent antibody molecule, a multivalent antibody molecule, a monospecific molecule, or a multispecific antibody molecule.

52. The pharmaceutical composition of claim 39, wherein the antibody molecule comprises a heavy chain constant region of an IgG chosen from IgG1, IgG2, IgG3, or IgG4, and/or a light chain constant region of kappa or lambda light chain.

53. The pharmaceutical composition of claim 39, wherein the non-antibody moiety comprises a therapeutic agent or label.

54. The pharmaceutical composition of claim 53, wherein the therapeutic agent comprises a cytotoxic agent.

55. The pharmaceutical composition of claim 54, wherein the cytotoxic agent is a tubulin polymerase inhibitor, an agent associated with tubulin depolymerization, an agent associated with DNA cleavage, a DNA minor groove alkylating agent, a DNA minor groove cross-linker, or an RNA polymerase II inhibitor.

56. The pharmaceutical composition of claim 54, wherein the cytotoxic agent comprises α-amanitin, a cryptophycin analog, calicheamicin, centanamycin, a dolastatin, duocarmycin or a duocarmycin analogue, maytansine, monomethyl auristatin E (MMAE), monomethyl auristatin F (MMAF), or a pyrrolobenzodiazepine (PBD).

57. The pharmaceutical composition of claim 39, wherein:
(i) the non-antibody moiety or coupled to the antibody molecule directly or indirectly;
(ii) the non-antibody moiety is coupled to the antibody molecule through a linker;
(iii) the non-antibody moiety is coupled to the antibody molecule by a covalent bond, a peptide bond, or non-peptide bond;
(iv) the non-antibody moiety is coupled to the backbone or a side chain of the antibody molecule; and/or
(v) two, three, four, five, six, seven, eight, or more non-antibody moieties are coupled to the antibody molecule.

58. The pharmaceutical composition of claim 39, wherein the linker comprises a hydrazone, a disulfide bond, a peptide, or a thioether bond.

59. The pharmaceutical composition of claim 39, wherein the linker is a non-cleavable linker or cleavable linker.

60. A kit comprising the pharmaceutical composition of claim 39 and instructions to use of the pharmaceutical composition.

61. A container comprising the pharmaceutical composition of claim 39.

62. A method of treating a cancer, the method comprising administering to a subject in need thereof an effective amount of an ADC, thereby treating the cancer,
wherein the ADC comprises an anti-CD138 antibody molecule coupled to a non-antibody moiety, and wherein the antibody molecule comprises:
(a) a VH, wherein the VH comprises three heavy chain complementarity determining regions (HCDR1, HCDR2, and HCDR3), wherein the VH comprises:
(i) an HCDR1 comprising the amino acid sequence of SEQ ID NO: 350, 355, or 356, an HCDR2 comprising the amino acid sequence of SEQ ID NO: 351, and an HCDR3 comprising the amino acid sequence of FVY; or
(ii) an HCDR1 comprising the amino acid sequence of SEQ ID NO: 380, an HCDR2 comprising the amino acid sequence of SEQ ID NO: 381 or 382, and an HCDR3 comprising the amino acid sequence of FVY; and
(b) a VL, wherein the VL comprises three light chain complementarity determining regions (LCDR1, LCDR2, and LCDR3), wherein the VL comprises:
an LCDR1 comprising the amino acid sequence of SEQ ID NO: 352 or an amino acid sequence that differs by no more than 1 amino acid residue therefrom; an LCDR2 comprising the amino acid sequence of SEQ ID NO: 353; and an LCDR3 comprising the amino acid sequence of SEQ ID NO: 354.

63. The method of claim 62, wherein the cancer is a myeloma.

64. A method of treating a precancerous condition or preventing a cancer, the method comprising administering to a subject in need thereof an effective amount of an ADC, thereby treating the precancerous condition or preventing the cancer,
wherein the ADC comprises an anti-CD138 antibody molecule coupled to a non-antibody moiety, and wherein the antibody molecule comprises:
(a) a VH, wherein the VH comprises three heavy chain complementarity determining regions (HCDR1, HCDR2, and HCDR3), wherein the VH comprises:
(i) an HCDR1 comprising the amino acid sequence of SEQ ID NO: 350, 355, or 356, an HCDR2 comprising the amino acid sequence of SEQ ID NO: 351, and an HCDR3 comprising the amino acid sequence of FVY; or
(ii) an HCDR1 comprising the amino acid sequence of SEQ ID NO: 380, an HCDR2 comprising the amino acid sequence of SEQ ID NO: 381 or 382, and an HCDR3 comprising the amino acid sequence of FVY; and
(b) a VL, wherein the VL comprises three light chain complementarity determining regions (LCDR1, LCDR2, and LCDR3), wherein the VL comprises:
an LCDR1 comprising the amino acid sequence of SEQ ID NO: 352 or an amino acid sequence that differs by no more than 1 amino acid residue therefrom; an LCDR2 comprising the amino acid sequence of SEQ ID NO: 353; and an LCDR3 comprising the amino acid sequence of SEQ ID NO: 354.

65. The method of claim 64, wherein the precancerous condition is smoldering myeloma or monoclonal gammopathy of undetermined significance (MGUS).
